(19) United States
(12) Patent Application Publication
(10) Pub. No.: US 2026/0033127 A1
(43) Pub. Date: Jan. 29, 2026

(54) COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Jiah YOON, Suwon-si (KR); Hanill LEE, Suwon-si (KR); Jiyun KWON, Suwon-si (KR); Wook KIM, Suwon-si (KR); Jonghoon KIM, Suwon-si (KR); Seungjae LEE, Suwon-si (KR); Seonyeong GWAK, Suwon-si (KR); Suheon IM, Suwon-si (KR); Minji PARK, Suwon-si (KR); Mijin LEE, Suwon-si (KR); Dalho HUH, Suwon-si (KR); Hyung Sun KIM, Suwon-si (KR)

(21) Appl. No.: 19/265,407

(22) Filed: Jul. 10, 2025

(30) Foreign Application Priority Data
Jul. 26, 2024 (KR) 10-2024-0099695

(51) Int. Cl. H10K 50/12; C07D 251/24; C07D 307/91; H10K 85/60

(52) U.S. Cl. CPC H10K 50/12; C07D 251/24; C07D 307/91; H10K 85/622; H10K 85/626; H10K 85/633; H10K 85/636; H10K 85/654; H10K 85/6572; H10K 85/6574; H10K 85/6576

(57) ABSTRACT

A compound and a composition including the compound for an organic optoelectronic device, an organic optoelectronic device including the compound or the composition, and a display device including the organic optoelectronic device, the compound being represented by Chemical Formula 1:

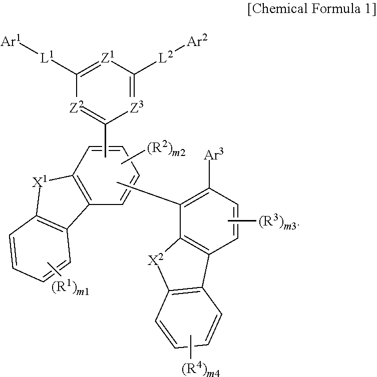

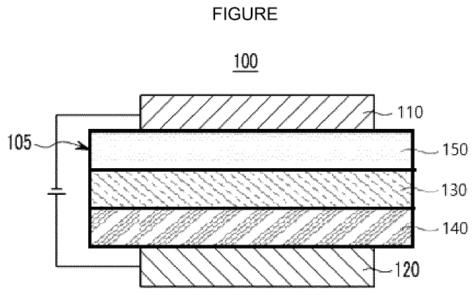

### CROSS-REFERENCE TO RELATED APPLICATION

This application claims priority to and the benefit of Korean Patent Application No. 10-2024-0099695 filed in the Korean Intellectual Property Office on Jul. 26, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively, and the other is light emitting device that generates light energy from electrical energy by supplying voltage or current to the electrodes.

Examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

The embodiments may be realized by providing a compound for an organic optoelectronic device, the compound being represented by Chemical Formula 1:

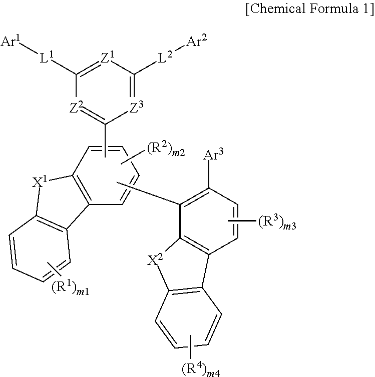

wherein, in Chemical Formula 1, Z¹ to Z³ are each independently N or CR^{a}, at least two of Z¹ to Z³ are N, X¹ and X² are each independently O, S, or SiR^{b}R^{c}, at least one of X¹ and X² is O or S, Ar¹ to Ar³ are each independently a substituted or unsubstituted C6 to C30 aryl group or a substituted or unsubstituted C2 to C30 heterocyclic group, L¹ and L² are each independently a single bond or a substituted or unsubstituted C6 to C30 arylene group, R^{a} and R¹ to R⁴ are each independently hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C12 aryl group, R^{b} and R^{c} are each independently a substituted or unsubstituted C1 to C20 alkyl group or a substituted or unsubstituted C6 to C20 aryl group, m1 and m4 are each independently an integer of 1 to 4, and m2 and m3 are each independently an integer of 1 or 2.

The embodiments may be realized by providing a composition for an organic optoelectronic device, the composition including a first compound, and a second compound, wherein the first compound may be the compound for an organic optoelectronic device according to an embodiment, and the second compound may be represented by Chemical Formula 2, a combination of Chemical Formula 3 and Chemical Formula 4, or Chemical Formula 5:

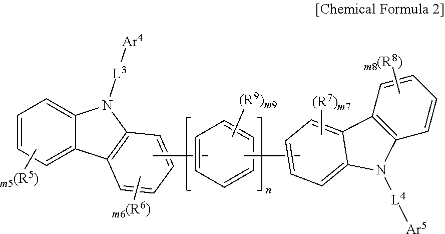

wherein, in Chemical Formula 2, R⁸ to R⁹ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁴ and Ar⁵ may each independently be a substituted or unsubstituted C6 to C20 aryl group or a substituted or unsubstituted C2 to C30 heterocyclic group, L³ and L⁴ may each independently be a single bond or a substituted or unsubstituted C6 to C20 arylene group, m5, m8, and m9 may each independently be an integer of 1 to 4, m6 and m7 may each independently be an integer 1 to 3, and n may be an integer of 0 to 2,

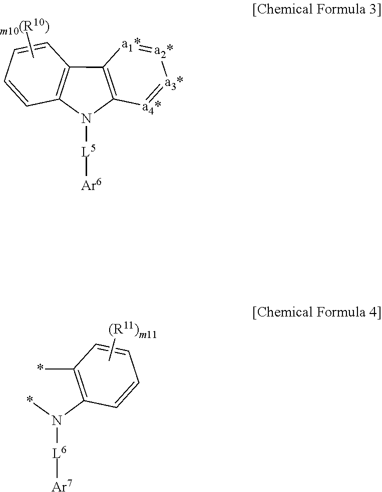

wherein, in Chemical Formula 3 and Chemical Formula 4, two adjacent ones of a₁* to a₄* of Chemical Formula 3, may be linking carbons linked at * of Chemical Formula 4, the remaining two of a₁* to a₄* of Chemical Formula 3 that are not linked at * of Chemical Formula 4 may be C-L^{a}-R^{d}, L^{a}, L⁵, and L⁶ may each independently be a single bond or a substituted or unsubstituted C6 to C20 arylene group, R^{d}, R¹⁰, and R¹¹ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁶ and Ar⁷ may each independently be a substituted or unsubstituted C6 to C20 aryl group or a substituted or unsubstituted C2 to C30 heterocyclic group, and m10 and m11 may each independently be an integer 1 to 4,

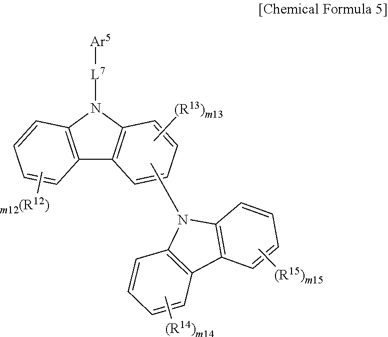

wherein, in Chemical Formula 5, L⁷ may be a single bond or a substituted or unsubstituted C6 to C20 arylene group, R¹² to R¹⁵ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁸ may be a substituted or unsubstituted C6 to C20 aryl group or a substituted or unsubstituted C2 to C30 heterocyclic group, m12, m14, and m15 may each independently be an integer of 1 to 4, and m13 may be an integer of 1 to 3.

The embodiments may be realized by providing an organic optoelectronic device including an anode and a cathode facing each other, and at least one organic layer between the anode and the cathode, wherein the organic layer includes the compound or composition for an organic optoelectronic device according to some embodiments.

The embodiments may be realized by providing a display device including the organic optoelectronic device according to some embodiments.

### BRIEF DESCRIPTION OF THE DRAWING

Features will become apparent to those of skill in the art by describing in detail exemplary embodiments with reference to the attached drawing in which:

the FIGURE is a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, or one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In an implementation, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In an implementation, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In an implementation, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).” For example, any hydrogen in any compound described herein may be protium, deuterium, or tritium (e.g., based on natural or artificial substitution). As used herein, the term “or” is not an exclusive term, e.g., “A or B” would include A, B, or A and B.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, e.g., a phenyl group, a naphthyl group, and the like, two or more hydrocarbon aromatic moieties may be linked by a sigma bond and may be, e.g., a biphenyl group, a terphenyl group, a quarterphenyl group, and the like, and two or more hydrocarbon aromatic moieties may be fused directly or indirectly to provide a non-aromatic fused ring, e.g., a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups may be linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

A compound for an organic optoelectronic device according to some example embodiments is represented by Chemical Formula 1.

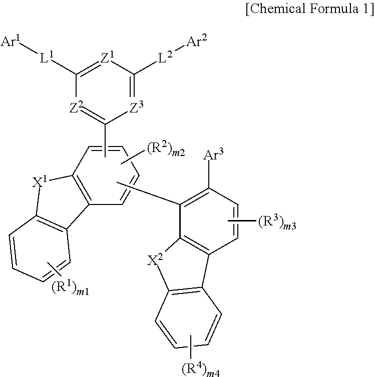

In Chemical Formula 1, Z¹ to Z³ may each independently be, e.g., N or CR^{a} and least two of Z¹ to Z³ may be, e.g., N.

X¹ and X² may each independently be, e.g., O, S, or SiR^{b}R^{c} and at least one of X¹ and X² may be, e.g., O or S.

Ar¹ to Ar³ may each independently be or include, e.g., a substituted or unsubstituted C6 to C30 aryl group or a substituted or unsubstituted C2 to C30 heterocyclic group.

L¹ and L² may each independently be, e.g., a single bond or a substituted or unsubstituted C6 to C30 arylene group.

R^{a} and R¹ to R⁴ may each independently be or include, e.g., hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C12 aryl group.

R^{b} and R^{c} may each independently be or include, e.g., a substituted or unsubstituted C1 to C20 alkyl group or a substituted or unsubstituted C6 to C20 aryl group.

m1 and m4 may each independently be, e.g., an integer of 1 to 4.

m2 and m3 may each independently be, e.g., an integer of 1 or 2.

The compound represented by Chemical Formula 1 may be a structure in which a dibenzofuran derivative directly linked to a triazine may be included, and the dibenzofuran derivative may be further substituted with a 4-dibenzofuran derivative, and a substituted or unsubstituted C6 to C30 aryl group or a substituted or unsubstituted C2 to C30 heterocyclic group may be substituted at the 3rd position of the 4-dibenzofuran derivative.

The compound represented by Chemical Formula 1 may have improved electron transport characteristics and a deep LUMO energy level by including the dibenzofuran derivative directly linked to triazine. Due to the deep LUMO energy level, electron mobility characteristics may be improved, but a band gap of the molecule may be narrowed, which may reduce the energy transfer path from the host to the dopant, which may lower the efficiency. However, by further being substituted with the 4-dibenzofuran derivative and designing it in the direction of increasing the steric hindrance, an appropriate LUMO energy level may be maintained, and thus a high-efficiency device may be implemented.

In addition, by including at least two dibenzofuran derivatives having high electron transport characteristics, the LUMO energy level may be prevented from becoming shallow by designing the number of unshared electron pairs to be two pairs, and as described above, the steric hinderance may be strengthened, resulting in a rigid structure for the entire molecule, thereby strengthening the electron transport characteristics. Molecules with a rigid structure consume less energy because the energy difference before and after electron transport may be small due to structural stabilization. As a result, the present embodiments may help implement a low-power driving device.

In addition, by including at least two dibenzofuran derivatives, degradation due to electron delocalization may be prevented, and by substituting an additional substituent at 3rd position of the 4-dibenzofuran derivative, electron delocalization may be effectively suppressed, so that efficiency and life-span characteristics may be significantly improved.

The addition of an additional substituent at 3rd position of the 4-dibenzofuran derivative may help significantly increase steric hindrance, which may also have the effect of lowering the deposition temperature. Accordingly, it is expected that high efficiency and long life-span characteristics may be realized.

In an implementation, Chemical Formula 1 may be represented, e.g., by one of Chemical Formula 1-1 to Chemical Formula 1-4 depending on the specific substitution position of the dibenzofuran derivative directly linked to the triazine derivative.

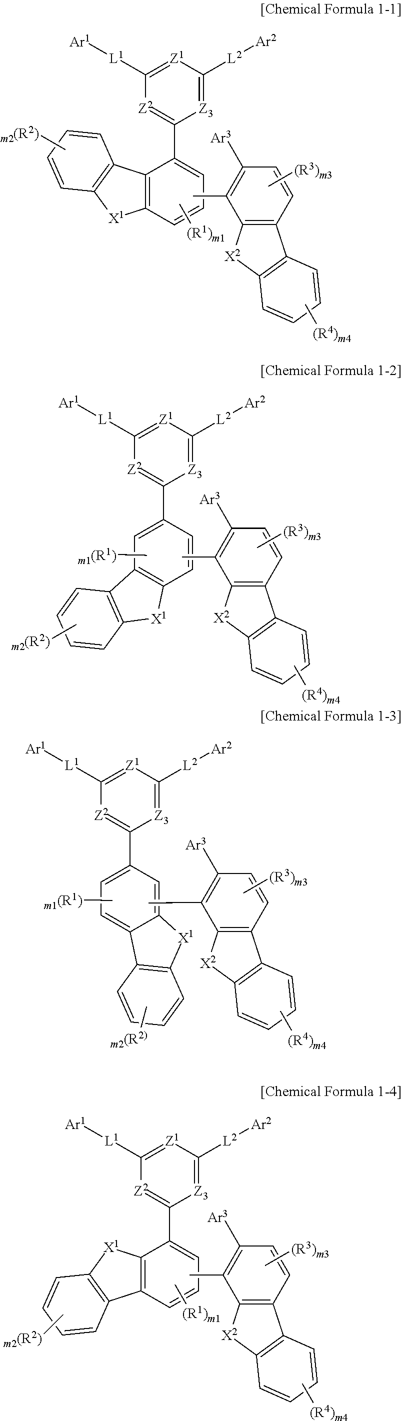

In Chemical Formula 1-1 to Chemical Formula 1-4, Z¹ to Z³, X¹ and X², Ar¹ to Ar³, L¹ and L², R¹ to R⁴ and m1 to m4 may be defined the same as those described above.

In an implementation Chemical Formula 1-1 may be represented, e.g., by one of Chemical Formula 1-1a to Chemical Formula 1-1c.

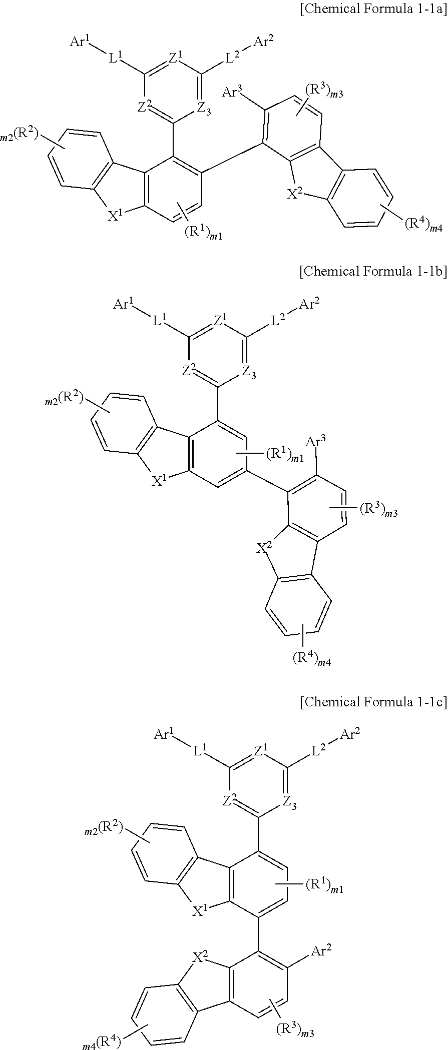

In an implementation, Chemical Formula 1-2 may be represented, e.g., by one of Chemical Formula 1-2a to Chemical Formula 1-2c.

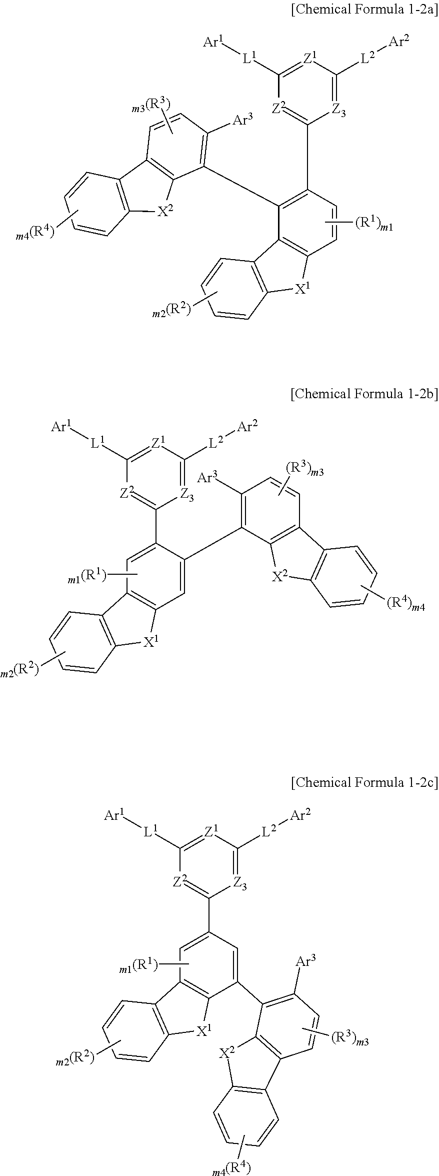

In an implementation, Chemical Formula 1-3 may be represented, e.g., by one of Chemical Formula 1-3a to Chemical Formula 1-3c.

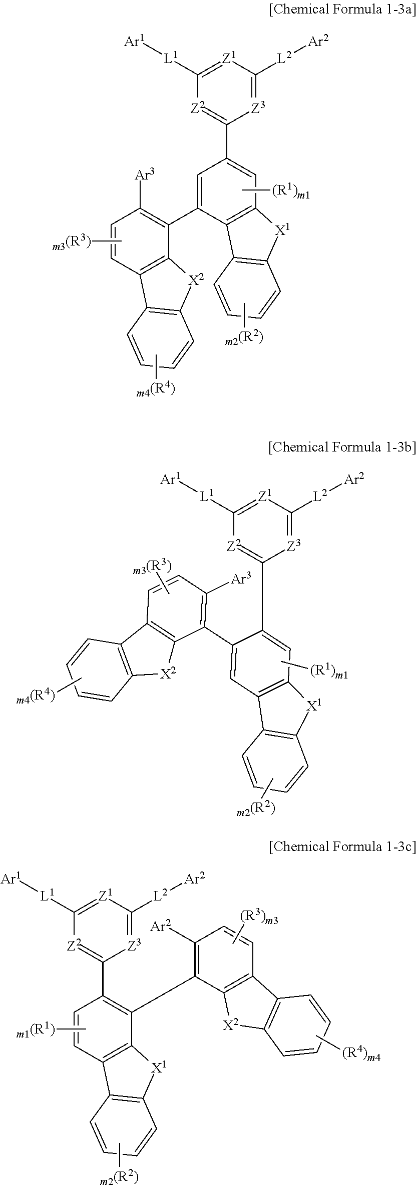

In an implementation, Chemical Formula 1-4 may be represented, e.g., by one of Chemical Formula 1-4a to Chemical Formula 1-4c.

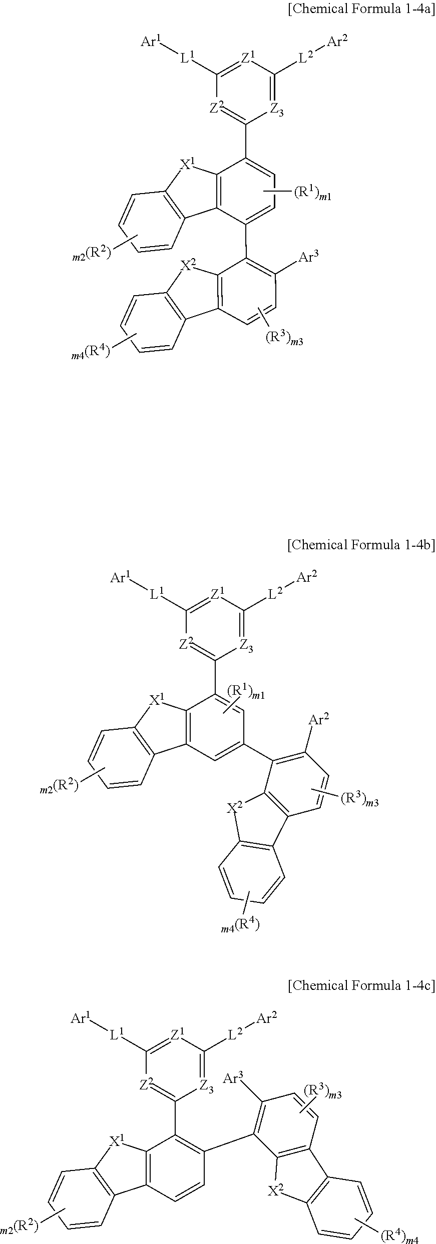

In Chemical Formula 1-1a to Chemical Formula 1-1c, Chemical Formula 1-2a to Chemical Formula 1-2c, Chemical Formula 1-3a to Chemical Formula 1-3c and Chemical Formula 1-4a to Chemical Formula 1-4c, Z¹ to Z³, X¹ and X², Ar¹ to Ar³, L¹ and L², R¹ to R⁴, and m1 to m4 are defined the same as those described above.

In an implementation, Chemical Formula 1 may be, e.g., represented by one of Chemical Formula 1-1, Chemical Formula 1-2, or Chemical Formula 1-3.

In an implementation, Chemical Formula 1 may be represented by one of Chemical Formula 1-1a, Chemical Formula 1-2c, Chemical Formula 1-3b, or Chemical Formula 1-3c.

In an implementation, at least one of Ar¹ and Ar² may be, e.g., a C10 to C30 aryl group or a substituted or unsubstituted C2 to C30 heterocyclic group.

In an implementation, Ar¹ and Ar² may each independently be, e.g., a substituted or unsubstituted phenyl group, a substituted or unsubstituted biphenyl group, or a substituted or unsubstituted carbazolyl group.

In an implementation, at least one of Ar¹ and Ar² may be, e.g., a substituted or unsubstituted biphenyl group or a substituted or unsubstituted carbazolyl group.

In an implementation, Ar³ may be a substituted or unsubstituted C6 to C30 aryl group.

In an implementation, Ar³ may be, e.g., a substituted or unsubstituted phenyl group, a substituted or unsubstituted biphenyl group, or a substituted or unsubstituted naphthyl group.

In an implementation, R¹ to R⁴ may each independently be, e.g., hydrogen, deuterium, a cyano group, a substituted or unsubstituted C1 to C5 alkyl group, a substituted or unsubstituted C1 to C5 alkylsilyl group, a substituted or unsubstituted C3 to C10 cycloalkyl group, or a substituted or unsubstituted C6 to C12 aryl group.

In an implementation, R¹ to R⁴ may each independently be, e.g., hydrogen, deuterium, a cyano group, a tert-butyl group, a trimethylsilyl group, a substituted or unsubstituted phenyl group, or a substituted or unsubstituted biphenyl group.

In an implementation, the compound represented by Chemical Formula 1 may be a compound of Group 1.

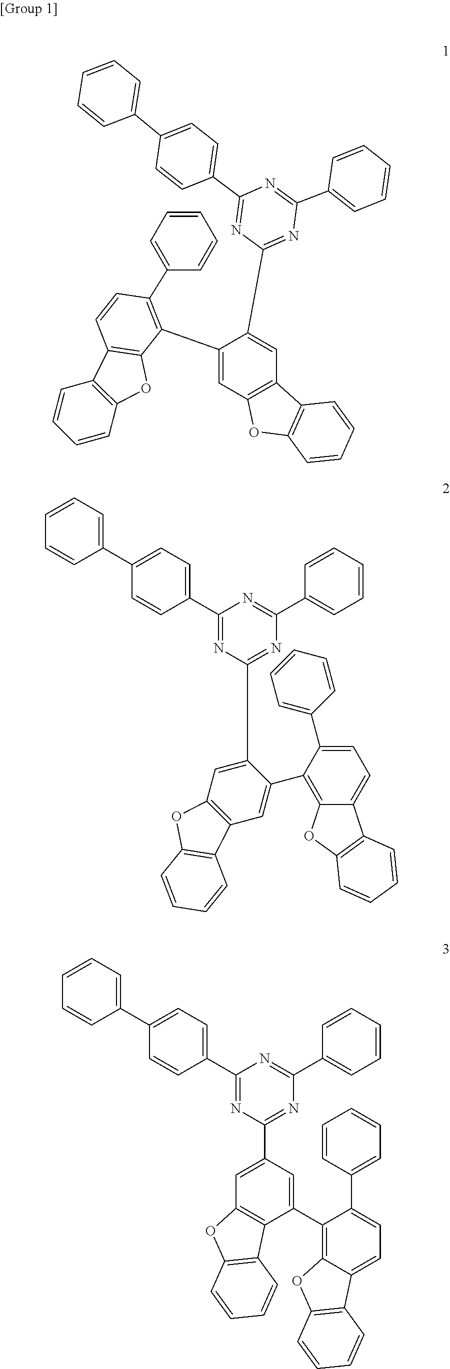 [SMILES: c1ccc(-c2ccc(-c3nc(-c4ccccc4)nc(-c4cc5c(cc4-c4c(-c6ccccc6)ccc6c4oc4ccccc46)oc4ccccc45)n3)cc2)cc1 ; c1ccc(-c2ccc(-c3nc(-c4ccccc4)nc(-c4cc5oc6ccccc6c5cc4-c4c(-c5ccccc5)ccc5c4oc4ccccc45)n3)cc2)cc1 ; c1ccc(-c2ccc(-c3nc(-c4ccccc4)nc(-c4cc(-c5c(-c6ccccc6)ccc6c5oc5ccccc56)c5c(c4)oc4ccccc45)n3)cc2)cc1]

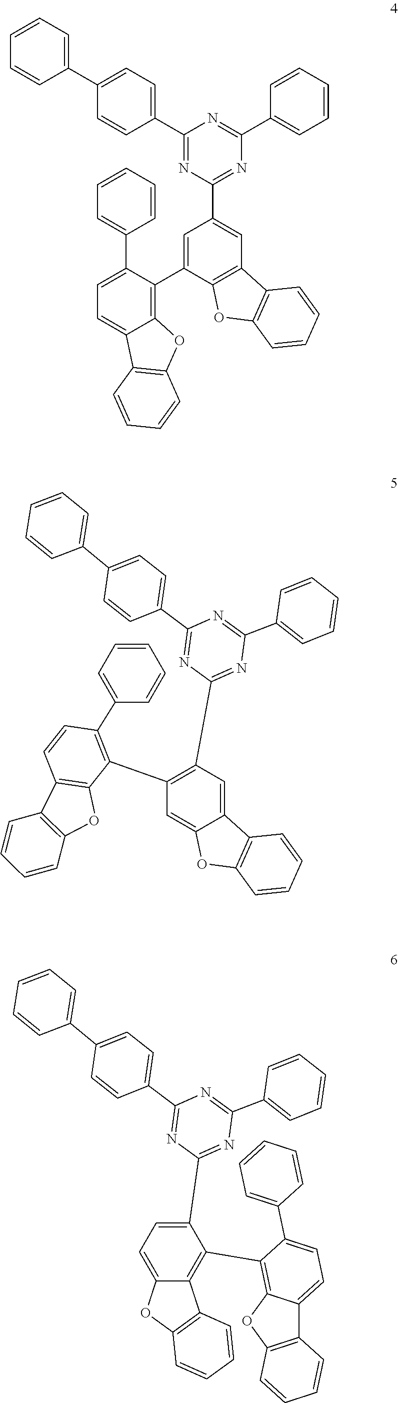 [SMILES: c1ccc(-c2ccc(-c3nc(-c4ccccc4)nc(-c4cc(-c5c(-c6ccccc6)ccc6c5oc5ccccc56)c5oc6ccccc6c5c4)n3)cc2)cc1 ; c1ccc(-c2ccc(-c3nc(-c4ccccc4)nc(-c4cc5c(cc4-c4c(-c6ccccc6)ccc6c4oc4ccccc46)oc4ccccc45)n3)cc2)cc1 ; c1ccc(-c2ccc(-c3nc(-c4ccccc4)nc(-c4ccc5oc6ccccc6c5c4-c4c(-c5ccccc5)ccc5c4oc4ccccc45)n3)cc2)cc1]

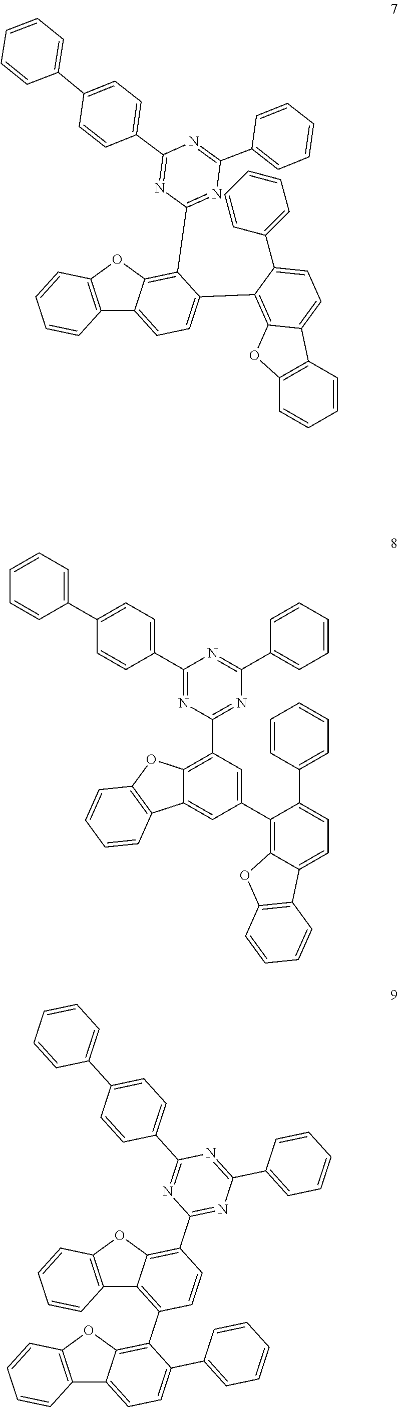 [SMILES: c1ccc(-c2ccc(-c3nc(-c4ccccc4)nc(-c4c(-c5c(-c6ccccc6)ccc6c5oc5ccccc56)ccc5c4oc4ccccc45)n3)cc2)cc1 ; c1ccc(-c2ccc(-c3nc(-c4ccccc4)nc(-c4cc(-c5c(-c6ccccc6)ccc6c5oc5ccccc56)cc5c4oc4ccccc45)n3)cc2)cc1 ; c1ccc(-c2ccc(-c3nc(-c4ccccc4)nc(-c4ccc(-c5c(-c6ccccc6)ccc6c5oc5ccccc56)c5c4oc4ccccc45)n3)cc2)cc1]

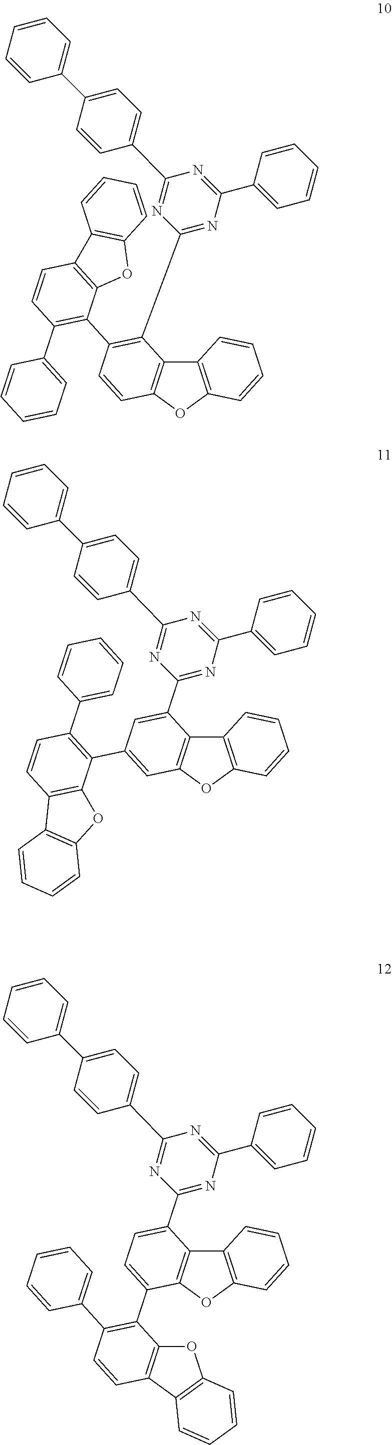 [SMILES: c1ccc(-c2ccc(-c3nc(-c4ccccc4)nc(-c4c(-c5c(-c6ccccc6)ccc6c5oc5ccccc56)ccc5oc6ccccc6c45)n3)cc2)cc1 ; c1ccc(-c2ccc(-c3nc(-c4ccccc4)nc(-c4cc(-c5c(-c6ccccc6)ccc6c5oc5ccccc56)cc5oc6ccccc6c45)n3)cc2)cc1 ; c1ccc(-c2ccc(-c3nc(-c4ccccc4)nc(-c4ccc(-c5c(-c6ccccc6)ccc6c5oc5ccccc56)c5oc6ccccc6c45)n3)cc2)cc1]

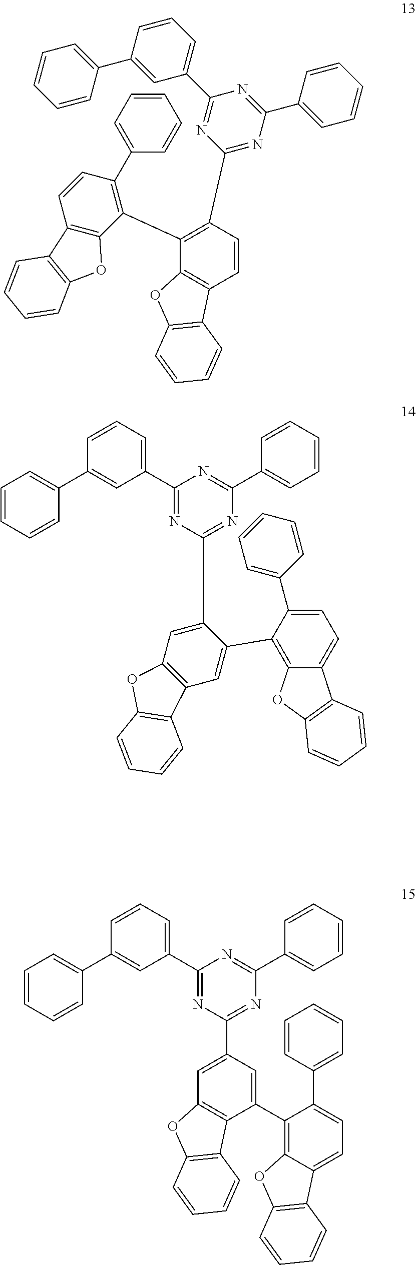 [SMILES: c1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; c1ccc(-c2cccc(-c3nc(-c4ccccc4)nc(-c4cc5oc6ccccc6c5cc4-c4c(-c5ccccc5)ccc5c4oc4ccccc45)n3)c2)cc1 ; c1ccc(-c2cccc(-c3nc(-c4ccccc4)nc(-c4cc(-c5c(-c6ccccc6)ccc6c5oc5ccccc56)c5c(c4)oc4ccccc45)n3)c2)cc1]

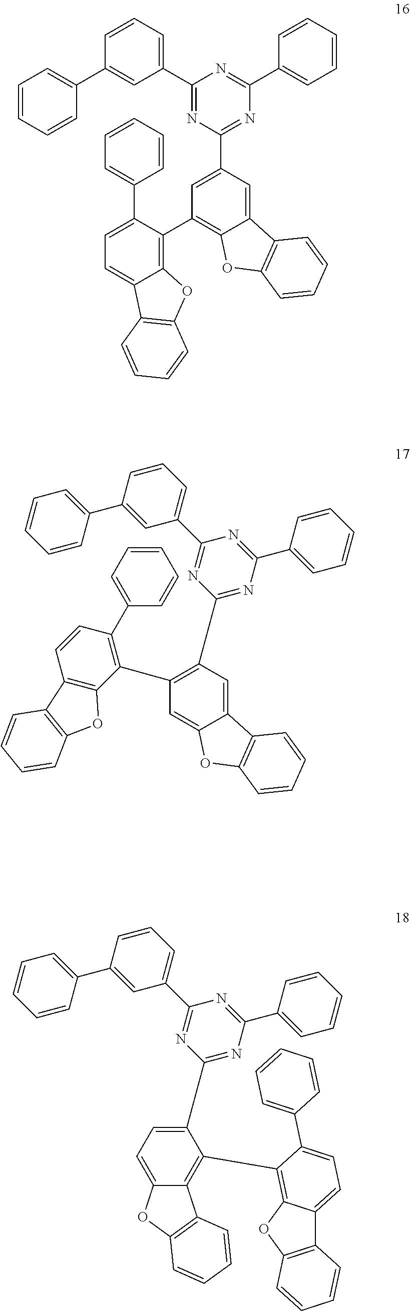 [SMILES: c1ccc(-c2cccc(-c3nc(-c4ccccc4)nc(-c4cc(-c5c(-c6ccccc6)ccc6c5oc5ccccc56)c5oc6ccccc6c5c4)n3)c2)cc1 ; c1ccc(-c2cccc(-c3nc(-c4ccccc4)nc(-c4cc5c(cc4-c4c(-c6ccccc6)ccc6c4oc4ccccc46)oc4ccccc45)n3)c2)cc1 ; c1ccc(-c2cccc(-c3nc(-c4ccccc4)nc(-c4ccc5oc6ccccc6c5c4-c4c(-c5ccccc5)ccc5c4oc4ccccc45)n3)c2)cc1]

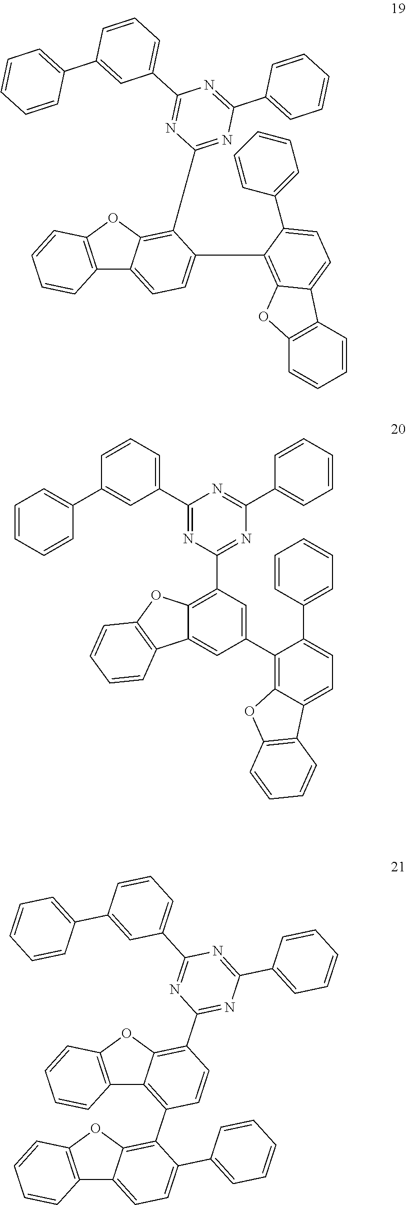 [SMILES: c1ccc(-c2cccc(-c3nc(-c4ccccc4)nc(-c4c(-c5c(-c6ccccc6)ccc6c5oc5ccccc56)ccc5c4oc4ccccc45)n3)c2)cc1 ; c1ccc(-c2cccc(-c3nc(-c4ccccc4)nc(-c4cc(-c5c(-c6ccccc6)ccc6c5oc5ccccc56)cc5c4oc4ccccc45)n3)c2)cc1 ; c1ccc(-c2cccc(-c3nc(-c4ccccc4)nc(-c4ccc(-c5c(-c6ccccc6)ccc6c5oc5ccccc56)c5c4oc4ccccc45)n3)c2)cc1]

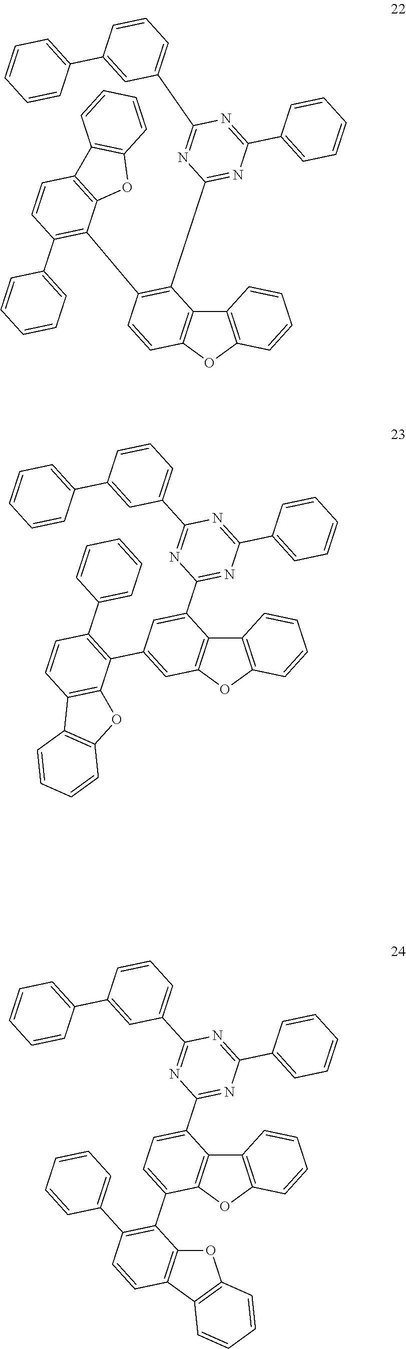 [SMILES: c1ccc(-c2cccc(-c3nc(-c4ccccc4)nc(-c4c(-c5c(-c6ccccc6)ccc6c5oc5ccccc56)ccc5oc6ccccc6c45)n3)c2)cc1 ; c1ccc(-c2cccc(-c3nc(-c4ccccc4)nc(-c4cc(-c5c(-c6ccccc6)ccc6c5oc5ccccc56)cc5oc6ccccc6c45)n3)c2)cc1 ; c1ccc(-c2cccc(-c3nc(-c4ccccc4)nc(-c4ccc(-c5c(-c6ccccc6)ccc6c5oc5ccccc56)c5oc6ccccc6c45)n3)c2)cc1]

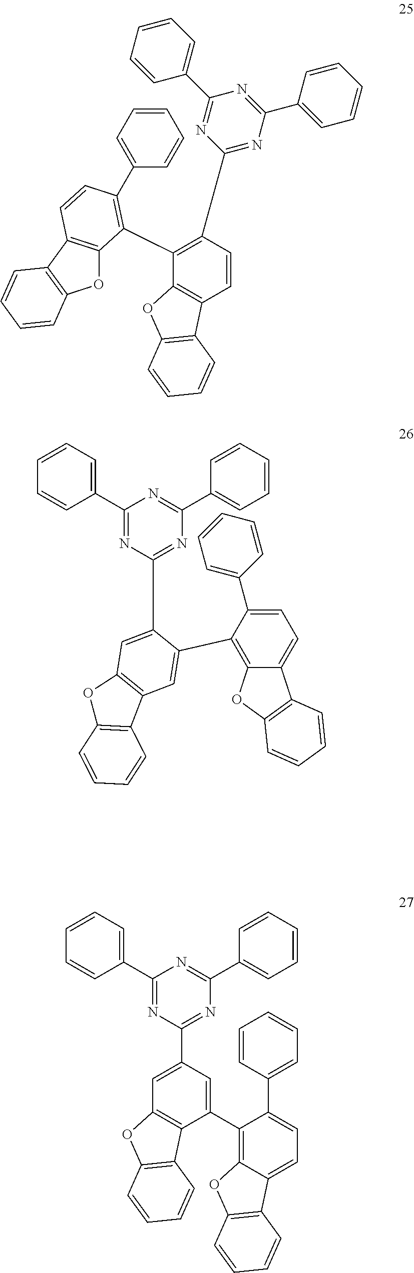 [SMILES: c1ccc(-c2nc(-c3ccccc3)nc(-c3ccc4c(oc5ccccc54)c3-c3c(-c4ccccc4)ccc4c3oc3ccccc34)n2)cc1 ; c1ccc(-c2nc(-c3ccccc3)nc(-c3cc4oc5ccccc5c4cc3-c3c(-c4ccccc4)ccc4c3oc3ccccc34)n2)cc1 ; c1ccc(-c2nc(-c3ccccc3)nc(-c3cc(-c4c(-c5ccccc5)ccc5c4oc4ccccc45)c4c(c3)oc3ccccc34)n2)cc1]

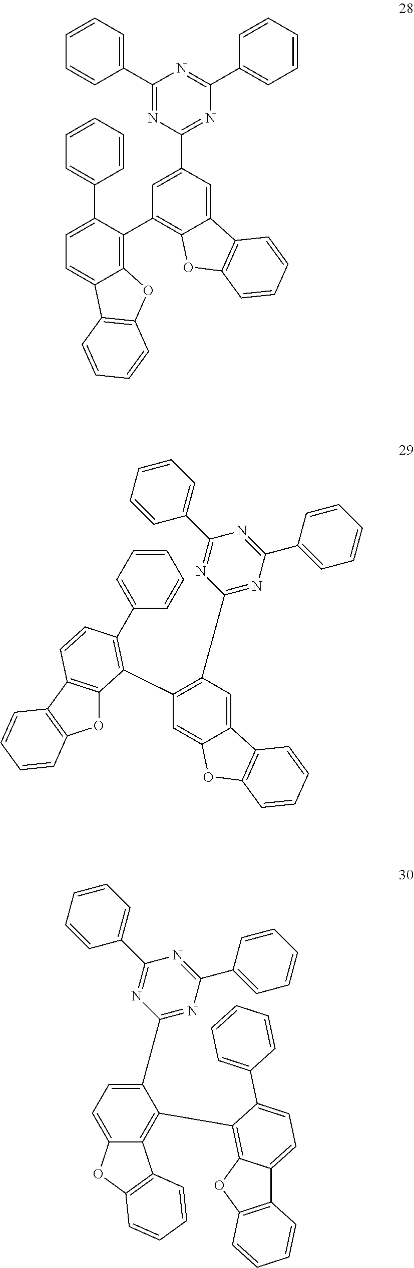 [SMILES: c1ccc(-c2nc(-c3ccccc3)nc(-c3cc(-c4c(-c5ccccc5)ccc5c4oc4ccccc45)c4oc5ccccc5c4c3)n2)cc1 ; c1ccc(-c2nc(-c3ccccc3)nc(-c3cc4c(cc3-c3c(-c5ccccc5)ccc5c3oc3ccccc35)oc3ccccc34)n2)cc1 ; c1ccc(-c2nc(-c3ccccc3)nc(-c3ccc4oc5ccccc5c4c3-c3c(-c4ccccc4)ccc4c3oc3ccccc34)n2)cc1]

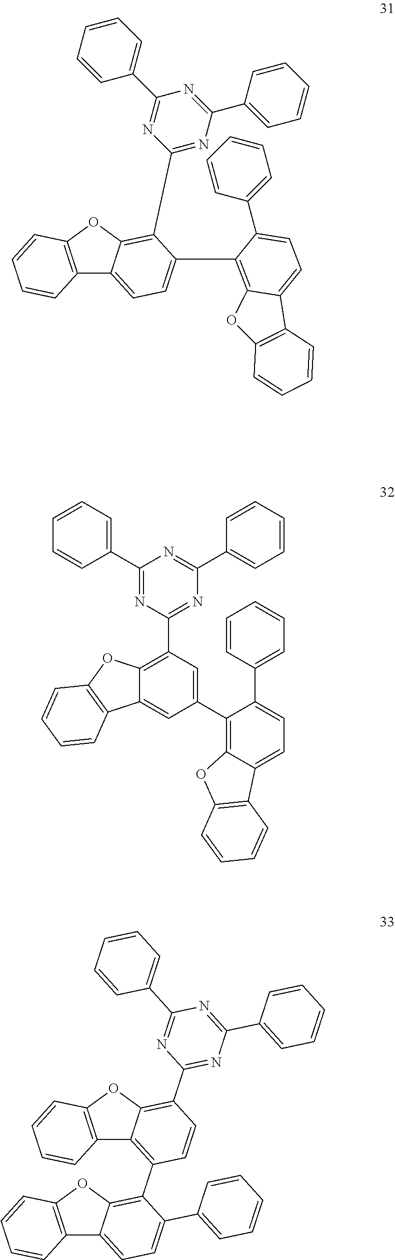 [SMILES: c1ccc(-c2nc(-c3ccccc3)nc(-c3c(-c4c(-c5ccccc5)ccc5c4oc4ccccc45)ccc4c3oc3ccccc34)n2)cc1 ; c1ccc(-c2nc(-c3ccccc3)nc(-c3cc(-c4c(-c5ccccc5)ccc5c4oc4ccccc45)cc4c3oc3ccccc34)n2)cc1 ; c1ccc(-c2nc(-c3ccccc3)nc(-c3ccc(-c4c(-c5ccccc5)ccc5c4oc4ccccc45)c4c3oc3ccccc34)n2)cc1]

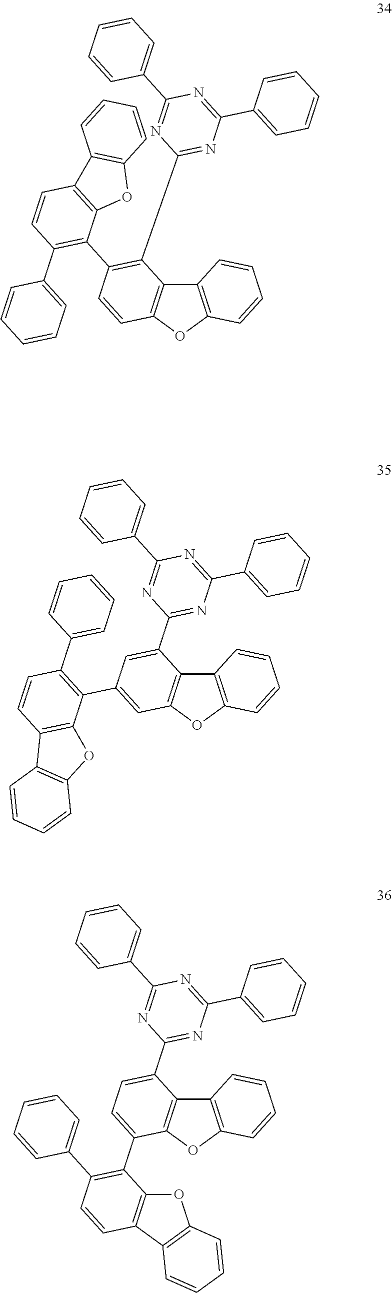 [SMILES: c1ccc(-c2nc(-c3ccccc3)nc(-c3c(-c4c(-c5ccccc5)ccc5c4oc4ccccc45)ccc4oc5ccccc5c34)n2)cc1 ; c1ccc(-c2nc(-c3ccccc3)nc(-c3cc(-c4c(-c5ccccc5)ccc5c4oc4ccccc45)cc4oc5ccccc5c34)n2)cc1 ; c1ccc(-c2nc(-c3ccccc3)nc(-c3ccc(-c4c(-c5ccccc5)ccc5c4oc4ccccc45)c4oc5ccccc5c34)n2)cc1]

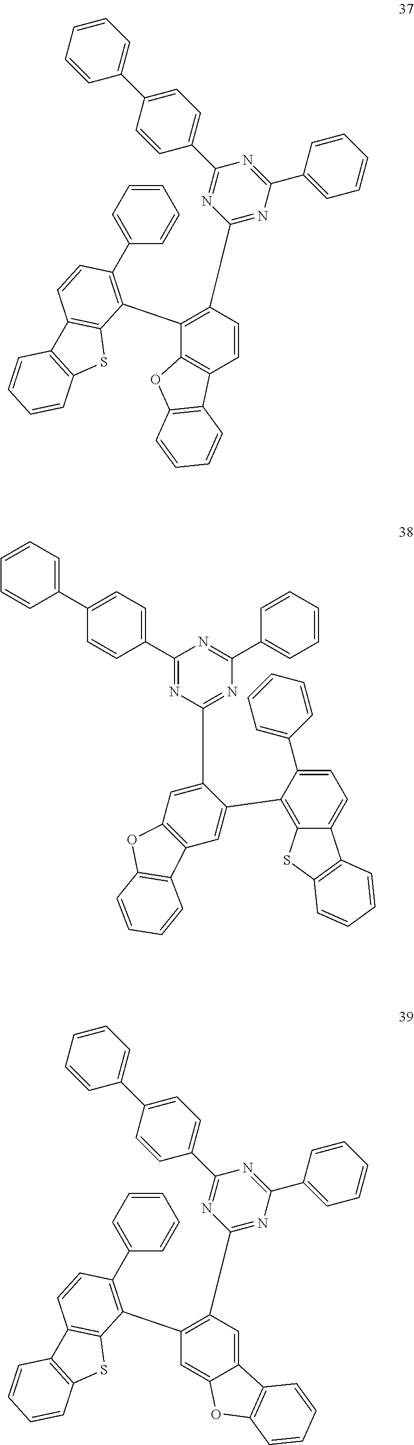 [SMILES: c1ccc(-c2ccc(-c3nc(-c4ccccc4)nc(-c4ccc5c(oc6ccccc65)c4-c4c(-c5ccccc5)ccc5c4sc4ccccc45)n3)cc2)cc1 ; c1ccc(-c2ccc(-c3nc(-c4ccccc4)nc(-c4cc5oc6ccccc6c5cc4-c4c(-c5ccccc5)ccc5c4sc4ccccc45)n3)cc2)cc1 ; c1ccc(-c2ccc(-c3nc(-c4ccccc4)nc(-c4cc5c(cc4-c4c(-c6ccccc6)ccc6c4sc4ccccc46)oc4ccccc45)n3)cc2)cc1]

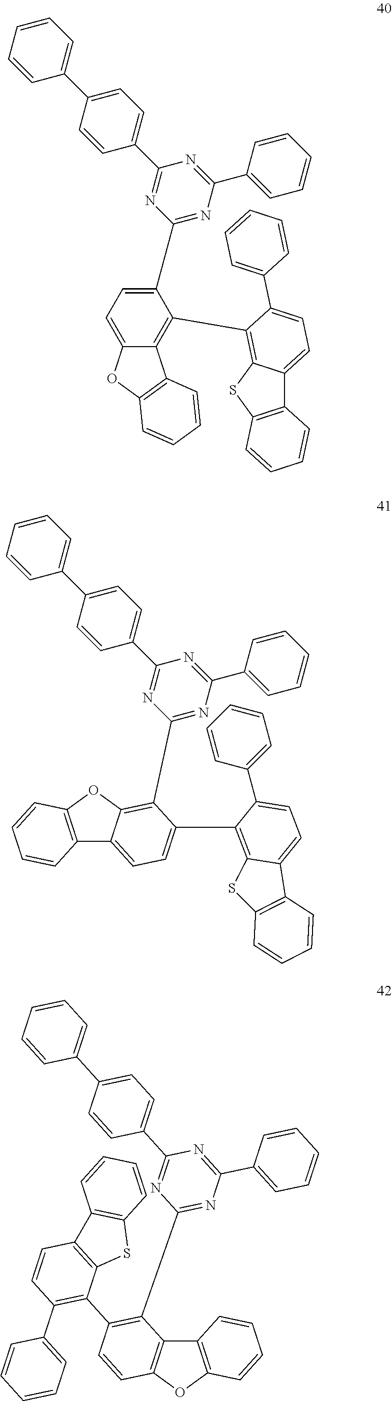 [SMILES: c1ccc(-c2ccc(-c3nc(-c4ccccc4)nc(-c4ccc5oc6ccccc6c5c4-c4c(-c5ccccc5)ccc5c4sc4ccccc45)n3)cc2)cc1 ; c1ccc(-c2ccc(-c3nc(-c4ccccc4)nc(-c4c(-c5c(-c6ccccc6)ccc6c5sc5ccccc56)ccc5c4oc4ccccc45)n3)cc2)cc1 ; c1ccc(-c2ccc(-c3nc(-c4ccccc4)nc(-c4c(-c5c(-c6ccccc6)ccc6c5sc5ccccc56)ccc5oc6ccccc6c45)n3)cc2)cc1]

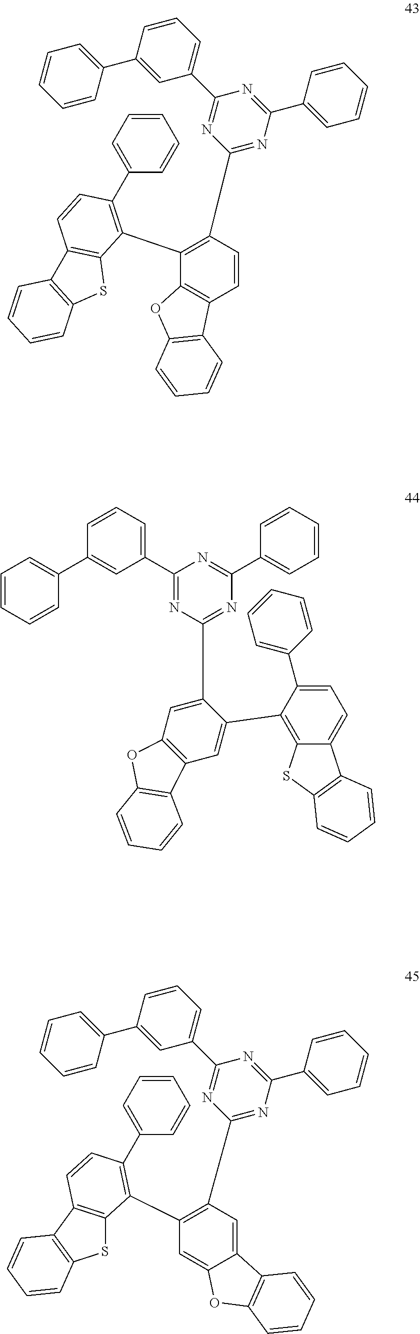 [SMILES: c1ccc(-c2cccc(-c3nc(-c4ccccc4)nc(-c4ccc5c(oc6ccccc65)c4-c4c(-c5ccccc5)ccc5c4sc4ccccc45)n3)c2)cc1 ; c1ccc(-c2cccc(-c3nc(-c4ccccc4)nc(-c4cc5oc6ccccc6c5cc4-c4c(-c5ccccc5)ccc5c4sc4ccccc45)n3)c2)cc1 ; c1ccc(-c2cccc(-c3nc(-c4ccccc4)nc(-c4cc5c(cc4-c4c(-c6ccccc6)ccc6c4sc4ccccc46)oc4ccccc45)n3)c2)cc1]

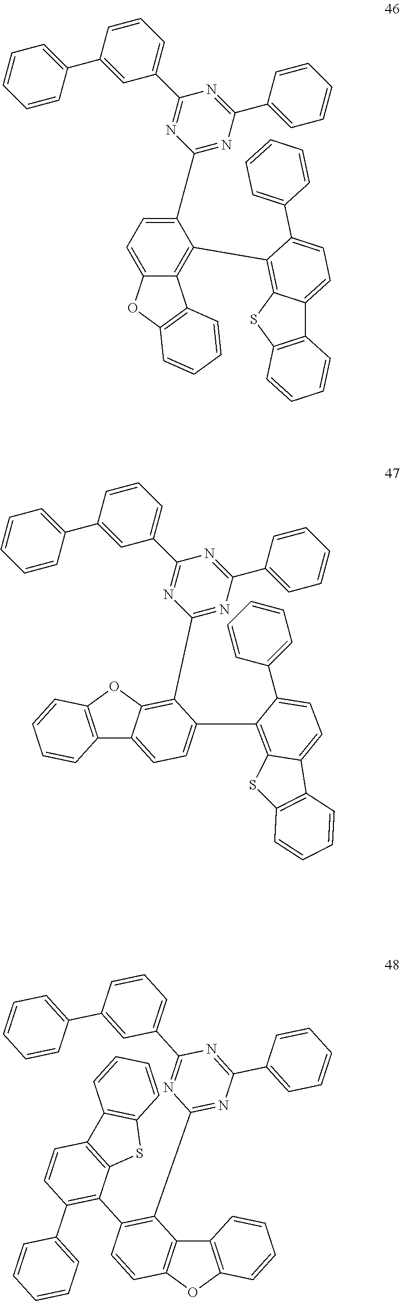 [SMILES: c1ccc(-c2cccc(-c3nc(-c4ccccc4)nc(-c4ccc5oc6ccccc6c5c4-c4c(-c5ccccc5)ccc5c4sc4ccccc45)n3)c2)cc1 ; c1ccc(-c2cccc(-c3nc(-c4ccccc4)nc(-c4c(-c5c(-c6ccccc6)ccc6c5sc5ccccc56)ccc5c4oc4ccccc45)n3)c2)cc1 ; c1ccc(-c2cccc(-c3nc(-c4ccccc4)nc(-c4c(-c5c(-c6ccccc6)ccc6c5sc5ccccc56)ccc5oc6ccccc6c45)n3)c2)cc1]

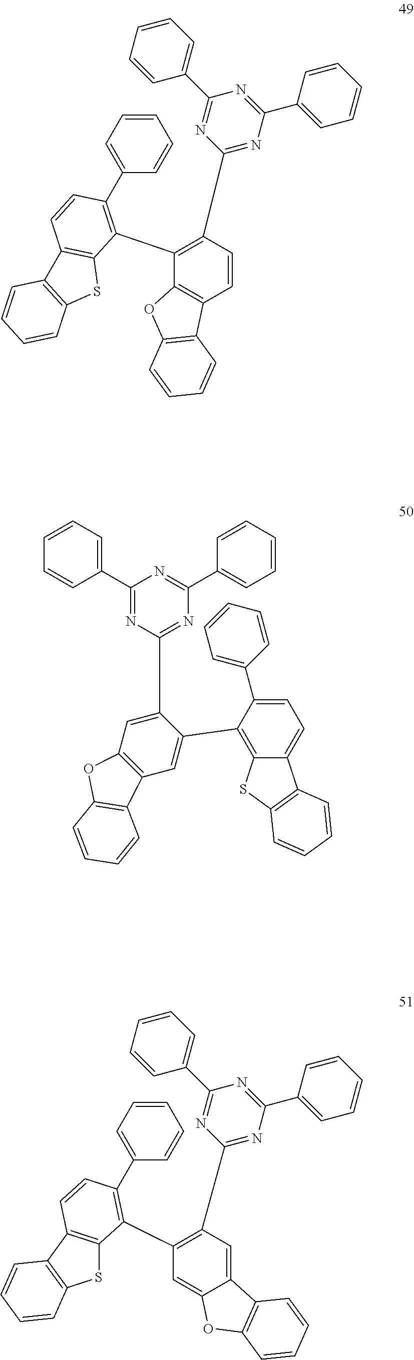 [SMILES: c1ccc(-c2nc(-c3ccccc3)nc(-c3ccc4c(oc5ccccc54)c3-c3c(-c4ccccc4)ccc4c3sc3ccccc34)n2)cc1 ; c1ccc(-c2nc(-c3ccccc3)nc(-c3cc4oc5ccccc5c4cc3-c3c(-c4ccccc4)ccc4c3sc3ccccc34)n2)cc1 ; c1ccc(-c2nc(-c3ccccc3)nc(-c3cc4c(cc3-c3c(-c5ccccc5)ccc5c3sc3ccccc35)oc3ccccc34)n2)cc1]

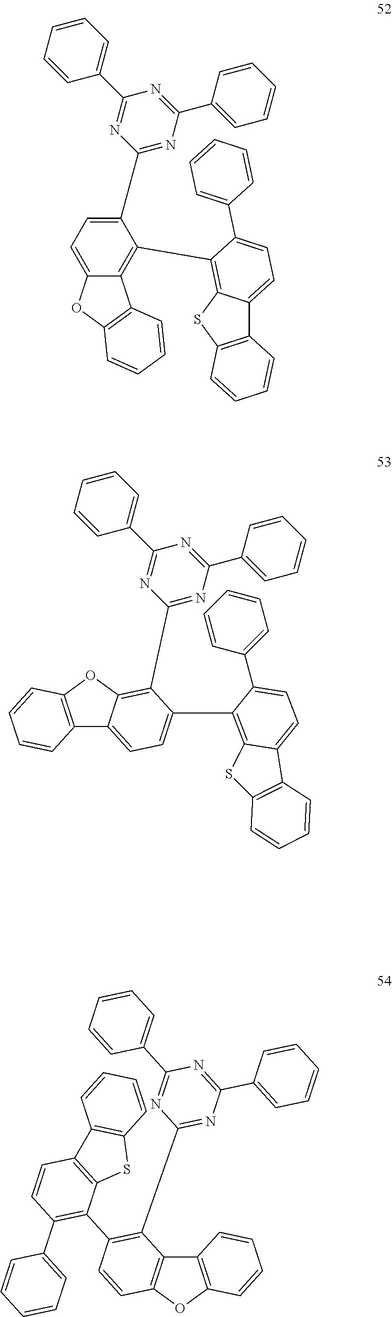 [SMILES: c1ccc(-c2nc(-c3ccccc3)nc(-c3ccc4oc5ccccc5c4c3-c3c(-c4ccccc4)ccc4c3sc3ccccc34)n2)cc1 ; c1ccc(-c2nc(-c3ccccc3)nc(-c3c(-c4c(-c5ccccc5)ccc5c4sc4ccccc45)ccc4c3oc3ccccc34)n2)cc1 ; c1ccc(-c2nc(-c3ccccc3)nc(-c3c(-c4c(-c5ccccc5)ccc5c4sc4ccccc45)ccc4oc5ccccc5c34)n2)cc1]

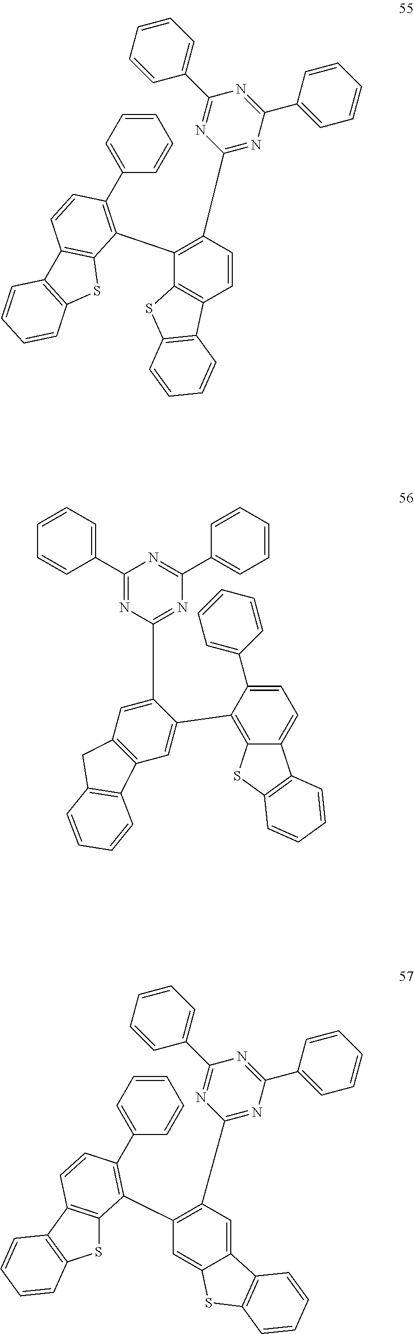 [SMILES: c1ccc(-c2nc(-c3ccccc3)nc(-c3ccc4c(sc5ccccc54)c3-c3c(-c4ccccc4)ccc4c3sc3ccccc34)n2)cc1 ; c1ccc(-c2nc(-c3ccccc3)nc(-c3cc4c(cc3-c3c(-c5ccccc5)ccc5c3sc3ccccc35)-c3ccccc3C4)n2)cc1 ; c1ccc(-c2nc(-c3ccccc3)nc(-c3cc4c(cc3-c3c(-c5ccccc5)ccc5c3sc3ccccc35)sc3ccccc34)n2)cc1]

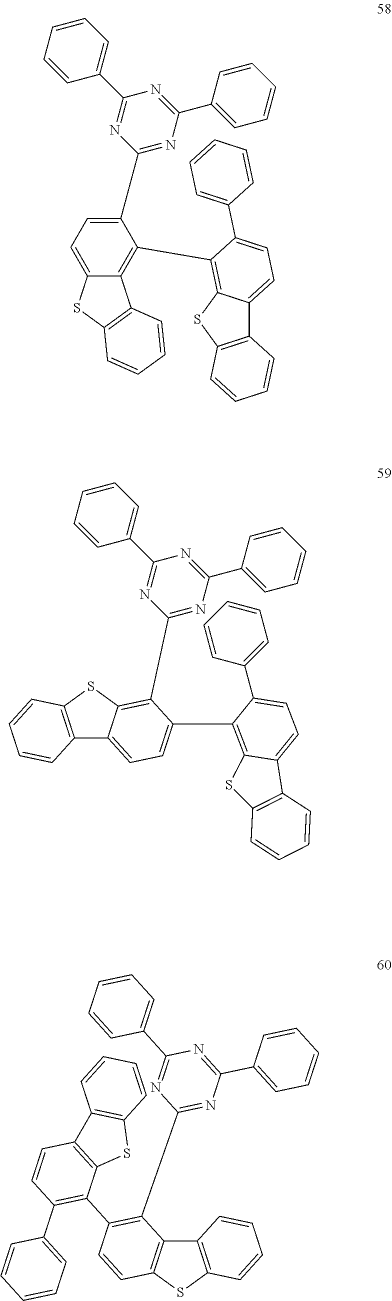 [SMILES: c1ccc(-c2nc(-c3ccccc3)nc(-c3ccc4sc5ccccc5c4c3-c3c(-c4ccccc4)ccc4c3sc3ccccc34)n2)cc1 ; c1ccc(-c2nc(-c3ccccc3)nc(-c3c(-c4c(-c5ccccc5)ccc5c4sc4ccccc45)ccc4c3sc3ccccc34)n2)cc1 ; c1ccc(-c2nc(-c3ccccc3)nc(-c3c(-c4c(-c5ccccc5)ccc5c4sc4ccccc45)ccc4sc5ccccc5c34)n2)cc1]

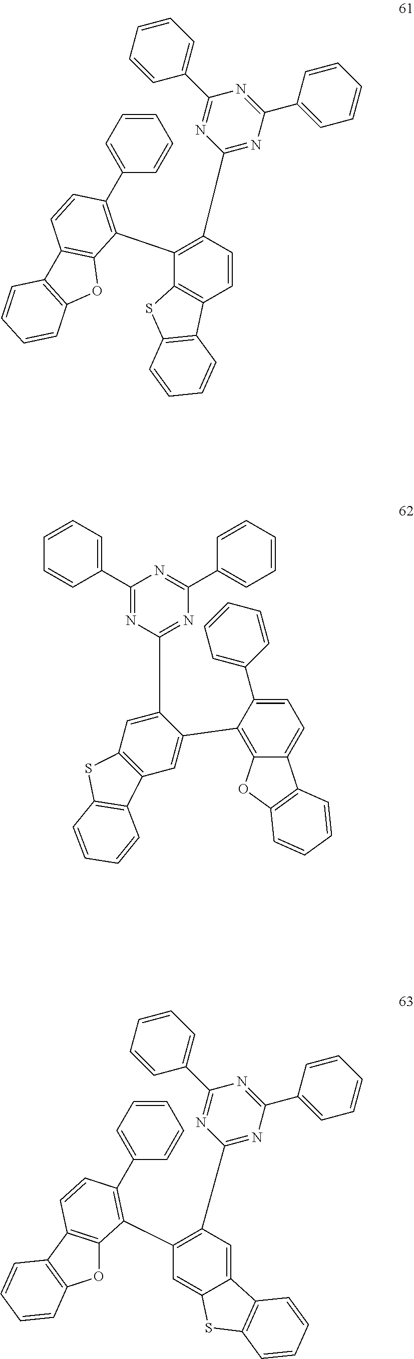 [SMILES: c1ccc(-c2nc(-c3ccccc3)nc(-c3ccc4c(sc5ccccc54)c3-c3c(-c4ccccc4)ccc4c3oc3ccccc34)n2)cc1 ; c1ccc(-c2nc(-c3ccccc3)nc(-c3cc4sc5ccccc5c4cc3-c3c(-c4ccccc4)ccc4c3oc3ccccc34)n2)cc1 ; c1ccc(-c2nc(-c3ccccc3)nc(-c3cc4c(cc3-c3c(-c5ccccc5)ccc5c3oc3ccccc35)sc3ccccc34)n2)cc1]

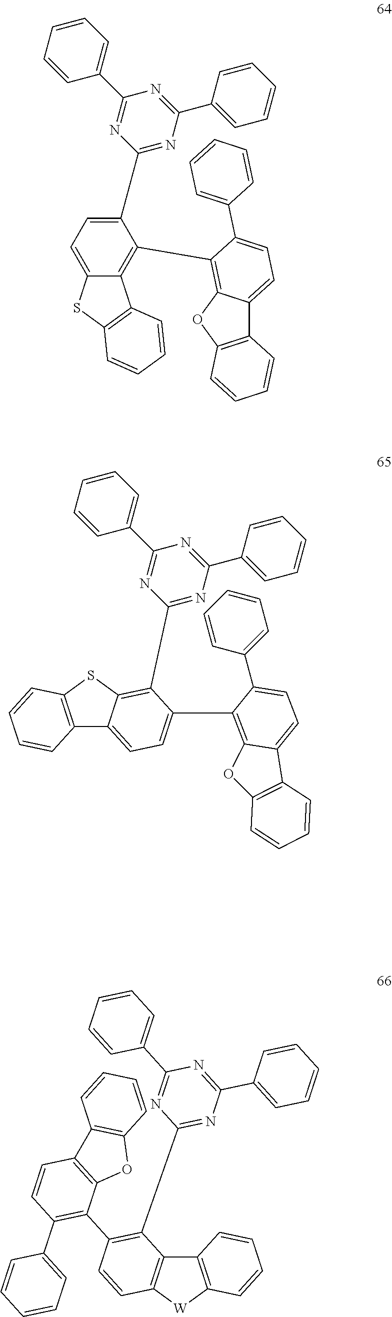

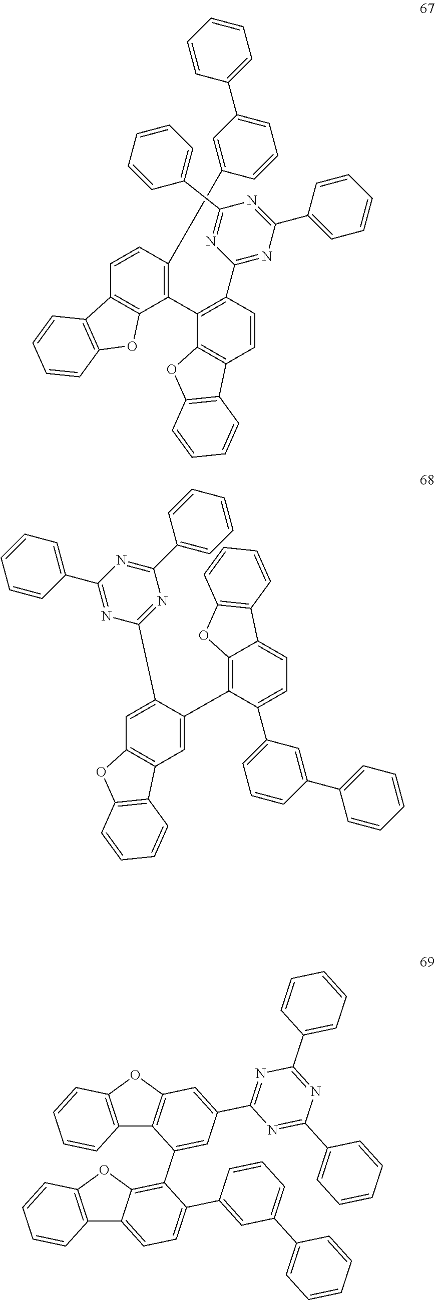

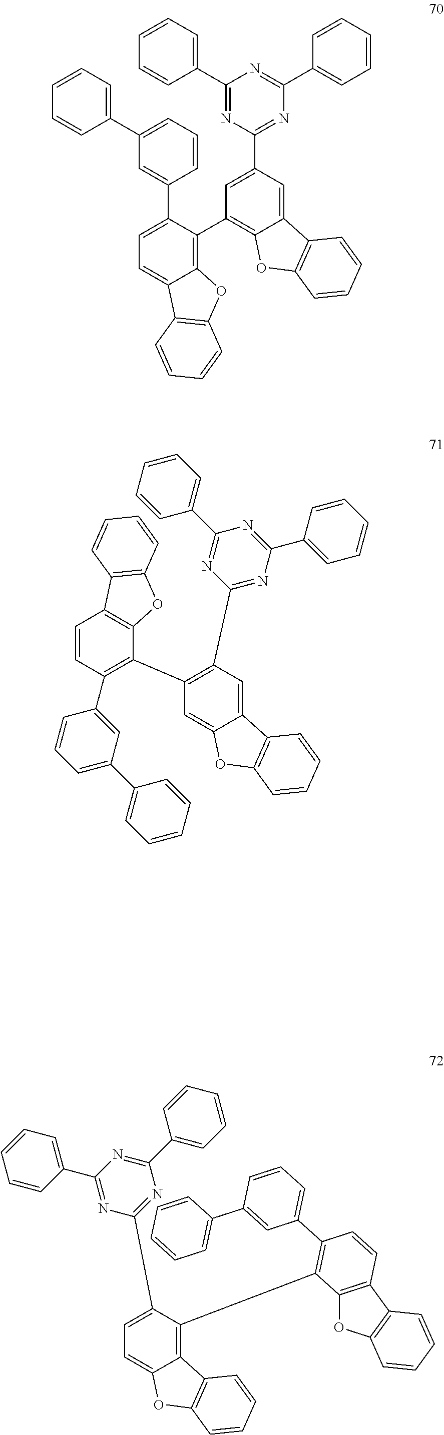 [SMILES: c1ccc(-c2cccc(-c3ccc4c(oc5ccccc54)c3-c3cc(-c4nc(-c5ccccc5)nc(-c5ccccc5)n4)cc4c3oc3ccccc34)c2)cc1 ; c1ccc(-c2cccc(-c3ccc4c(oc5ccccc54)c3-c3cc4oc5ccccc5c4cc3-c3nc(-c4ccccc4)nc(-c4ccccc4)n3)c2)cc1 ; c1ccc(-c2cccc(-c3ccc4c(oc5ccccc54)c3-c3c(-c4nc(-c5ccccc5)nc(-c5ccccc5)n4)ccc4oc5ccccc5c34)c2)cc1]

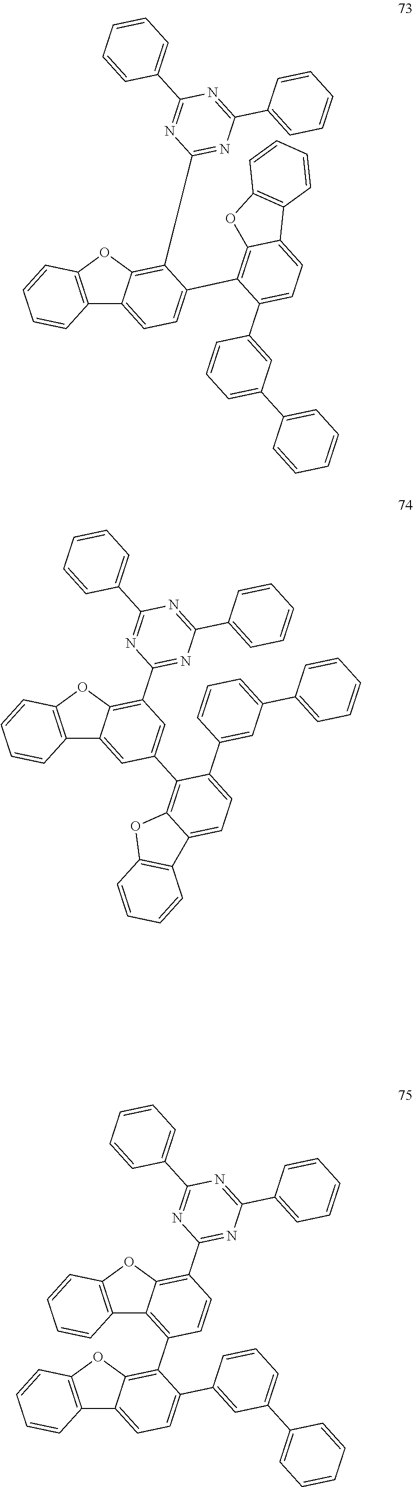 [SMILES: c1ccc(-c2cccc(-c3ccc4c(oc5ccccc54)c3-c3ccc4c(oc5ccccc54)c3-c3nc(-c4ccccc4)nc(-c4ccccc4)n3)c2)cc1 ; c1ccc(-c2cccc(-c3ccc4c(oc5ccccc54)c3-c3cc(-c4nc(-c5ccccc5)nc(-c5ccccc5)n4)c4oc5ccccc5c4c3)c2)cc1 ; c1ccc(-c2cccc(-c3ccc4c(oc5ccccc54)c3-c3ccc(-c4nc(-c5ccccc5)nc(-c5ccccc5)n4)c4oc5ccccc5c34)c2)cc1]

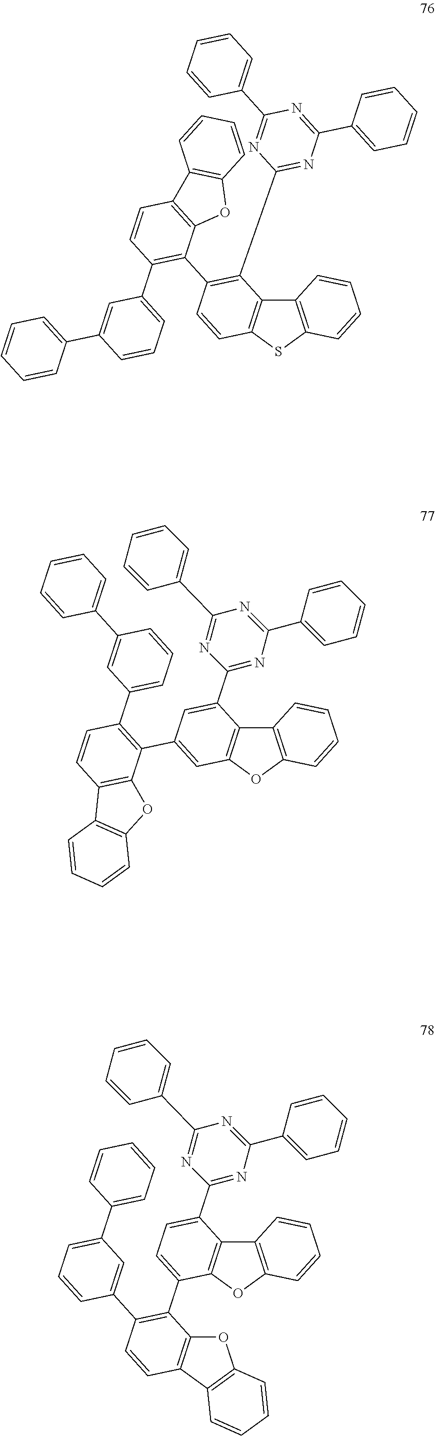 [SMILES: c1ccc(-c2cccc(-c3ccc4c(oc5ccccc54)c3-c3ccc4sc5ccccc5c4c3-c3nc(-c4ccccc4)nc(-c4ccccc4)n3)c2)cc1 ; c1ccc(-c2cccc(-c3ccc4c(oc5ccccc54)c3-c3cc(-c4nc(-c5ccccc5)nc(-c5ccccc5)n4)c4c(c3)oc3ccccc34)c2)cc1 ; c1ccc(-c2cccc(-c3ccc4c(oc5ccccc54)c3-c3ccc(-c4nc(-c5ccccc5)nc(-c5ccccc5)n4)c4c3oc3ccccc34)c2)cc1]

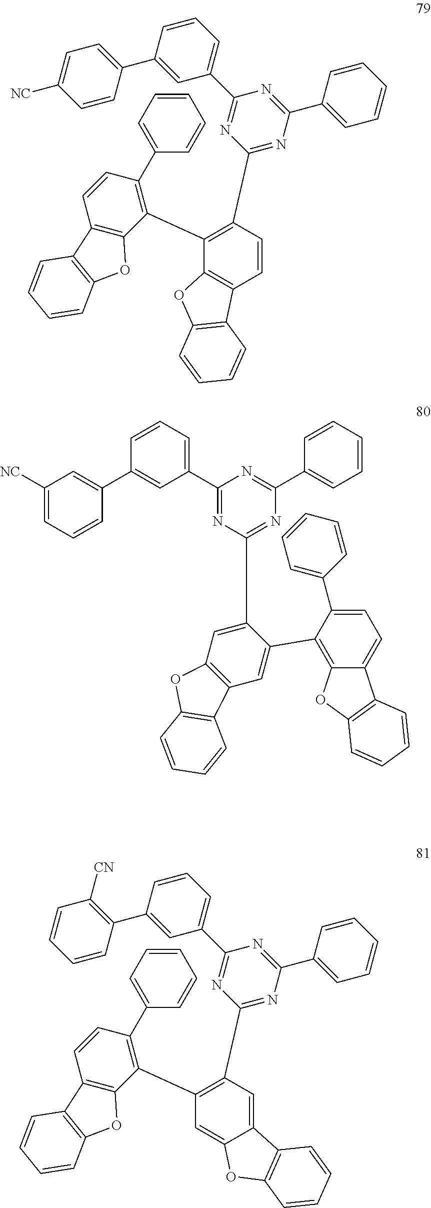 [SMILES: N#Cc1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; N#Cc1cccc(-c2cccc(-c3nc(-c4ccccc4)nc(-c4cc5oc6ccccc6c5cc4-c4c(-c5ccccc5)ccc5c4oc4ccccc45)n3)c2)c1 ; N#Cc1ccccc1-c1cccc(-c2nc(-c3ccccc3)nc(-c3cc4c(cc3-c3c(-c5ccccc5)ccc5c3oc3ccccc35)oc3ccccc34)n2)c1]

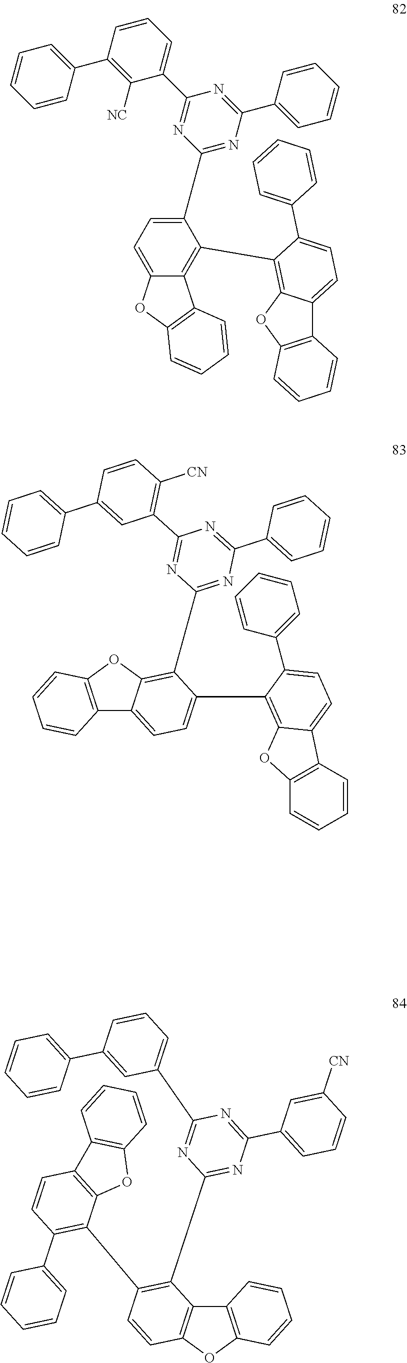 [SMILES: N#Cc1c(-c2ccccc2)cccc1-c1nc(-c2ccccc2)nc(-c2ccc3oc4ccccc4c3c2-c2c(-c3ccccc3)ccc3c2oc2ccccc23)n1 ; N#Cc1ccc(-c2ccccc2)cc1-c1nc(-c2ccccc2)nc(-c2c(-c3c(-c4ccccc4)ccc4c3oc3ccccc34)ccc3c2oc2ccccc23)n1 ; N#Cc1cccc(-c2nc(-c3cccc(-c4ccccc4)c3)nc(-c3c(-c4c(-c5ccccc5)ccc5c4oc4ccccc45)ccc4oc5ccccc5c34)n2)c1]

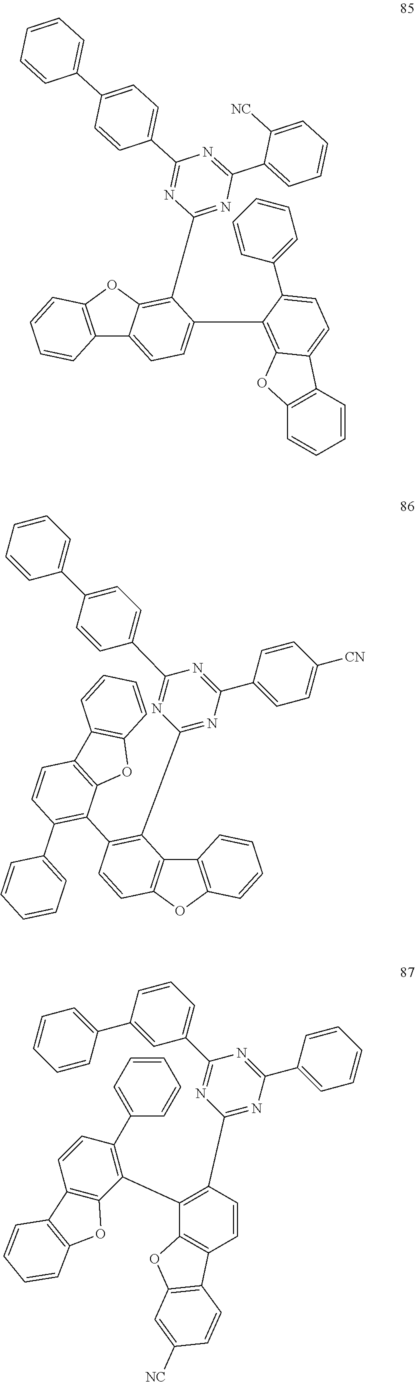 [SMILES: N#Cc1ccccc1-c1nc(-c2ccc(-c3ccccc3)cc2)nc(-c2c(-c3c(-c4ccccc4)ccc4c3oc3ccccc34)ccc3c2oc2ccccc23)n1 ; N#Cc1ccc(-c2nc(-c3ccc(-c4ccccc4)cc3)nc(-c3c(-c4c(-c5ccccc5)ccc5c4oc4ccccc45)ccc4oc5ccccc5c34)n2)cc1 ; N#Cc1ccc2c(c1)oc1c(-c3c(-c4ccccc4)ccc4c3oc3ccccc34)c(-c3nc(-c4ccccc4)nc(-c4cccc(-c5ccccc5)c4)n3)ccc12]

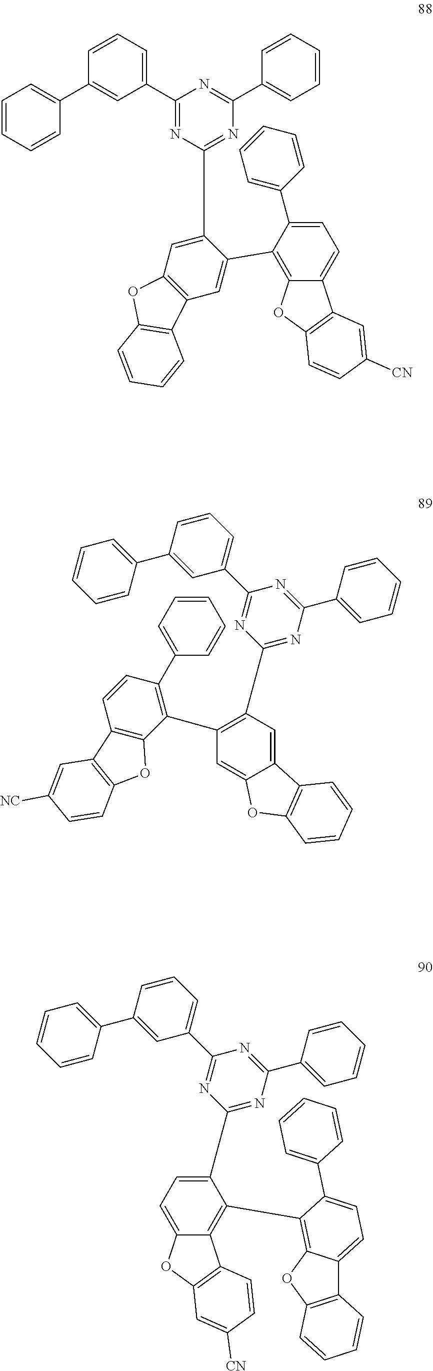 [SMILES: N#Cc1ccc2oc3c(-c4cc5c(cc4-c4nc(-c6ccccc6)nc(-c6cccc(-c7ccccc7)c6)n4)oc4ccccc45)c(-c4ccccc4)ccc3c2c1 ; N#Cc1ccc2oc3c(-c4cc5oc6ccccc6c5cc4-c4nc(-c5ccccc5)nc(-c5cccc(-c6ccccc6)c5)n4)c(-c4ccccc4)ccc3c2c1 ; N#Cc1ccc2c(c1)oc1ccc(-c3nc(-c4ccccc4)nc(-c4cccc(-c5ccccc5)c4)n3)c(-c3c(-c4ccccc4)ccc4c3oc3ccccc34)c12]

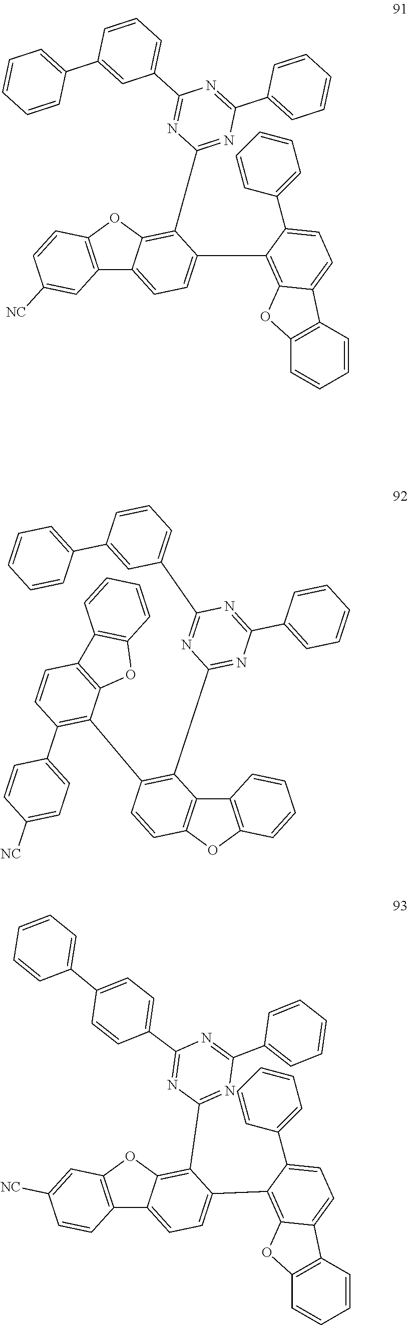 [SMILES: N#Cc1ccc2oc3c(-c4nc(-c5ccccc5)nc(-c5cccc(-c6ccccc6)c5)n4)c(-c4c(-c5ccccc5)ccc5c4oc4ccccc45)ccc3c2c1 ; N#Cc1ccc(-c2ccc3c(oc4ccccc43)c2-c2ccc3oc4ccccc4c3c2-c2nc(-c3ccccc3)nc(-c3cccc(-c4ccccc4)c3)n2)cc1 ; N#Cc1ccc2c(c1)oc1c(-c3nc(-c4ccccc4)nc(-c4ccc(-c5ccccc5)cc4)n3)c(-c3c(-c4ccccc4)ccc4c3oc3ccccc34)ccc12]

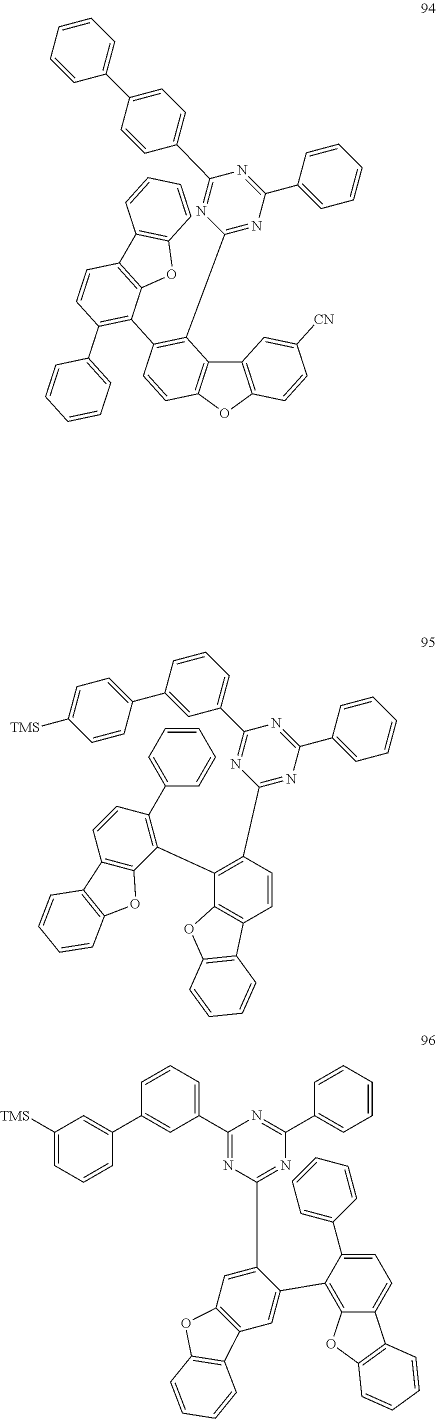 [SMILES: N#Cc1ccc2oc3ccc(-c4c(-c5ccccc5)ccc5c4oc4ccccc45)c(-c4nc(-c5ccccc5)nc(-c5ccc(-c6ccccc6)cc5)n4)c3c2c1 ; C[Si](C)(C)c1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; C[Si](C)(C)c1cccc(-c2cccc(-c3nc(-c4ccccc4)nc(-c4cc5oc6ccccc6c5cc4-c4c(-c5ccccc5)ccc5c4oc4ccccc45)n3)c2)c1]

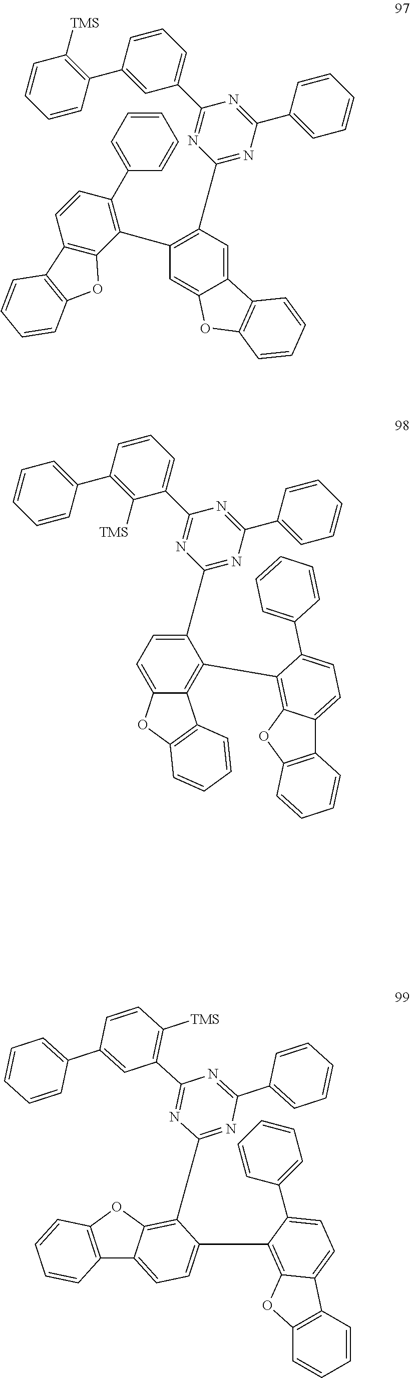 [SMILES: C[Si](C)(C)c1ccccc1-c1cccc(-c2nc(-c3ccccc3)nc(-c3cc4c(cc3-c3c(-c5ccccc5)ccc5c3oc3ccccc35)oc3ccccc34)n2)c1 ; C[Si](C)(C)c1c(-c2ccccc2)cccc1-c1nc(-c2ccccc2)nc(-c2ccc3oc4ccccc4c3c2-c2c(-c3ccccc3)ccc3c2oc2ccccc23)n1 ; C[Si](C)(C)c1ccc(-c2ccccc2)cc1-c1nc(-c2ccccc2)nc(-c2c(-c3c(-c4ccccc4)ccc4c3oc3ccccc34)ccc3c2oc2ccccc23)n1]

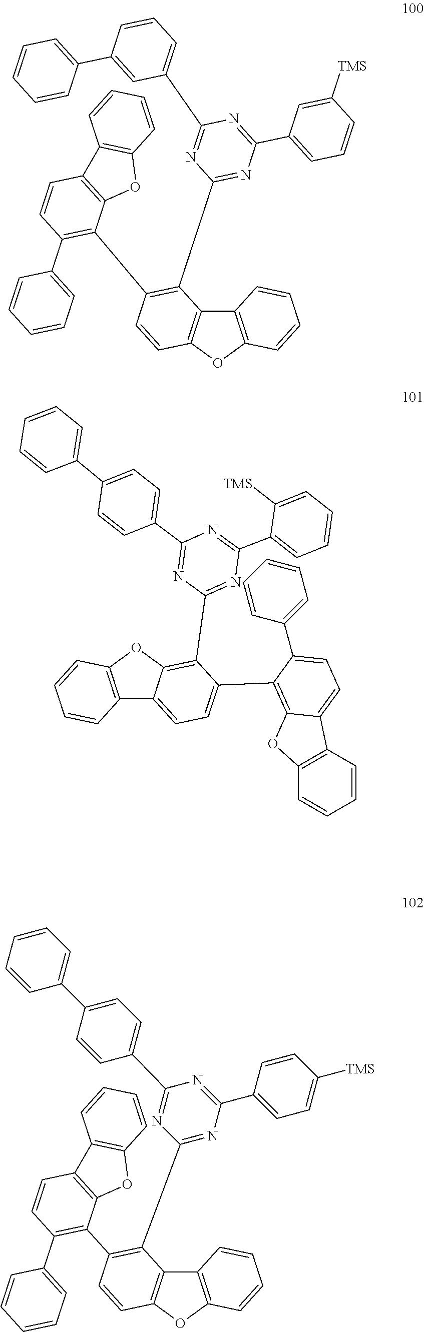 [SMILES: C[Si](C)(C)c1cccc(-c2nc(-c3cccc(-c4ccccc4)c3)nc(-c3c(-c4c(-c5ccccc5)ccc5c4oc4ccccc45)ccc4oc5ccccc5c34)n2)c1 ; C[Si](C)(C)c1ccccc1-c1nc(-c2ccc(-c3ccccc3)cc2)nc(-c2c(-c3c(-c4ccccc4)ccc4c3oc3ccccc34)ccc3c2oc2ccccc23)n1 ; C[Si](C)(C)c1ccc(-c2nc(-c3ccc(-c4ccccc4)cc3)nc(-c3c(-c4c(-c5ccccc5)ccc5c4oc4ccccc45)ccc4oc5ccccc5c34)n2)cc1]

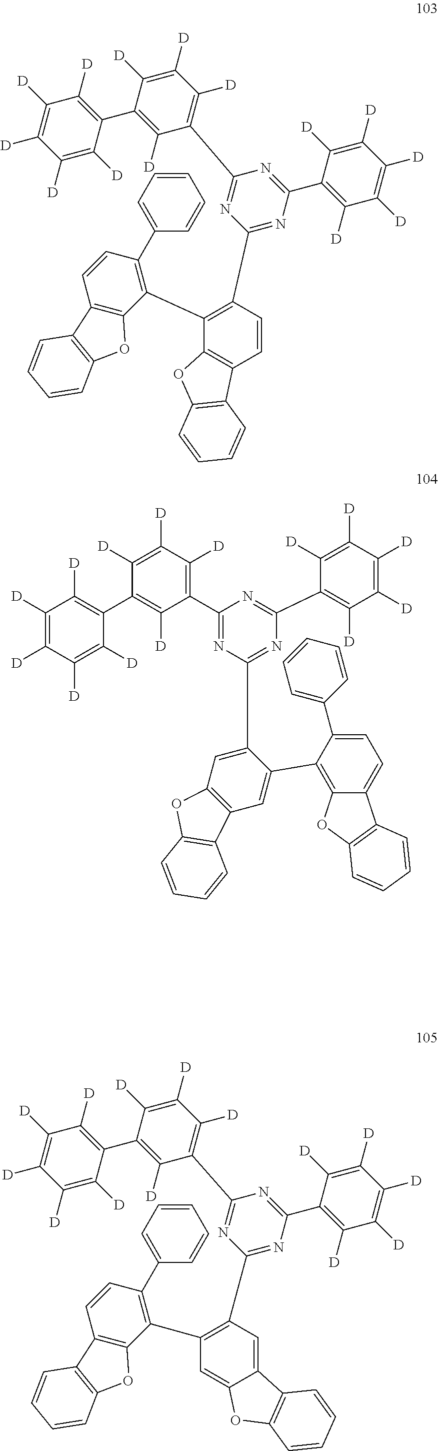

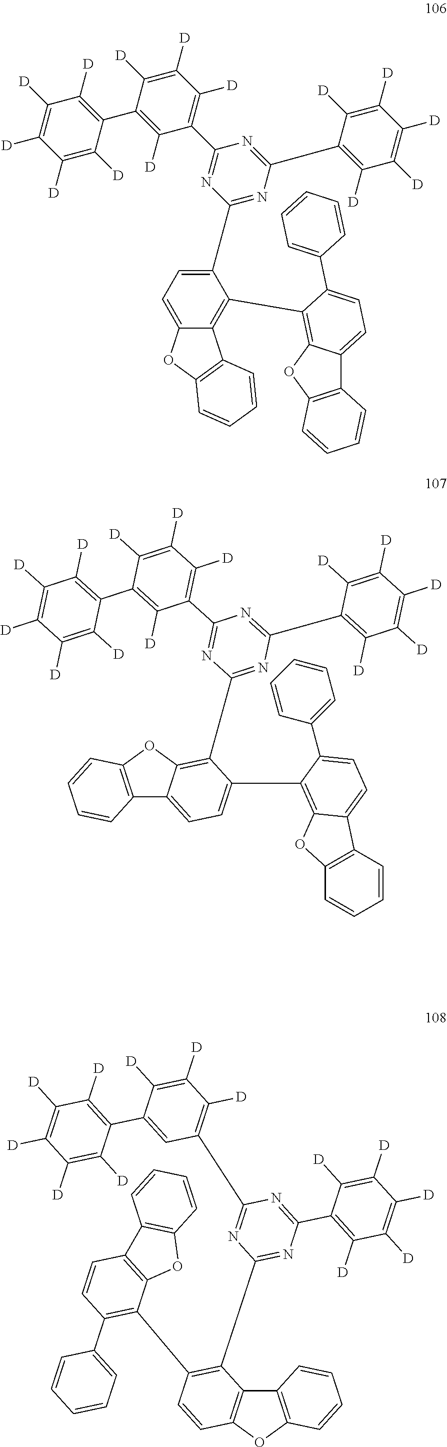

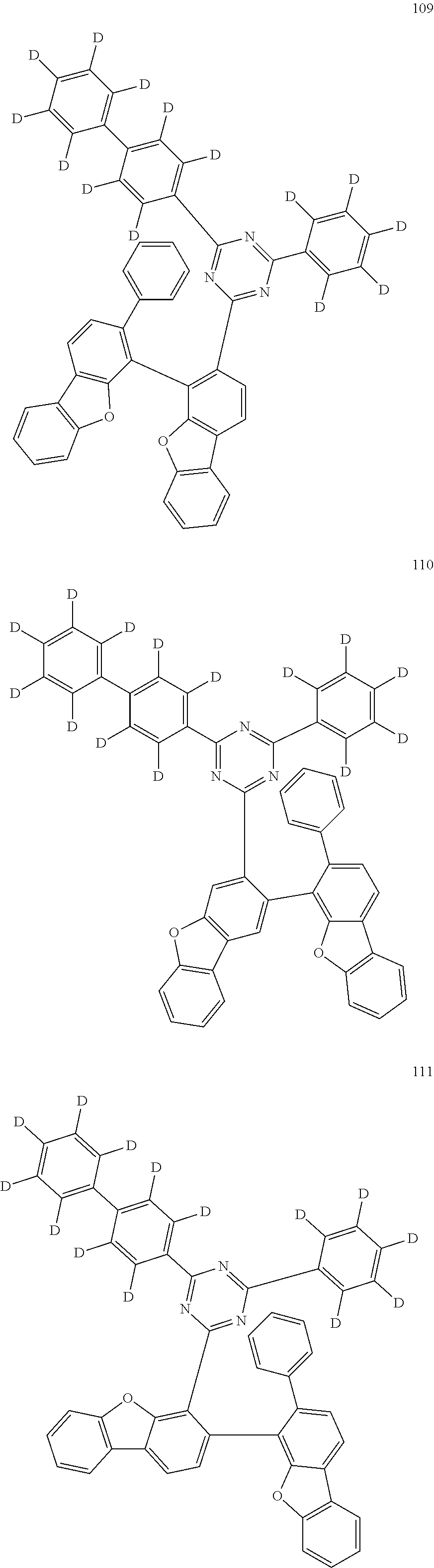

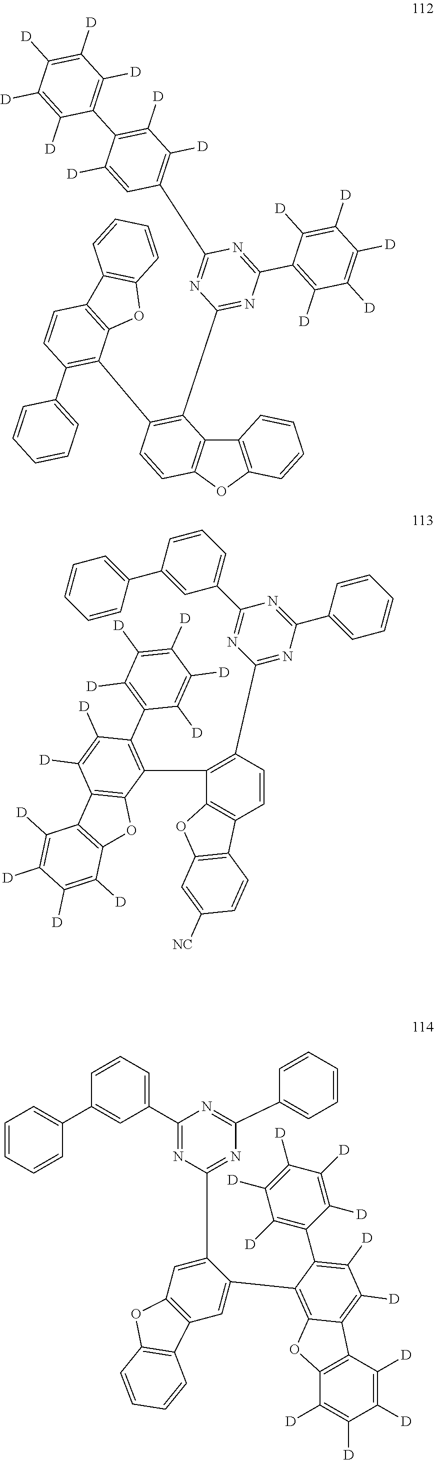

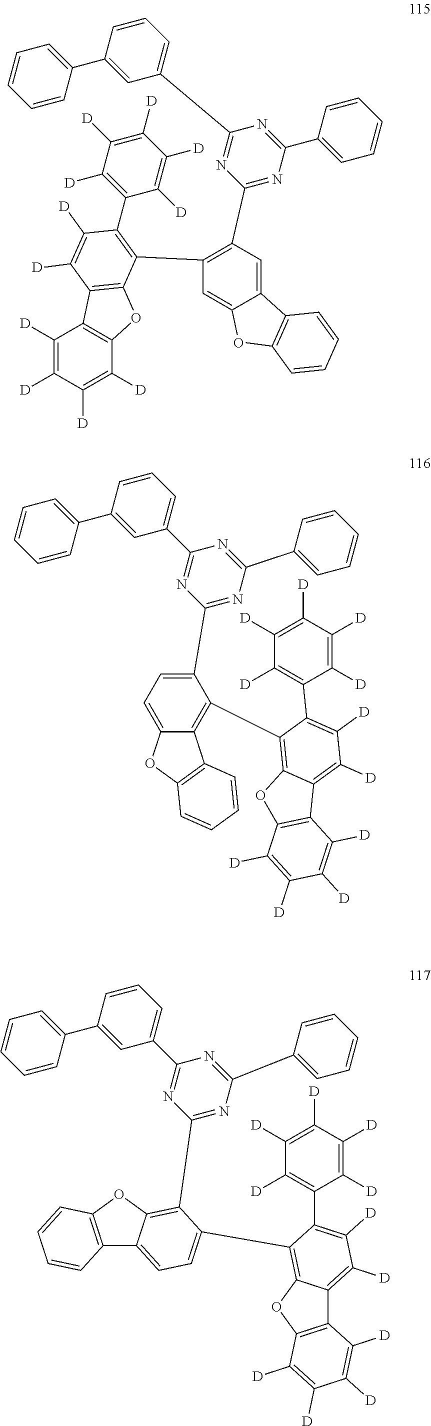 [SMILES: [2H]c1c([2H])c([2H])c(-c2c([2H])c([2H])c3c(oc4c([2H])c([2H])c([2H])c([2H])c43)c2-c2cc3oc4ccccc4c3cc2-c2nc(-c3ccccc3)nc(-c3cccc(-c4ccccc4)c3)n2)c([2H])c1[2H] ; [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)ccc3oc4ccccc4c23)c([2H])c1[2H] ; [2H]c1c([2H])c([2H])c(-c2c([2H])c([2H])c3c(oc4c([2H])c([2H])c([2H])c([2H])c43)c2-c2ccc3c(oc4ccccc43)c2-c2nc(-c3ccccc3)nc(-c3cccc(-c4ccccc4)c3)n2)c([2H])c1[2H]]

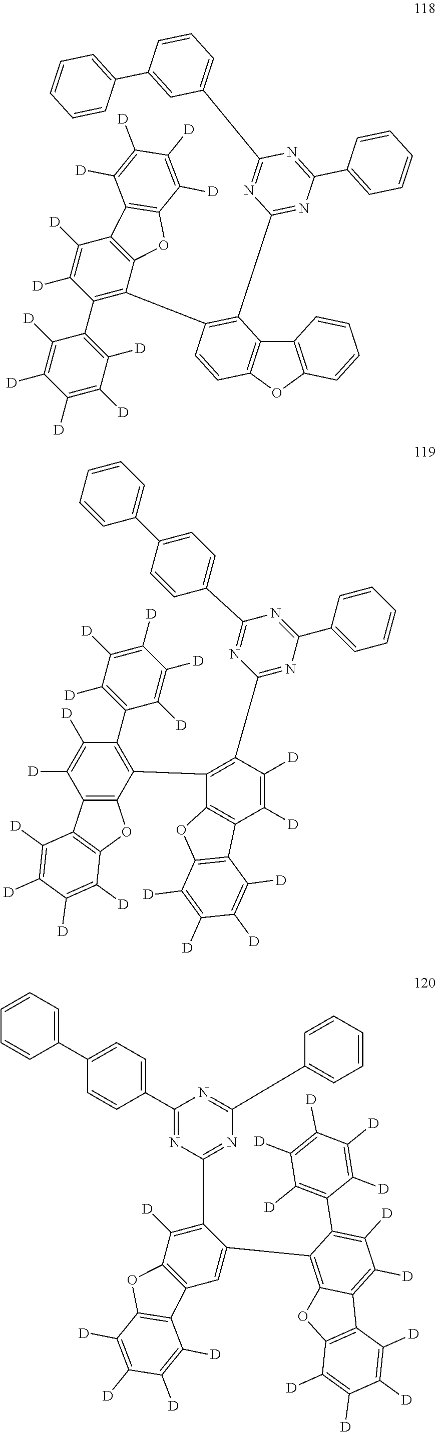

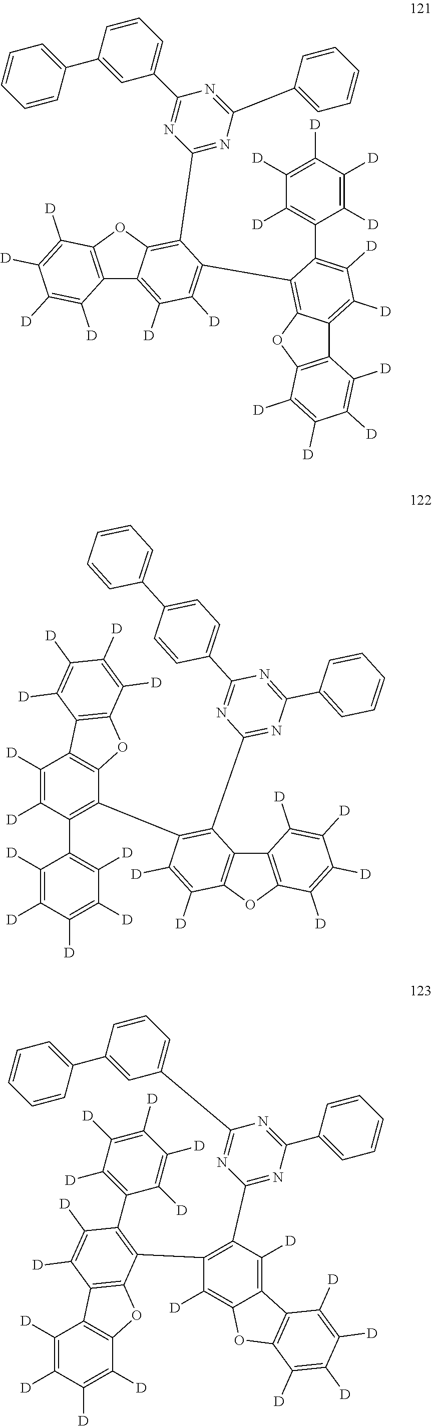

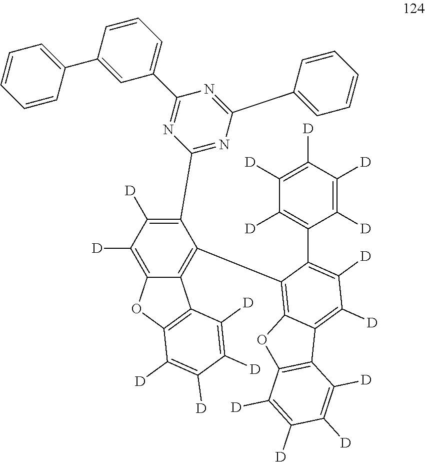 [SMILES: [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)c([2H])c([2H])c3oc4c([2H])c([2H])c([2H])c([2H])c4c23)c([2H])c1[2H]]

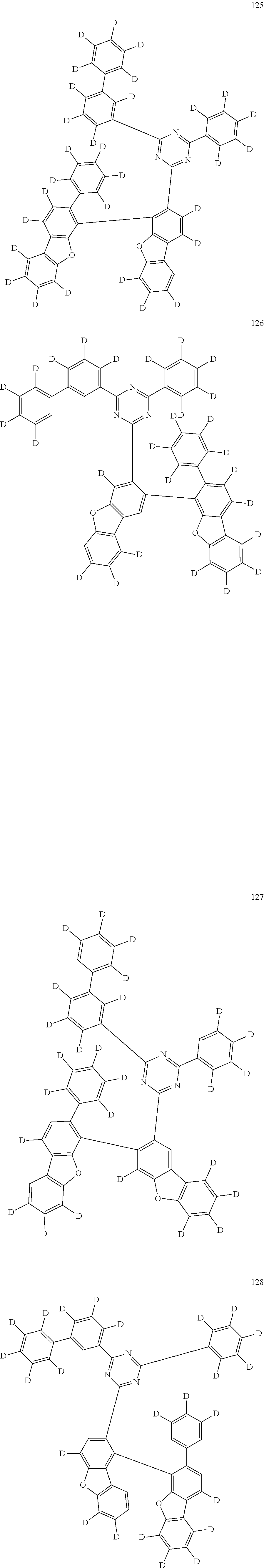

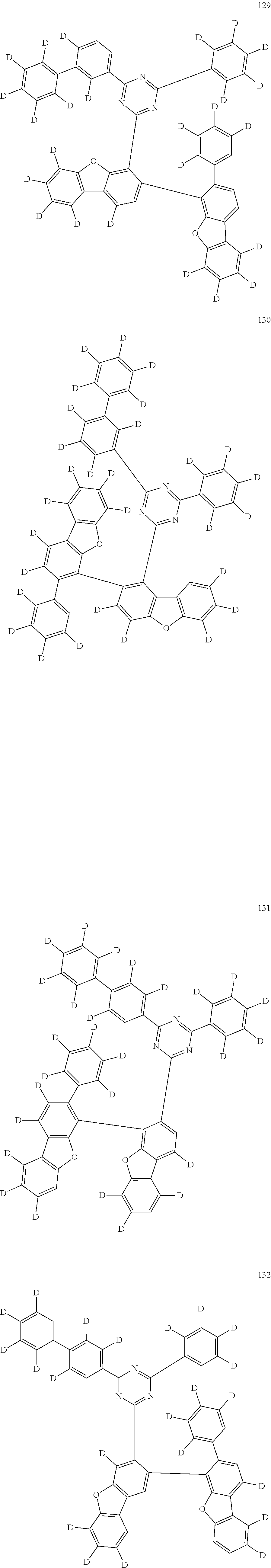

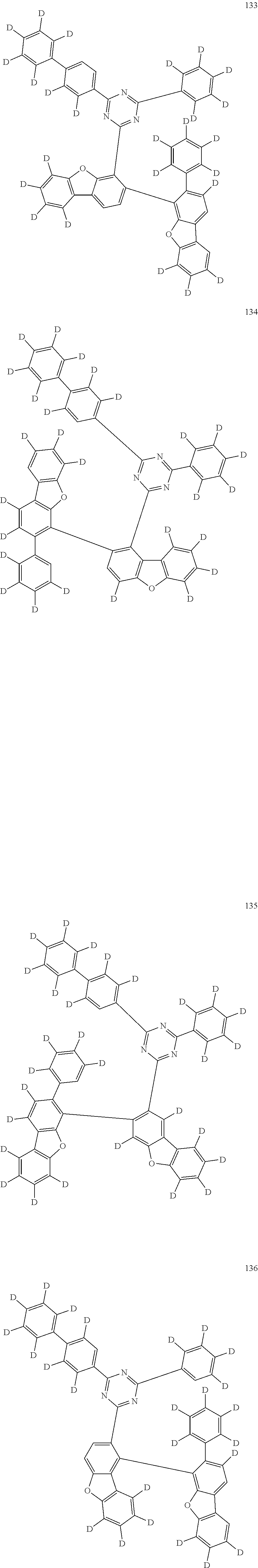

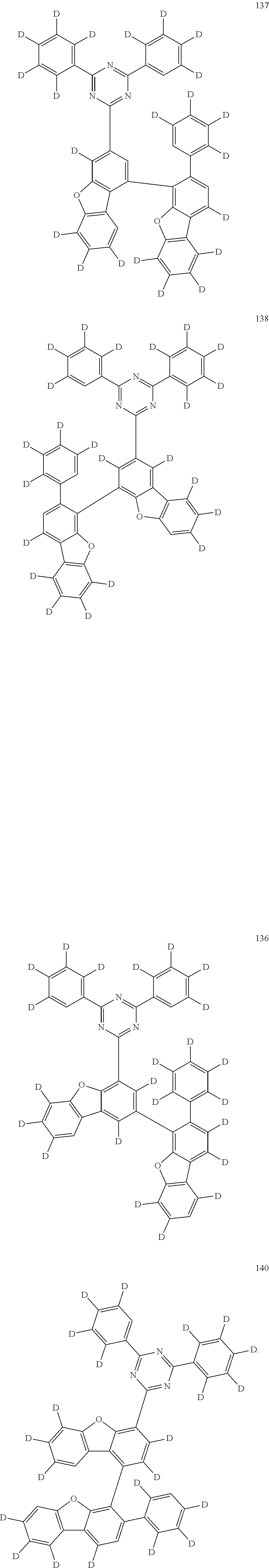

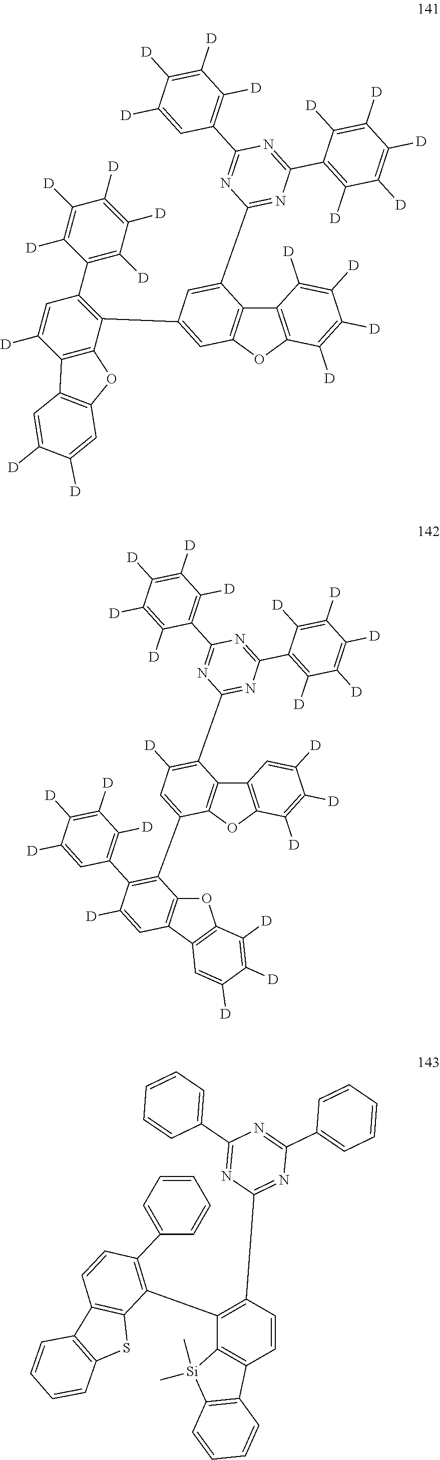

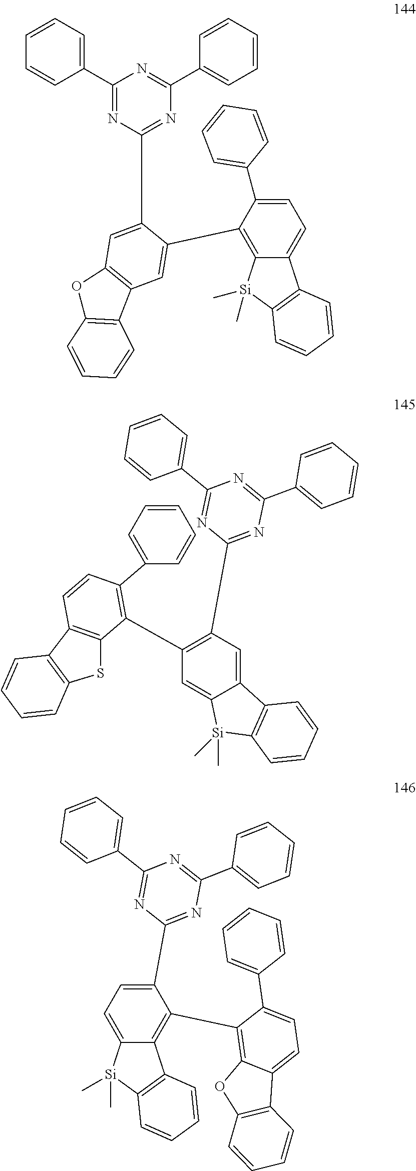 [SMILES: C[Si]1(C)c2ccccc2-c2ccc(-c3ccccc3)c(-c3cc4c(cc3-c3nc(-c5ccccc5)nc(-c5ccccc5)n3)oc3ccccc34)c21 ; C[Si]1(C)c2ccccc2-c2cc(-c3nc(-c4ccccc4)nc(-c4ccccc4)n3)c(-c3c(-c4ccccc4)ccc4c3sc3ccccc34)cc21 ; C[Si]1(C)c2ccccc2-c2c1ccc(-c1nc(-c3ccccc3)nc(-c3ccccc3)n1)c2-c1c(-c2ccccc2)ccc2c1oc1ccccc12]

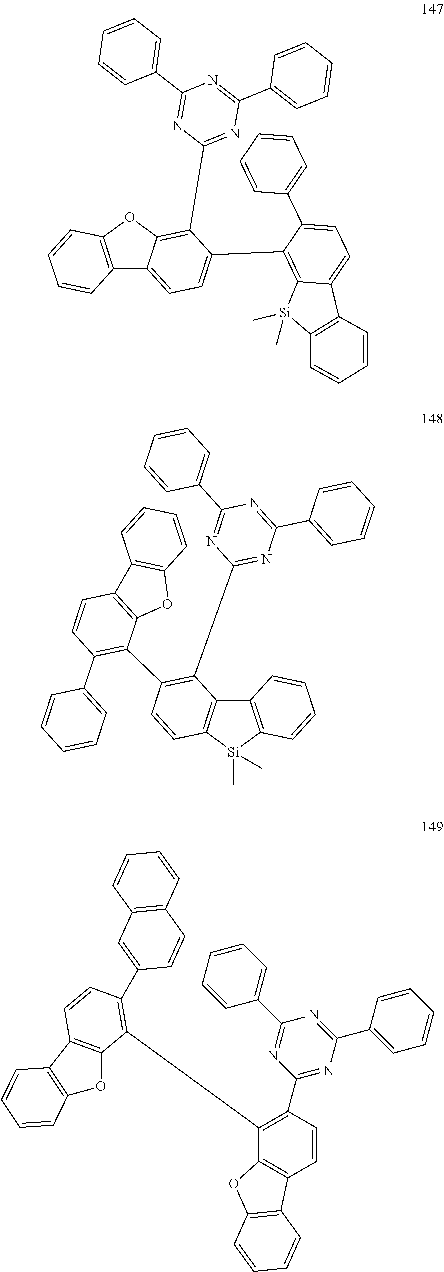 [SMILES: C[Si]1(C)c2ccccc2-c2ccc(-c3ccccc3)c(-c3ccc4c(oc5ccccc54)c3-c3nc(-c4ccccc4)nc(-c4ccccc4)n3)c21 ; C[Si]1(C)c2ccccc2-c2c1ccc(-c1c(-c3ccccc3)ccc3c1oc1ccccc13)c2-c1nc(-c2ccccc2)nc(-c2ccccc2)n1 ; c1ccc(-c2nc(-c3ccccc3)nc(-c3ccc4c(oc5ccccc54)c3-c3c(-c4ccc5ccccc5c4)ccc4c3oc3ccccc34)n2)cc1]

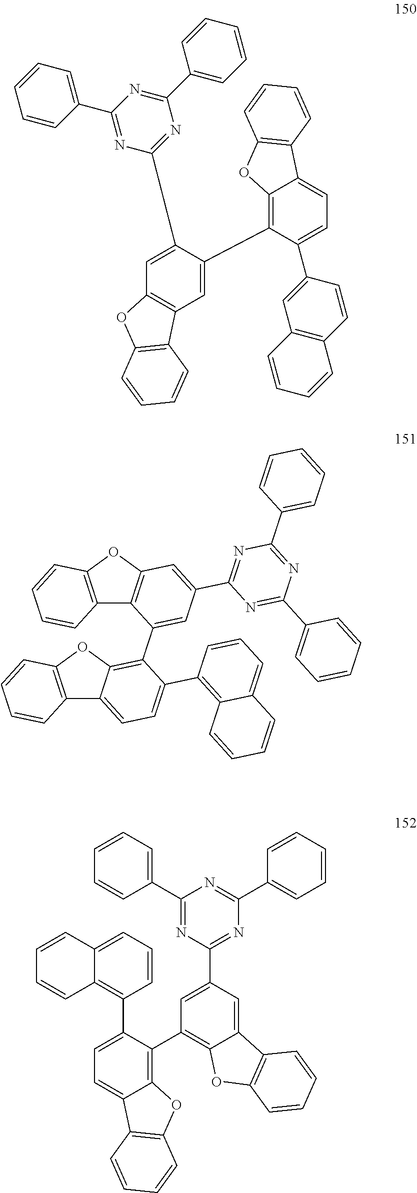 [SMILES: c1ccc(-c2nc(-c3ccccc3)nc(-c3cc4oc5ccccc5c4cc3-c3c(-c4ccc5ccccc5c4)ccc4c3oc3ccccc34)n2)cc1 ; c1ccc(-c2nc(-c3ccccc3)nc(-c3cc(-c4c(-c5cccc6ccccc56)ccc5c4oc4ccccc45)c4c(c3)oc3ccccc34)n2)cc1 ; c1ccc(-c2nc(-c3ccccc3)nc(-c3cc(-c4c(-c5cccc6ccccc56)ccc5c4oc4ccccc45)c4oc5ccccc5c4c3)n2)cc1]

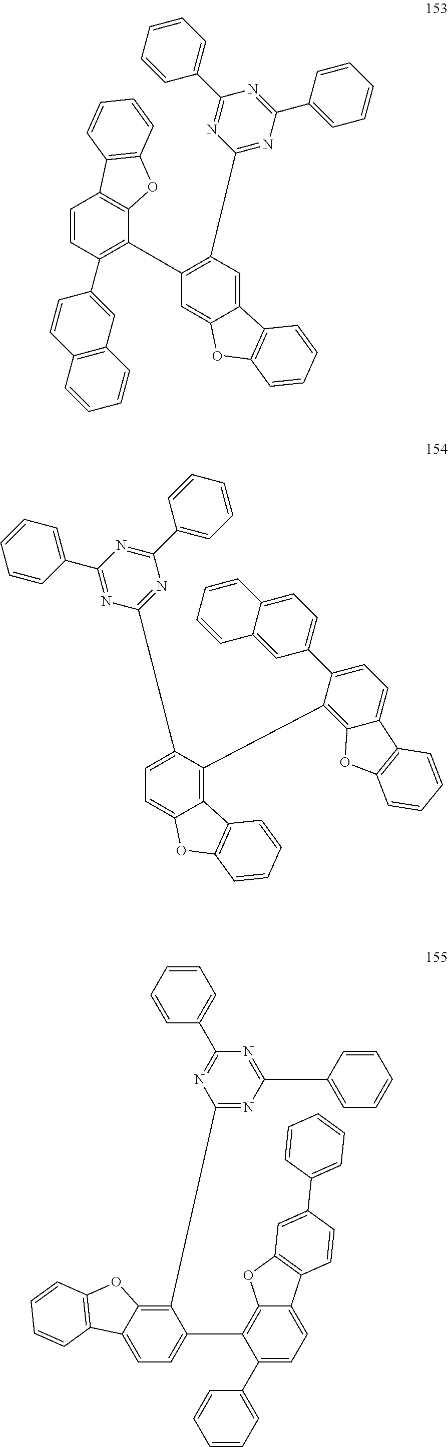 [SMILES: c1ccc(-c2nc(-c3ccccc3)nc(-c3cc4c(cc3-c3c(-c5ccc6ccccc6c5)ccc5c3oc3ccccc35)oc3ccccc34)n2)cc1 ; c1ccc(-c2nc(-c3ccccc3)nc(-c3ccc4oc5ccccc5c4c3-c3c(-c4ccc5ccccc5c4)ccc4c3oc3ccccc34)n2)cc1 ; c1ccc(-c2ccc3c(c2)oc2c(-c4ccc5c(oc6ccccc65)c4-c4nc(-c5ccccc5)nc(-c5ccccc5)n4)c(-c4ccccc4)ccc23)cc1]

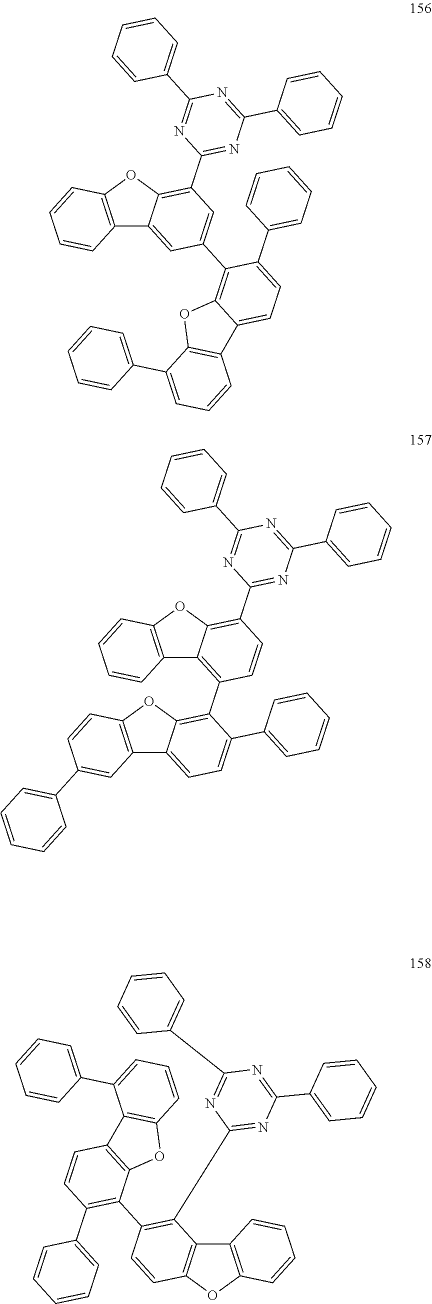

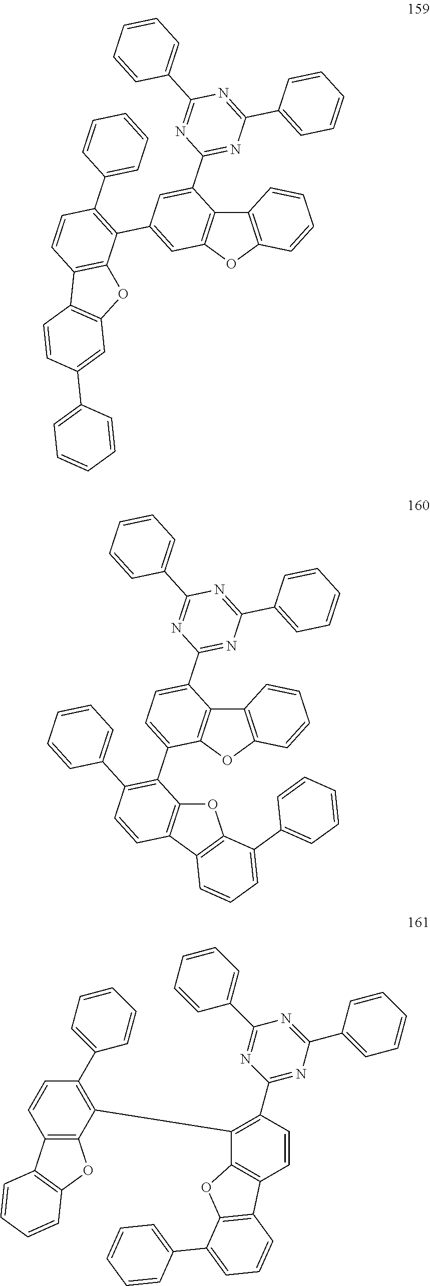 [SMILES: c1ccc(-c2ccc3c(c2)oc2c(-c4cc(-c5nc(-c6ccccc6)nc(-c6ccccc6)n5)c5c(c4)oc4ccccc45)c(-c4ccccc4)ccc23)cc1 ; c1ccc(-c2nc(-c3ccccc3)nc(-c3ccc(-c4c(-c5ccccc5)ccc5c4oc4c(-c6ccccc6)cccc45)c4oc5ccccc5c34)n2)cc1 ; c1ccc(-c2nc(-c3ccccc3)nc(-c3ccc4c(oc5c(-c6ccccc6)cccc54)c3-c3c(-c4ccccc4)ccc4c3oc3ccccc34)n2)cc1]

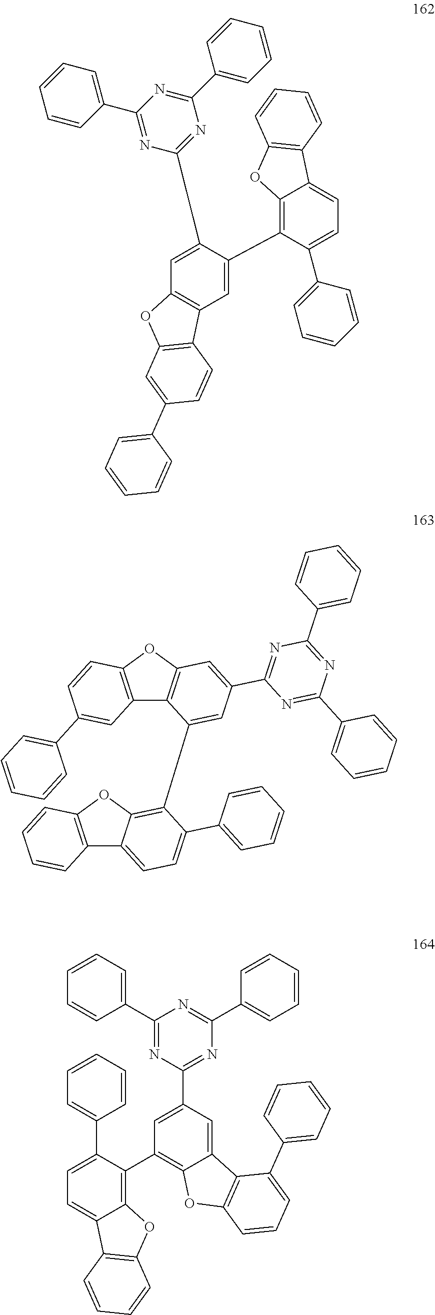 [SMILES: c1ccc(-c2ccc3c(c2)oc2cc(-c4nc(-c5ccccc5)nc(-c5ccccc5)n4)c(-c4c(-c5ccccc5)ccc5c4oc4ccccc45)cc23)cc1 ; c1ccc(-c2ccc3oc4cc(-c5nc(-c6ccccc6)nc(-c6ccccc6)n5)cc(-c5c(-c6ccccc6)ccc6c5oc5ccccc56)c4c3c2)cc1 ; c1ccc(-c2nc(-c3ccccc3)nc(-c3cc(-c4c(-c5ccccc5)ccc5c4oc4ccccc45)c4oc5cccc(-c6ccccc6)c5c4c3)n2)cc1]

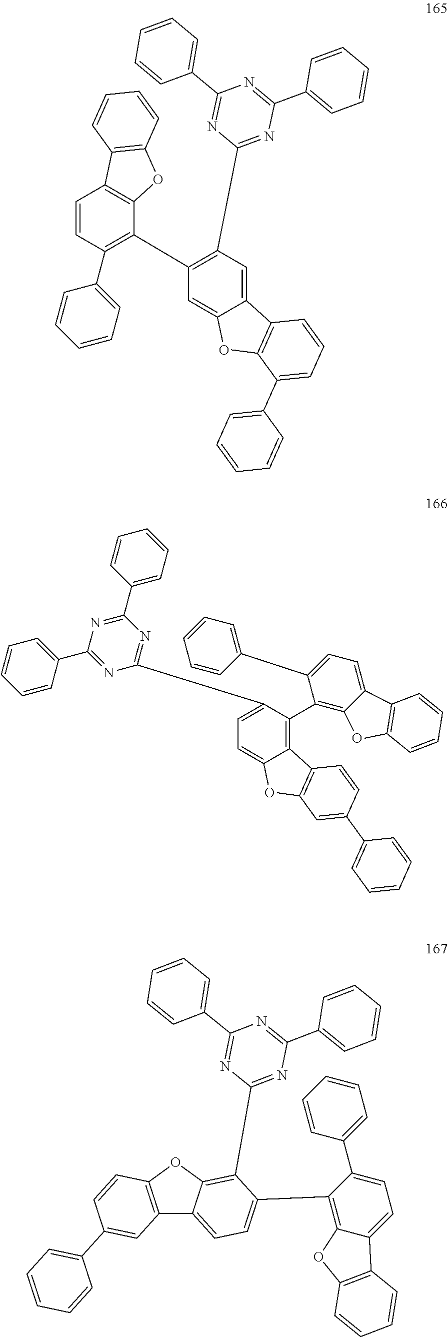 [SMILES: c1ccc(-c2nc(-c3ccccc3)nc(-c3cc4c(cc3-c3c(-c5ccccc5)ccc5c3oc3ccccc35)oc3c(-c5ccccc5)cccc34)n2)cc1 ; c1ccc(-c2ccc3c(c2)oc2ccc(-c4nc(-c5ccccc5)nc(-c5ccccc5)n4)c(-c4c(-c5ccccc5)ccc5c4oc4ccccc45)c23)cc1 ; c1ccc(-c2ccc3oc4c(-c5nc(-c6ccccc6)nc(-c6ccccc6)n5)c(-c5c(-c6ccccc6)ccc6c5oc5ccccc56)ccc4c3c2)cc1]

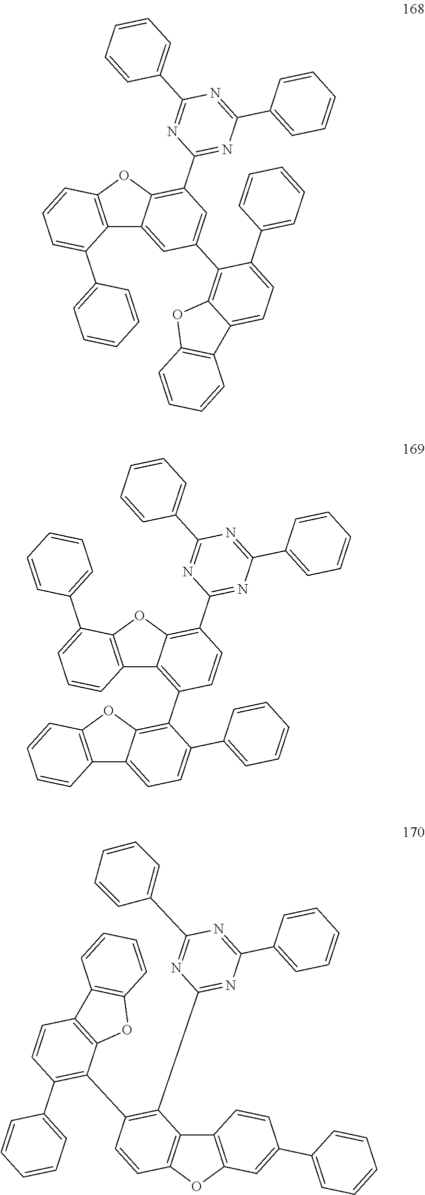 [SMILES: c1ccc(-c2nc(-c3ccccc3)nc(-c3cc(-c4c(-c5ccccc5)ccc5c4oc4ccccc45)cc4c3oc3cccc(-c5ccccc5)c34)n2)cc1 ; c1ccc(-c2nc(-c3ccccc3)nc(-c3ccc(-c4c(-c5ccccc5)ccc5c4oc4ccccc45)c4c3oc3c(-c5ccccc5)cccc34)n2)cc1 ; c1ccc(-c2ccc3c(c2)oc2ccc(-c4c(-c5ccccc5)ccc5c4oc4ccccc45)c(-c4nc(-c5ccccc5)nc(-c5ccccc5)n4)c23)cc1]

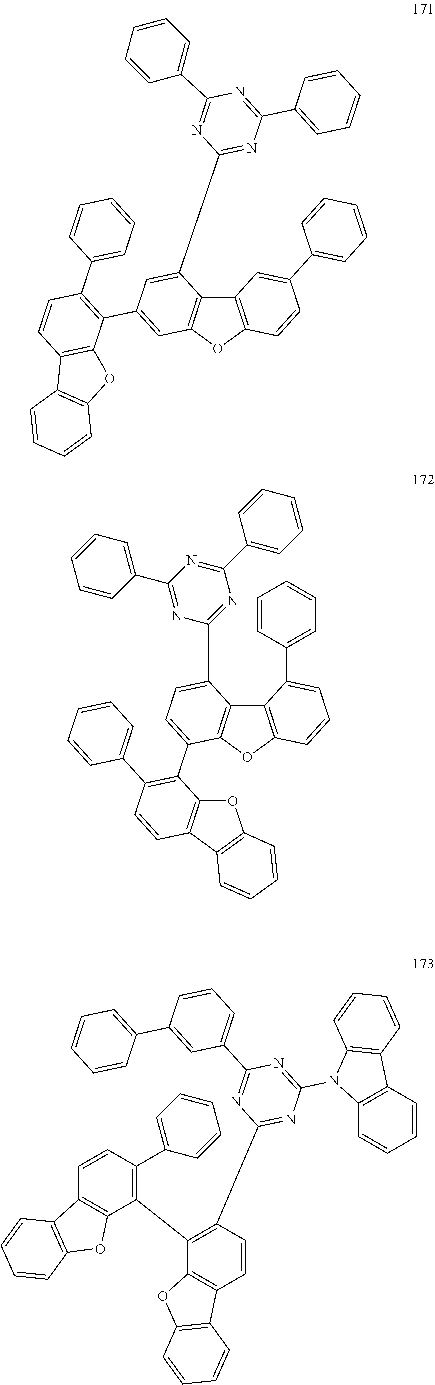 [SMILES: c1ccc(-c2ccc3oc4cc(-c5c(-c6ccccc6)ccc6c5oc5ccccc56)cc(-c5nc(-c6ccccc6)nc(-c6ccccc6)n5)c4c3c2)cc1 ; c1ccc(-c2nc(-c3ccccc3)nc(-c3ccc(-c4c(-c5ccccc5)ccc5c4oc4ccccc45)c4oc5cccc(-c6ccccc6)c5c34)n2)cc1 ; c1ccc(-c2cccc(-c3nc(-c4ccc5c(oc6ccccc65)c4-c4c(-c5ccccc5)ccc5c4oc4ccccc45)nc(-n4c5ccccc5c5ccccc54)n3)c2)cc1]

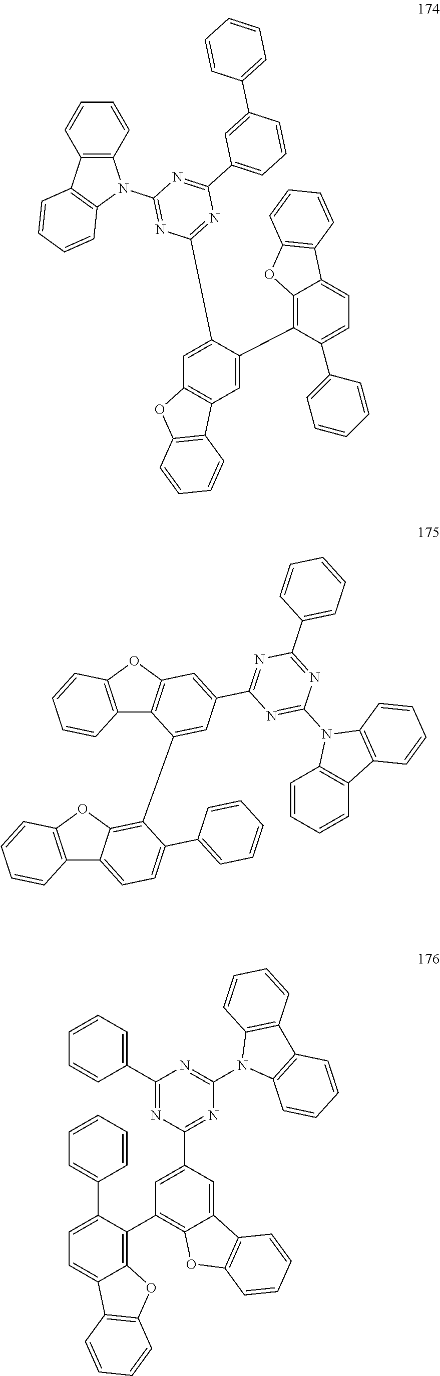 [SMILES: c1ccc(-c2cccc(-c3nc(-c4cc5oc6ccccc6c5cc4-c4c(-c5ccccc5)ccc5c4oc4ccccc45)nc(-n4c5ccccc5c5ccccc54)n3)c2)cc1 ; c1ccc(-c2nc(-c3cc(-c4c(-c5ccccc5)ccc5c4oc4ccccc45)c4c(c3)oc3ccccc34)nc(-n3c4ccccc4c4ccccc43)n2)cc1 ; c1ccc(-c2nc(-c3cc(-c4c(-c5ccccc5)ccc5c4oc4ccccc45)c4oc5ccccc5c4c3)nc(-n3c4ccccc4c4ccccc43)n2)cc1]

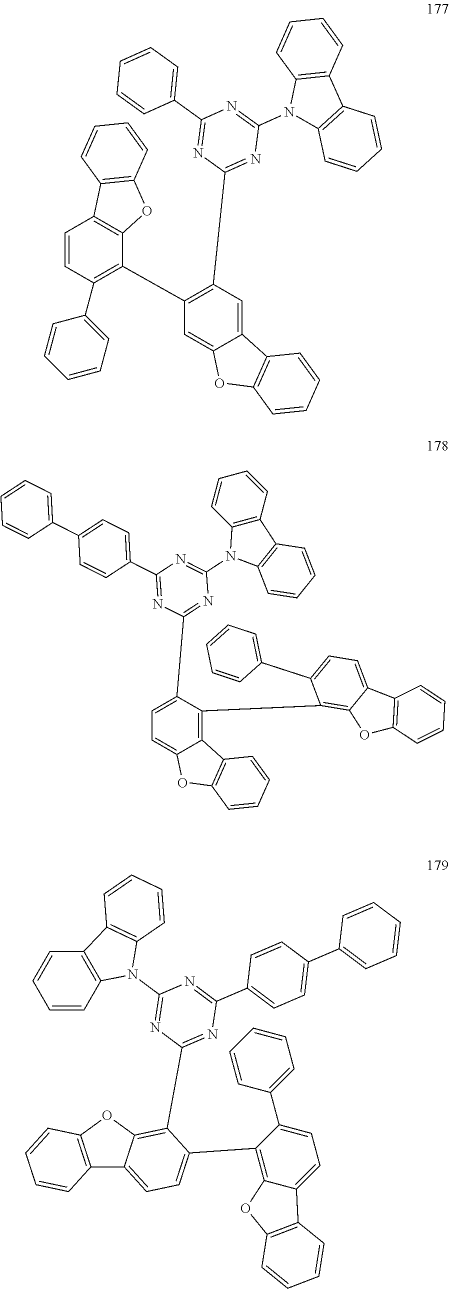 [SMILES: c1ccc(-c2nc(-c3cc4c(cc3-c3c(-c5ccccc5)ccc5c3oc3ccccc35)oc3ccccc34)nc(-n3c4ccccc4c4ccccc43)n2)cc1 ; c1ccc(-c2ccc(-c3nc(-c4ccc5oc6ccccc6c5c4-c4c(-c5ccccc5)ccc5c4oc4ccccc45)nc(-n4c5ccccc5c5ccccc54)n3)cc2)cc1 ; c1ccc(-c2ccc(-c3nc(-c4c(-c5c(-c6ccccc6)ccc6c5oc5ccccc56)ccc5c4oc4ccccc45)nc(-n4c5ccccc5c5ccccc54)n3)cc2)cc1]

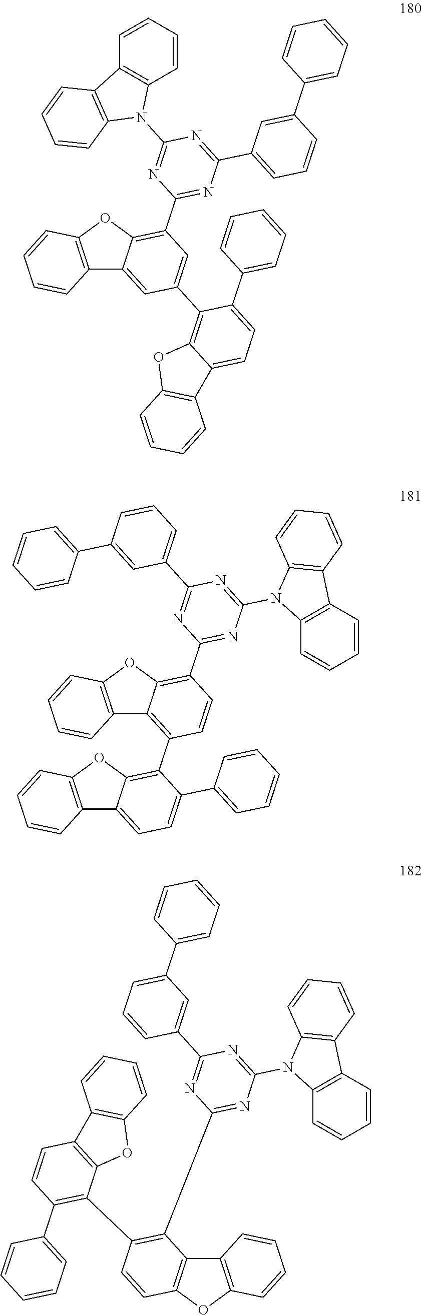 [SMILES: c1ccc(-c2cccc(-c3nc(-c4cc(-c5c(-c6ccccc6)ccc6c5oc5ccccc56)cc5c4oc4ccccc45)nc(-n4c5ccccc5c5ccccc54)n3)c2)cc1 ; c1ccc(-c2cccc(-c3nc(-c4ccc(-c5c(-c6ccccc6)ccc6c5oc5ccccc56)c5c4oc4ccccc45)nc(-n4c5ccccc5c5ccccc54)n3)c2)cc1 ; c1ccc(-c2cccc(-c3nc(-c4c(-c5c(-c6ccccc6)ccc6c5oc5ccccc56)ccc5oc6ccccc6c45)nc(-n4c5ccccc5c5ccccc54)n3)c2)cc1]

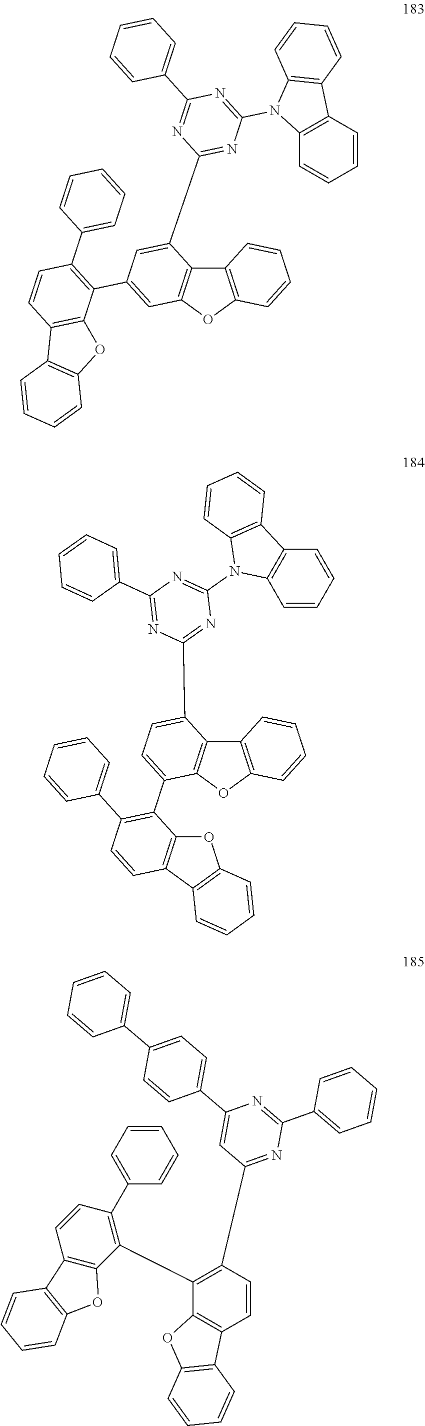 [SMILES: c1ccc(-c2nc(-c3cc(-c4c(-c5ccccc5)ccc5c4oc4ccccc45)cc4oc5ccccc5c34)nc(-n3c4ccccc4c4ccccc43)n2)cc1 ; c1ccc(-c2nc(-c3ccc(-c4c(-c5ccccc5)ccc5c4oc4ccccc45)c4oc5ccccc5c34)nc(-n3c4ccccc4c4ccccc43)n2)cc1 ; c1ccc(-c2ccc(-c3cc(-c4ccc5c(oc6ccccc65)c4-c4c(-c5ccccc5)ccc5c4oc4ccccc45)nc(-c4ccccc4)n3)cc2)cc1]

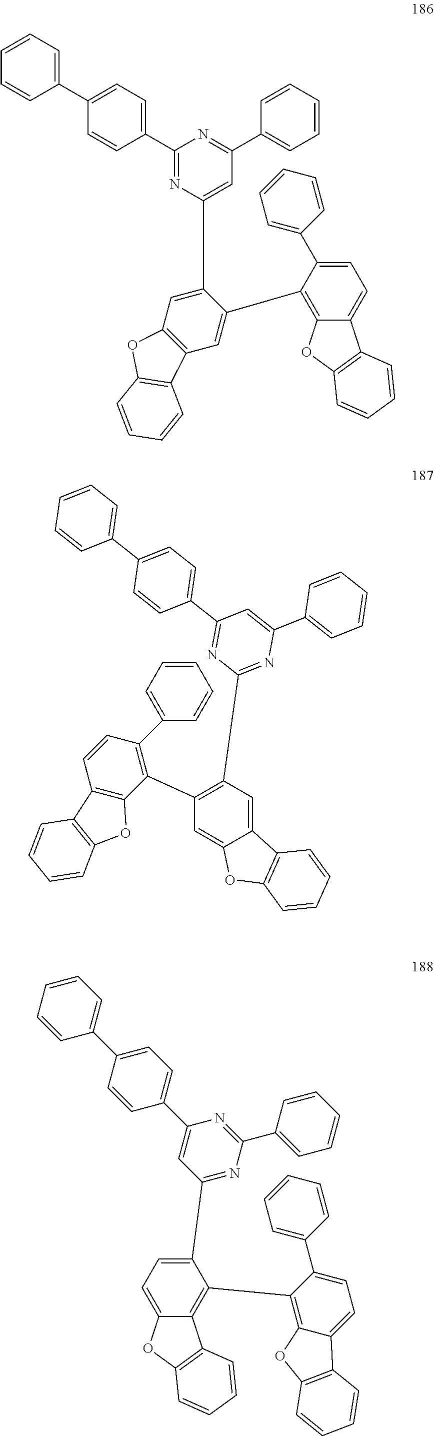 [SMILES: c1ccc(-c2ccc(-c3nc(-c4ccccc4)cc(-c4cc5oc6ccccc6c5cc4-c4c(-c5ccccc5)ccc5c4oc4ccccc45)n3)cc2)cc1 ; c1ccc(-c2ccc(-c3cc(-c4ccccc4)nc(-c4cc5c(cc4-c4c(-c6ccccc6)ccc6c4oc4ccccc46)oc4ccccc45)n3)cc2)cc1 ; c1ccc(-c2ccc(-c3cc(-c4ccc5oc6ccccc6c5c4-c4c(-c5ccccc5)ccc5c4oc4ccccc45)nc(-c4ccccc4)n3)cc2)cc1]

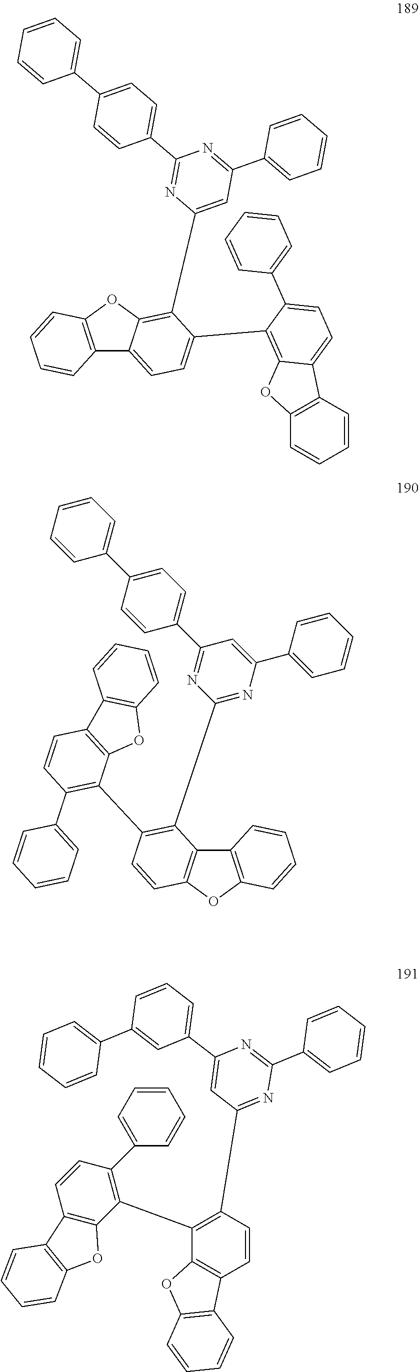 [SMILES: c1ccc(-c2ccc(-c3nc(-c4ccccc4)cc(-c4c(-c5c(-c6ccccc6)ccc6c5oc5ccccc56)ccc5c4oc4ccccc45)n3)cc2)cc1 ; c1ccc(-c2ccc(-c3cc(-c4ccccc4)nc(-c4c(-c5c(-c6ccccc6)ccc6c5oc5ccccc56)ccc5oc6ccccc6c45)n3)cc2)cc1 ; c1ccc(-c2cccc(-c3cc(-c4ccc5c(oc6ccccc65)c4-c4c(-c5ccccc5)ccc5c4oc4ccccc45)nc(-c4ccccc4)n3)c2)cc1]

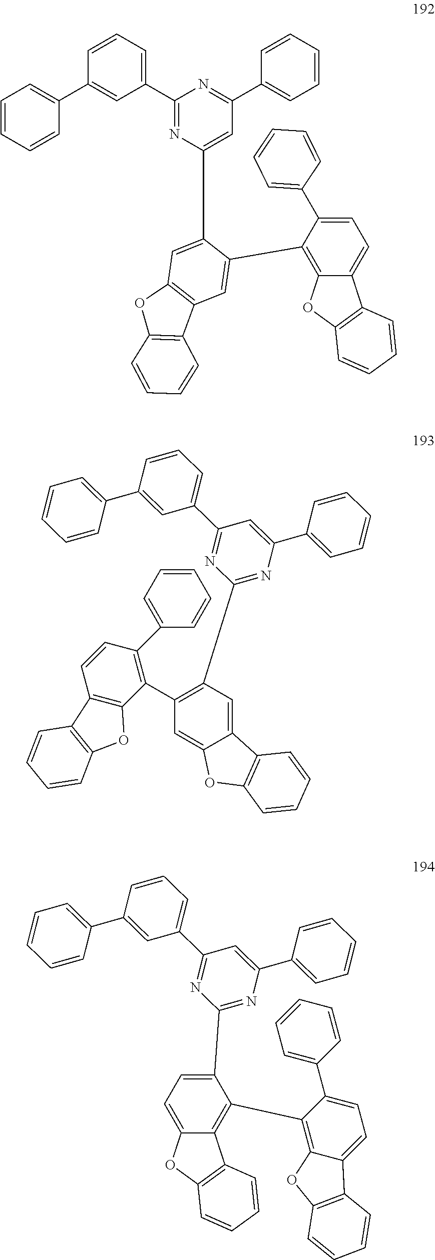 [SMILES: c1ccc(-c2cccc(-c3nc(-c4ccccc4)cc(-c4cc5oc6ccccc6c5cc4-c4c(-c5ccccc5)ccc5c4oc4ccccc45)n3)c2)cc1 ; c1ccc(-c2cccc(-c3cc(-c4ccccc4)nc(-c4cc5c(cc4-c4c(-c6ccccc6)ccc6c4oc4ccccc46)oc4ccccc45)n3)c2)cc1 ; c1ccc(-c2cccc(-c3cc(-c4ccccc4)nc(-c4ccc5oc6ccccc6c5c4-c4c(-c5ccccc5)ccc5c4oc4ccccc45)n3)c2)cc1]

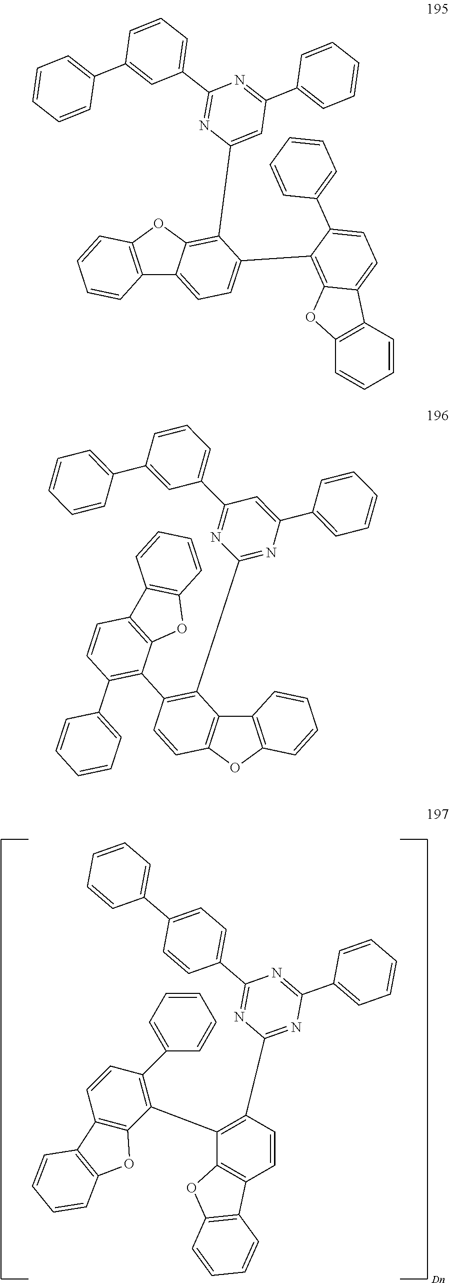

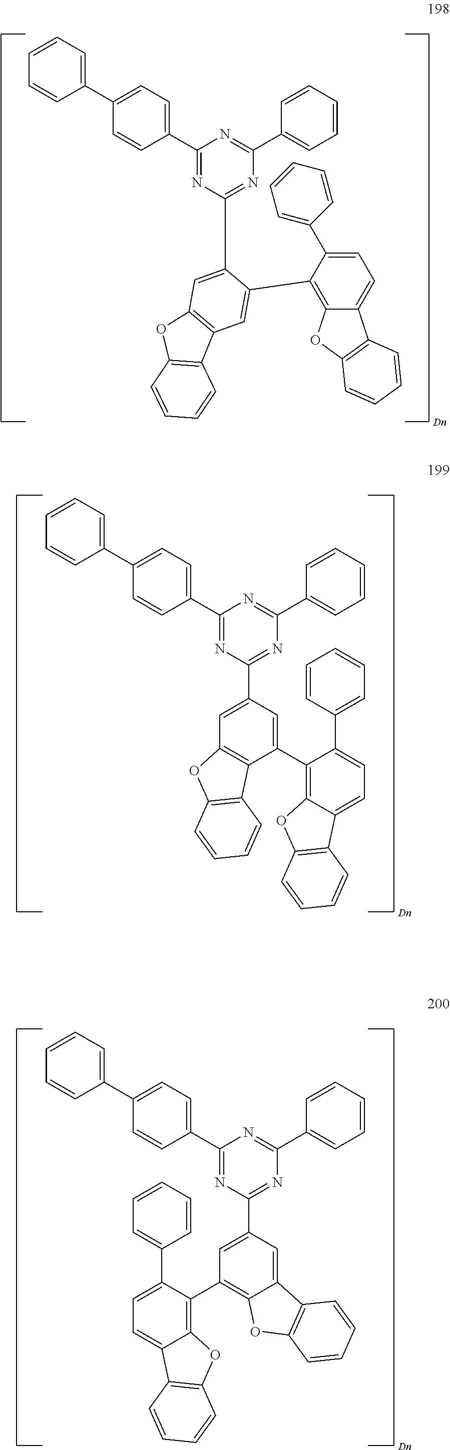

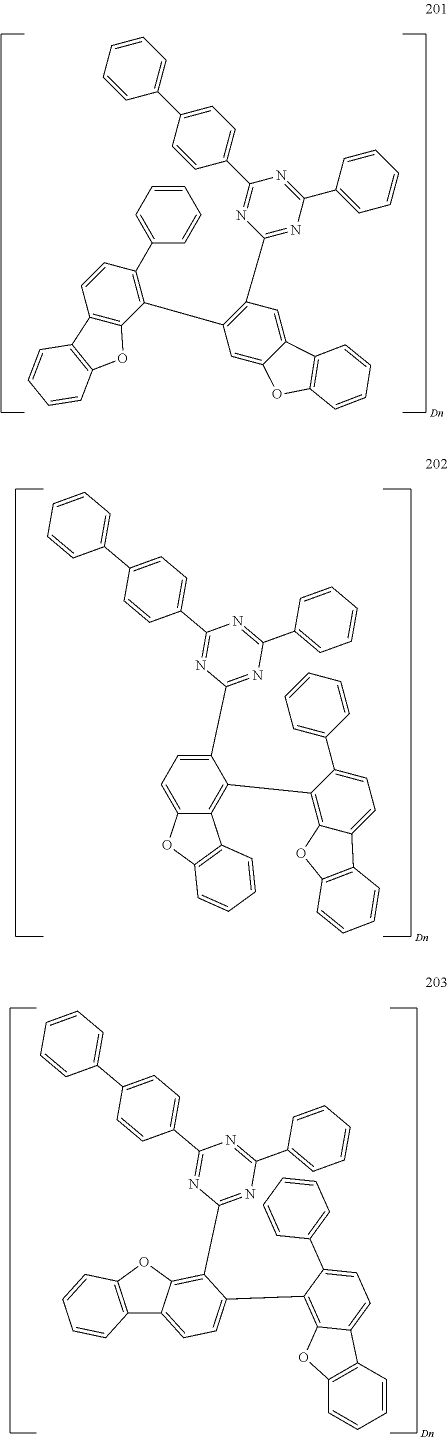

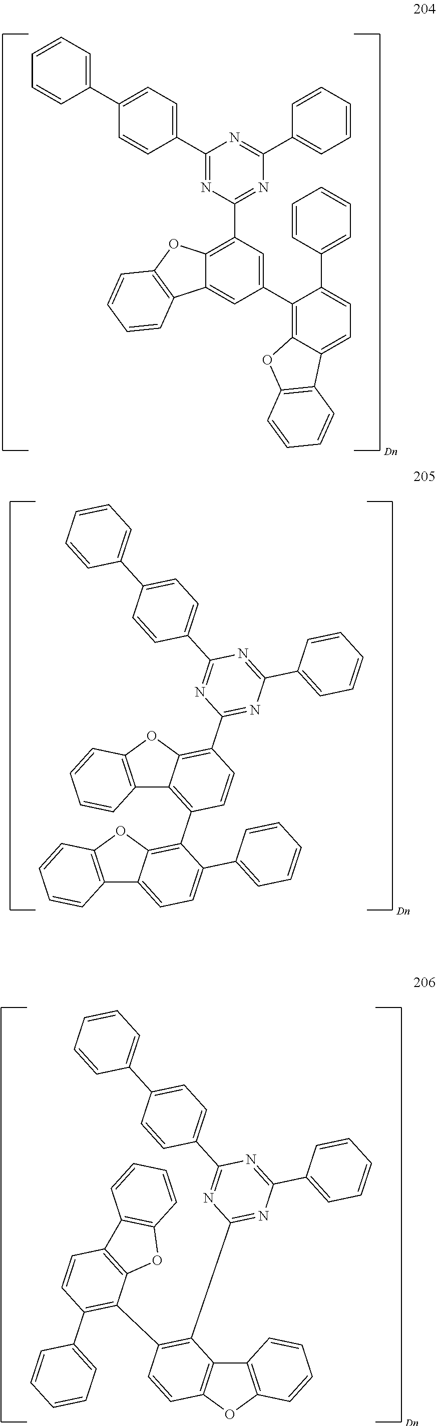

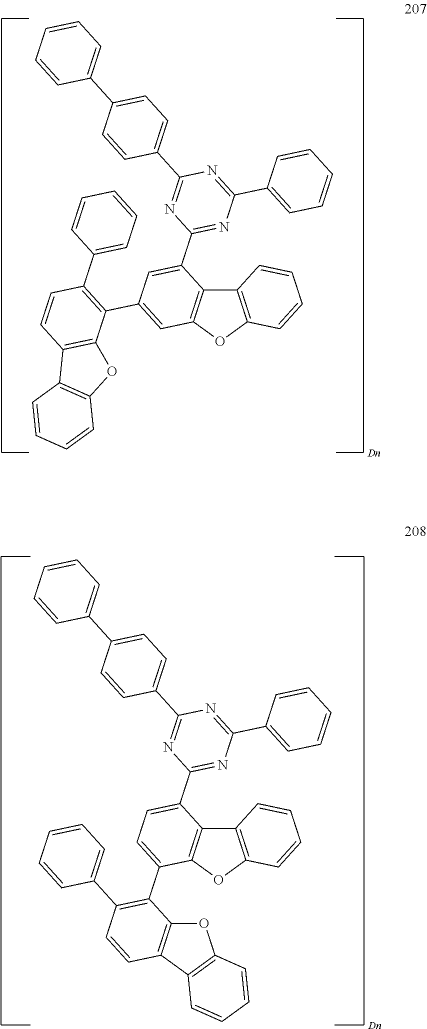

(Dn refers to the number of deuterium atoms substituted, and n is an integer greater than or equal to 1)

A composition for an organic optoelectronic device according to some example embodiments may include a first compound, and a second compound, wherein the first compound may be the aforementioned compound for an organic optoelectronic device and the second compound may be represented by Chemical Formula 2; a combination of Chemical Formula 3 and Chemical Formula 4; or Chemical Formula 5.

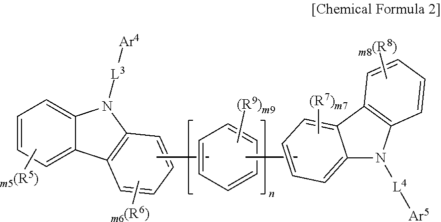

In Chemical Formula 2, R⁸ to R⁹ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁴ and Ar⁵ may each independently be, e.g., a substituted or unsubstituted C6 to C20 aryl group or a substituted or unsubstituted C2 to C30 heterocyclic group.

L³ and L⁴ may each independently be, e.g., a single bond or a substituted or unsubstituted C6 to C20 arylene group.

m5, m8, and m9 may each independently be, e.g., an integer of 1 to 4.

m6 and m7 may each independently be, e.g., an integer of 1 to 3.

n may be an integer of 0 to 2.

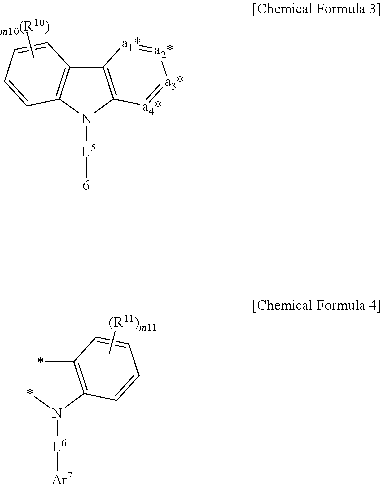

In Chemical Formula 3 and Chemical Formula 4, two adjacent ones of a₁* to a₄* of Chemical Formula 3, may be linking carbons linked at * of Chemical Formula 4.

The remaining two of a₁* to a₄* of Chemical Formula 3 that are not linked at * of Chemical Formula 4 may be, e.g., C-L^{a}-R^{d}.

L^{a}, L⁵, and L⁶ may each independently be, e.g., a single bond or a substituted or unsubstituted C6 to C20 arylene group.

R^{d}, R¹⁰, and R¹¹ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁶ and Ar⁷ may each independently be, e.g., a substituted or unsubstituted C6 to C20 aryl group or a substituted or unsubstituted C2 to C30 heterocyclic group.

m10 and m11 may each independently be, e.g., an integer of 1 to 4.

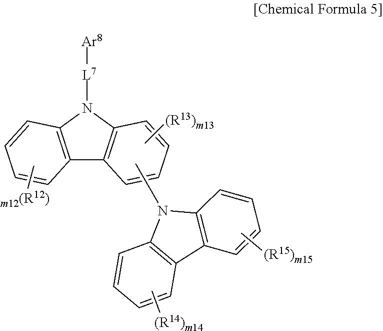

In Chemical Formula 5, L⁷ may be, e.g., a single bond or a substituted or unsubstituted C6 to C20 arylene group.

R¹² to R¹⁵ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁸ may be, e.g., a substituted or unsubstituted C6 to C20 aryl group or a substituted or unsubstituted C2 to C30 heterocyclic group.

m12, m14, and m15 may each independently be, e.g., an integer of 1 to 4.

m13 may be, e.g., an integer of 1 to 3.

The second compound may be used in the light emitting layer together with the first compound to improve luminous efficiency and life-span characteristics by increasing charge mobility and stability.

In an implementation, m5 may be 2, 3, or 4, and each R⁵ may be the same or different from each other.

In an implementation, m6 may be 2 or 3, and each R⁶ may be the same or different from each other.

In an implementation, m7 may be 2 or 3, and each R⁷ may be the same or different from each other.

In an implementation, m8 may be 2, 3, or 4, and each R⁸ may be the same or different from each other.

In an implementation, m9 may be 2, 3, or 4, and each R⁹ may be the same or different from each other.

In an implementation, m10 may be 2, 3, or 4, and each R¹⁰ may be the same or different from each other.

In an implementation, m11 may be 2, 3, or 4, and each R¹¹ may be the same or different from each other.

In an implementation, R^{d} may be present two or more times, and each R^{d} may be the same or different from each other.

In an implementation, m12 may be 2, 3, or 4, and each R¹² may be the same or different from each other.

In an implementation, m13 may be 2 or 3, and each R¹³ may be the same or different from each other.

In an implementation, m14 may be 2, 3, or 4, and each R¹⁴ may be the same or different from each other.

In an implementation, m15 may be 2, 3, or 4, and each R¹⁵ may be the same or different from each other.

In an implementation, in Chemical Formula 2, Ar⁴ and Ar⁵ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In Chemical Formula 2, L³ and L⁴ may each independently be a single bond, a substituted or unsubstituted phenylene group, or a substituted or unsubstituted biphenylene group.

In Chemical Formula 2, R⁸ to R⁹ may each independently be hydrogen, deuterium, or a substituted or unsubstituted C6 to C12 aryl group.

n may be 0 or 1.

In an implementation, in Chemical Formula 2, “substituted” may refer to replacement of at least one hydrogen by deuterium, a C1 to C4 alkyl group, a C6 to C18 aryl group, or a C2 to C30 heteroaryl group.

In an implementation, in Chemical Formula 2, Ar⁴ and Ar⁵ may each independently be a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by one of Chemical Formula 2-1 to Chemical Formula 2-15.

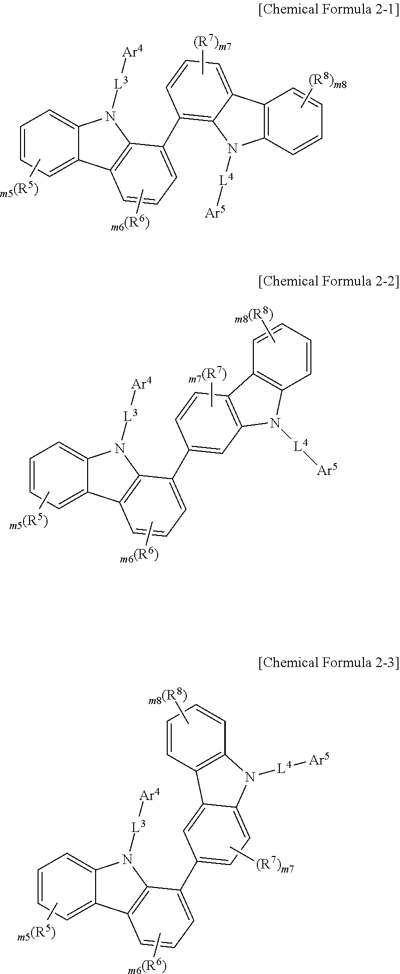

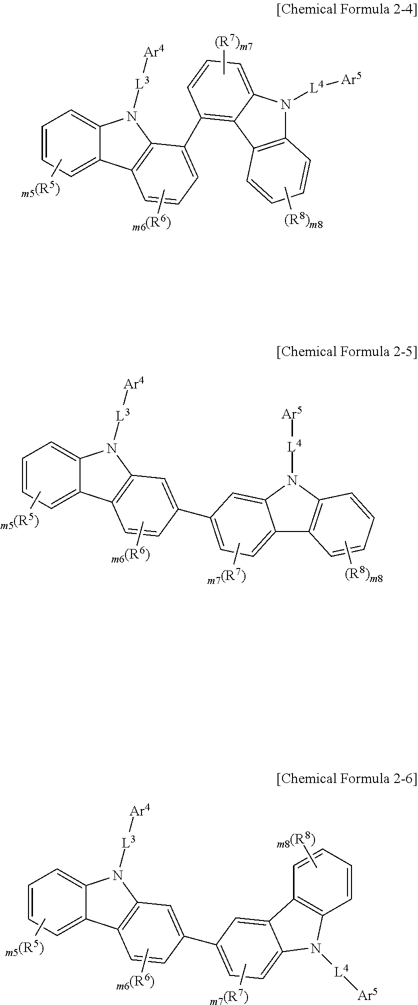

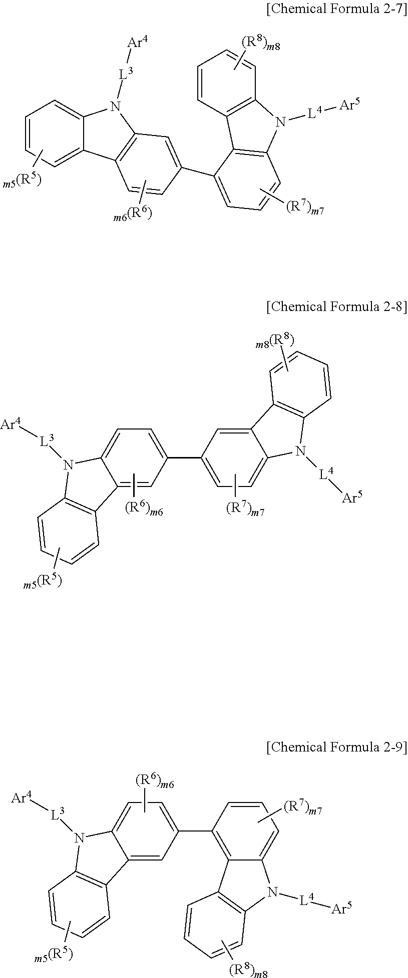

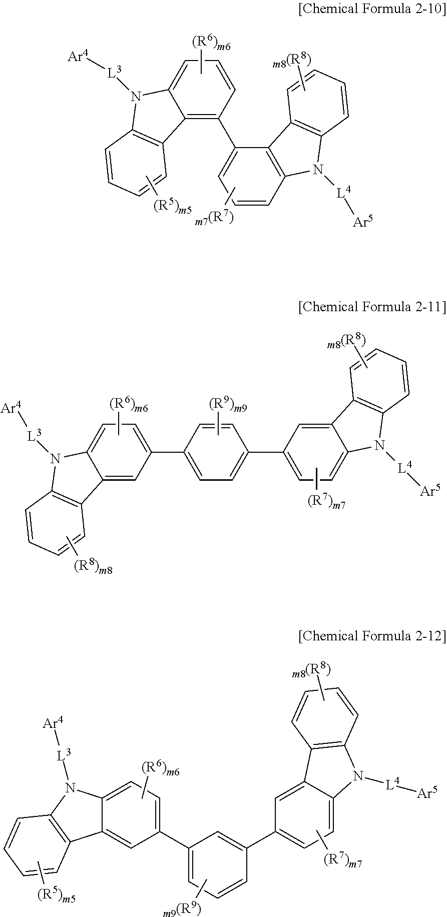

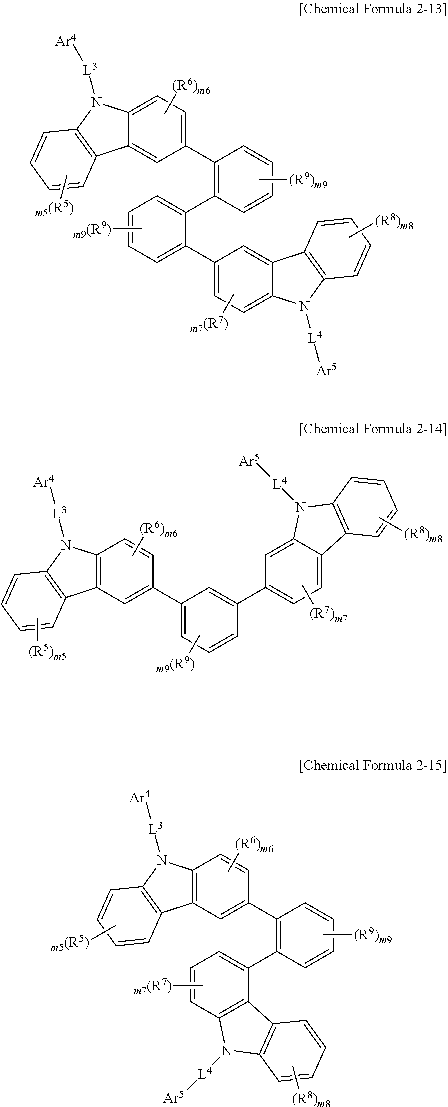

In Chemical Formula 2-1 to Chemical Formula 2-15, R⁵ to R⁹ may each independently be hydrogen, deuterium, or a substituted or unsubstituted C6 to C12 aryl group, and moieties *-L³-Ar⁴ and *-L⁴-Ar⁵ may each independently be a moiety of Group I.

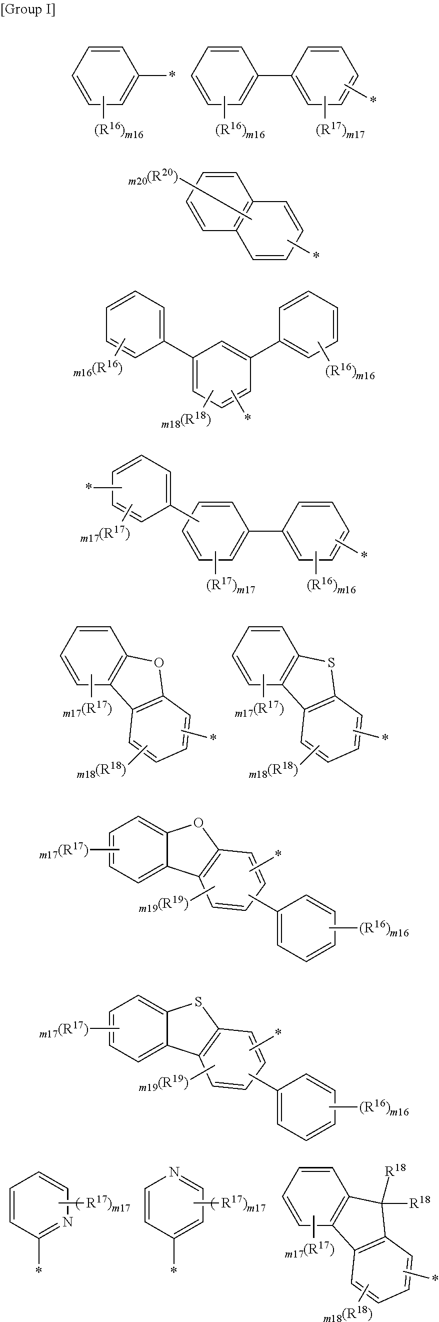

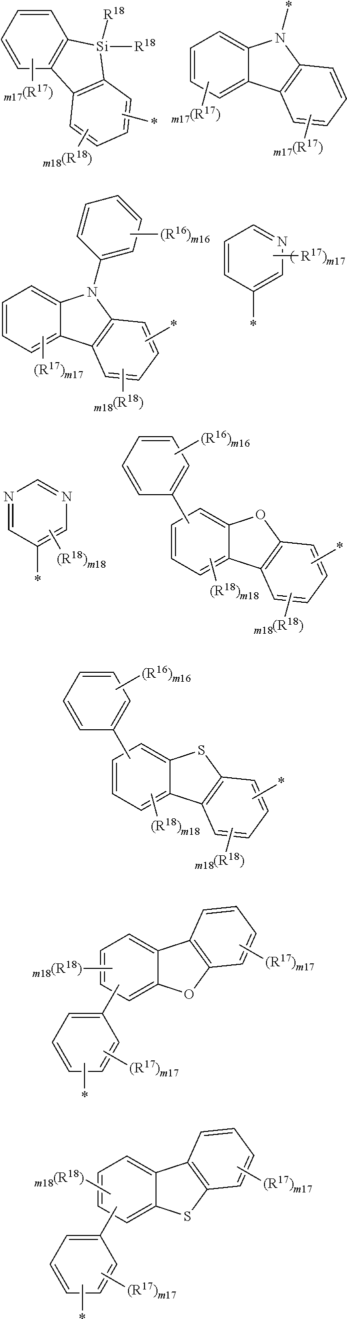

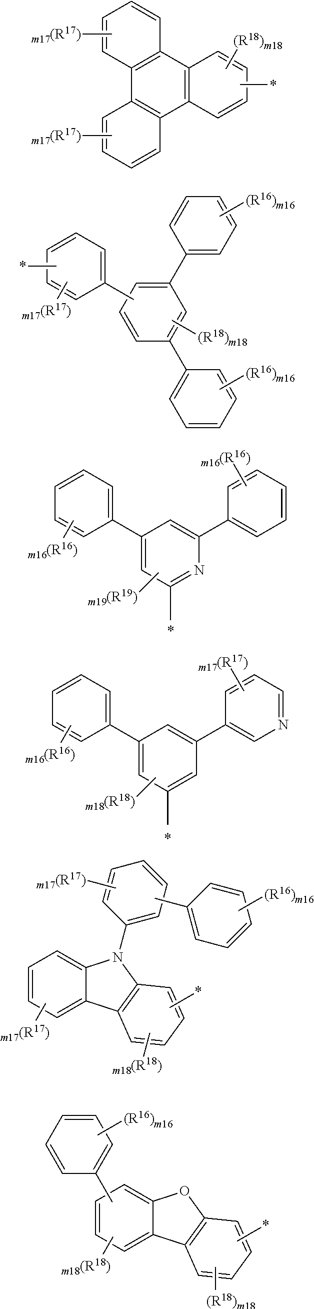

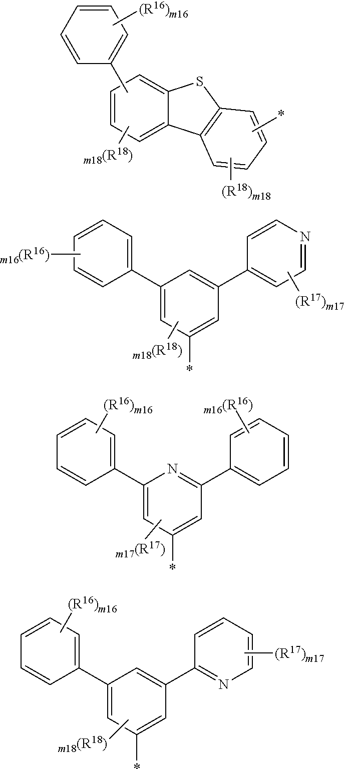

In Group I, R¹⁶ to R²⁰ may each independently be, e.g., hydrogen, deuterium, a cyano group, a substituted or unsubstituted C1 to C10 alkyl group, or a substituted or unsubstituted C6 to C12 aryl group.

m16 may be, e.g., an integer of 1 to 5.

m17 may be, e.g., an integer of 1 to 4.

m18 may be, e.g., an integer of 1 to 3.

m19 may be, e.g., an integer of 1 or 2.

m20 may be, e.g., an integer of 1 to 7.

* is a linking point.

In an implementation, m16 may be 2, 3, 4, or 5, and each R¹⁶ may be the same or different from each other.

In an implementation, m17 may be 2, 3, or 4, and each R¹⁷ may be the same or different from each other.

In an implementation, m18 may be 2 or 3, and each R¹⁸ may be the same or different from each other.

In an implementation, m19 may be 2, and each R¹⁹ may be the same or different from each other.

In an implementation, m20 may be 2, 3, 4, 5, 6, or 7, and each R²⁰ may be the same or different from each other.

The combination of Chemical Formula 3 and Chemical Formula 4 may be represented, e.g., by one of Chemical Formula 3A, Chemical Formula 3B, Chemical Formula 3C, Chemical Formula 3D, or Chemical Formula 3E.

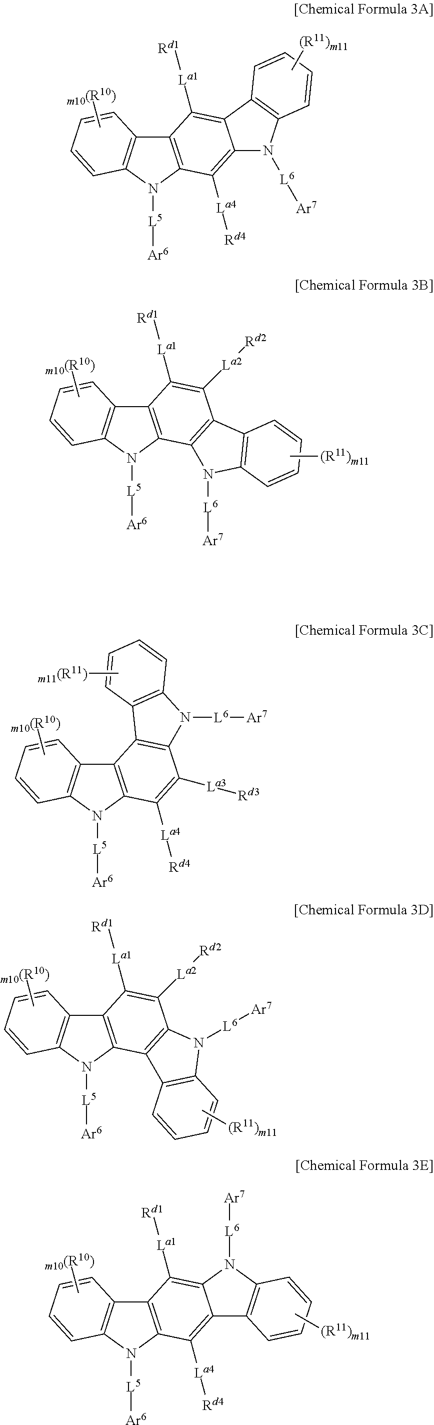

In Chemical Formula 3A to Chemical Formula 3E, L⁵, L⁶, Ar⁶, Ar⁷, R¹⁰, R¹¹, m10, and m11 may be defined the same as those described above,

L^{a1} to L^{a4} may be defined the same as L⁵ and L⁶, and

R^{d1} to R^{d4} may be defined the same as R¹⁰ and R¹¹.

In an implementation, in Chemical Formulas 3 and 4, Ar⁶ and Ar⁷ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R^{d1} to R^{d4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formulas 3 and 4, moieties *-L⁵-Ar⁶ and *-L⁶-Ar⁷ may each independently be, e.g., a moiety of Group I.

In an implementation, R^{d1} to R^{d4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{d1} to R^{d4}, R¹⁰, and R¹¹ may each independently be hydrogen, deuterium, a cyano group, or a substituted or unsubstituted phenyl group.

In an implementation, R^{d1} to R^{d4}, R¹⁰, and R¹¹ may each independently be hydrogen, deuterium, or a substituted or unsubstituted phenyl group.

In an implementation, the second compound may be represented by Chemical Formula 3C, and in Chemical Formula 3C, L^{a3} and L^{a4} may each be a single bond, L⁵ and L⁶ may each independently be, e.g., a single bond or a substituted or unsubstituted C6 to C12 arylene group, R¹⁰, R¹¹, R^{d3}, and R^{d4} may each independently be, e.g., hydrogen, deuterium, or a phenyl group, and Ar⁶ and Ar⁷ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3C, L^{a3} and L^{a4} may each be a single bond, R¹⁰, R¹¹, R^{d3}, and R^{d4} may each independently be, e.g., hydrogen, deuterium, or a substituted or unsubstituted C6 to C12 aryl group, and moieties *-L⁵-Ar⁶ and *-L⁶-Ar⁷ may each independently be, e.g., a moiety of Group I.

Chemical Formula 5 may be, e.g., represented by one of Chemical Formula 5-1 to Chemical Formula 5-4.

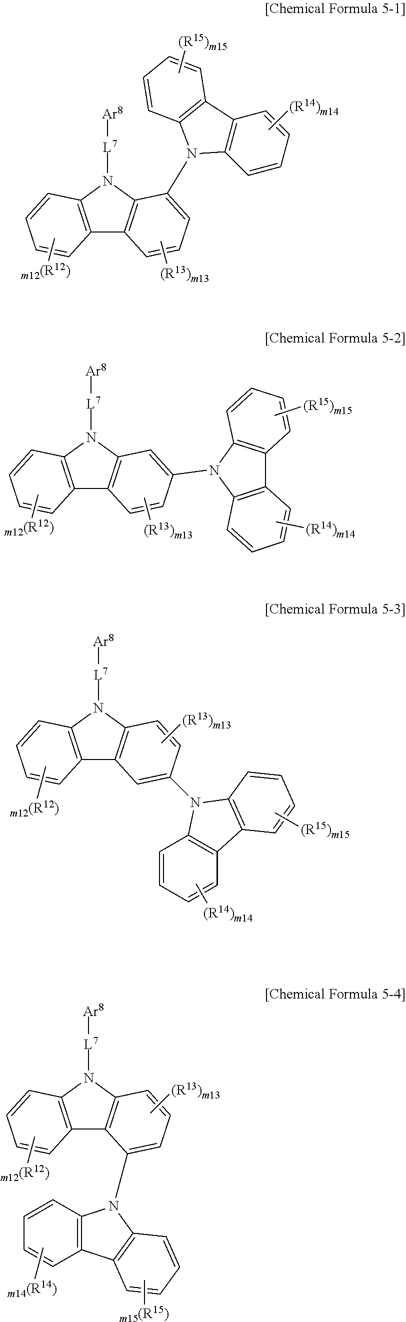

In Chemical Formula 5-1 to Chemical Formula 5-4, L⁷, Ar⁸, R¹² to R¹⁵, and m12 to m15 may be defined the same as those described above.

In an implementation, in Chemical Formula 5, Ar¹ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R¹² to R¹⁵ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, moiety *-L⁷-Ar¹ may be, e.g., a moiety of Group I.

In an implementation, R¹² to R¹⁵ may each independently be, e.g., hydrogen, deuterium, a cyano group, or a substituted or unsubstituted phenyl group.

In an implementation, the second compound for an organic optoelectronic device may be a compound of Group 2.

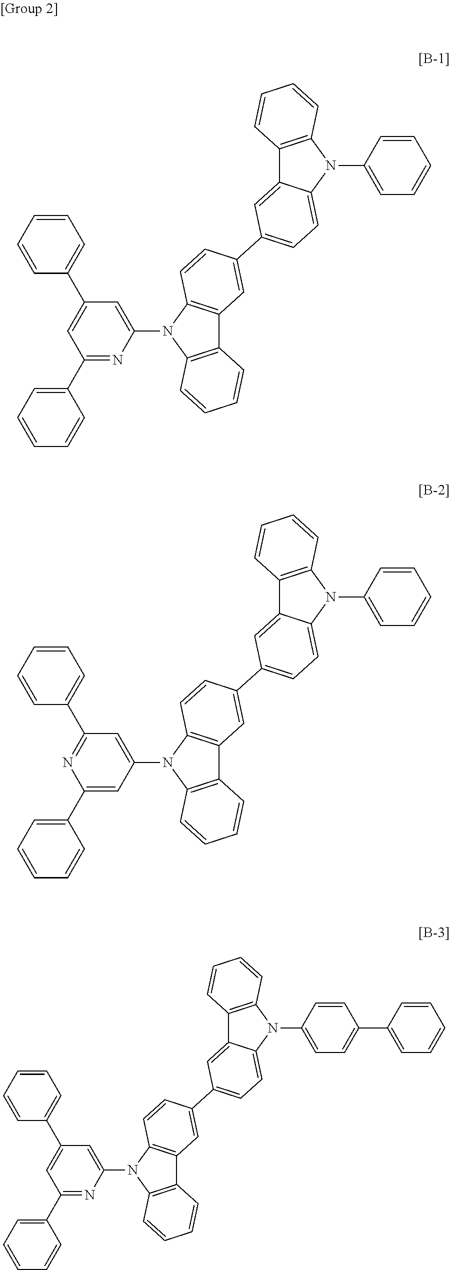 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

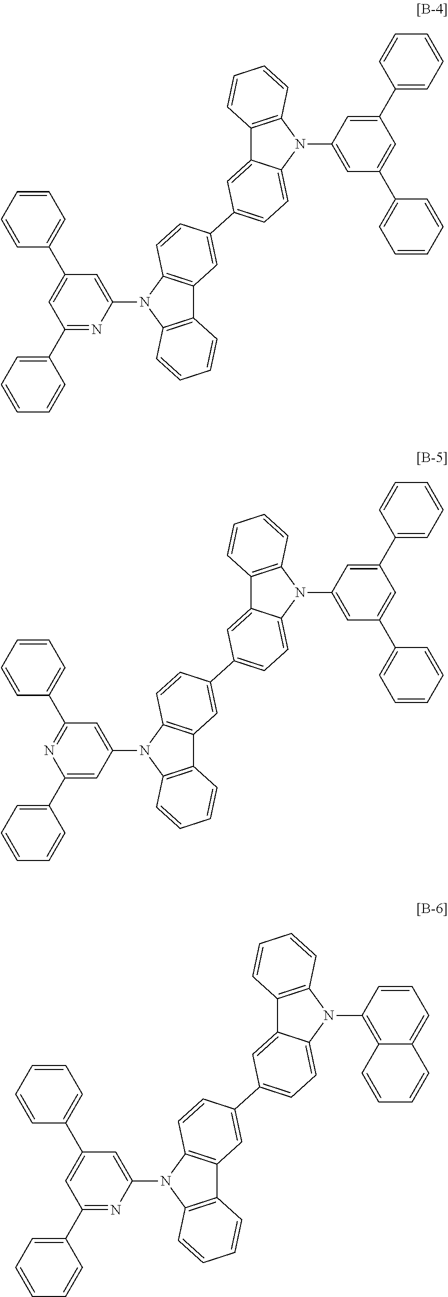 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

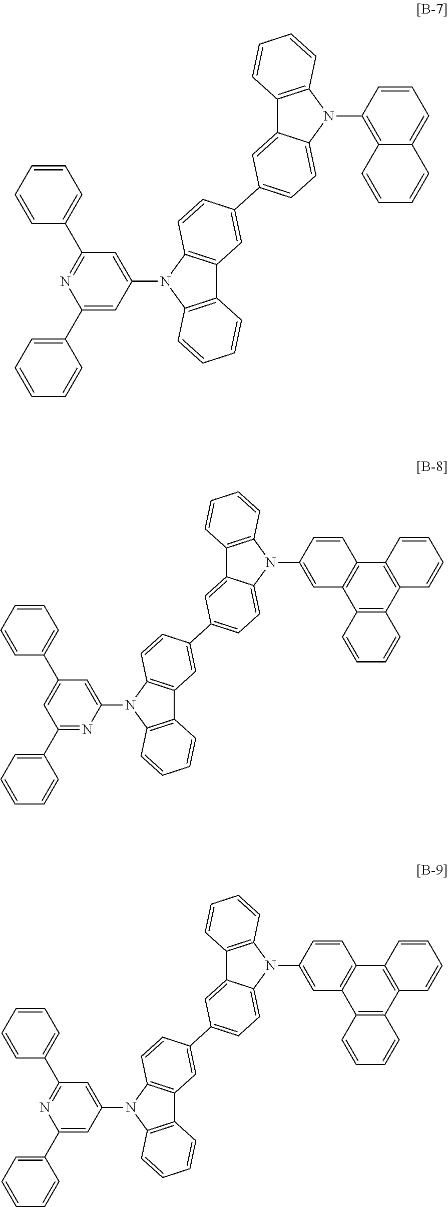 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1]

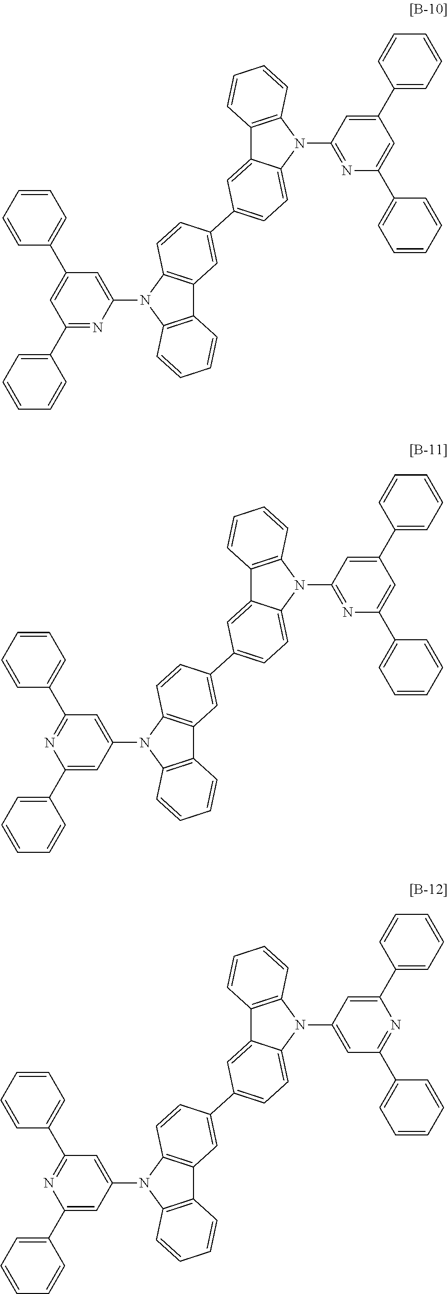 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

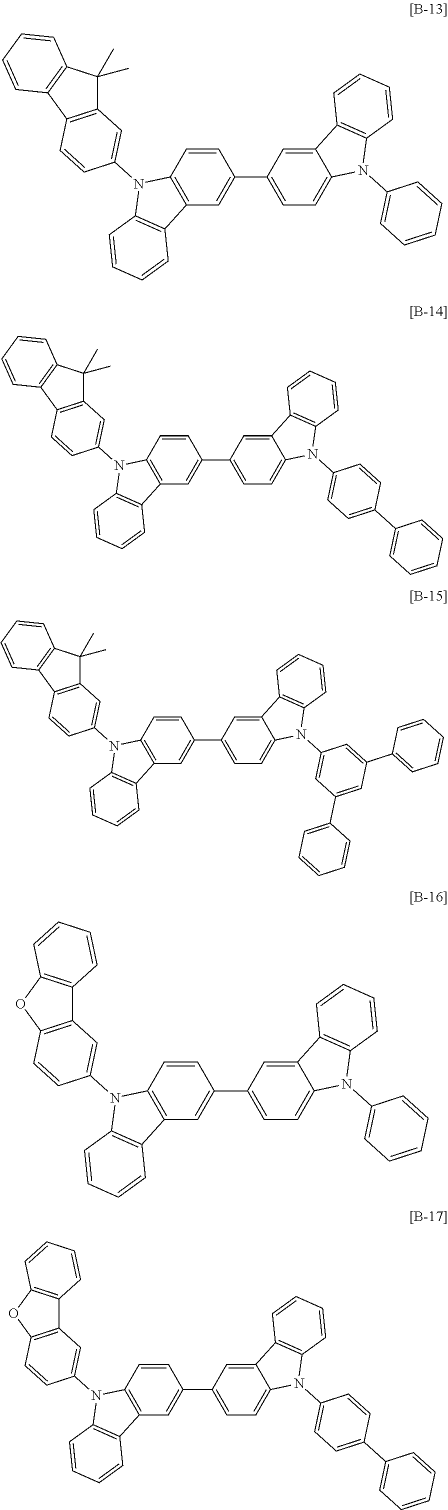 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

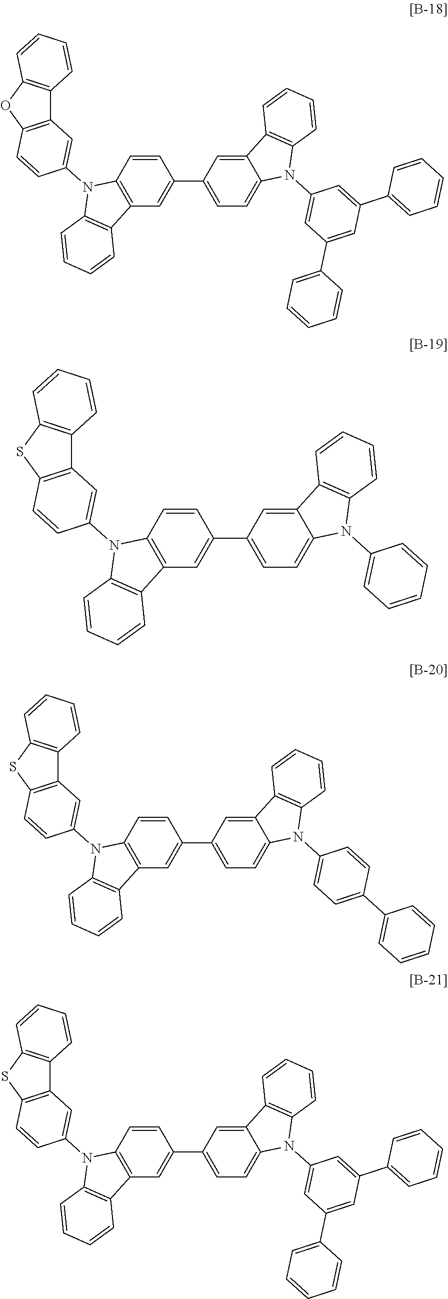 [SMILES: c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

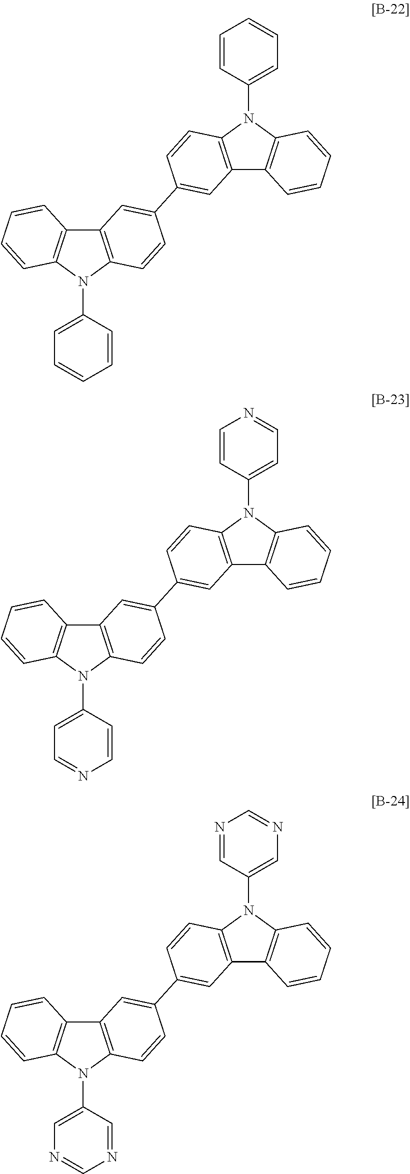 [SMILES: c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1]

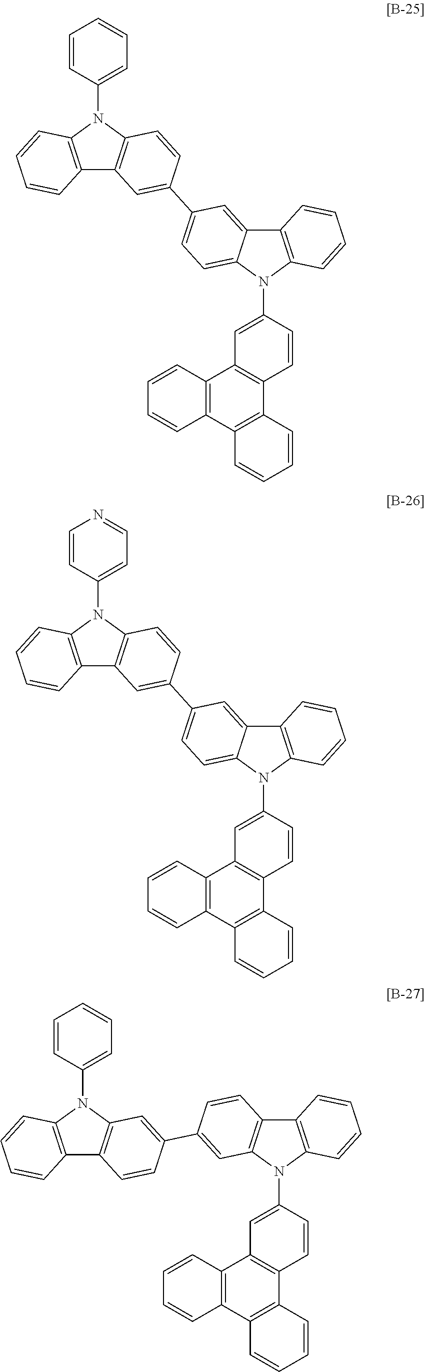 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1]

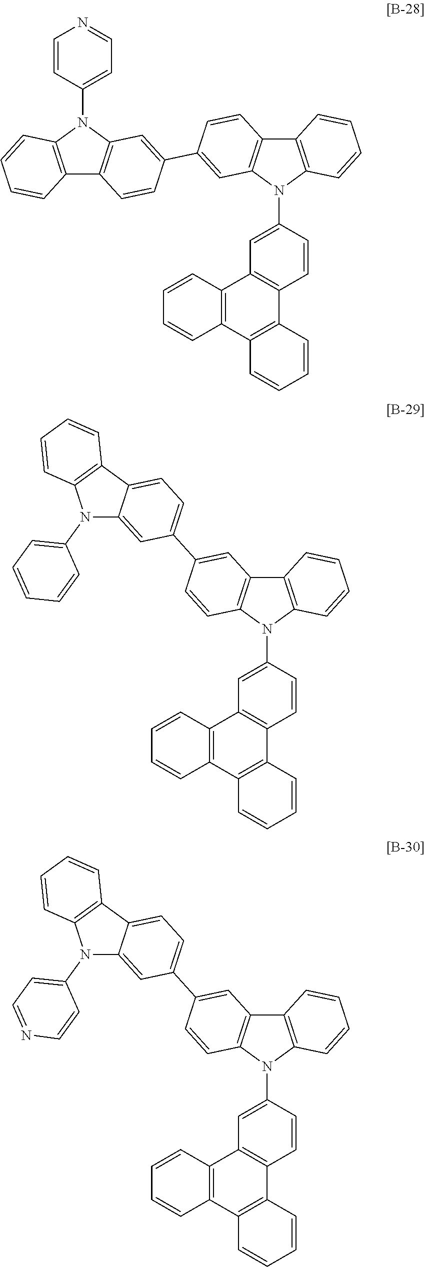 [SMILES: c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

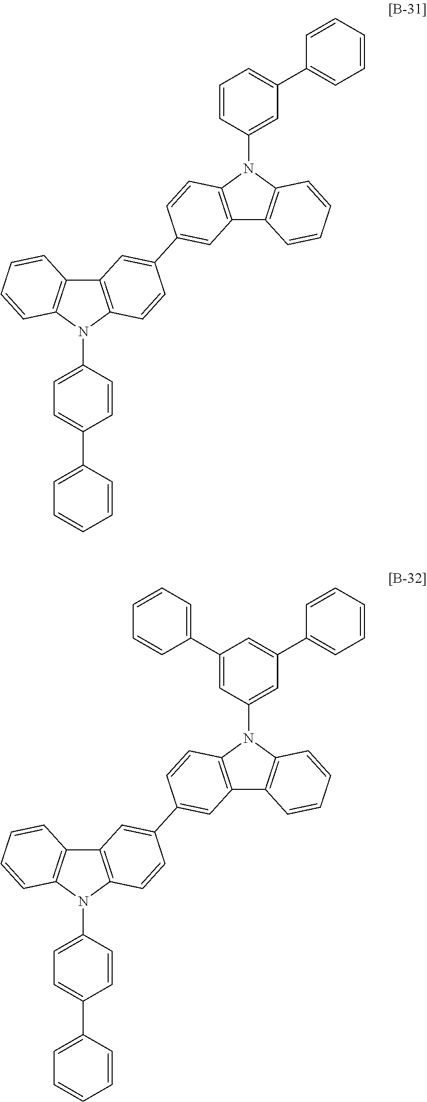 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

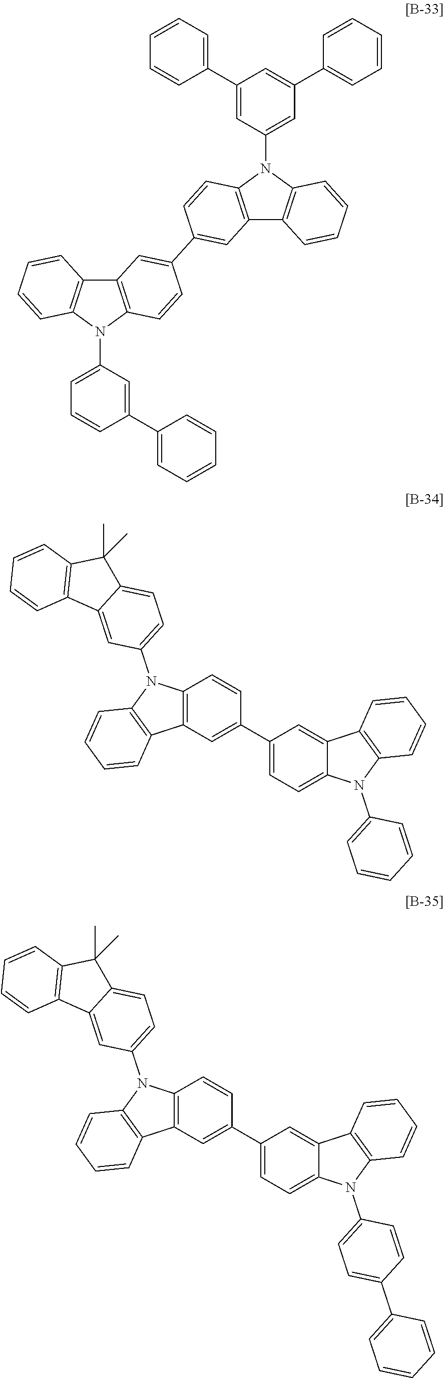 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

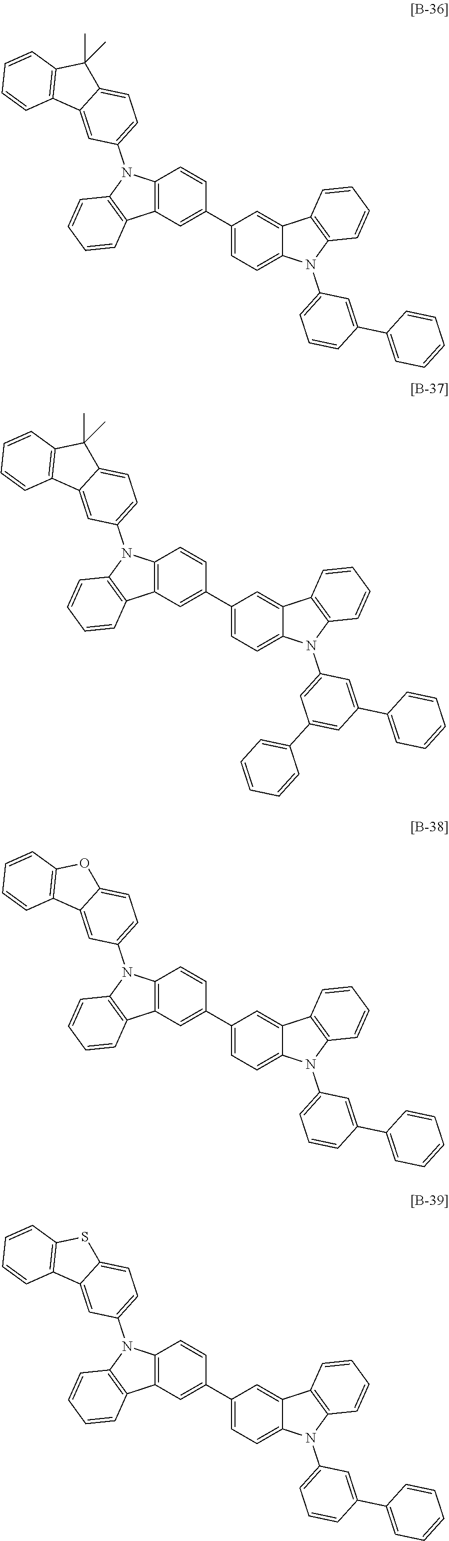 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

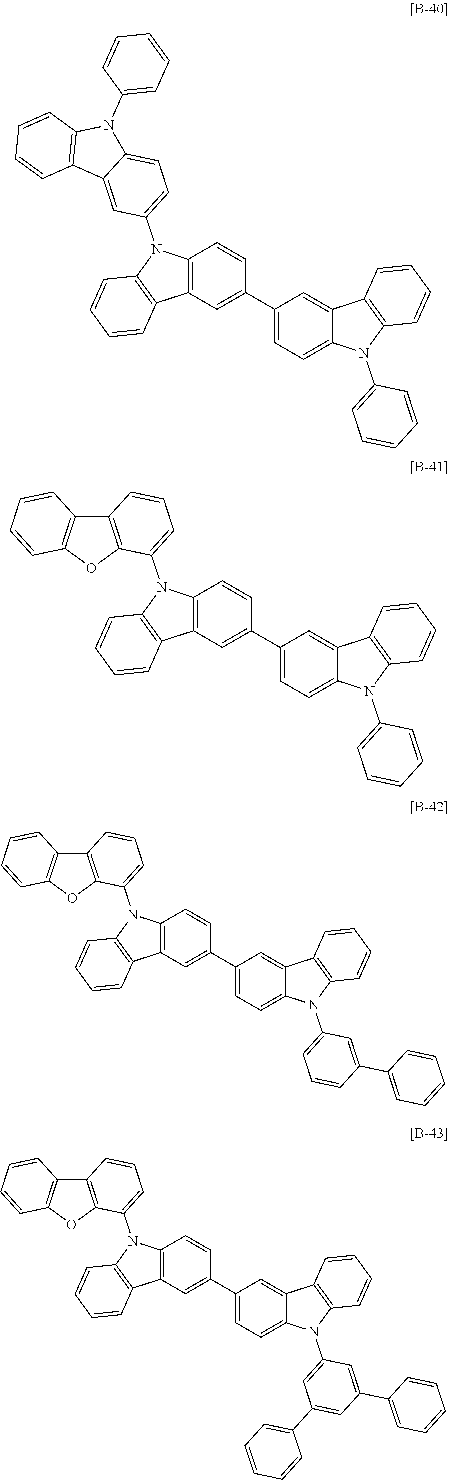 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

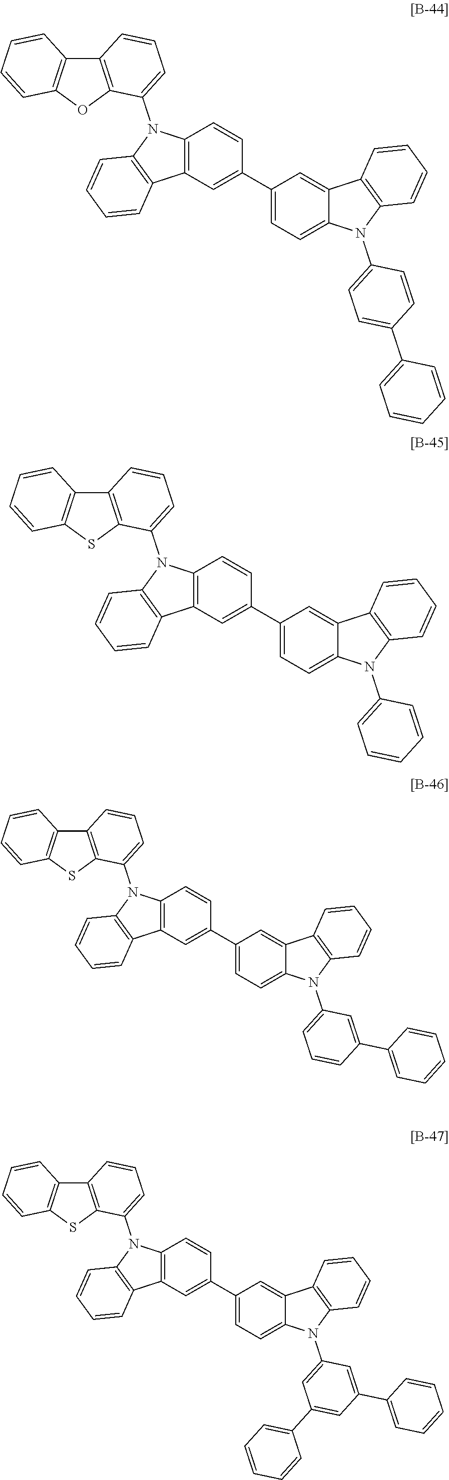 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

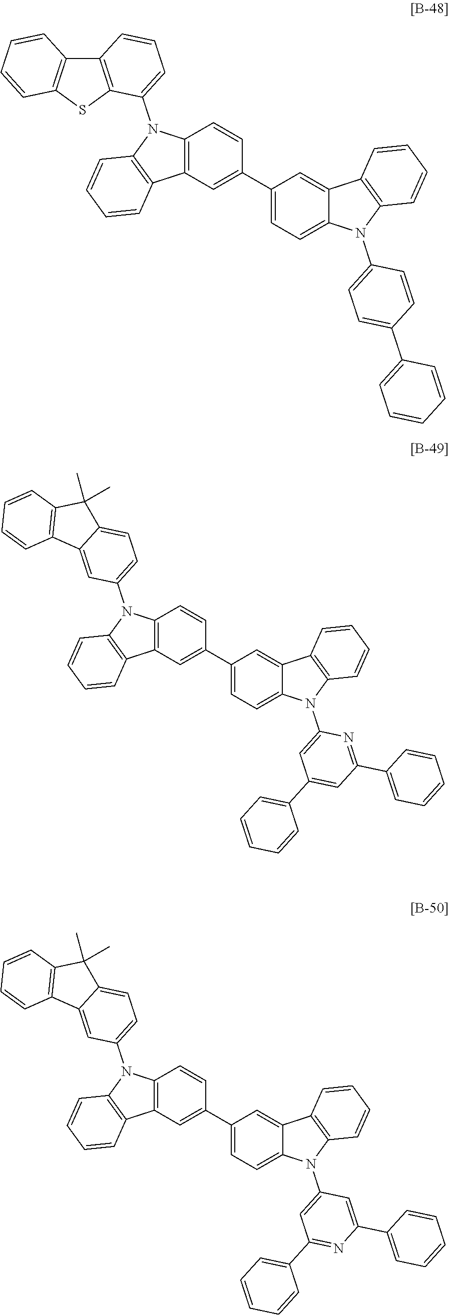 [SMILES: c1ccc(-c2ccc(-n3c4ccccc4c4cc(-c5ccc6c(c5)c5ccccc5n6-c5cccc6c5sc5ccccc56)ccc43)cc2)cc1 ; CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

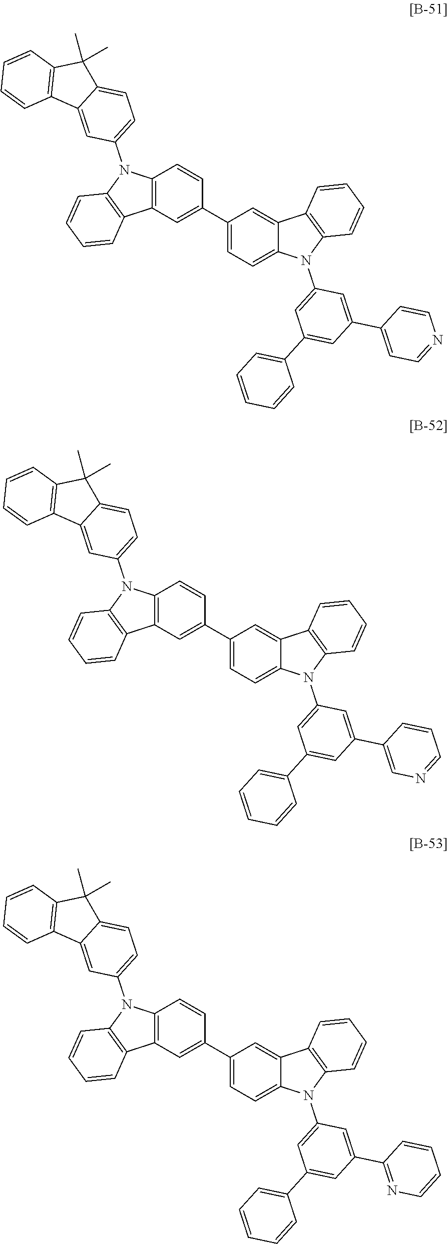 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

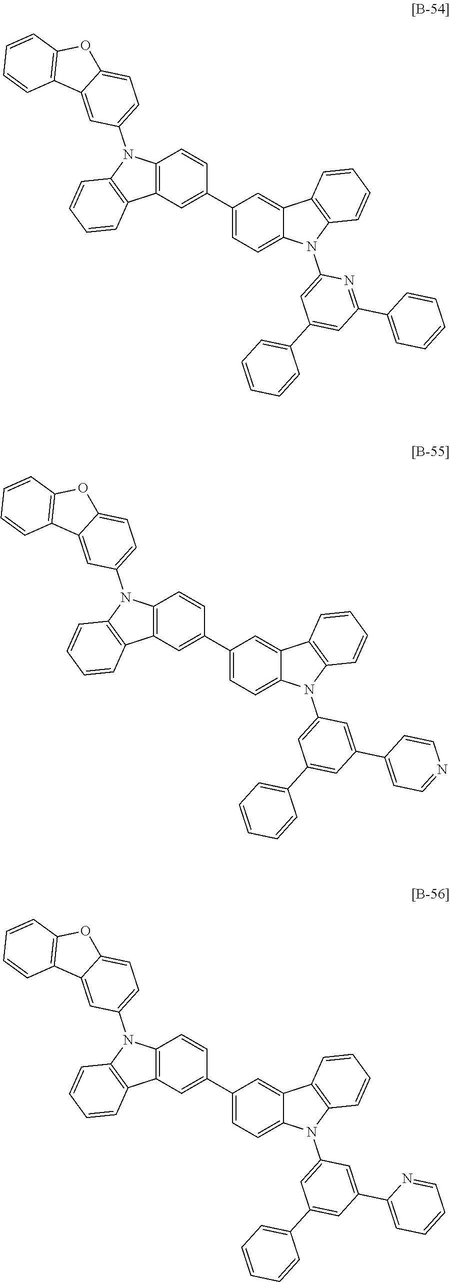 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

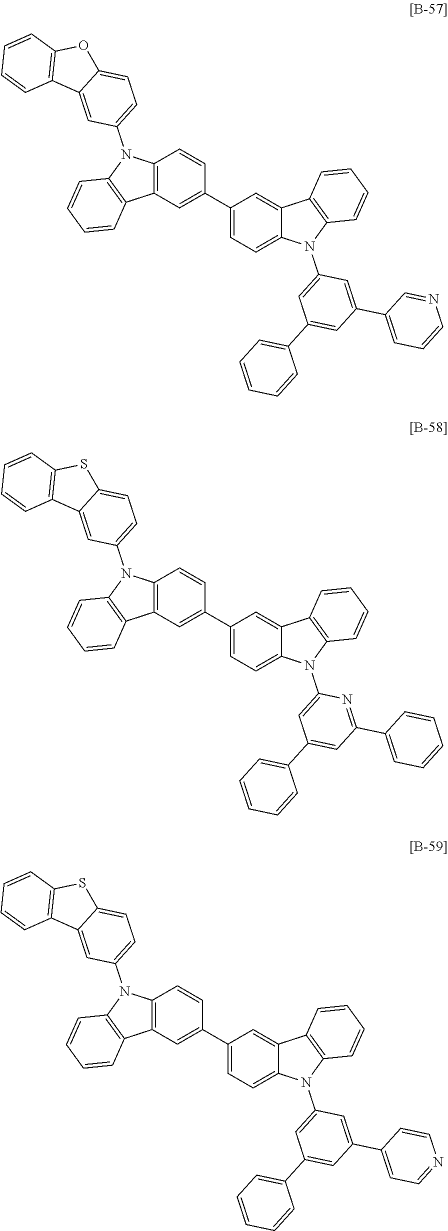 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

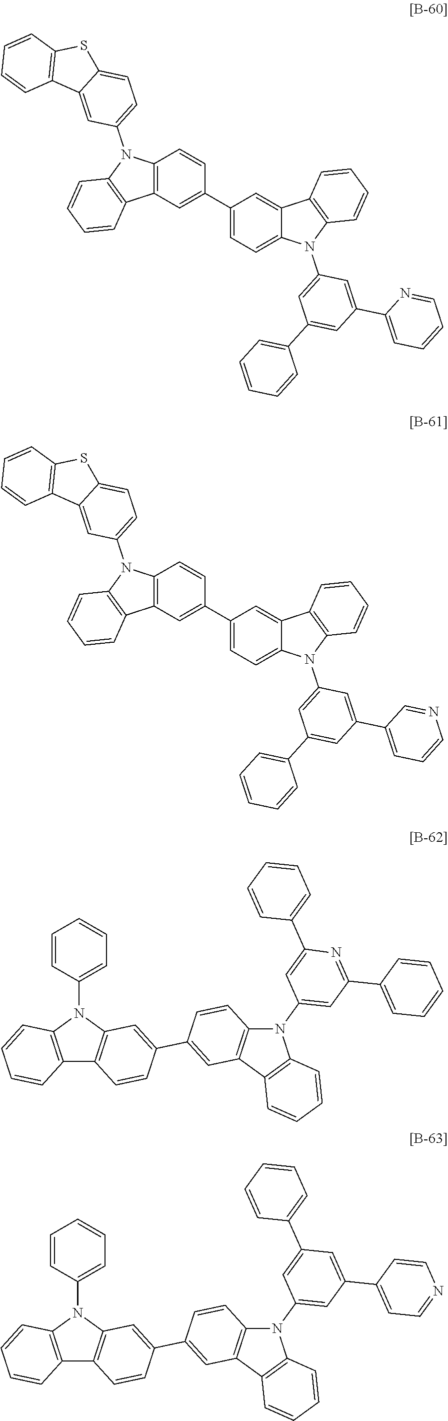 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1]

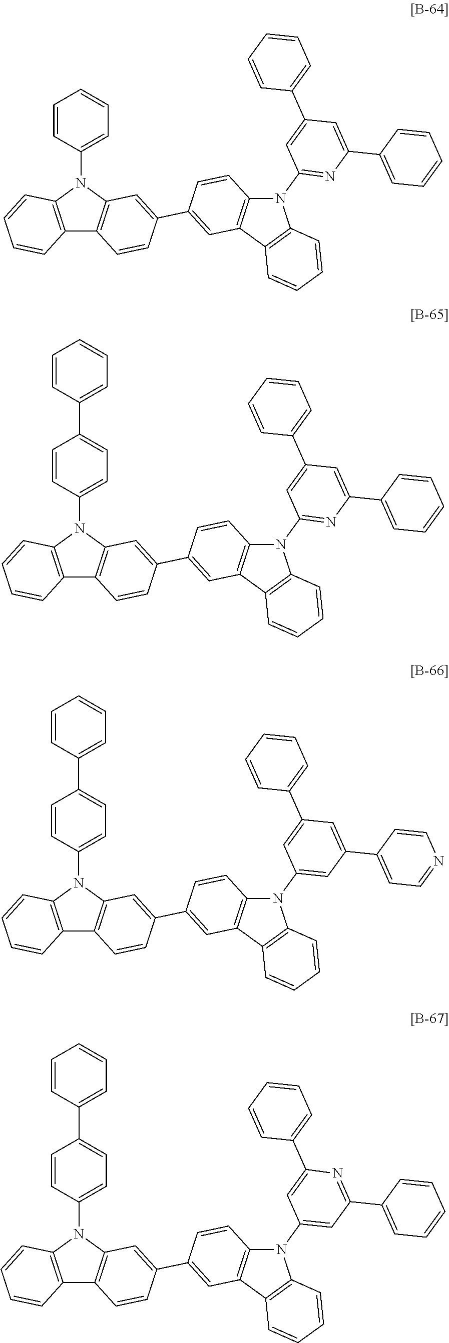 [SMILES: c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1]

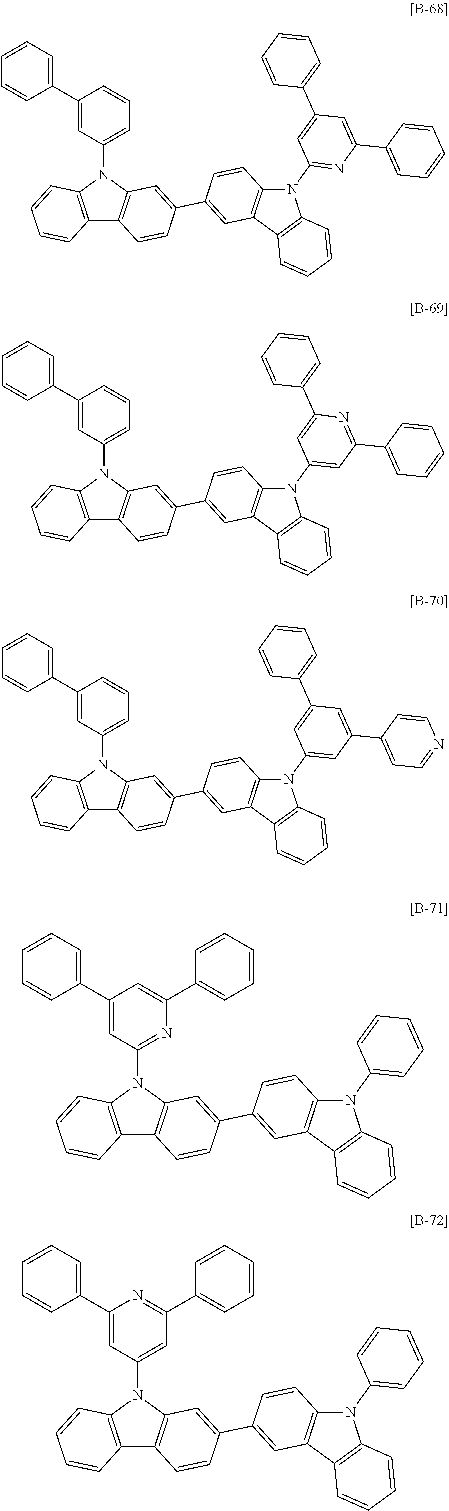 [SMILES: c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1]

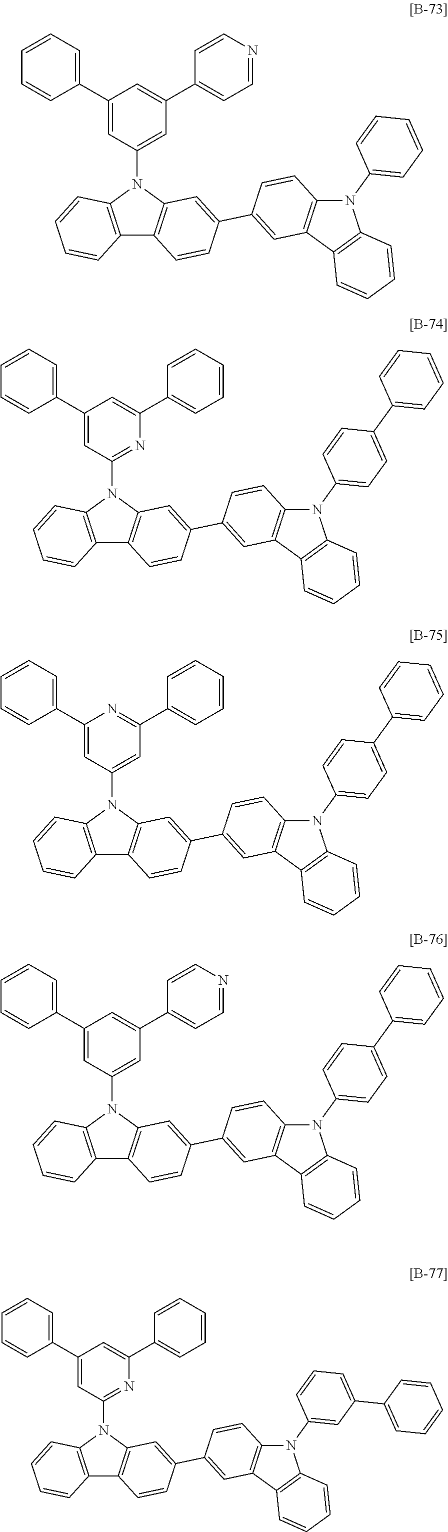 [SMILES: c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1]

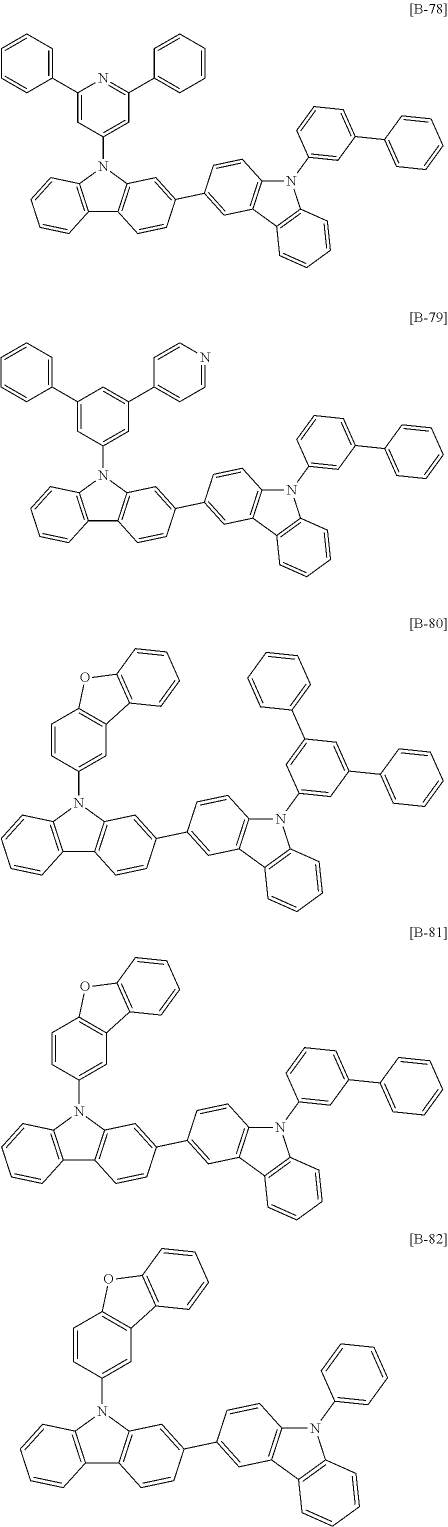 [SMILES: c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1]

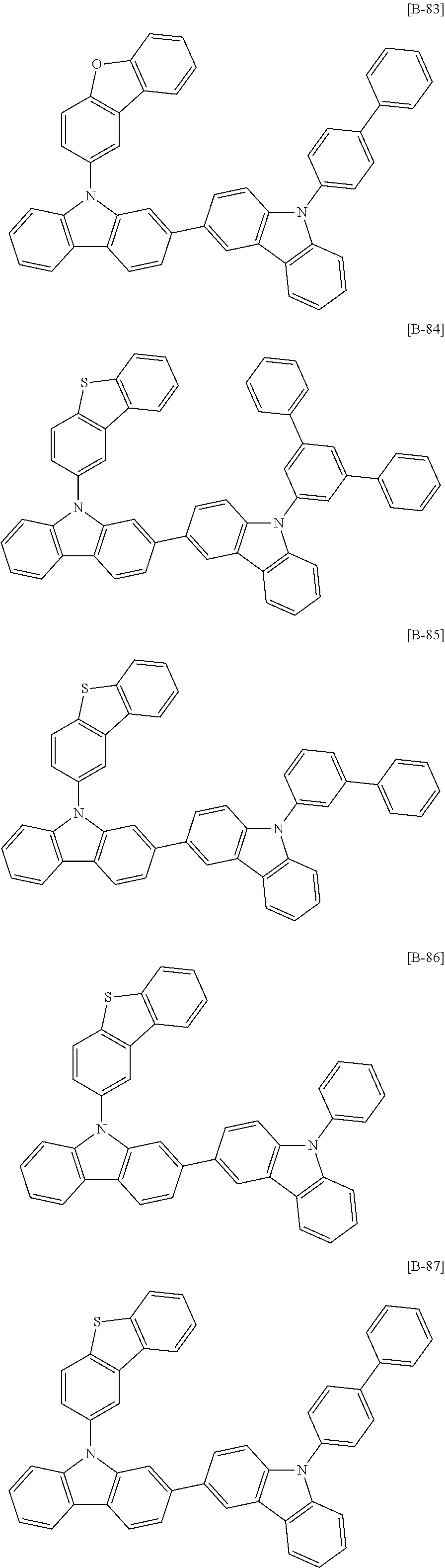 [SMILES: c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1]

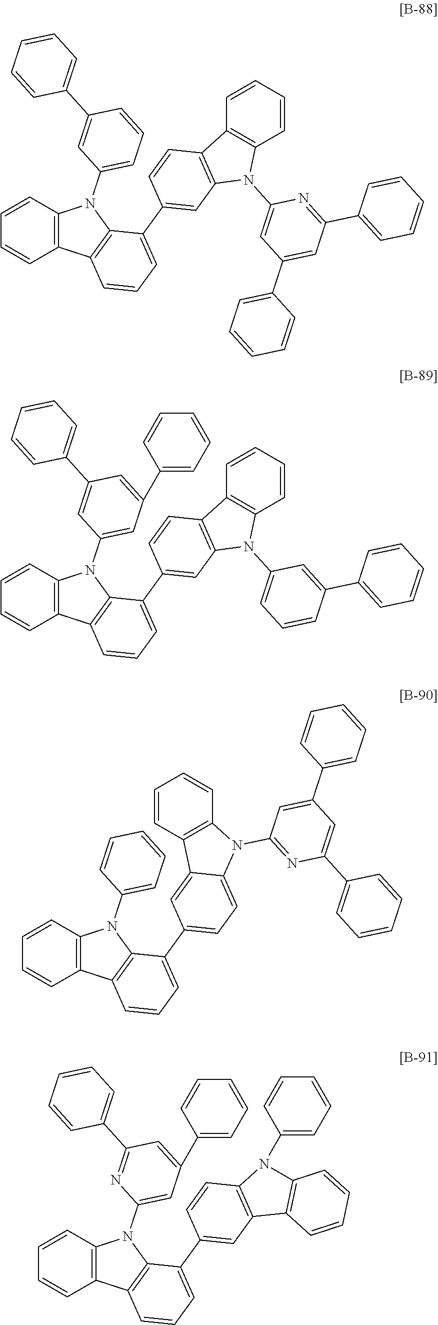 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)c7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1 ; c1ccc(-c2cc(-c3ccccc3)nc(-n3c4ccccc4c4cccc(-c5ccc6c(c5)c5ccccc5n6-c5ccccc5)c43)c2)cc1]

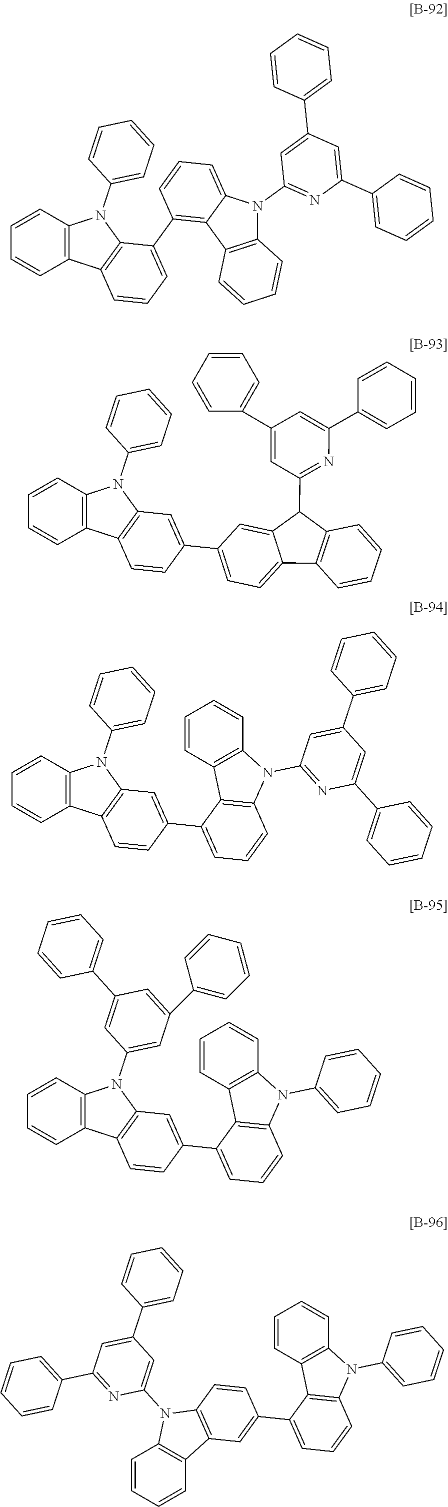 [SMILES: c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)cc(-n3c4ccccc4c4ccc(-c5cccc6c5c5ccccc5n6-c5ccccc5)cc43)c2)cc1 ; c1ccc(-c2cc(-c3ccccc3)nc(-n3c4ccccc4c4cc(-c5cccc6c5c5ccccc5n6-c5ccccc5)ccc43)c2)cc1]

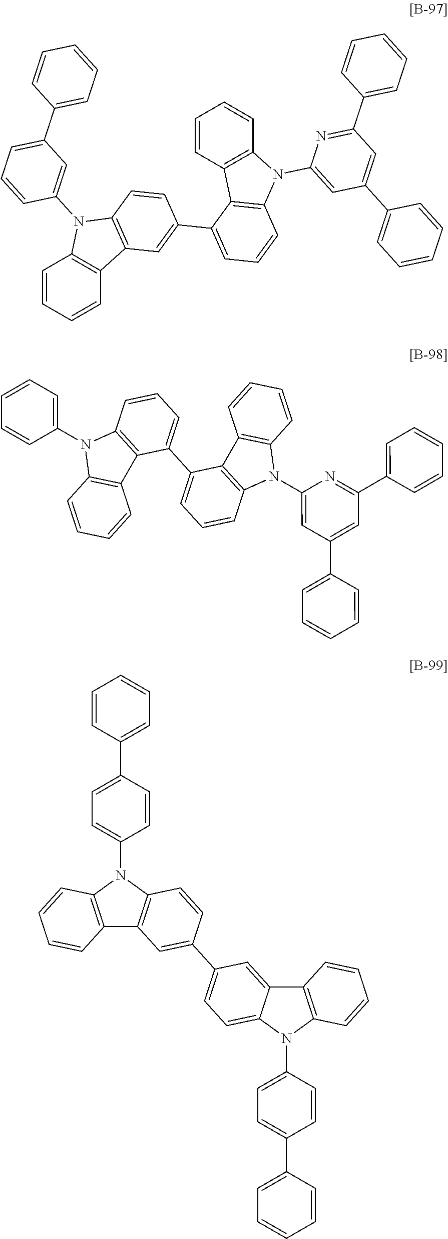 [SMILES: c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

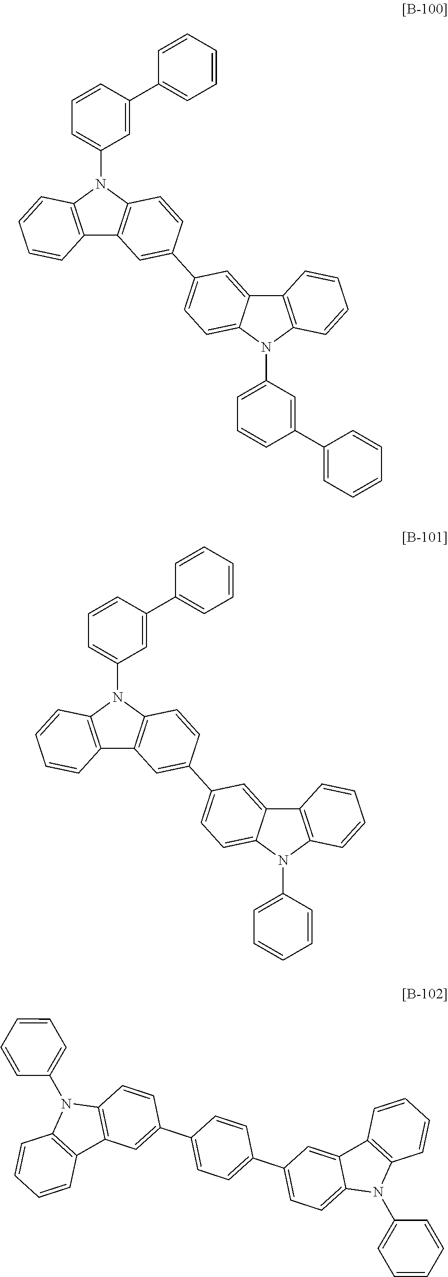 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

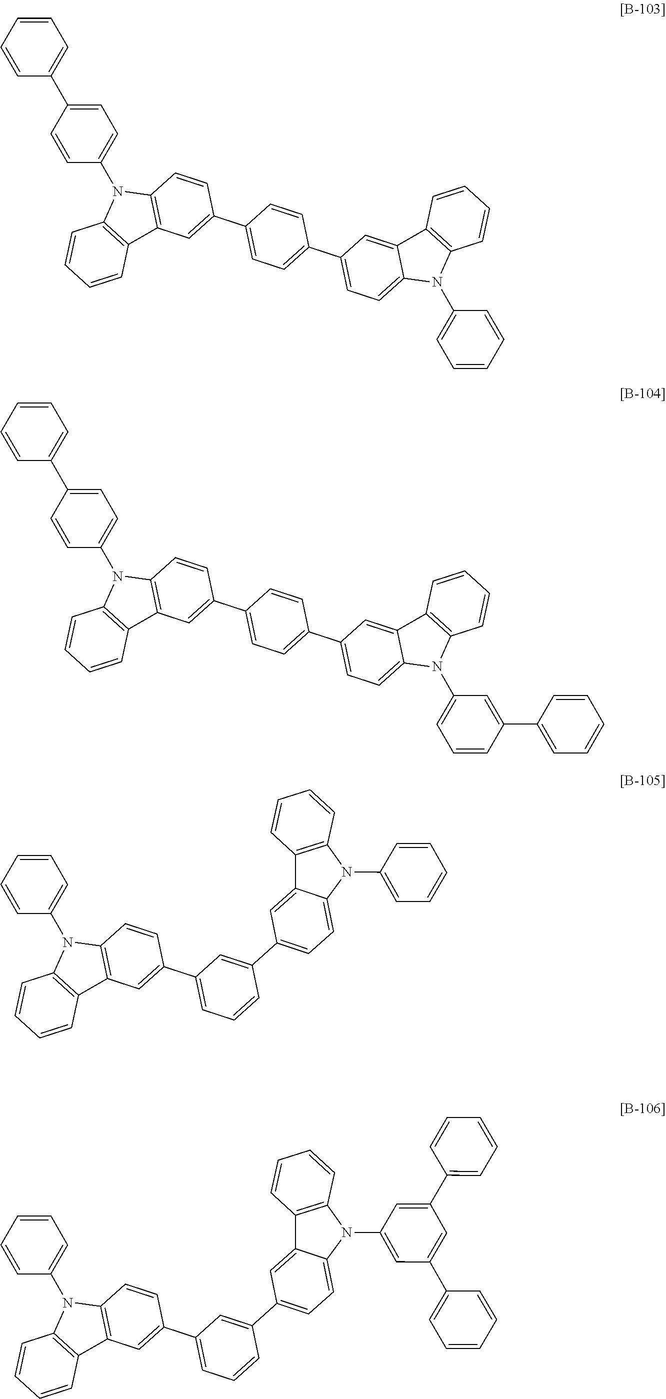 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

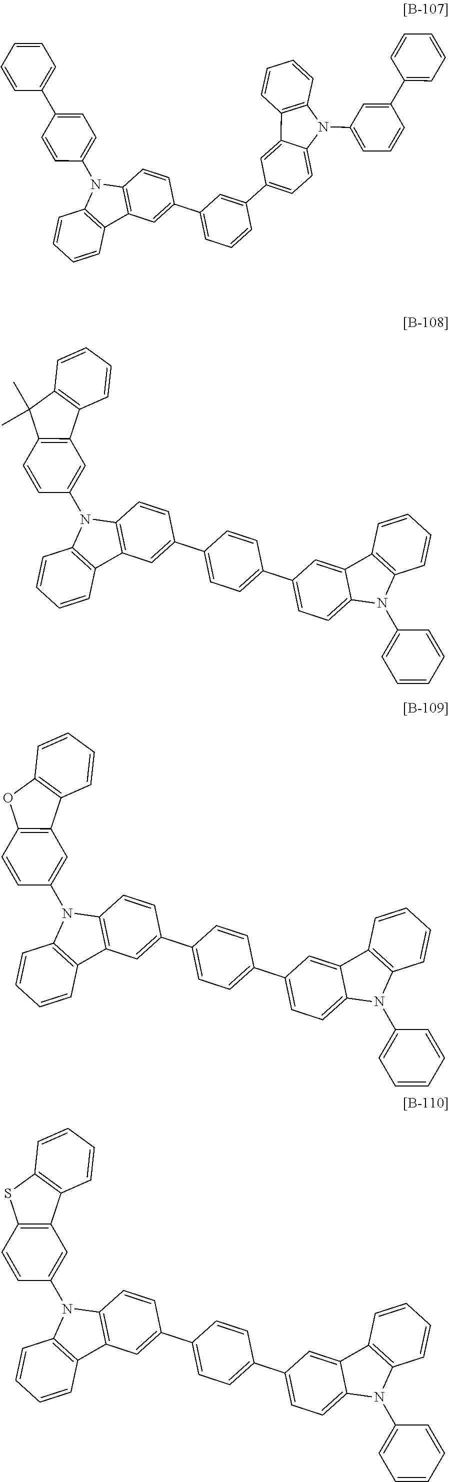 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1]

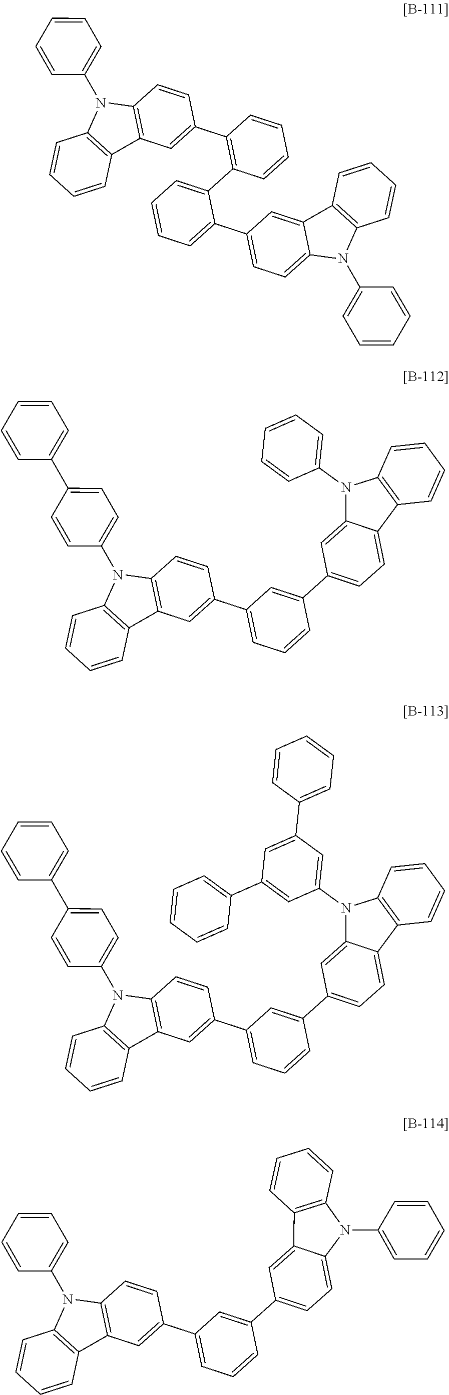 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1 ; c1ccc(-n2c3ccccc3c3cc(-c4cccc(-c5ccc6c(c5)c5ccccc5n6-c5ccccc5)c4)ccc32)cc1]

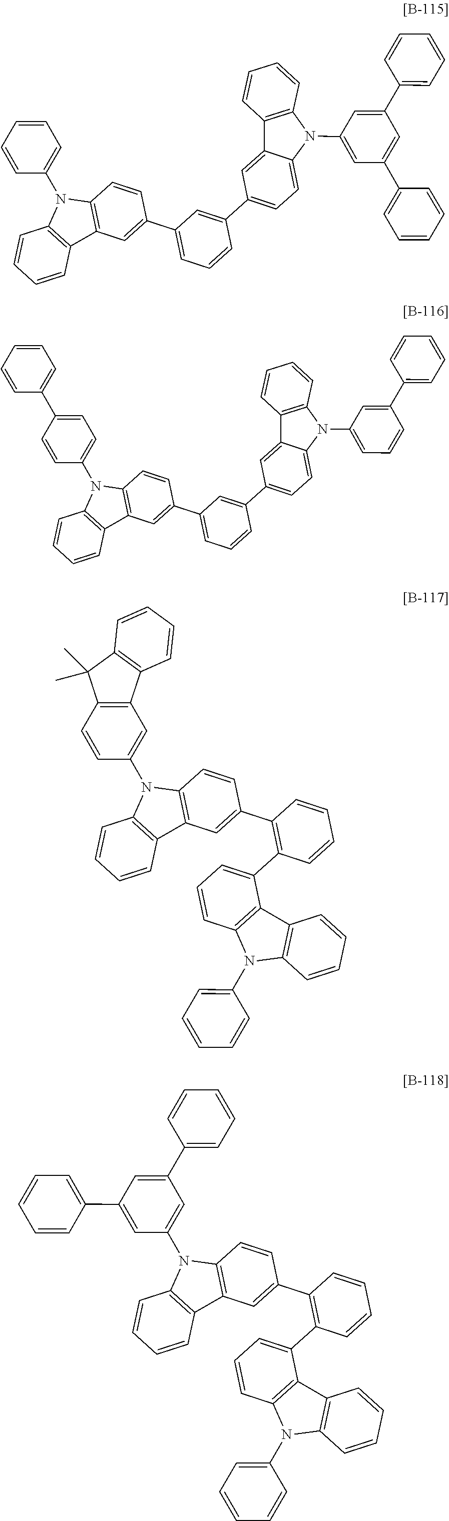 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

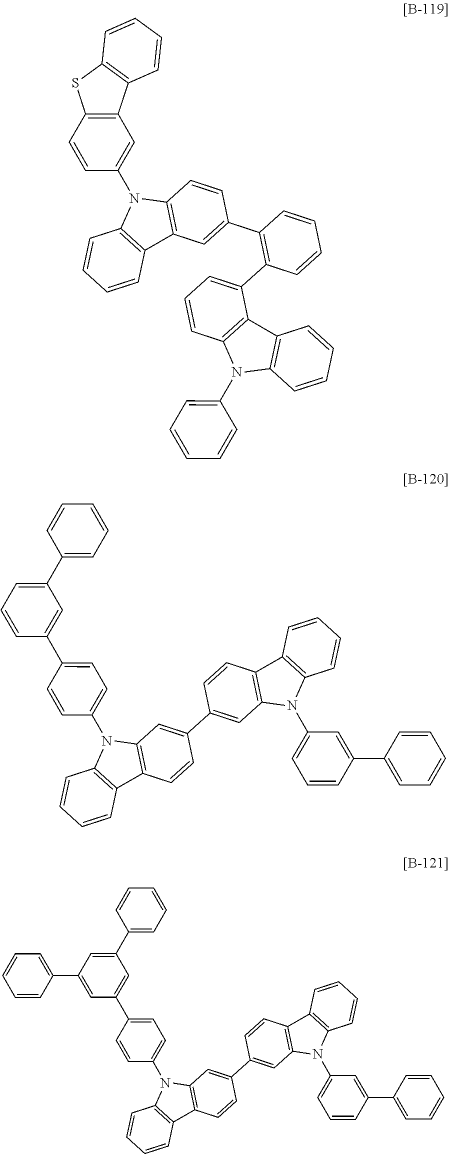 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

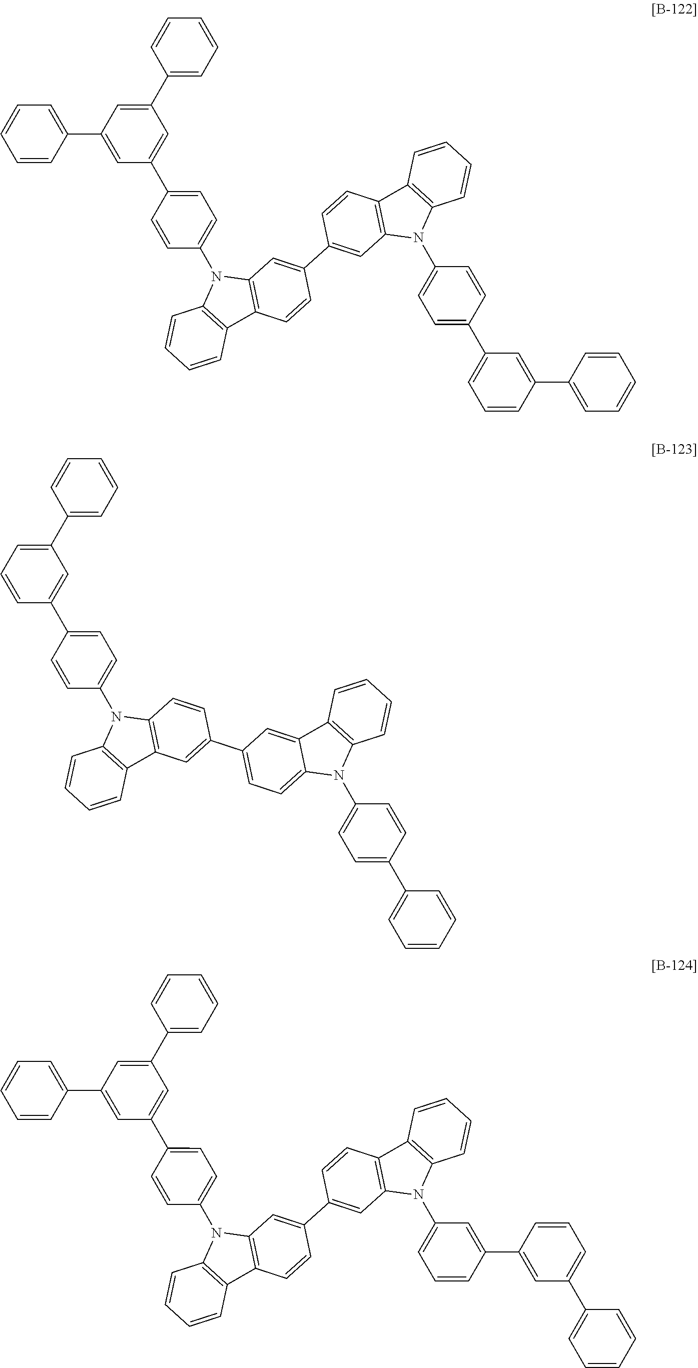 [SMILES: c1ccc(-c2cccc(-c3ccc(-n4c5ccccc5c5ccc(-c6ccc7c8ccccc8n(-c8ccc(-c9cc(-c%10ccccc%10)cc(-c%10ccccc%10)c9)cc8)c7c6)cc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

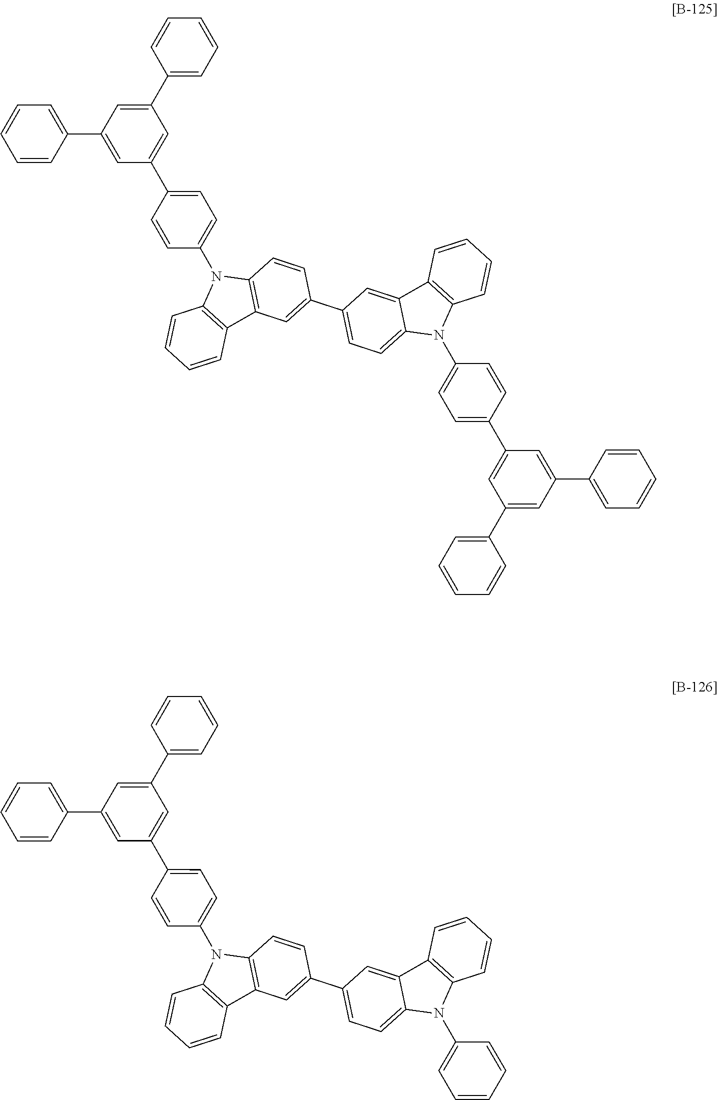 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

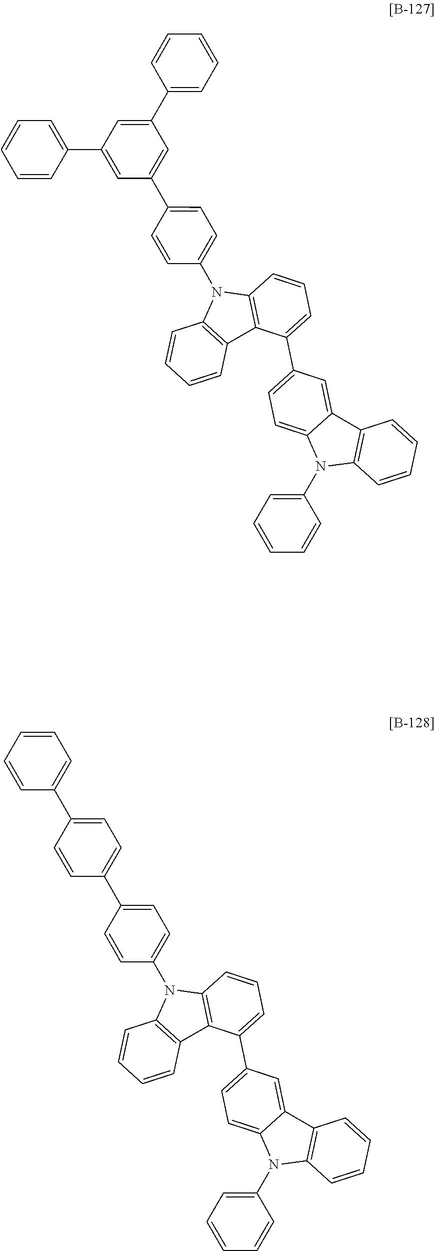 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

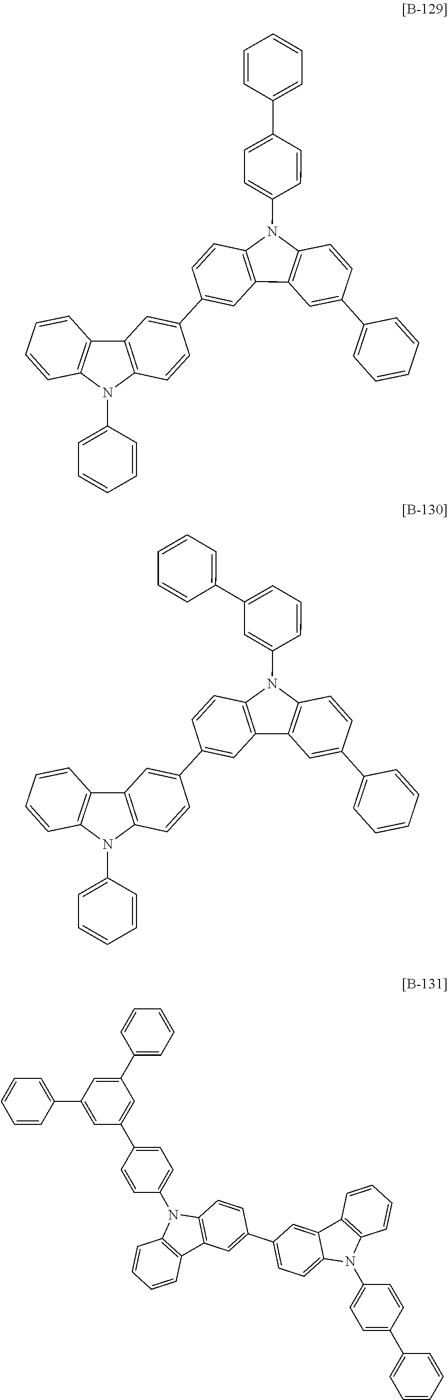 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

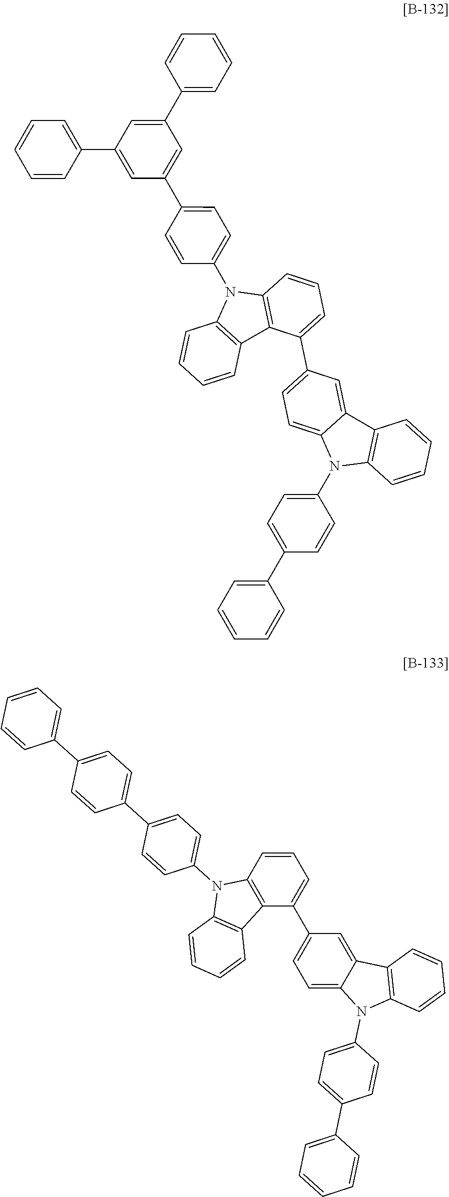 [SMILES: c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1]

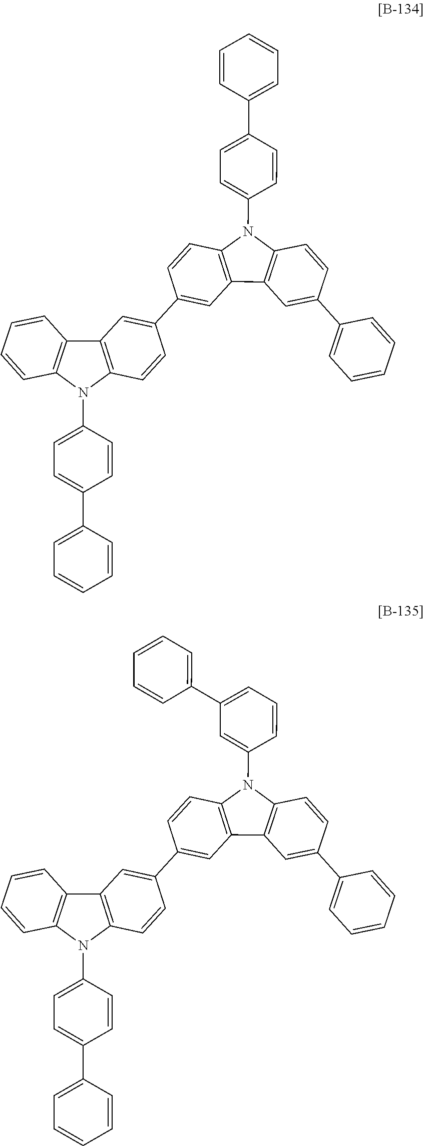 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1]

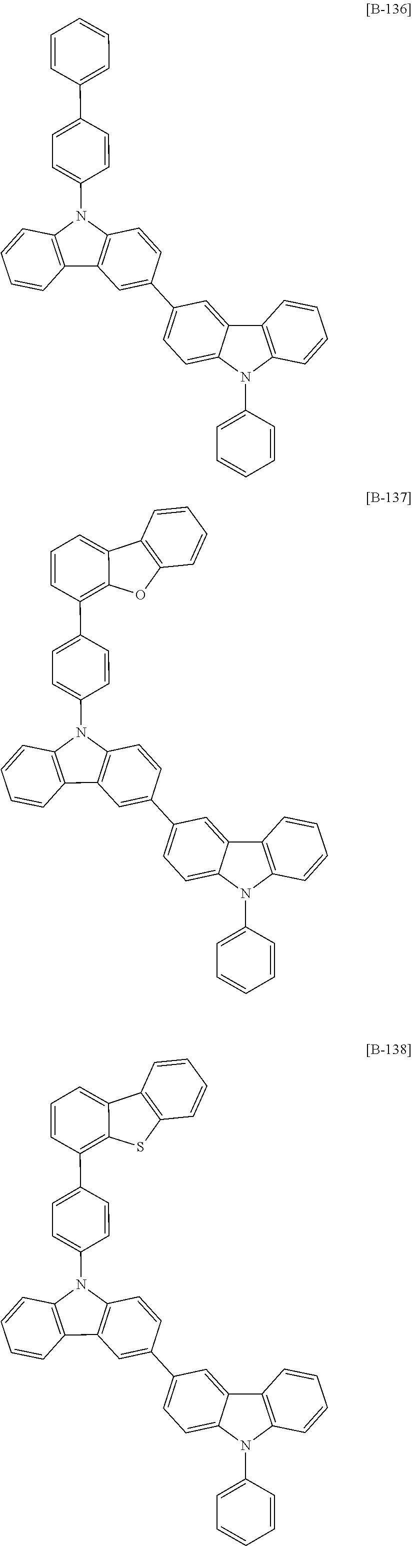 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

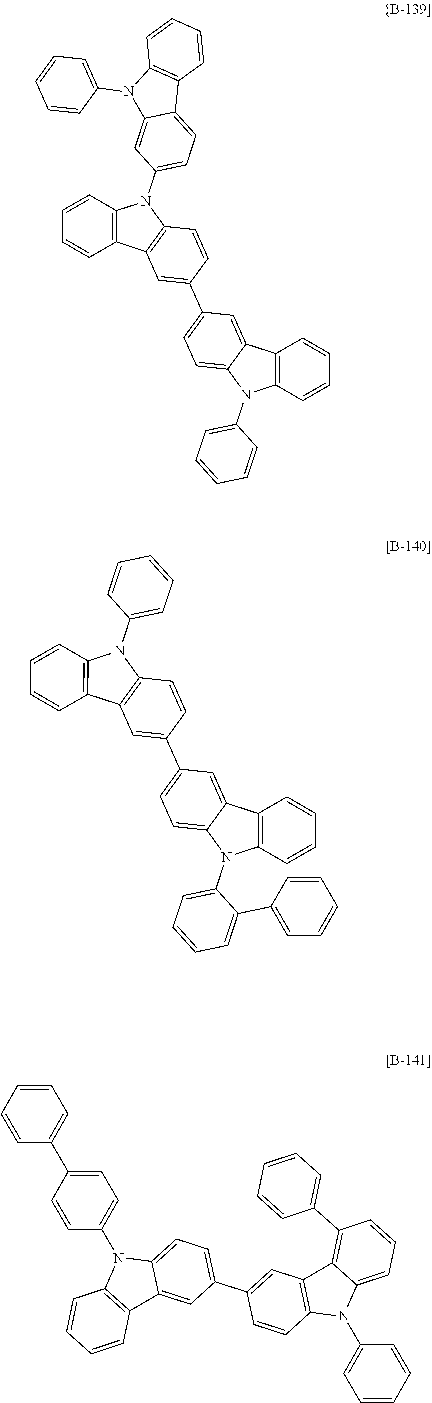 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1]

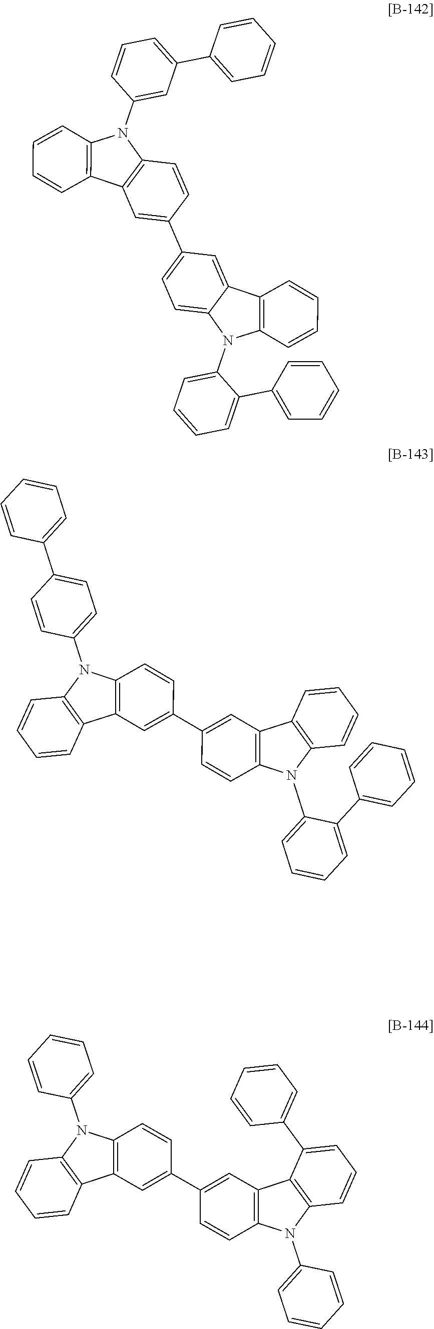 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc3c2c2cc(-c4ccc5c(c4)c4ccccc4n5-c4ccccc4)ccc2n3-c2ccccc2)cc1]

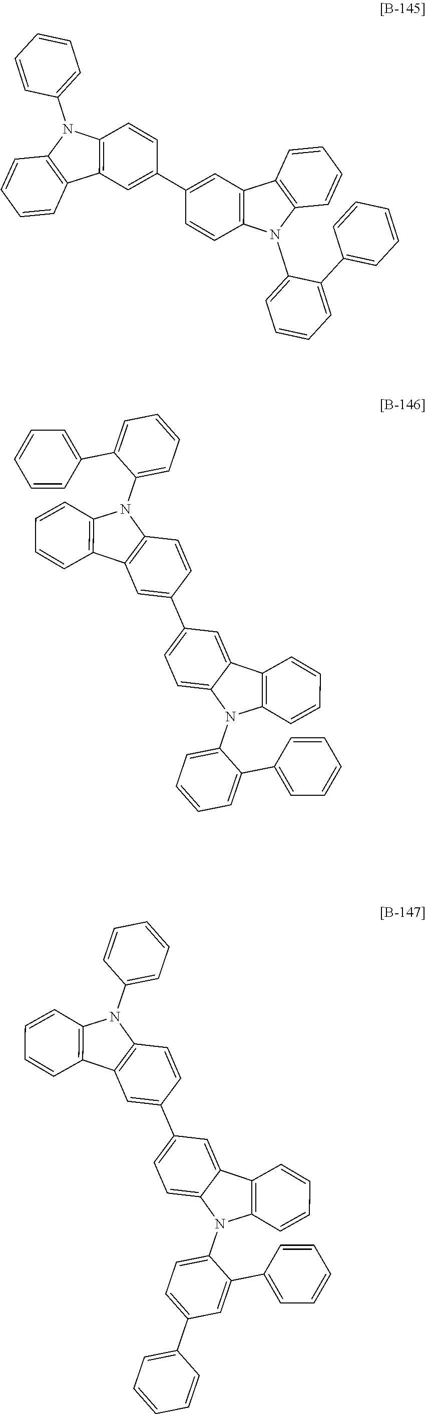 [SMILES: c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1]

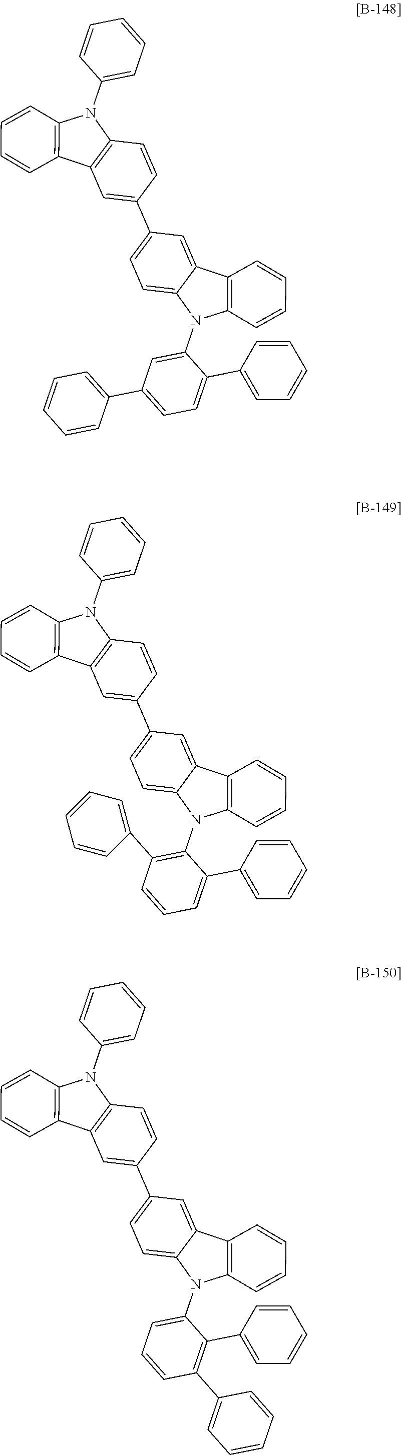 [SMILES: c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2-c2ccccc2)cc1]

Additionally, examples of Compound B-1 to Compound B-150 listed in Group 2 in which at least one hydrogen is replaced with deuterium are given below.

In an implementation, deuterium may be substituted, e.g., as shown in Compound B-151 to Compound B-195, exemplified below.

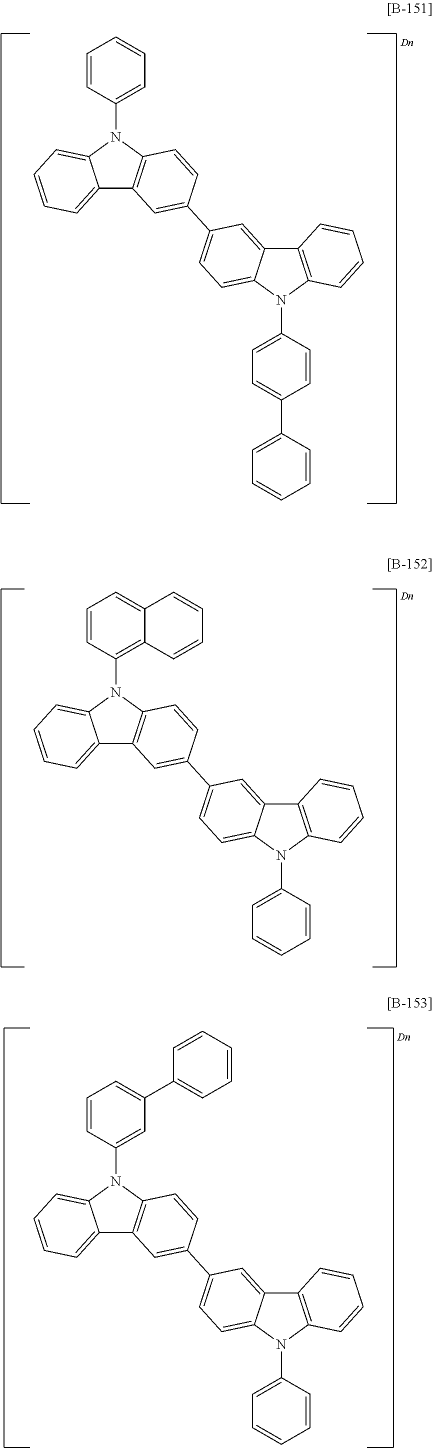

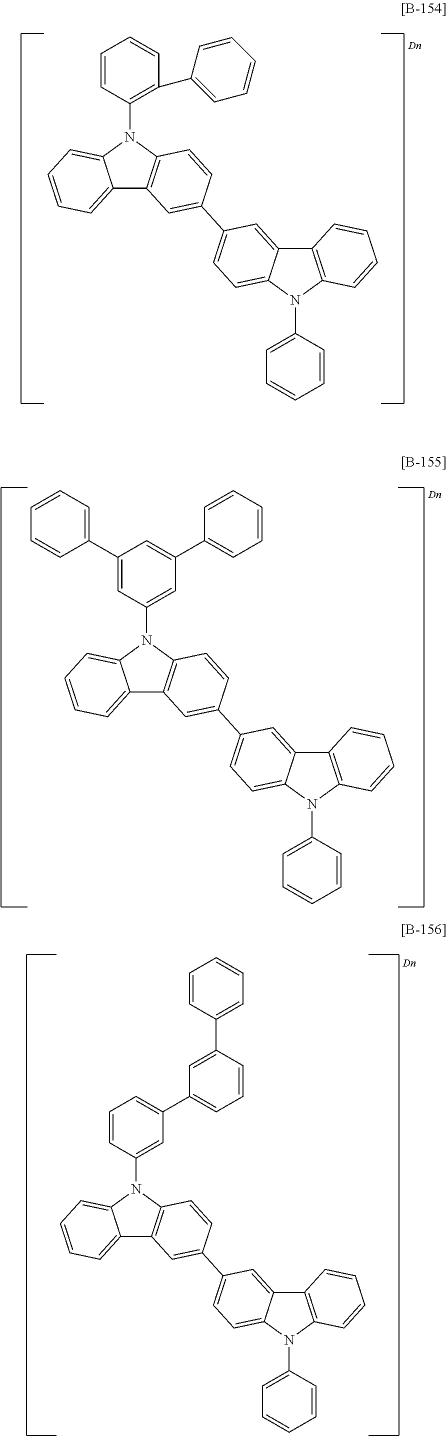

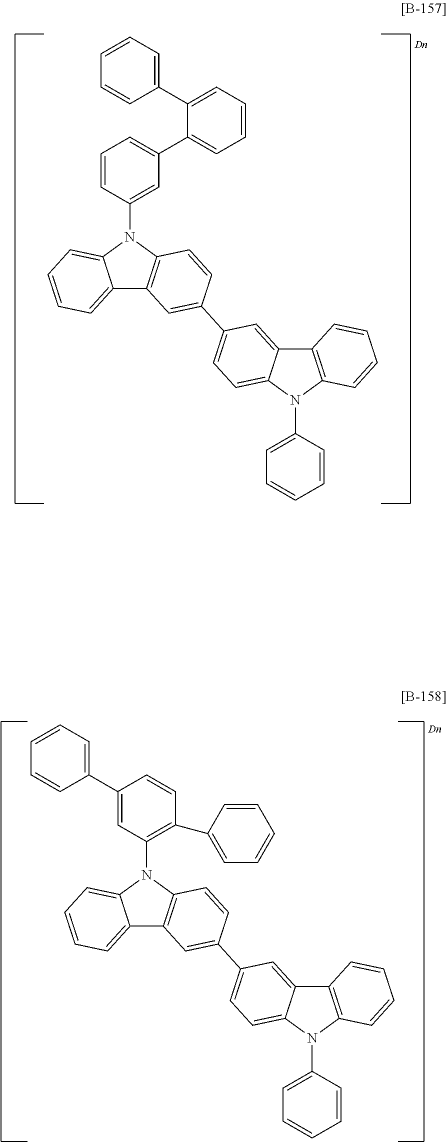

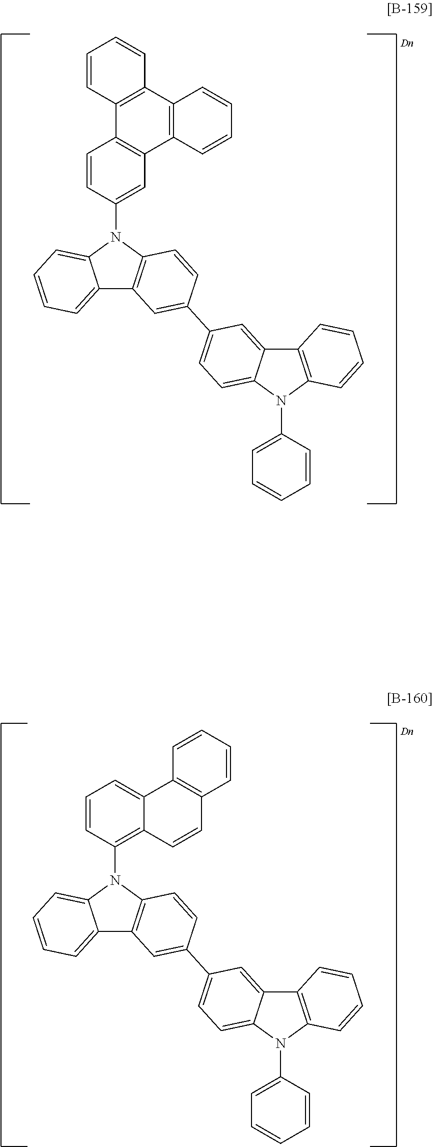

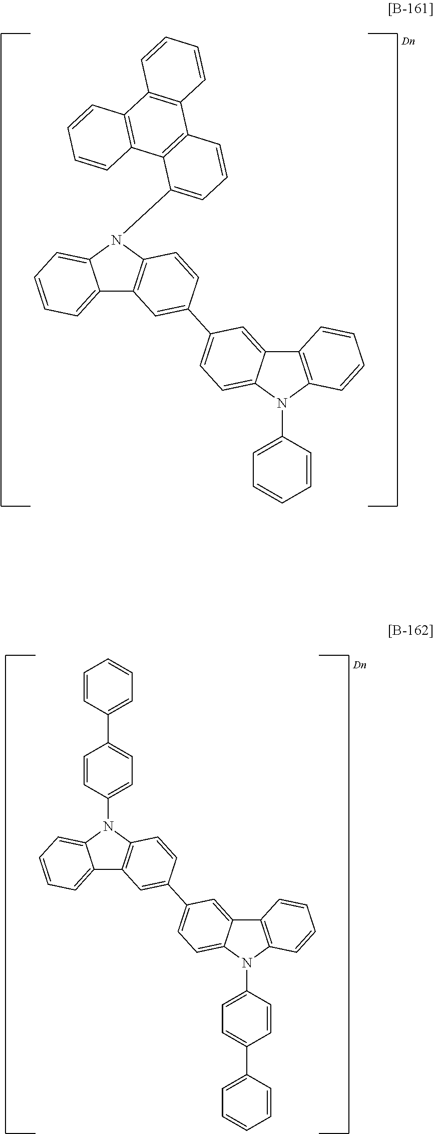

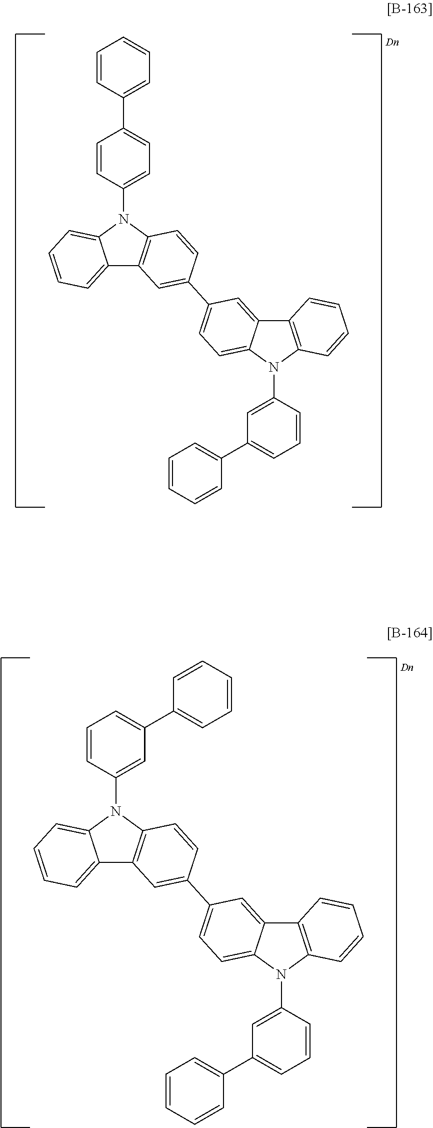

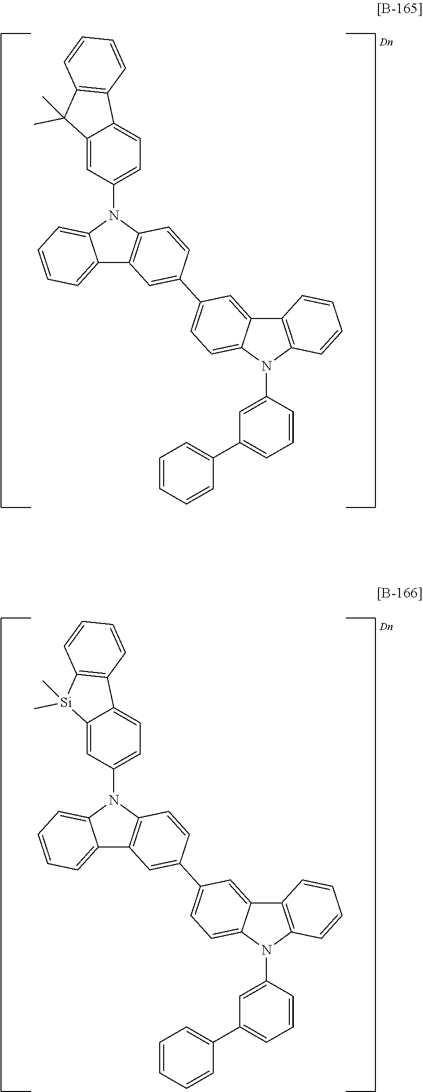

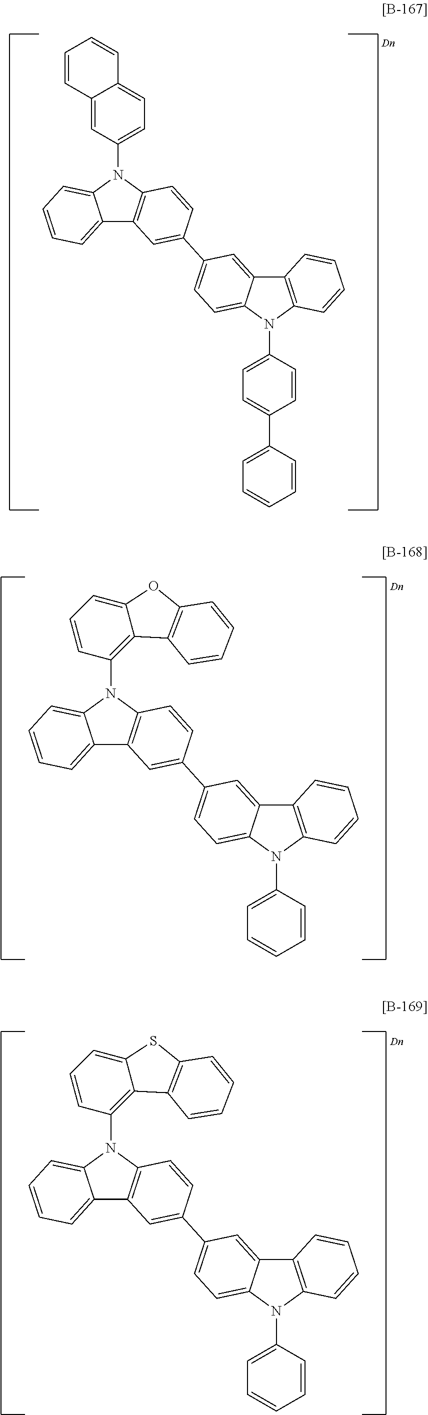

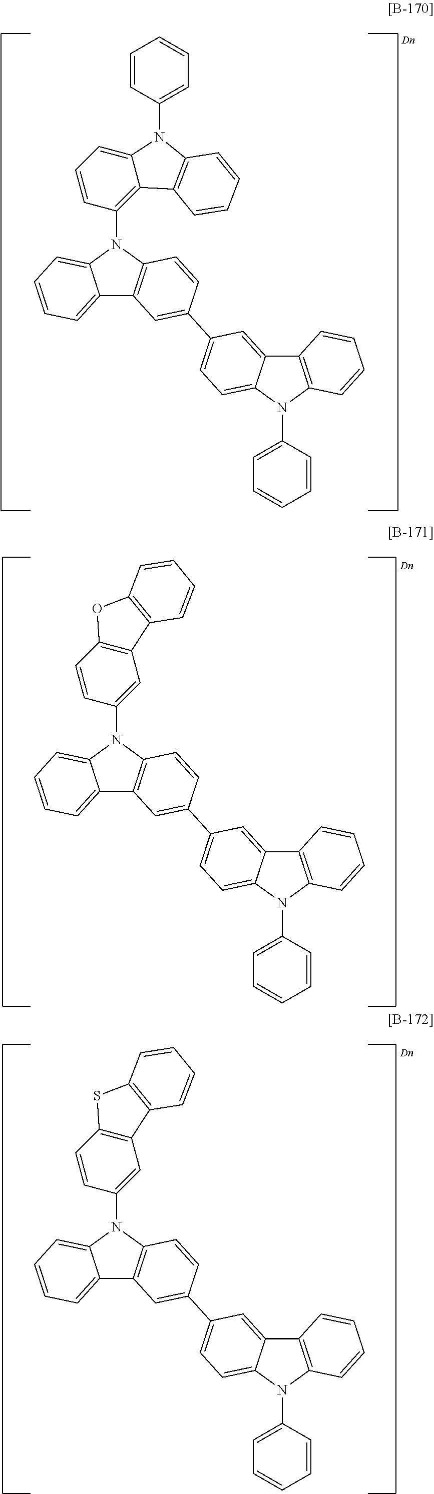

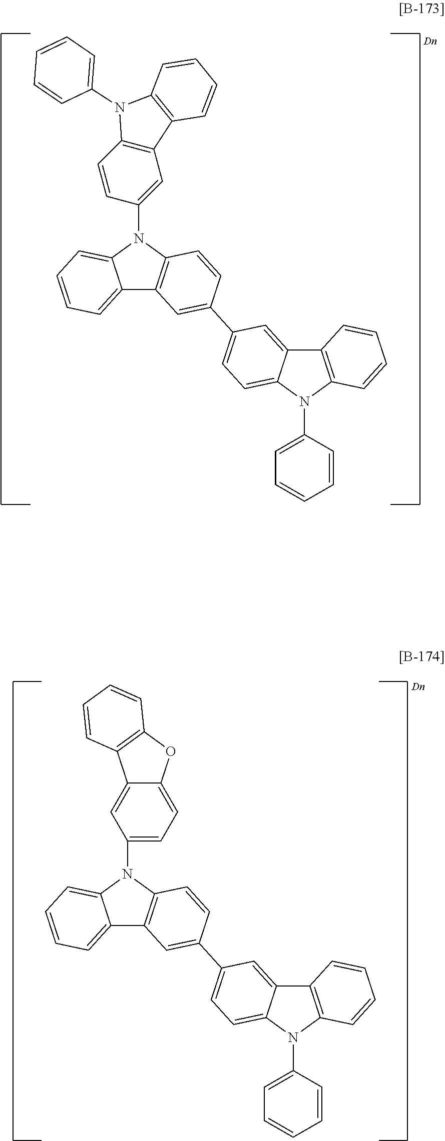

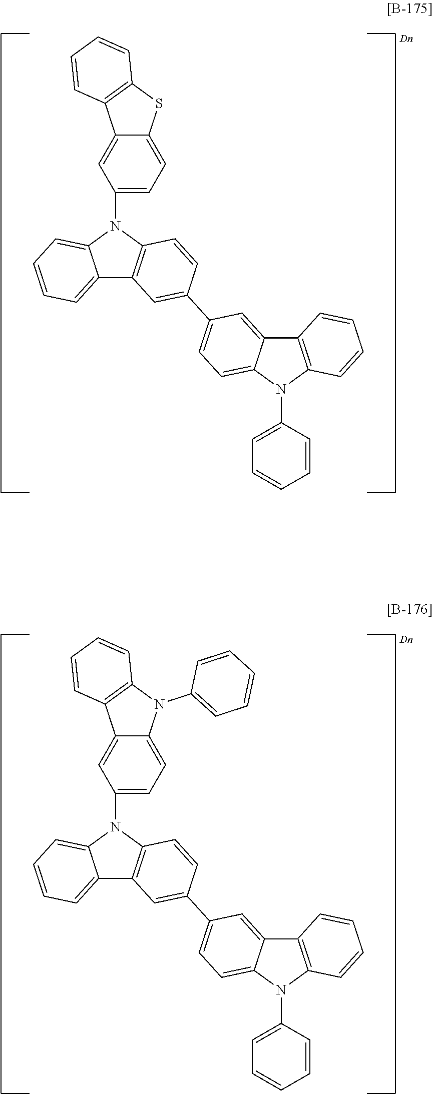

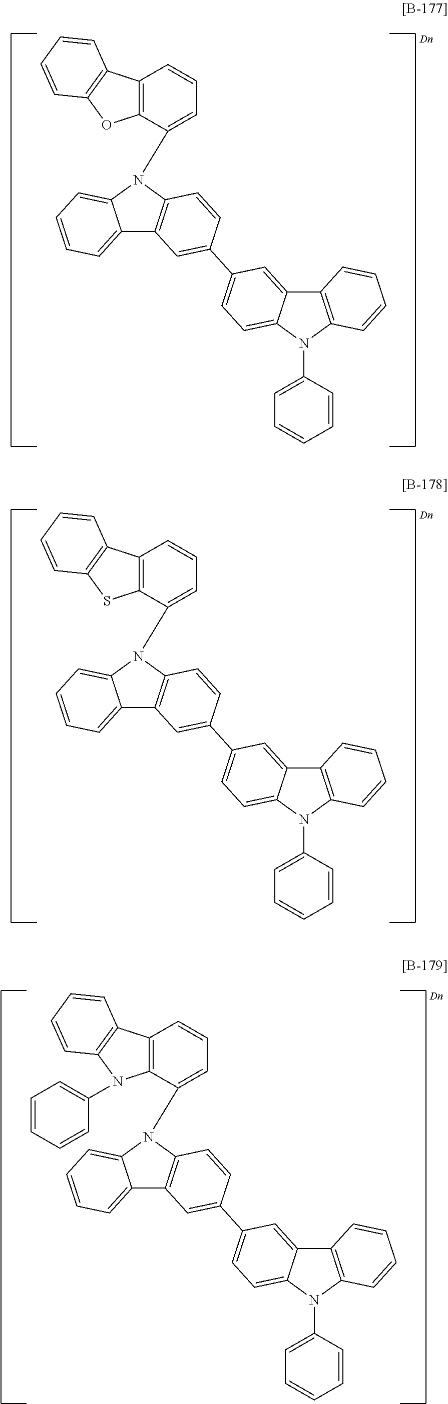

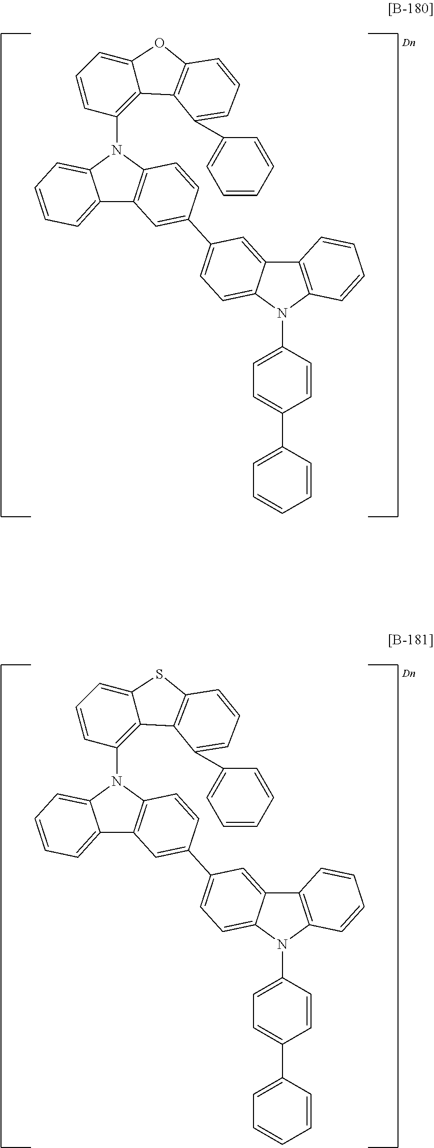

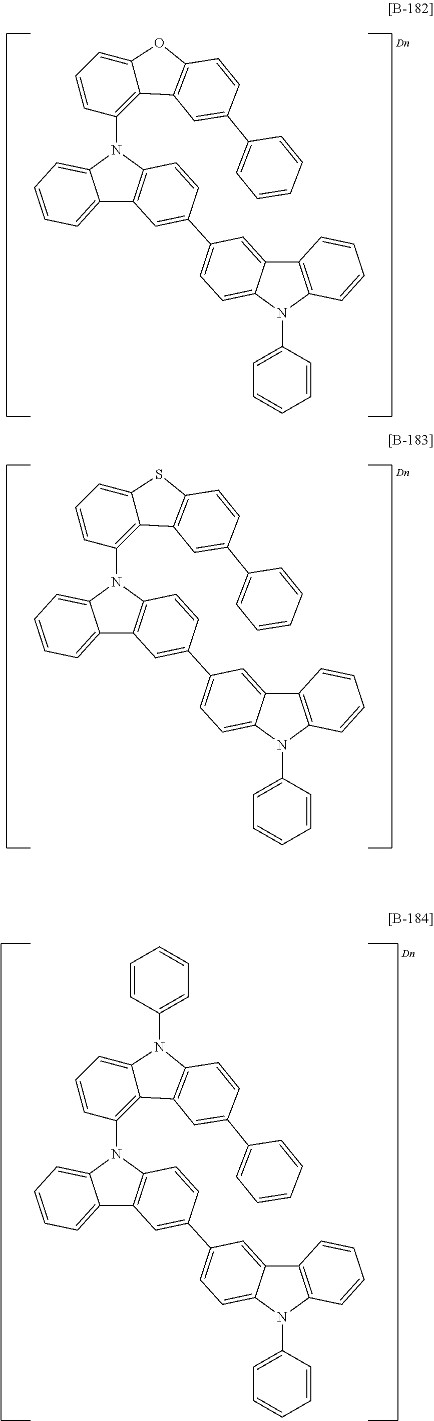

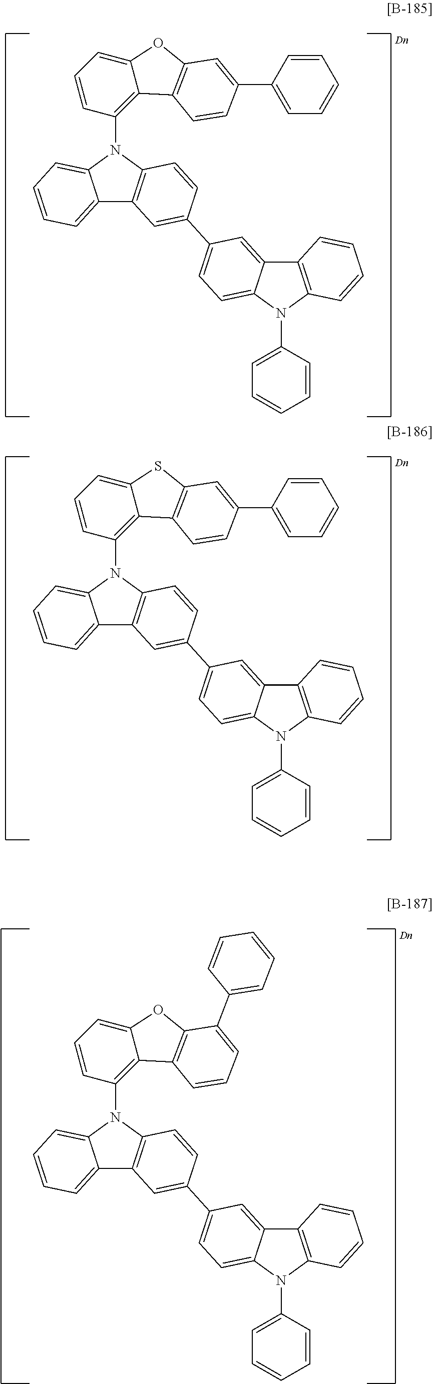

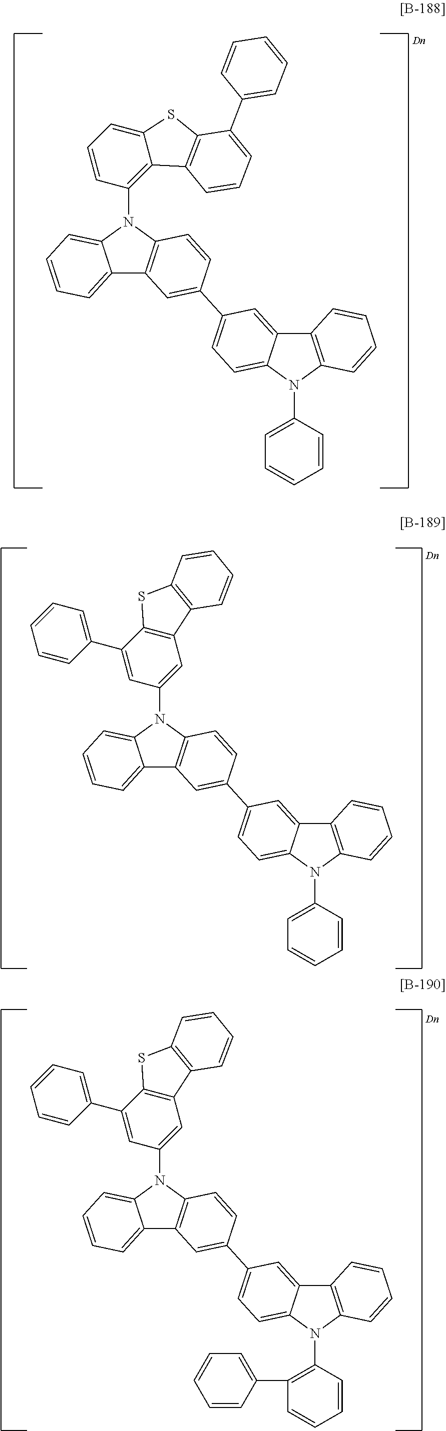

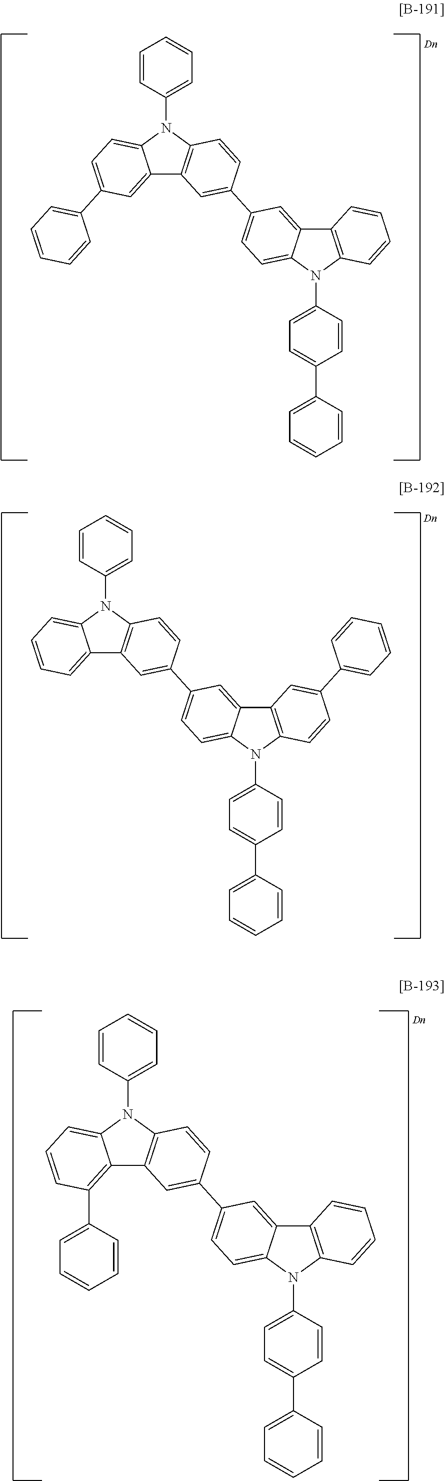

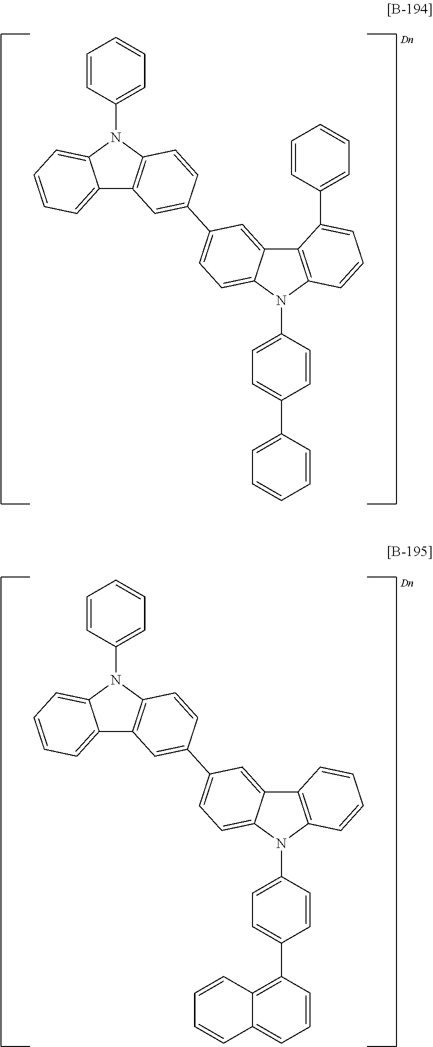

(Dn refers to the number of deuterium substitutions and indicates a structure substituted with one or more deuterium atoms)

The deuterium substitution position and deuterium substitution ratio may include all changeable ranges within the range of Compound B-1 to Compound B-195 (e.g., any hydrogen in any compound may be a protium or a deuterium).

The most specific structures for Compound B-151 to Compound B-195 of Group 2 are presented below as examples according to the position and substitution rate of deuterium substitution.

In an implementation, deuterium may be substituted, e.g., as shown in Compound B-196 to Compound B-234.

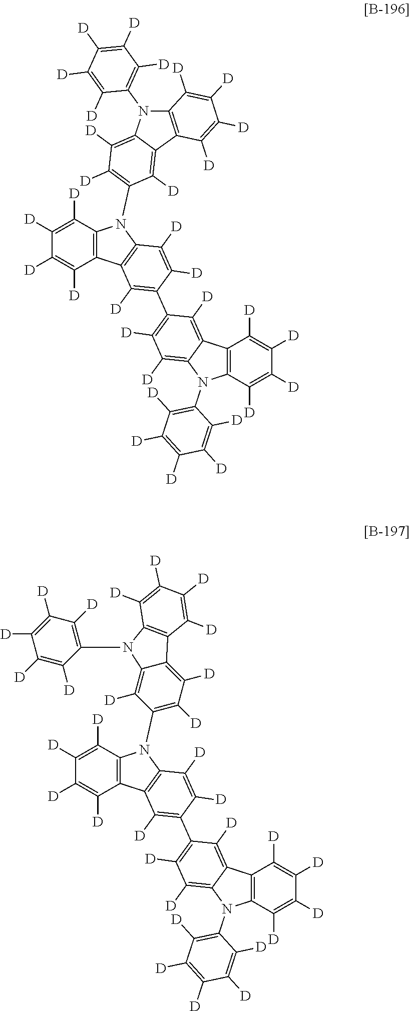

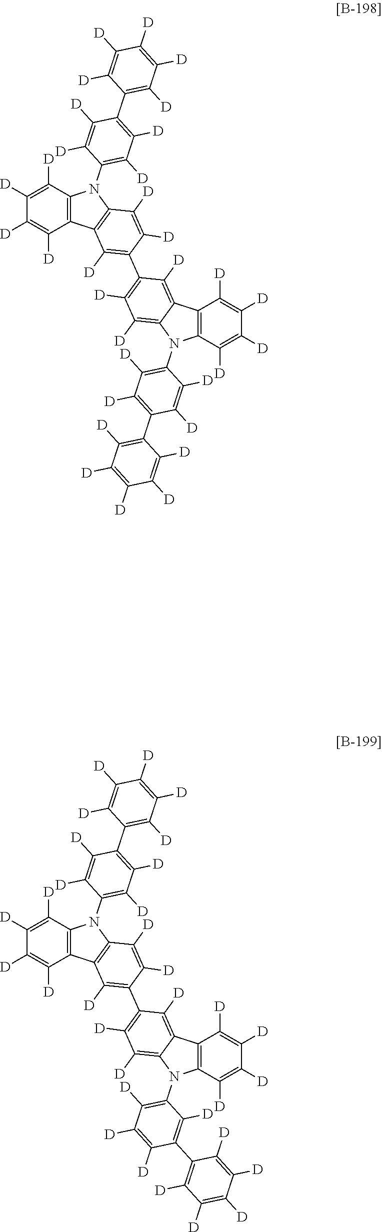

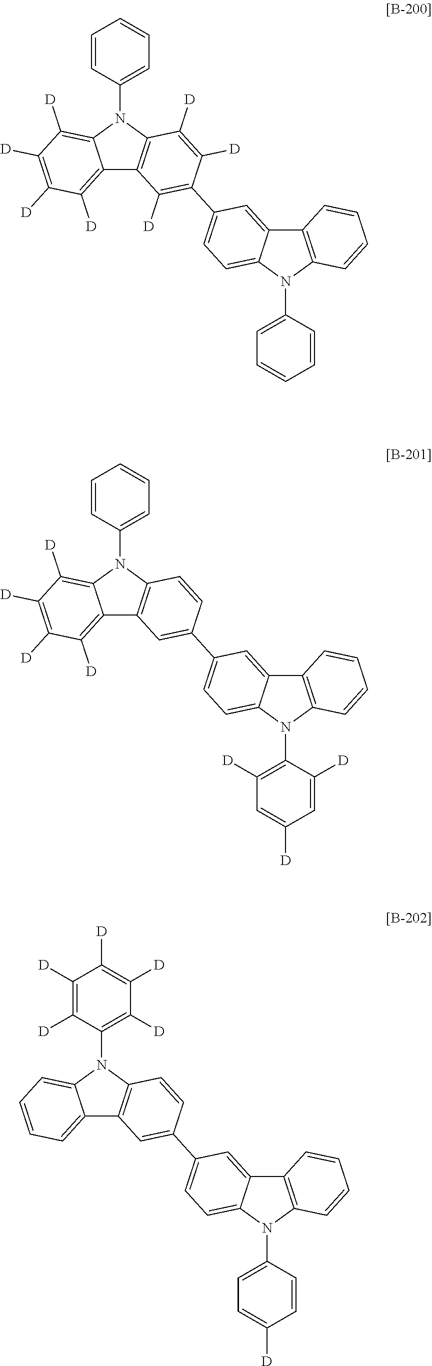 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

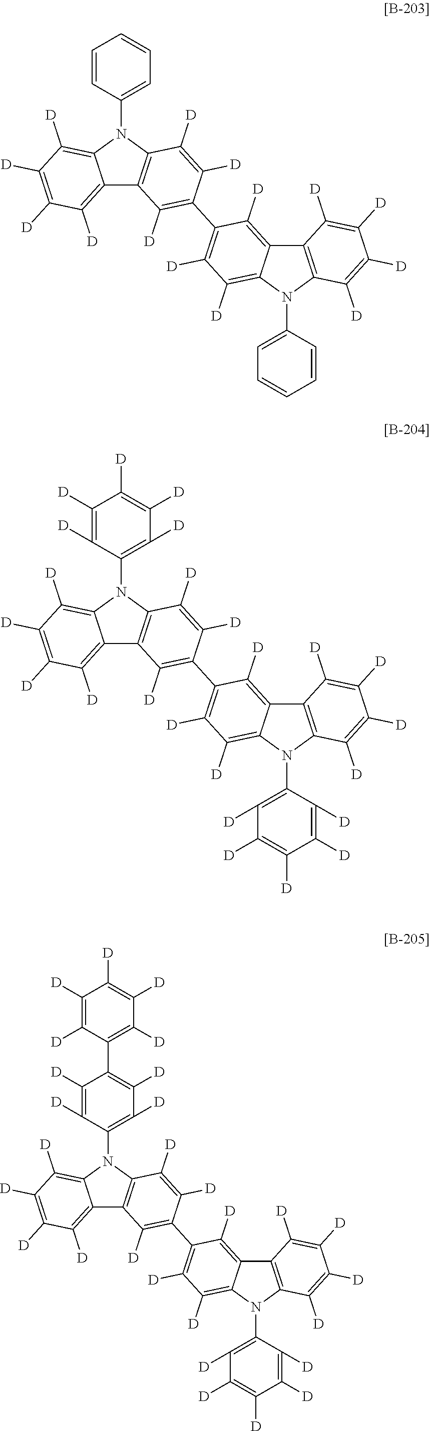

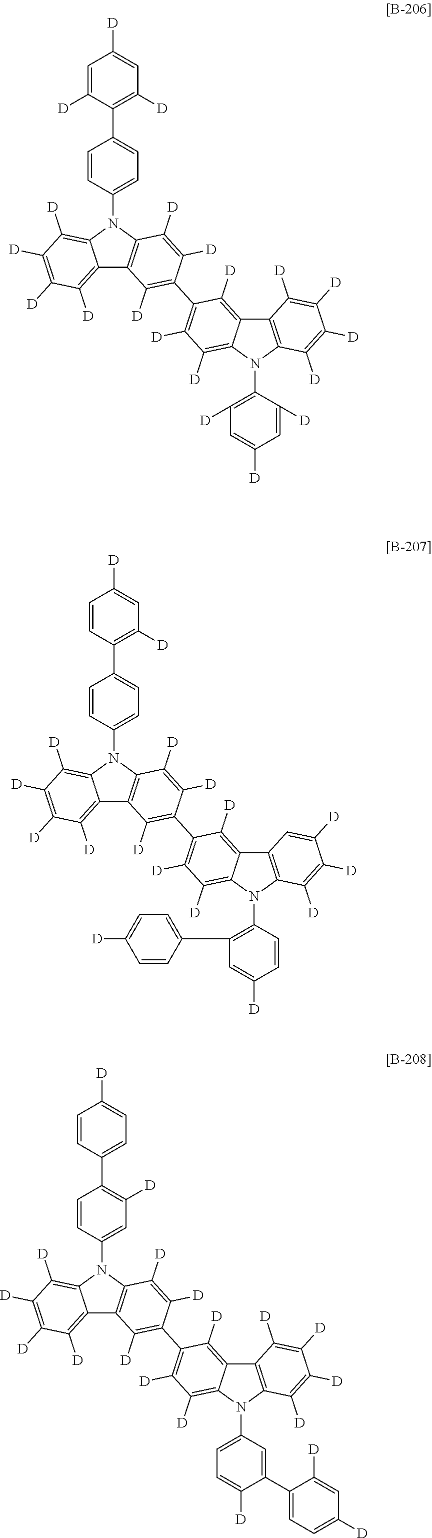

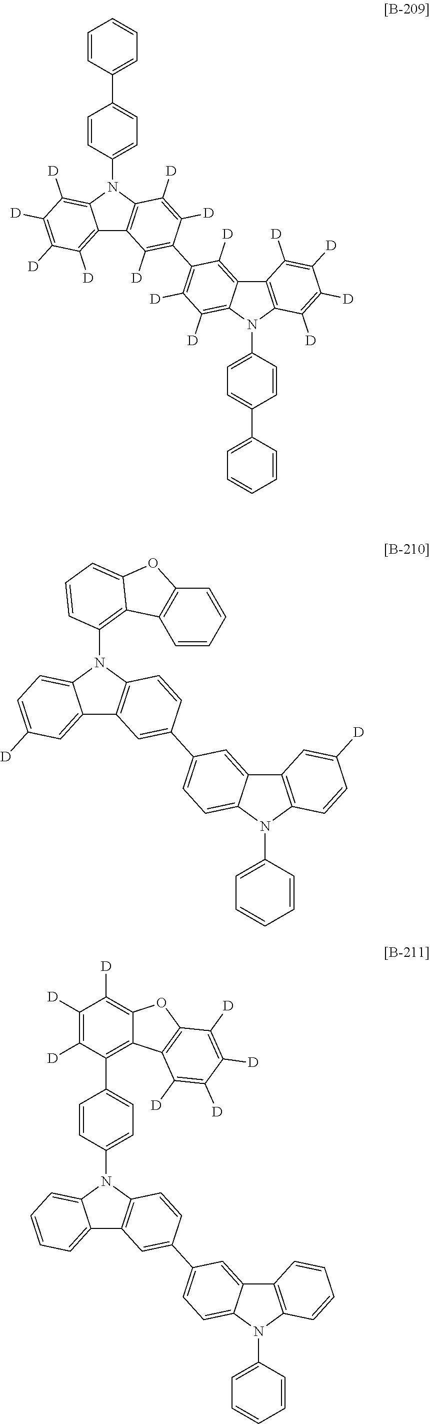 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1 ; [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H]]

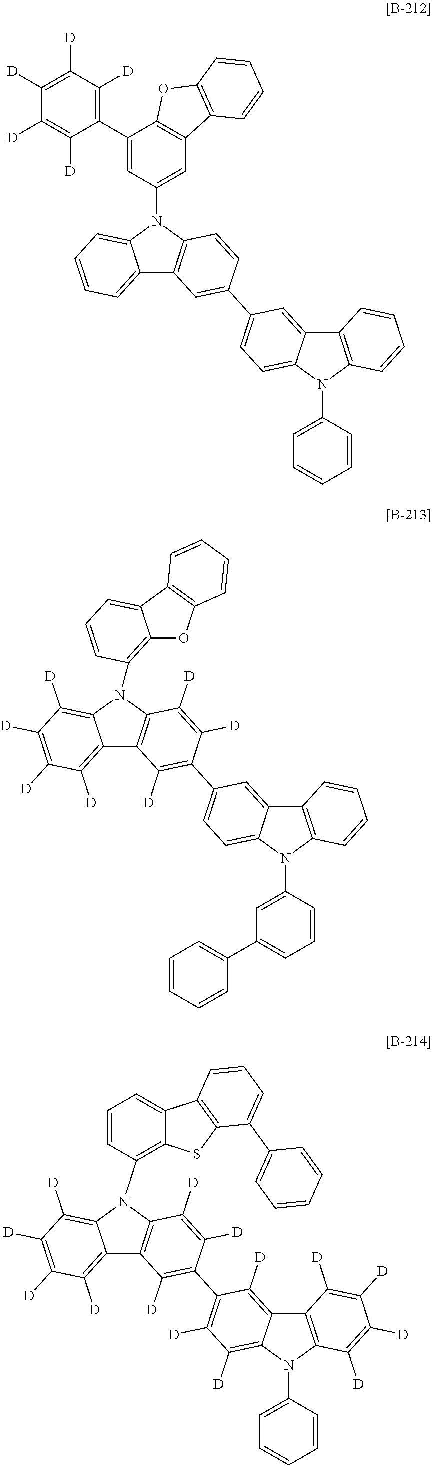 [SMILES: [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H] ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

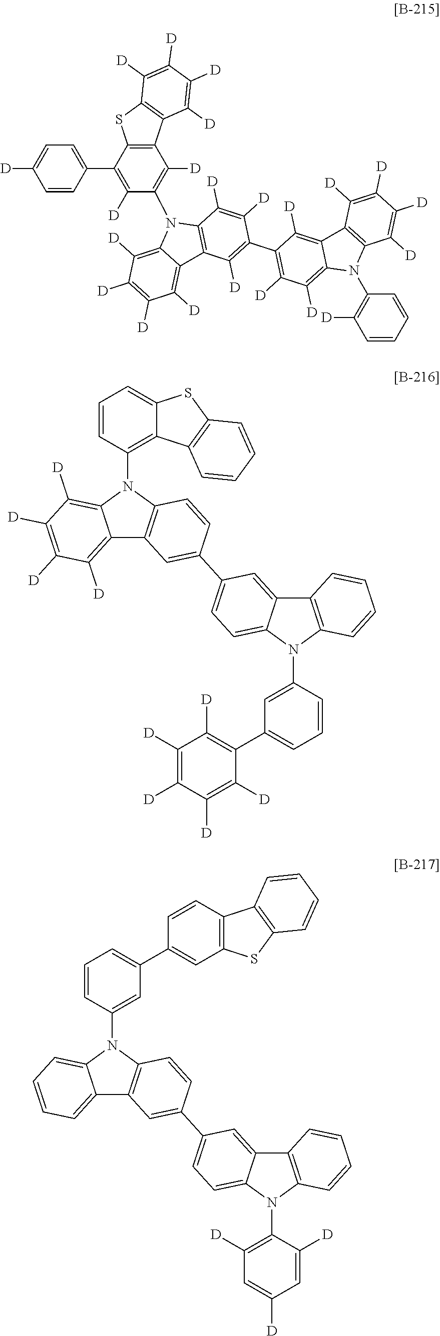 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1 ; [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

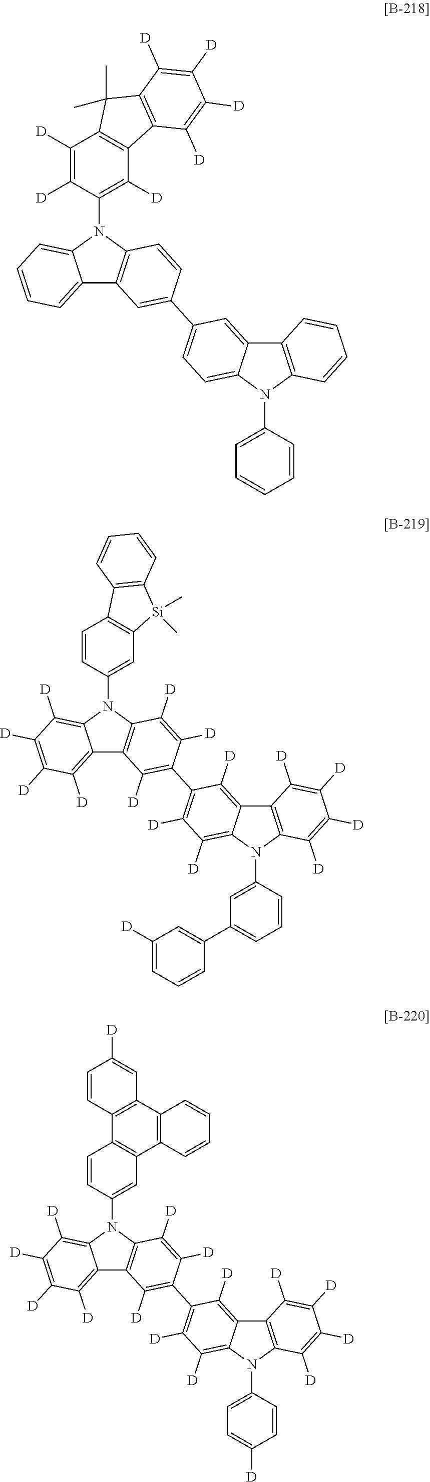 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1]

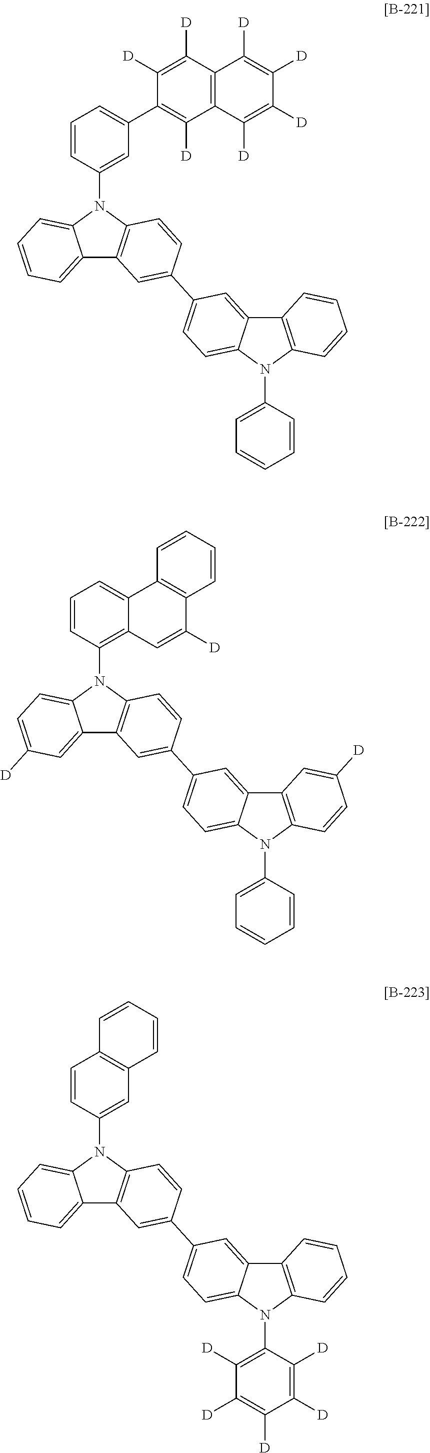 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

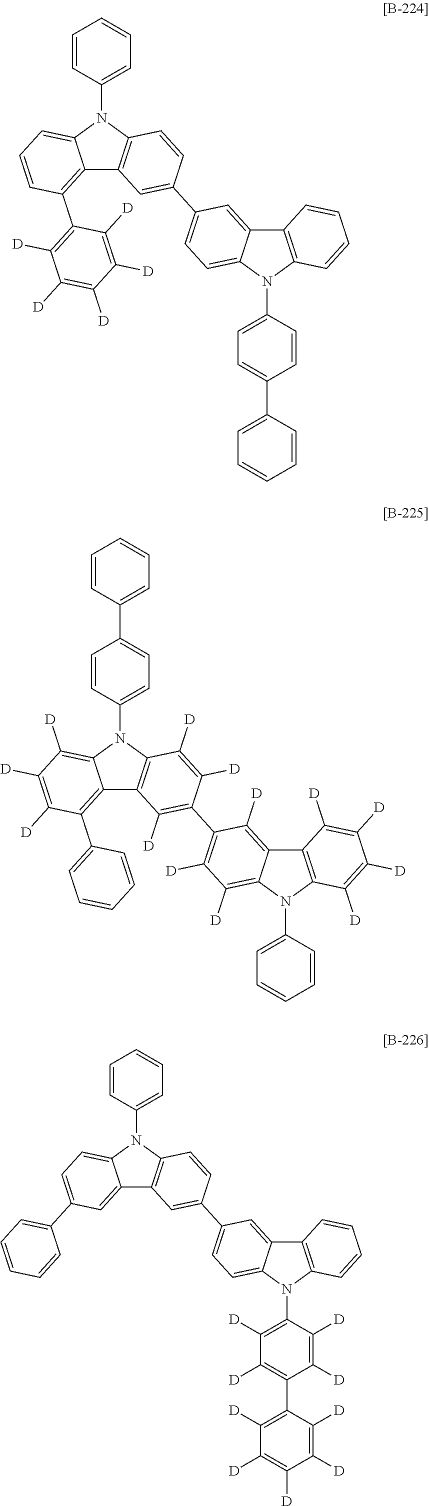 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

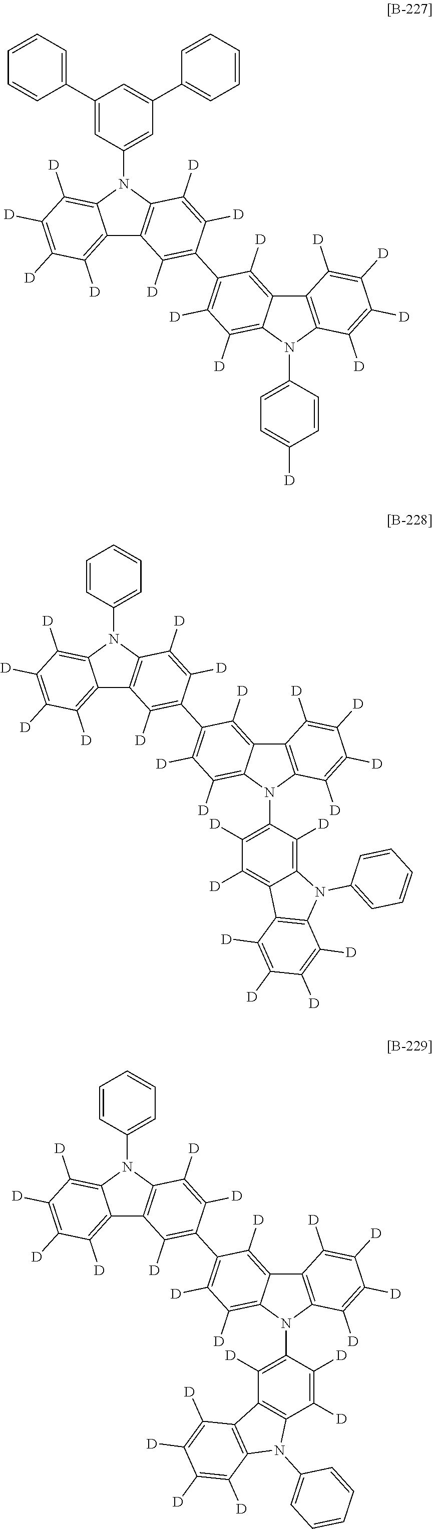

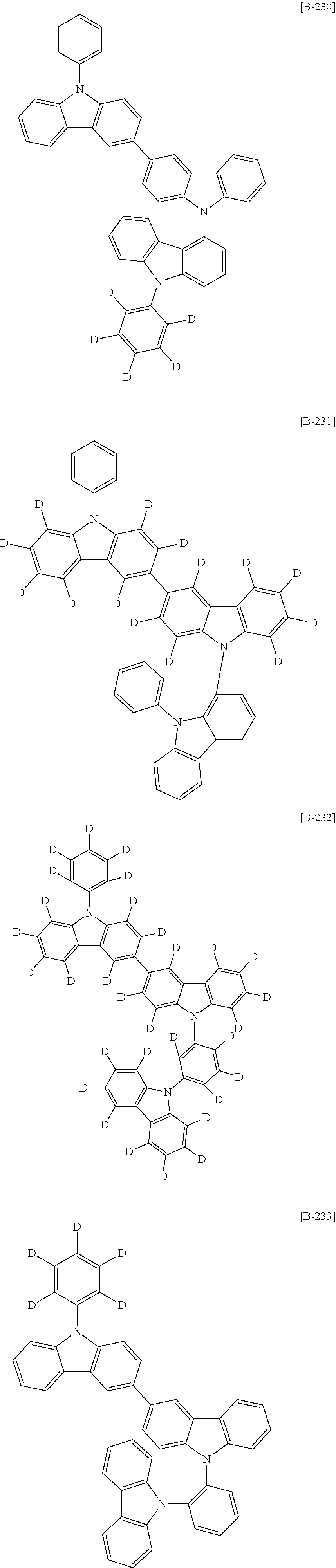

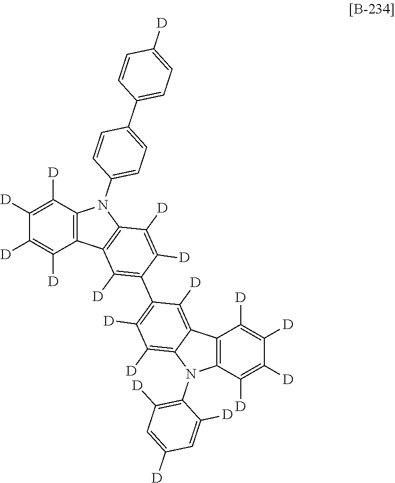 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

In an implementation, the second compound for organic optoelectronic device may be, e.g., a compound of Group 3.

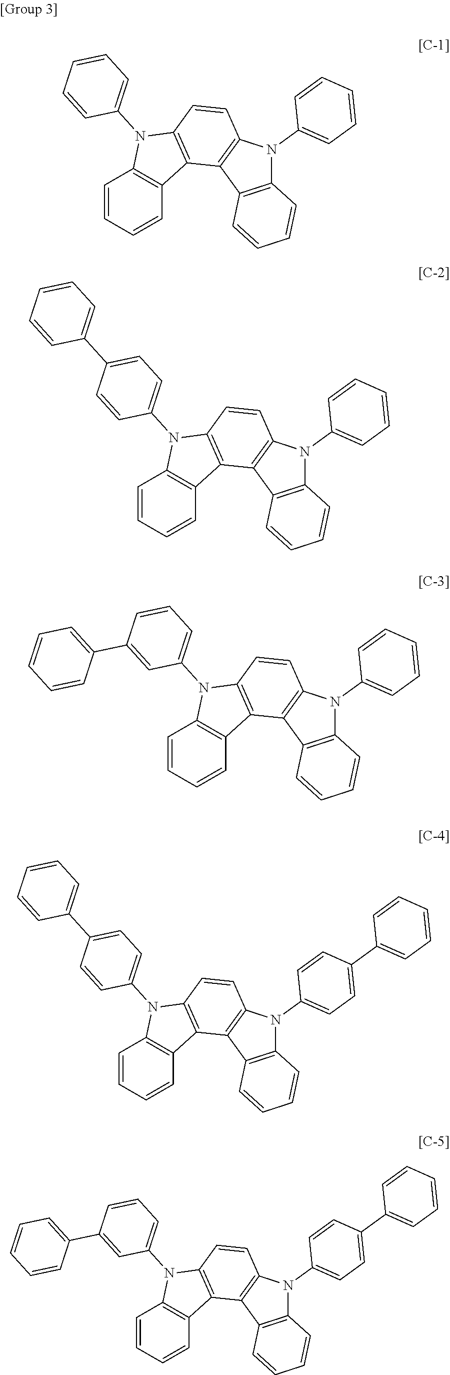 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1]

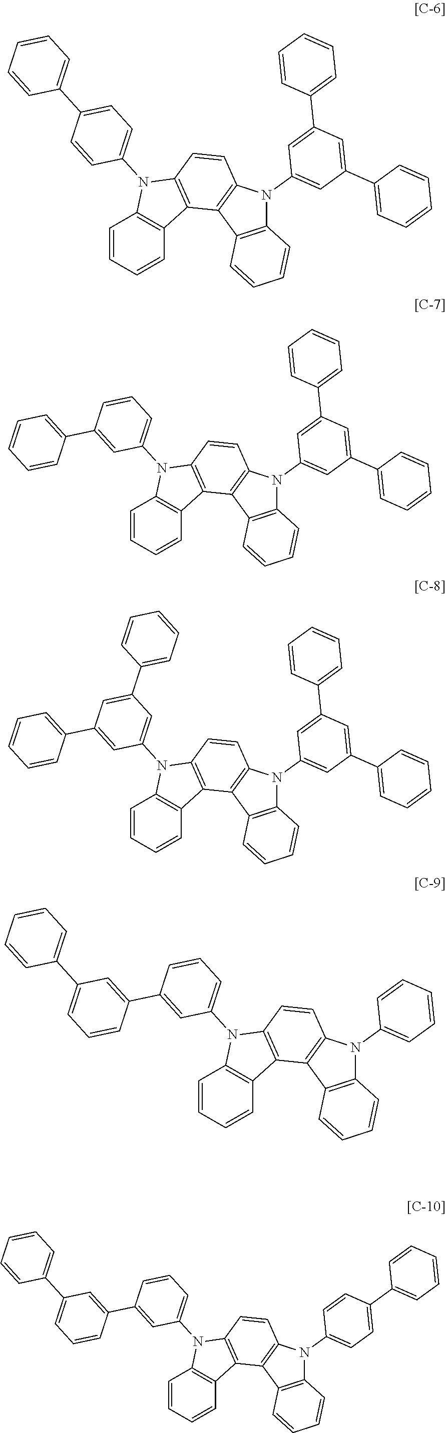 [SMILES: c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1]

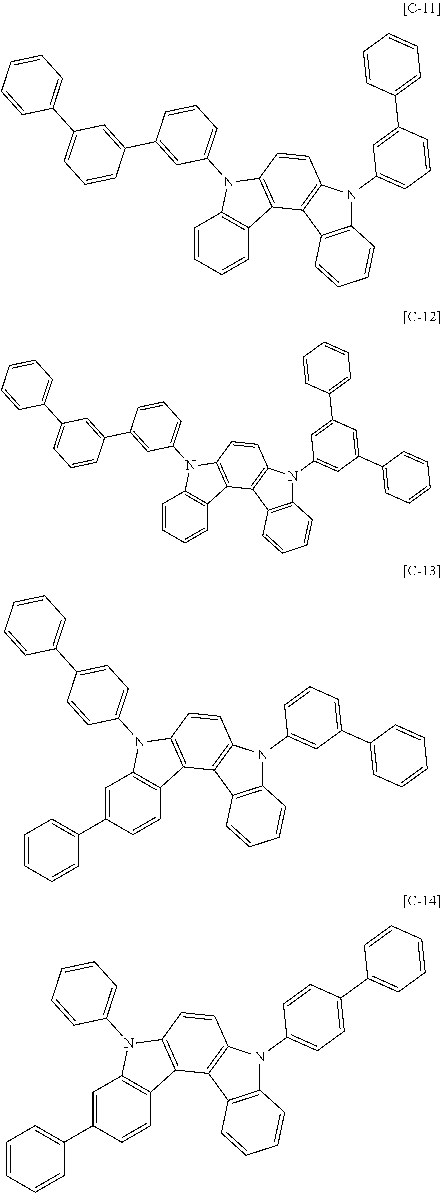 [SMILES: c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1 ; c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1]

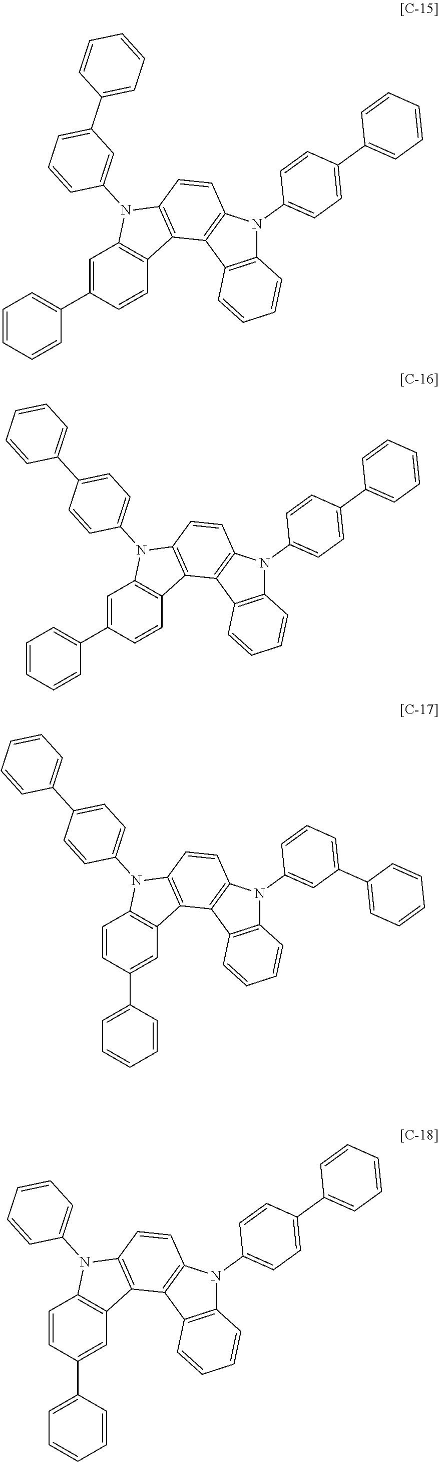 [SMILES: c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1 ; c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1]

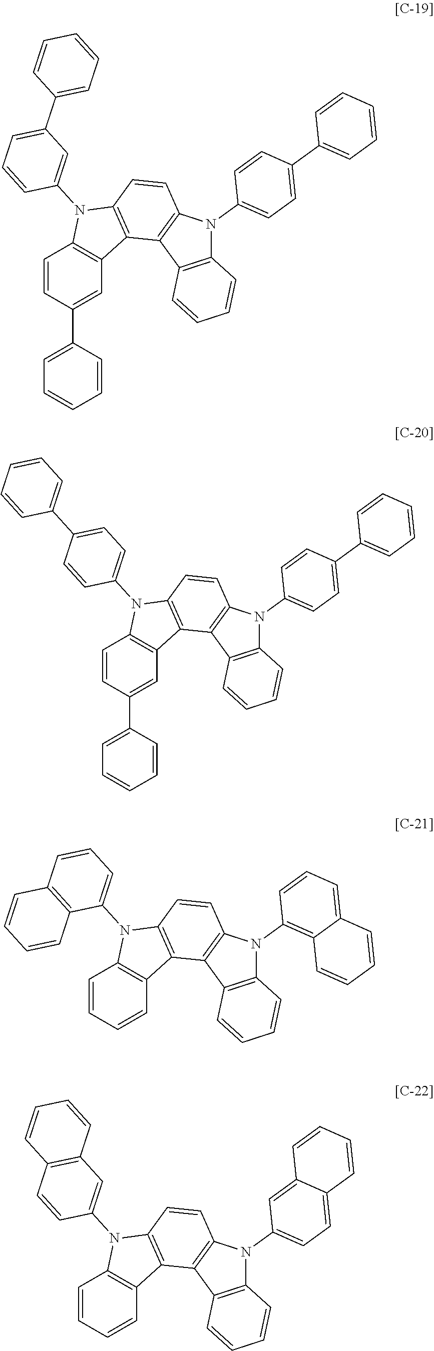 [SMILES: c1ccc(-c2ccc(-n3c4ccccc4c4c5c6cc(-c7ccccc7)ccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1]

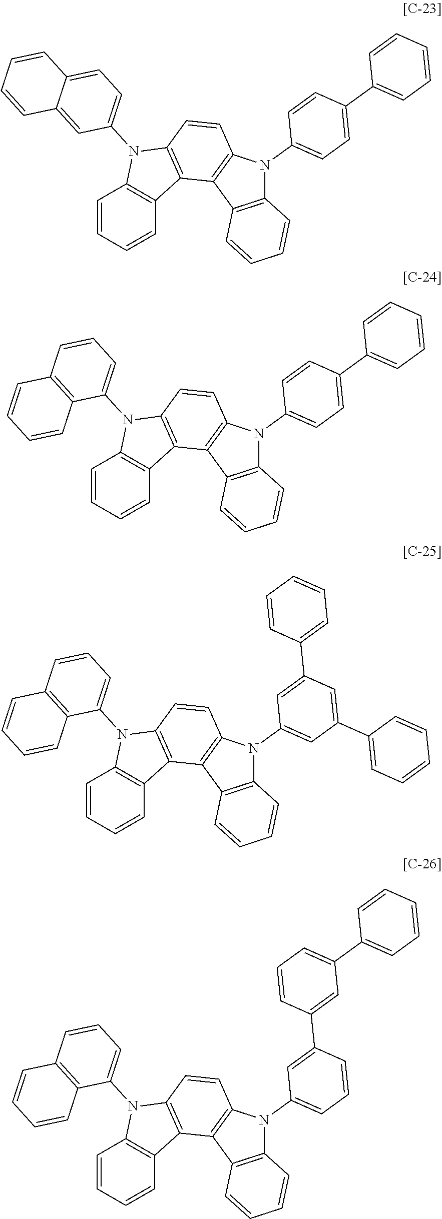 [SMILES: c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1]

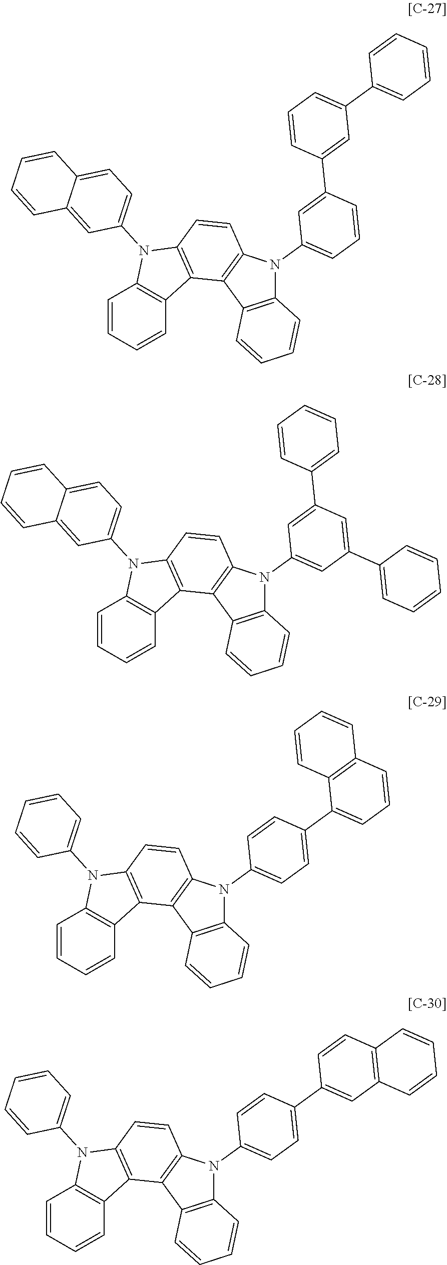 [SMILES: c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1]

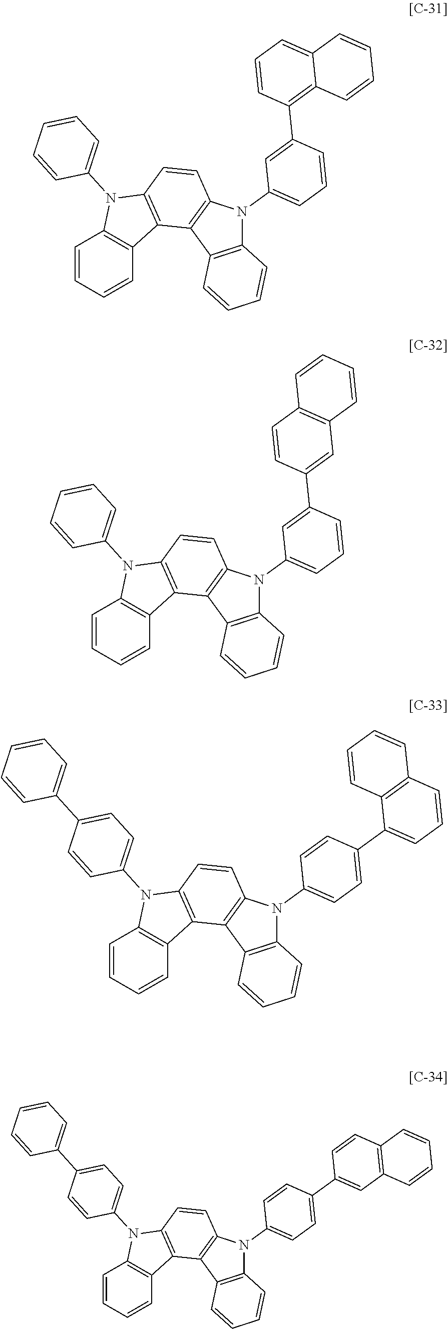 [SMILES: c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1]

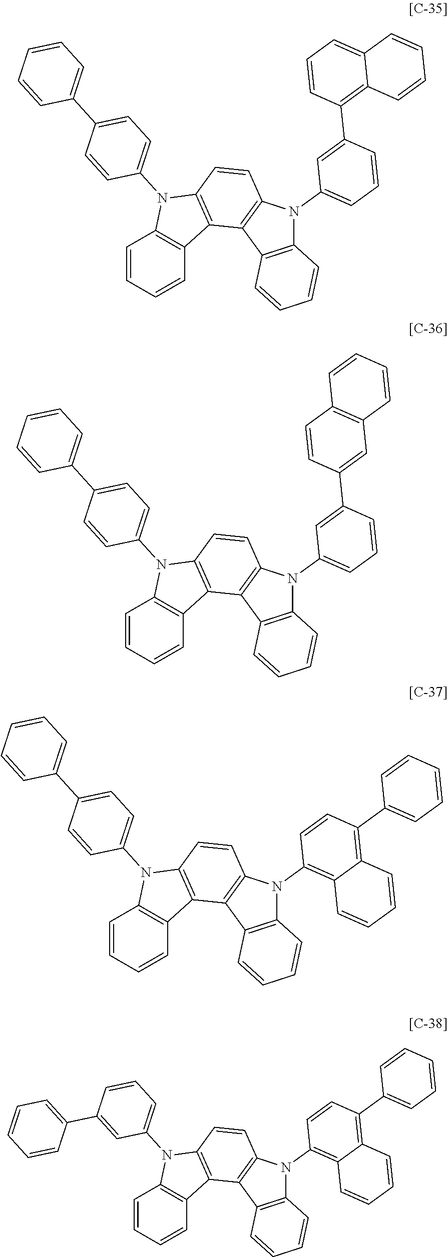 [SMILES: c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1]

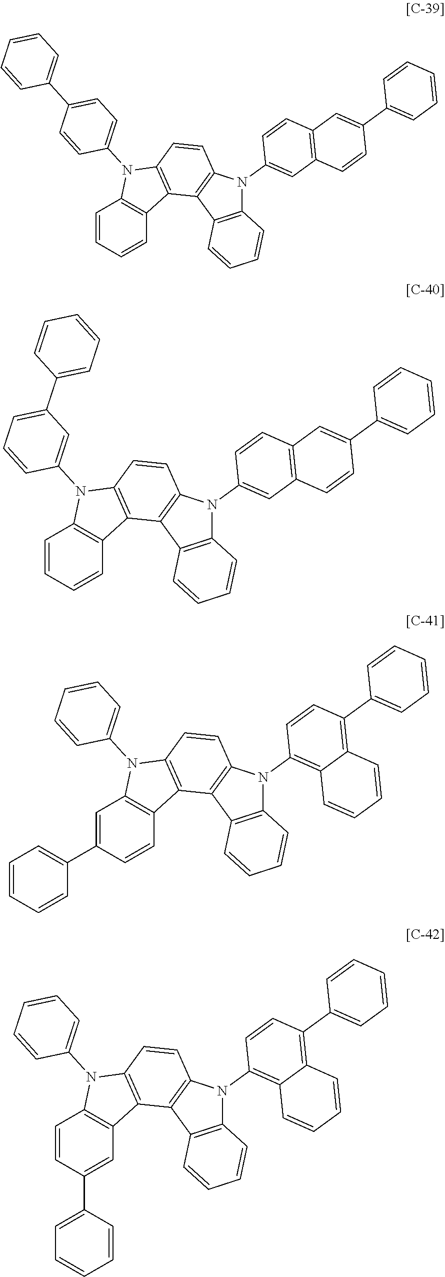 [SMILES: c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1]

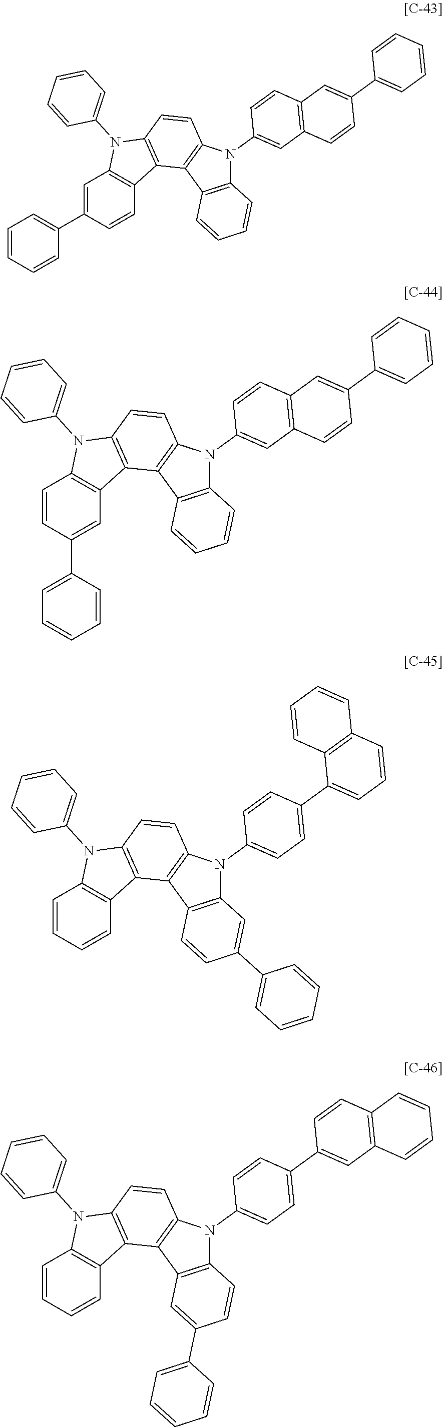 [SMILES: c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1]

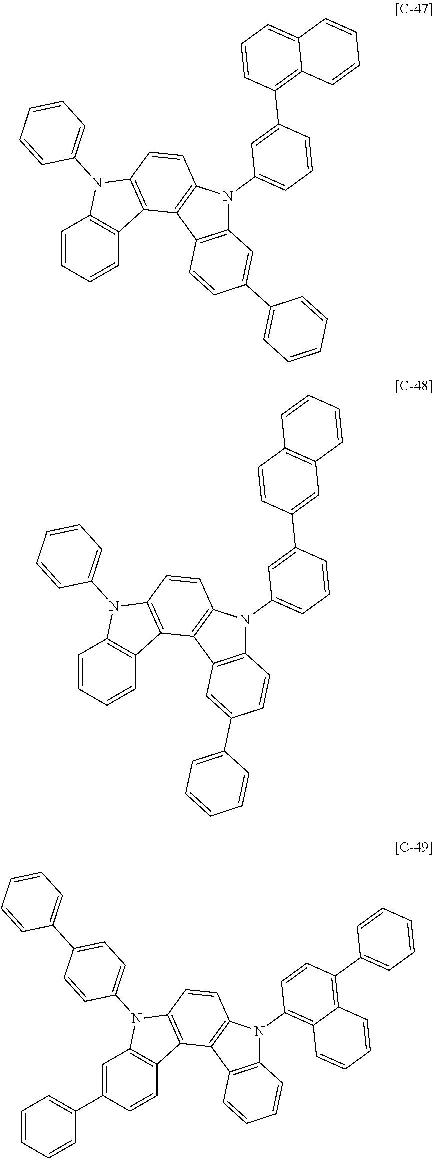

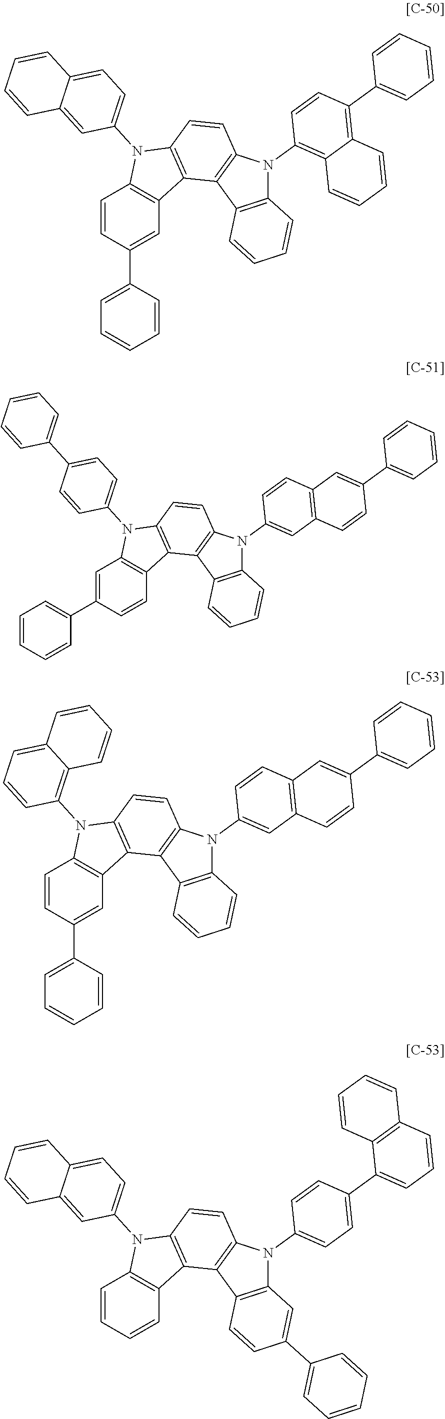 [SMILES: c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1 ; c1ccc(-c2ccc3c4c5c6ccccc6n(-c6ccc7ccccc7c6)c5ccc4n(-c4ccc(-c5cccc6ccccc56)cc4)c3c2)cc1]

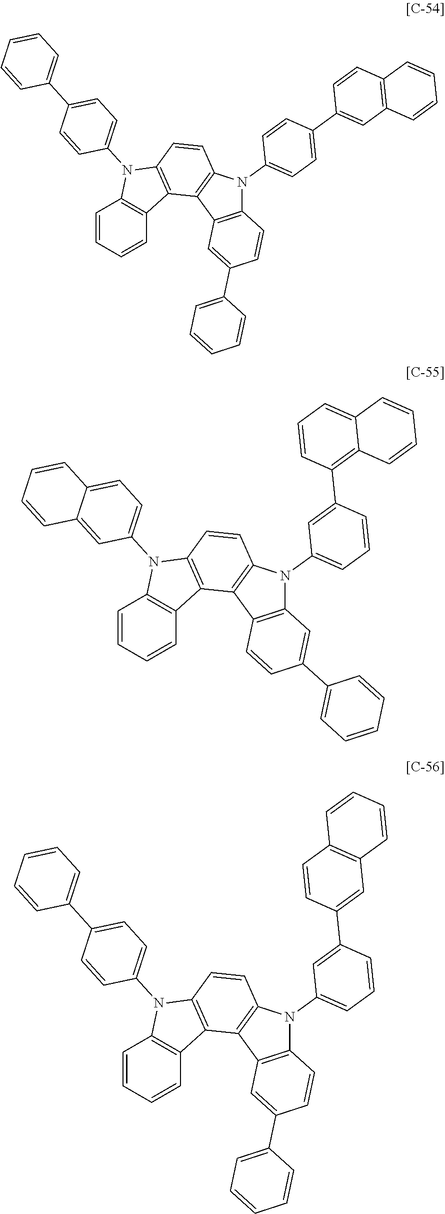 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

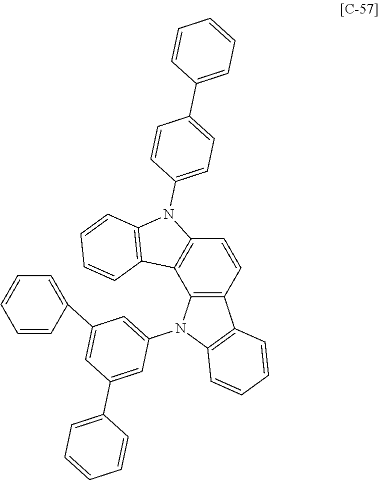 [SMILES: c1ccc(-c2ccc(-n3c4ccccc4c4c3ccc3c5ccccc5n(-c5cc(-c6ccccc6)cc(-c6ccccc6)c5)c34)cc2)cc1]

In an implementation, examples of Compound C-1 to Compound C-57 listed in Group 3 in which at least one hydrogen is replaced with deuterium are given below. In an implementation, deuterium may be substituted, e.g., as shown in Compound C-58 to Compound C-72, exemplified below.

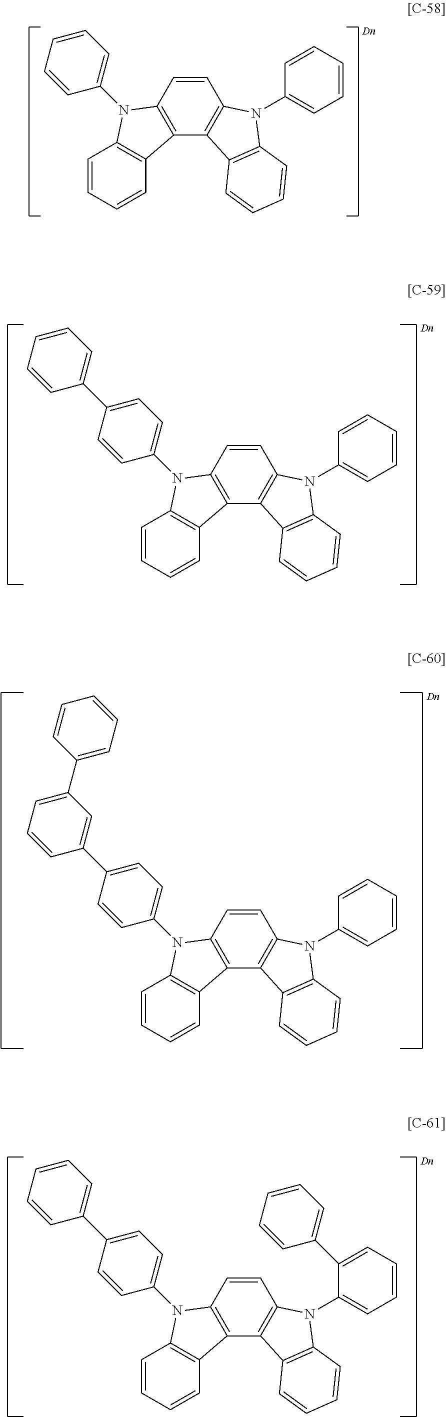

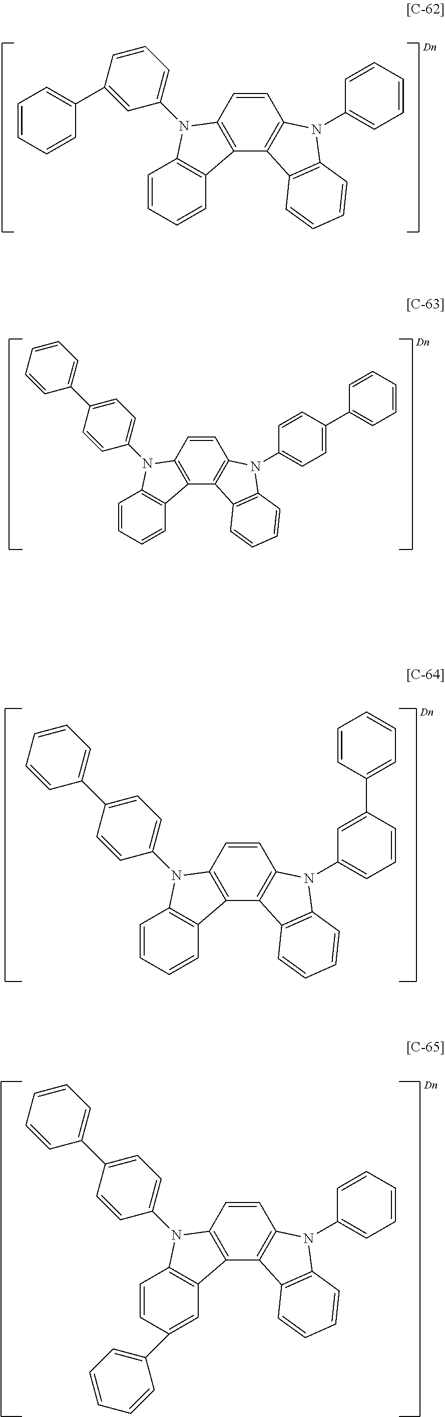

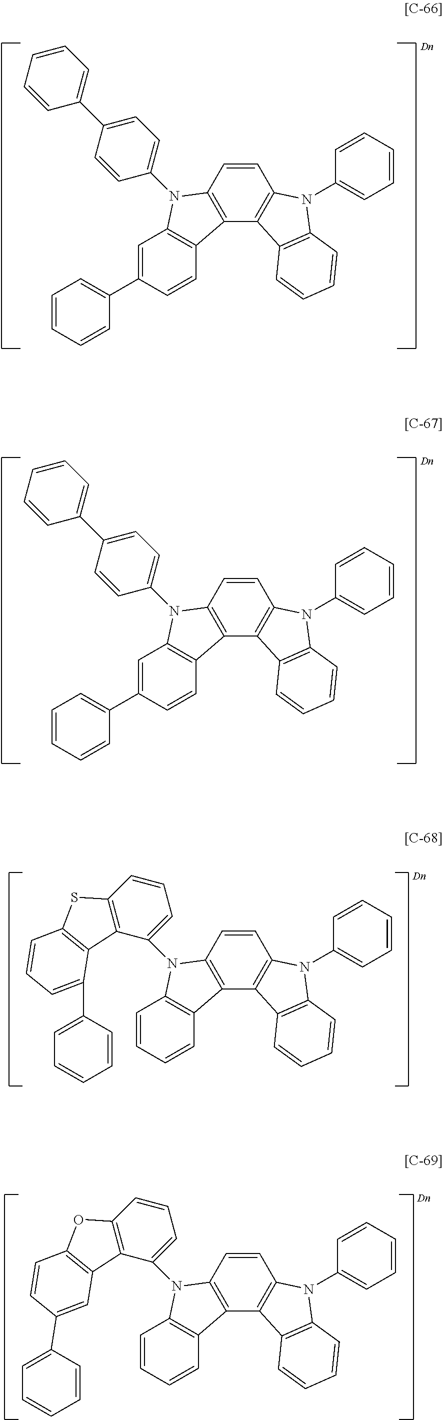

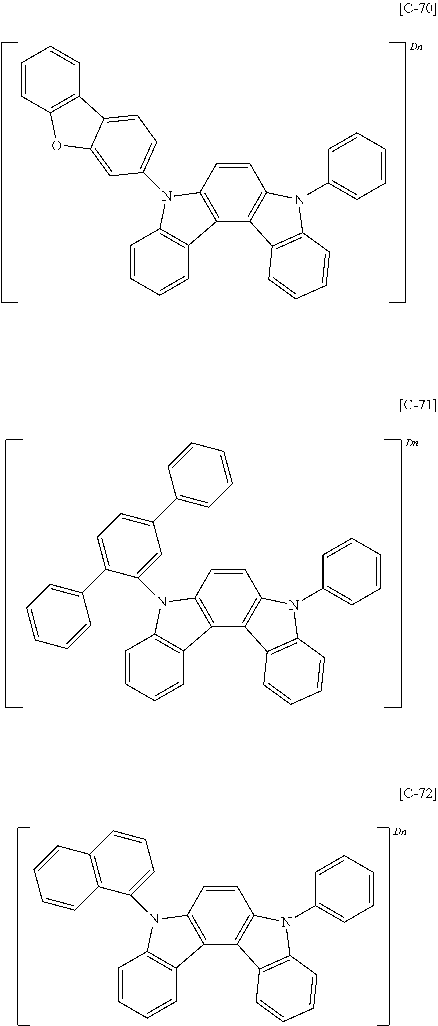

(Dn refers to the number of deuterium substitutions and indicates a structure substituted with one or more deuterium atoms)

The deuterium substitution position and deuterium substitution ratio may include all changeable ranges within the range of Compound C-1 to Compound C-72 (e.g., any hydrogen in any compound may be a protium or a deuterium).

The most specific structures for Compound C-58 to Compound C-72 of Group 3 are presented below as examples according to the position and substitution rate of deuterium substitution.

In an implementation, deuterium may be substituted, as shown in Compound C-73 to Compound C-102.

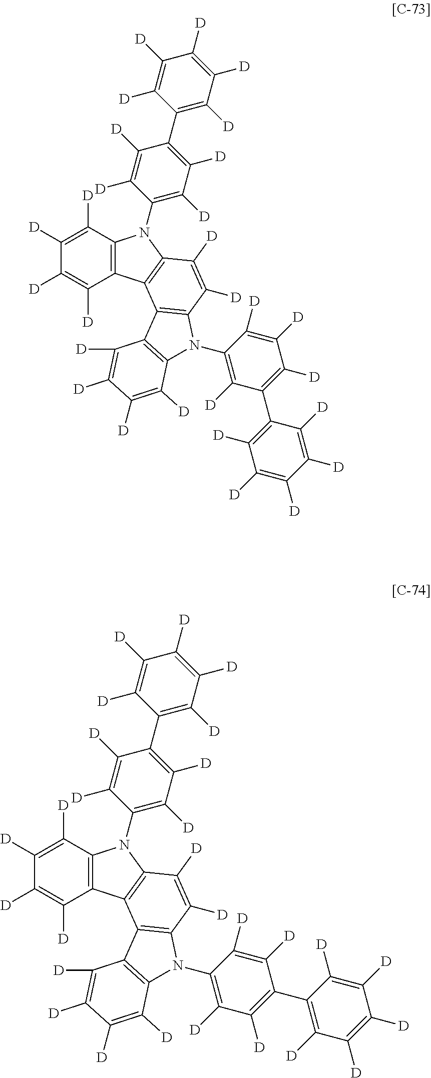 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

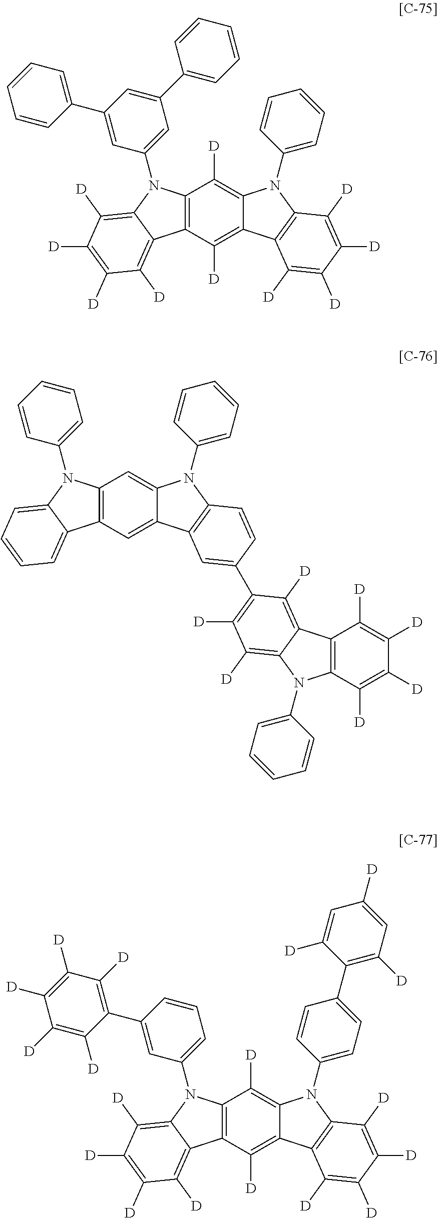 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

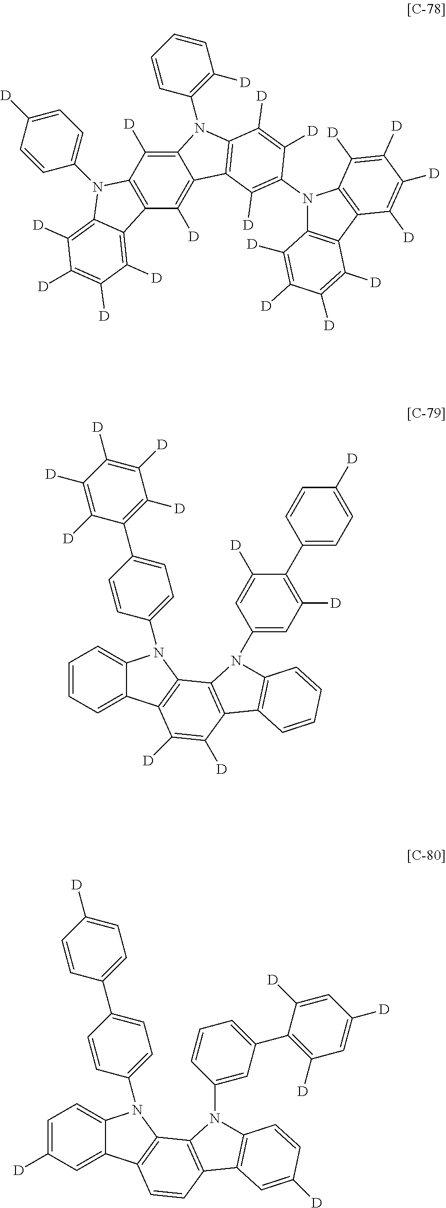 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1 ; [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

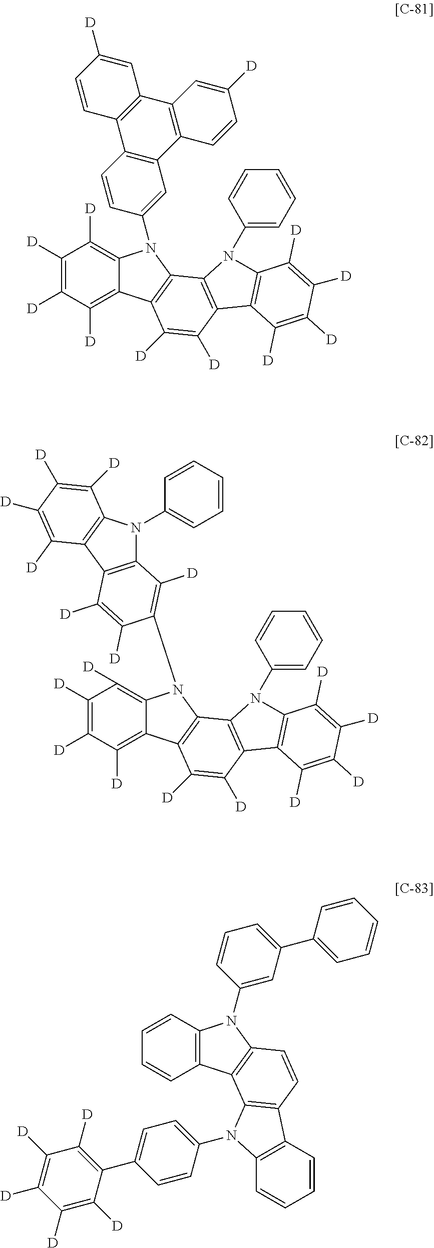 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1 ; [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H]]

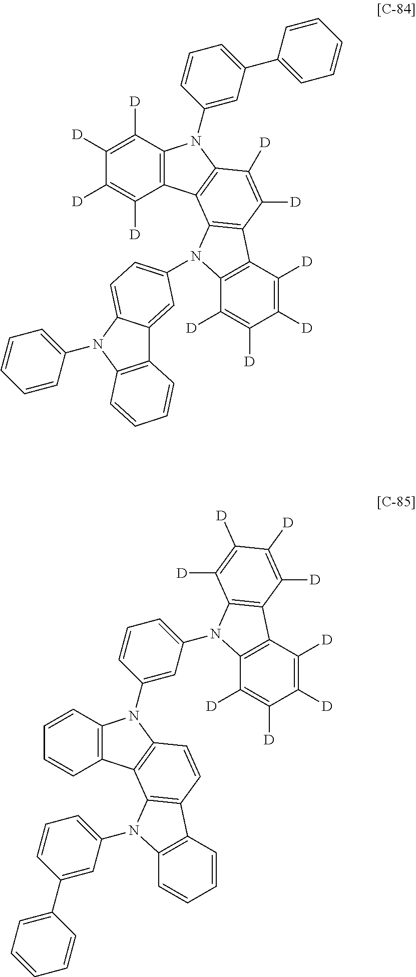 [SMILES: [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

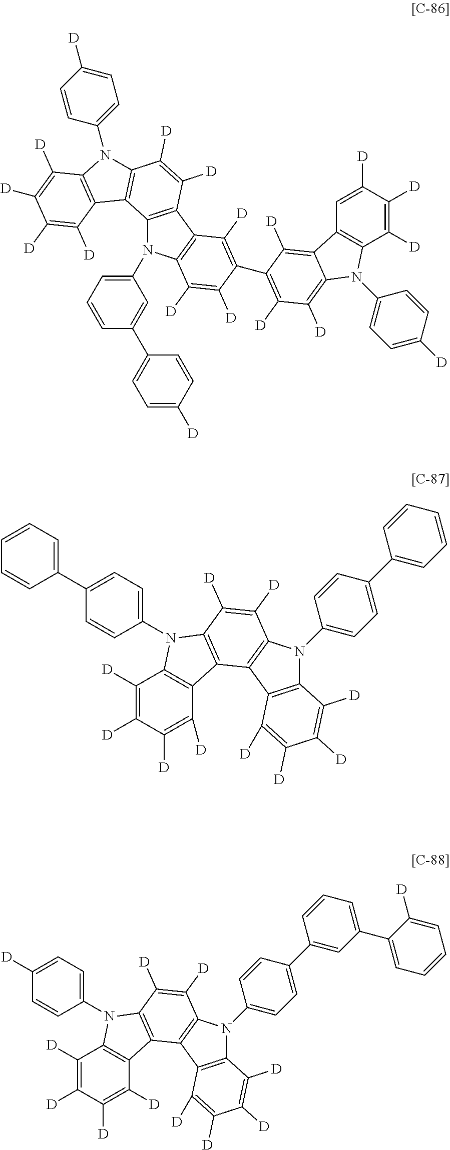 [SMILES: [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1ccc(-n2c3c([2H])c([2H])c([2H])c([2H])c3c3c4c5c([2H])c([2H])c([2H])c([2H])c5n(-c5ccc(-c6cccc(-c7ccccc7[2H])c6)cc5)c4c([2H])c([2H])c32)cc1]

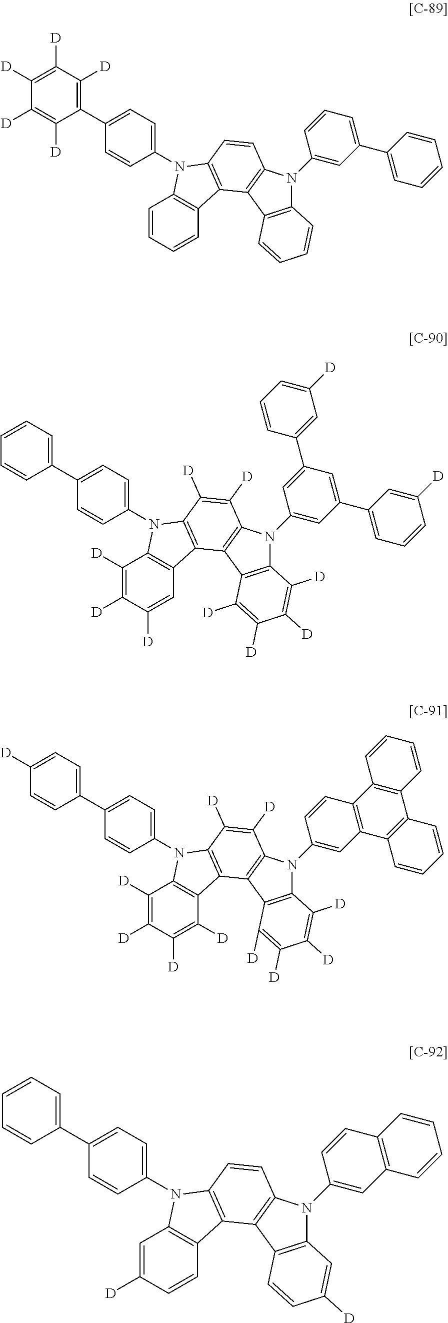 [SMILES: [2H]c1c([2H])c([2H])c(-c2ccc(-n3c4ccccc4c4c5c6ccccc6n(-c6cccc(-c7ccccc7)c6)c5ccc43)cc2)c([2H])c1[2H] ; [2H]c1cccc(-c2cc(-c3cccc([2H])c3)cc(-n3c4c([2H])c([2H])c([2H])c([2H])c4c4c5c6cc([2H])c([2H])c([2H])c6n(-c6ccc(-c7ccccc7)cc6)c5c([2H])c([2H])c43)c2)c1 ; [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1]

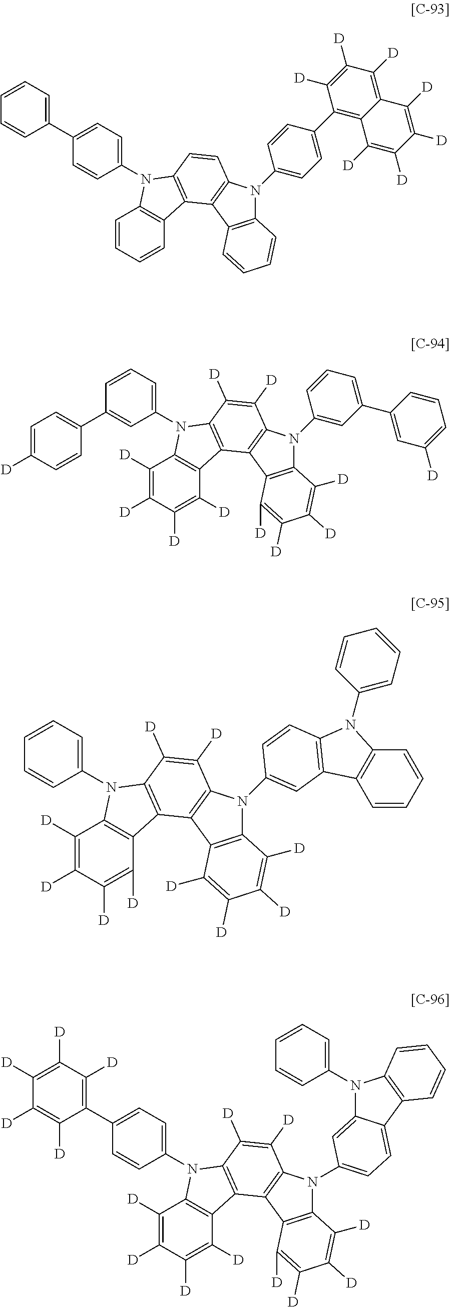

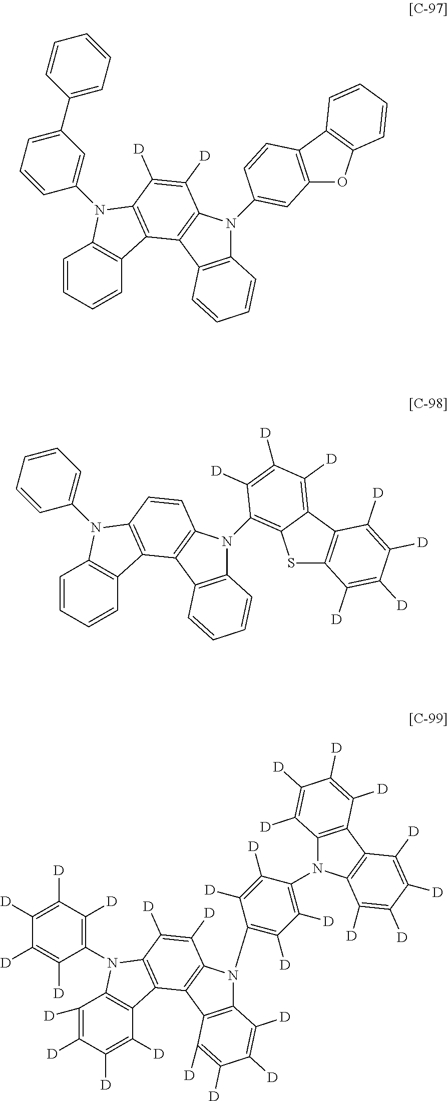 [SMILES: [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H]]

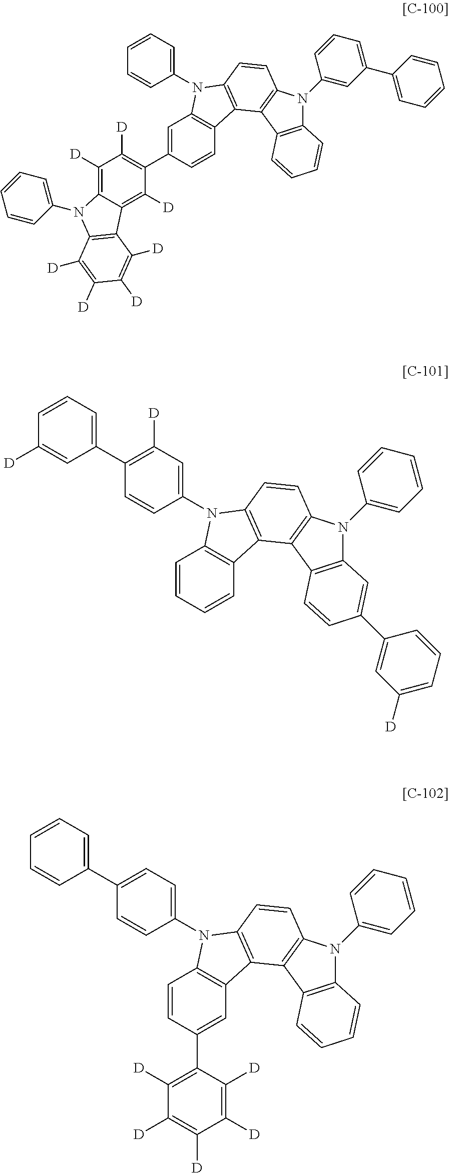 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

In an implementation, the second compound for organic optoelectronic device may be, e.g., a compound of Group 4.

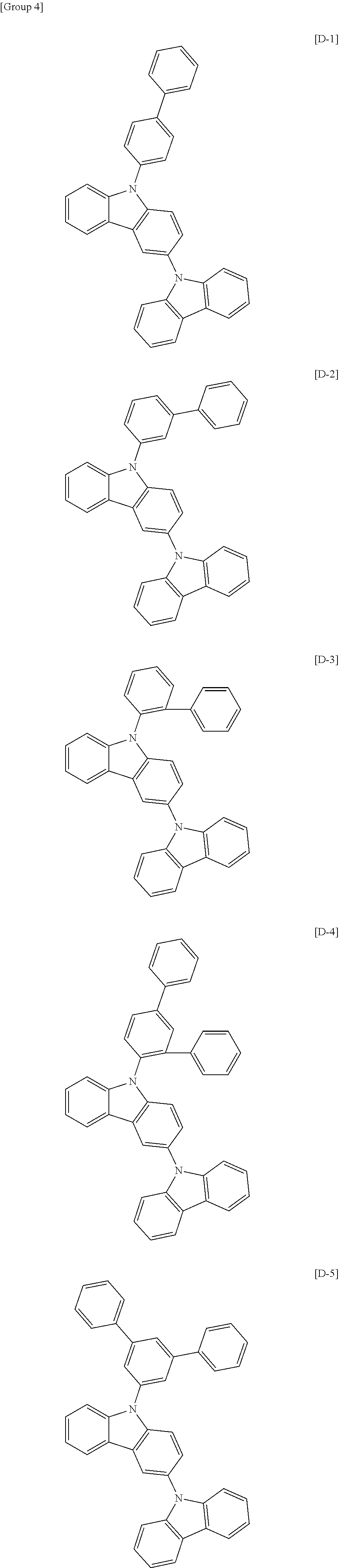 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1 ; c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1]

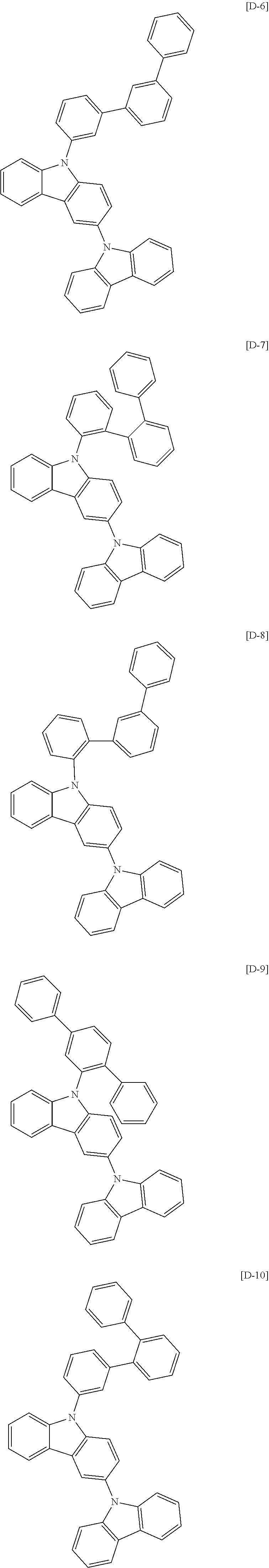 [SMILES: c1ccc(-c2cccc(-c3cccc(-n4c5ccccc5c5cc(-n6c7ccccc7c7ccccc76)ccc54)c3)c2)cc1 ; c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1 ; c1ccc(-c2ccccc2-c2cccc(-n3c4ccccc4c4cc(-n5c6ccccc6c6ccccc65)ccc43)c2)cc1]

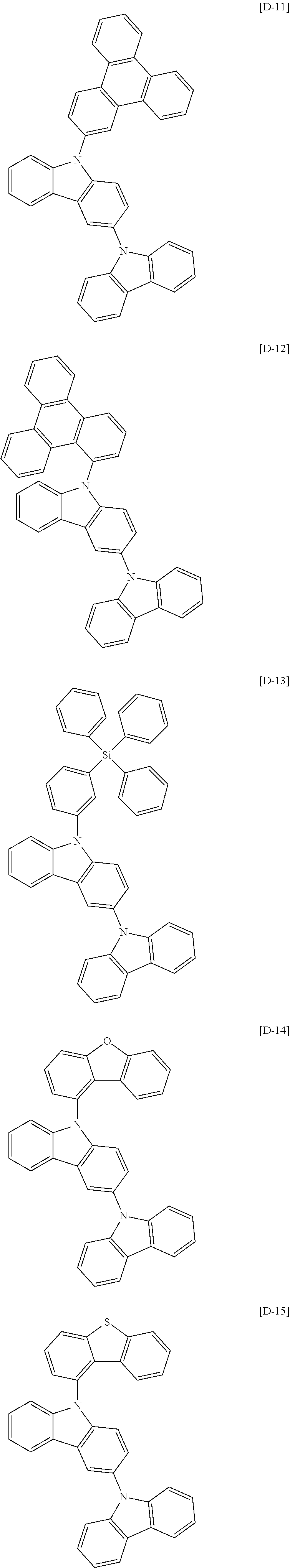 [SMILES: c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21 ; c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

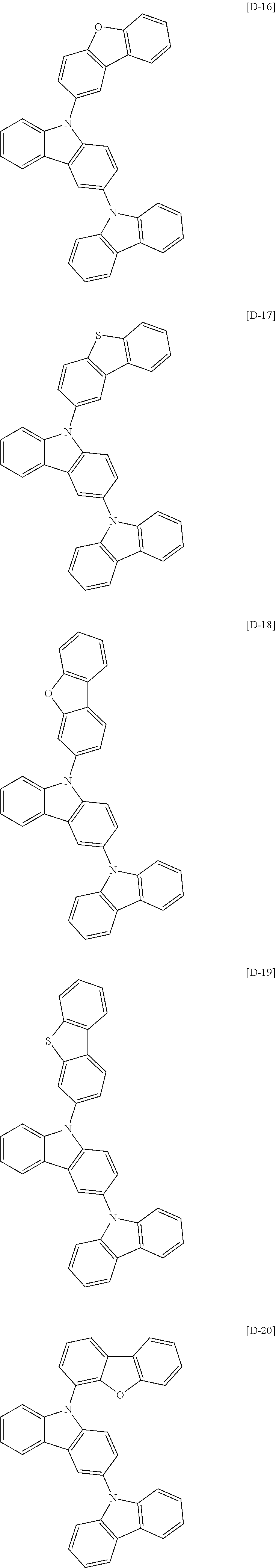 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12 ; c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12]

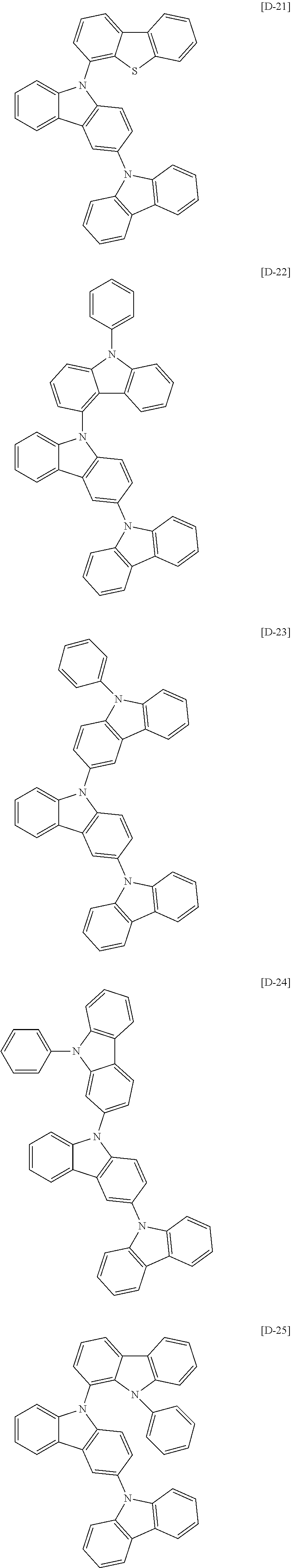 [SMILES: c1ccc2c(c1)sc1c(-n3c4ccccc4c4cc(-n5c6ccccc6c6ccccc65)ccc43)cccc12 ; c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1 ; c1ccc(-n2c3ccccc3c3cccc(-n4c5ccccc5c5cc(-n6c7ccccc7c7ccccc76)ccc54)c32)cc1]

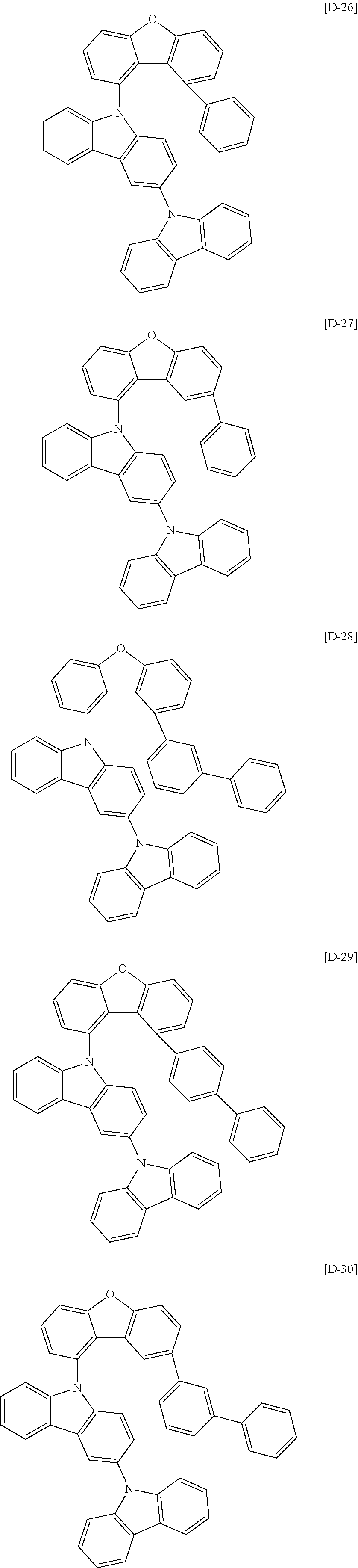 [SMILES: c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1 ; c1ccc(-c2cccc(-c3cccc4oc5cccc(-n6c7ccccc7c7cc(-n8c9ccccc9c9ccccc98)ccc76)c5c34)c2)cc1 ; c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1]

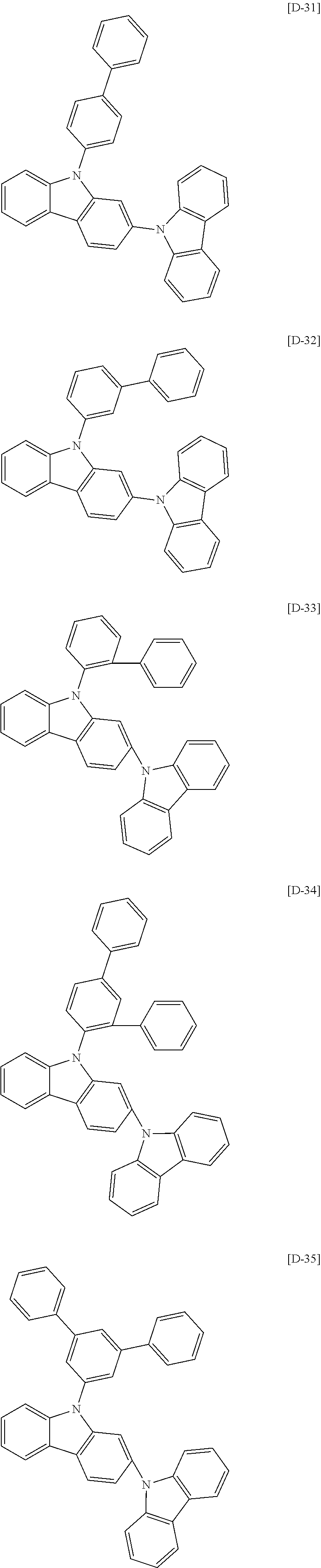 [SMILES: c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1 ; c1ccc(-c2ccc(-n3c4ccccc4c4ccc(-n5c6ccccc6c6ccccc65)cc43)c(-c3ccccc3)c2)cc1 ; c1ccc(-c2cc(-c3ccccc3)cc(-n3c4ccccc4c4ccc(-n5c6ccccc6c6ccccc65)cc43)c2)cc1]

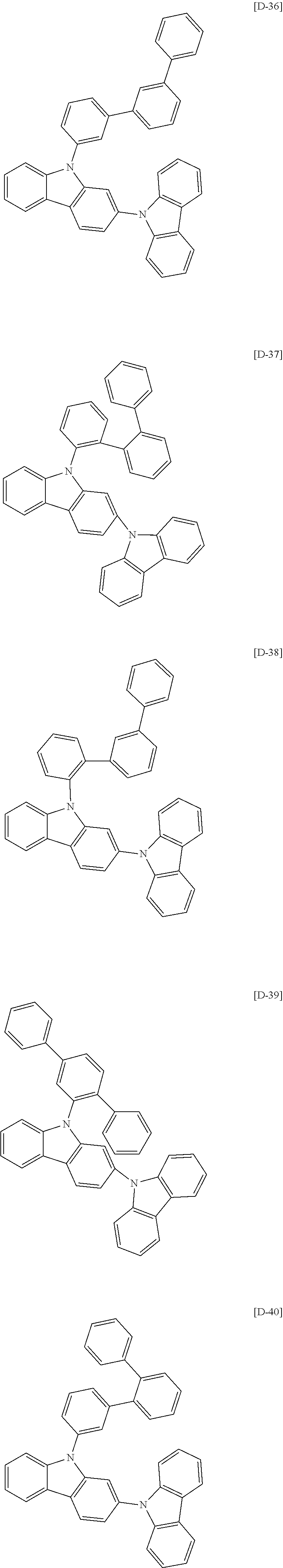 [SMILES: c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1 ; c1ccc(-c2cccc(-c3ccccc3-n3c4ccccc4c4ccc(-n5c6ccccc6c6ccccc65)cc43)c2)cc1 ; c1ccc(-c2ccc(-c3ccccc3)c(-n3c4ccccc4c4ccc(-n5c6ccccc6c6ccccc65)cc43)c2)cc1 ; c1ccc(-c2ccccc2-c2cccc(-n3c4ccccc4c4ccc(-n5c6ccccc6c6ccccc65)cc43)c2)cc1]

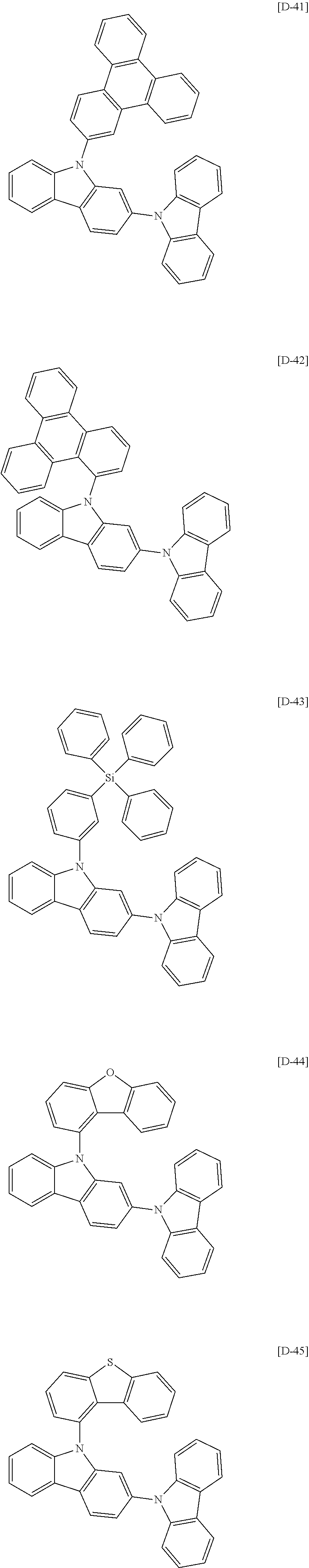 [SMILES: c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12]

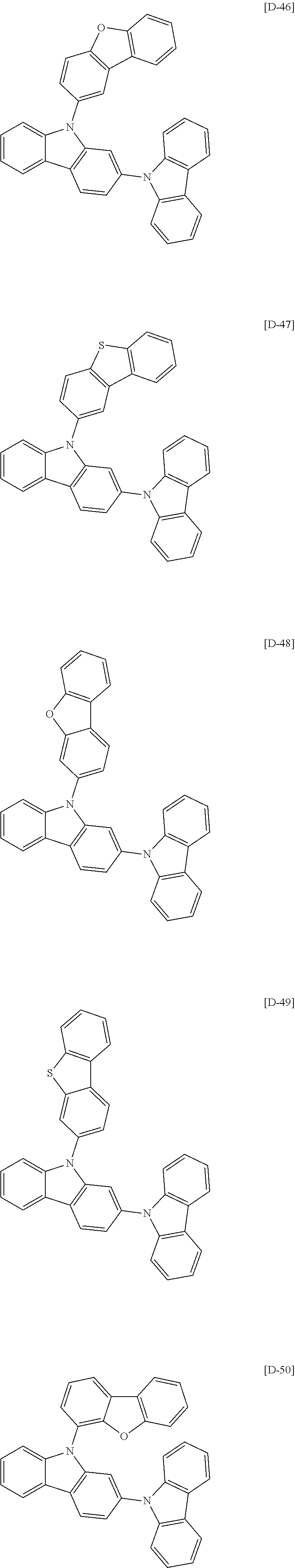 [SMILES: c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12 ; c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12]

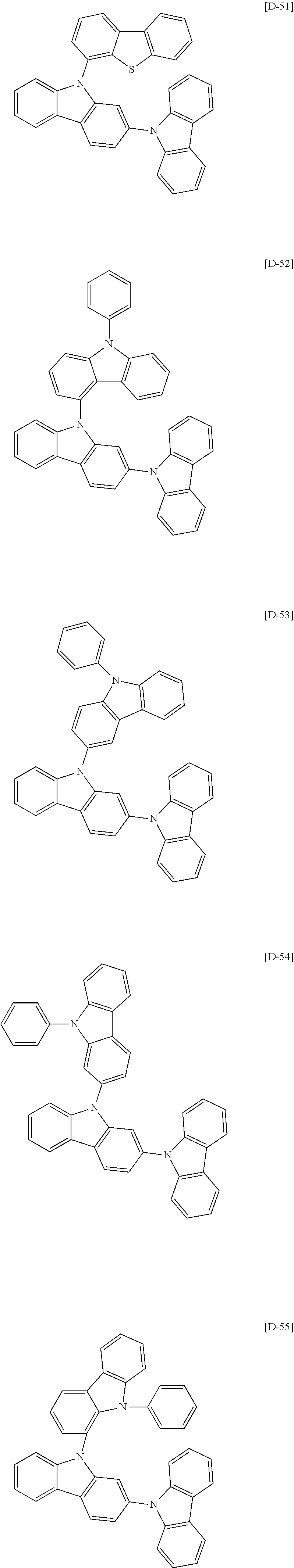 [SMILES: c1ccc2c(c1)sc1c(-n3c4ccccc4c4ccc(-n5c6ccccc6c6ccccc65)cc43)cccc12 ; c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1 ; c1ccc(-n2c3ccccc3c3cccc(-n4c5ccccc5c5ccc(-n6c7ccccc7c7ccccc76)cc54)c32)cc1]

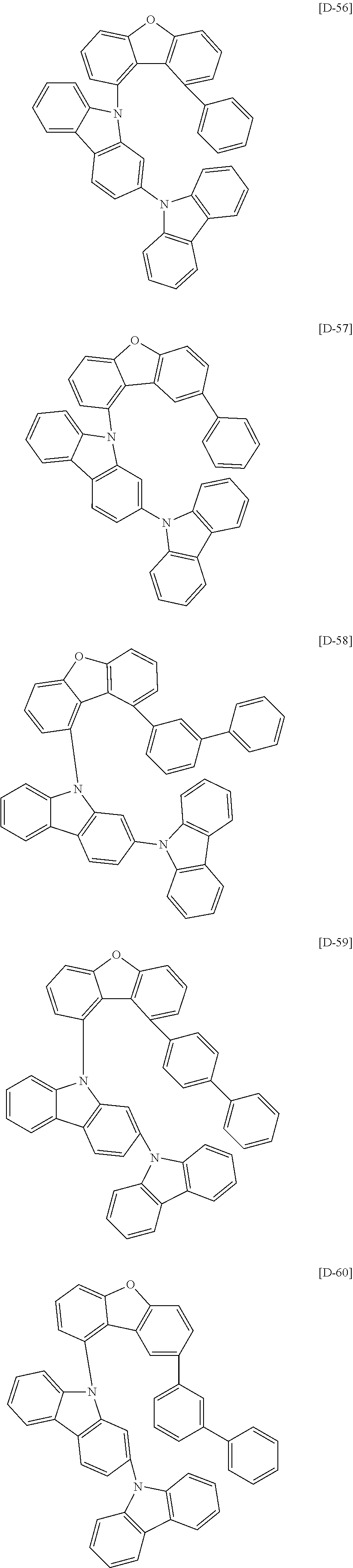 [SMILES: c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1 ; c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1]

Additionally, examples of Compound D-1 to Compound D-60 listed in Group 4 in which at least one hydrogen is replaced with deuterium are given below.

In an implementation, deuterium may be substituted, e.g., as shown in Compound D-61 to Compound D-120 exemplified below.

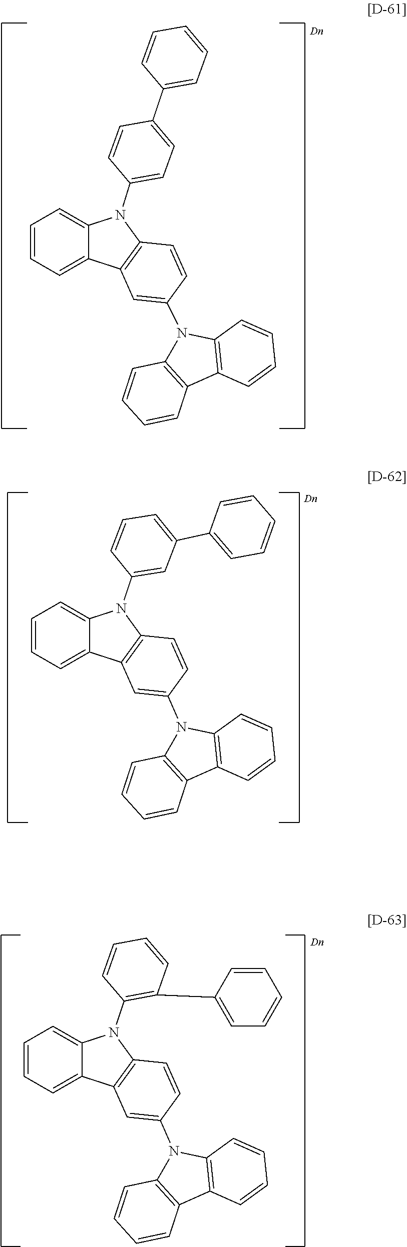

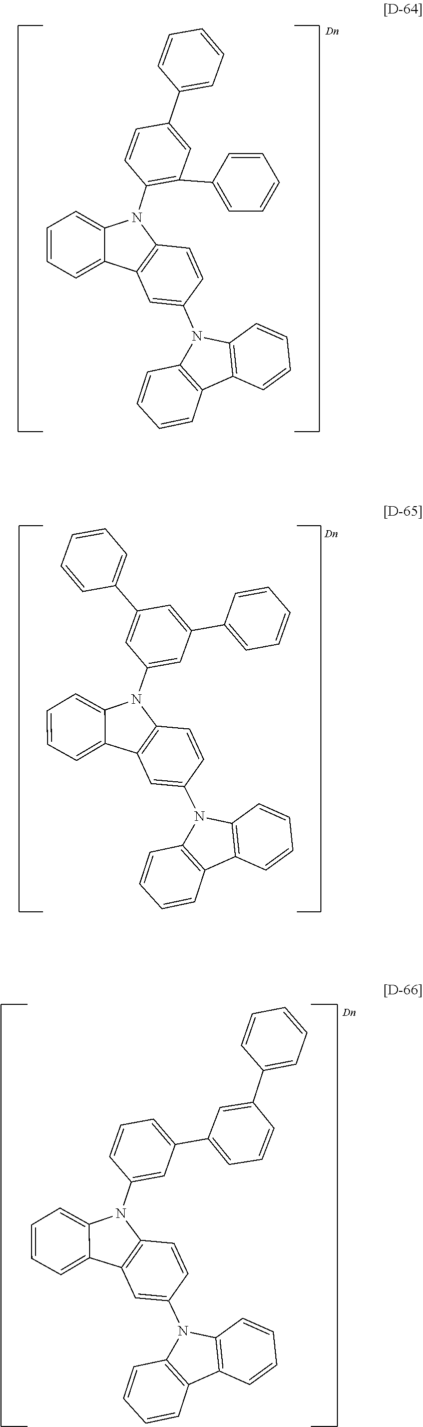

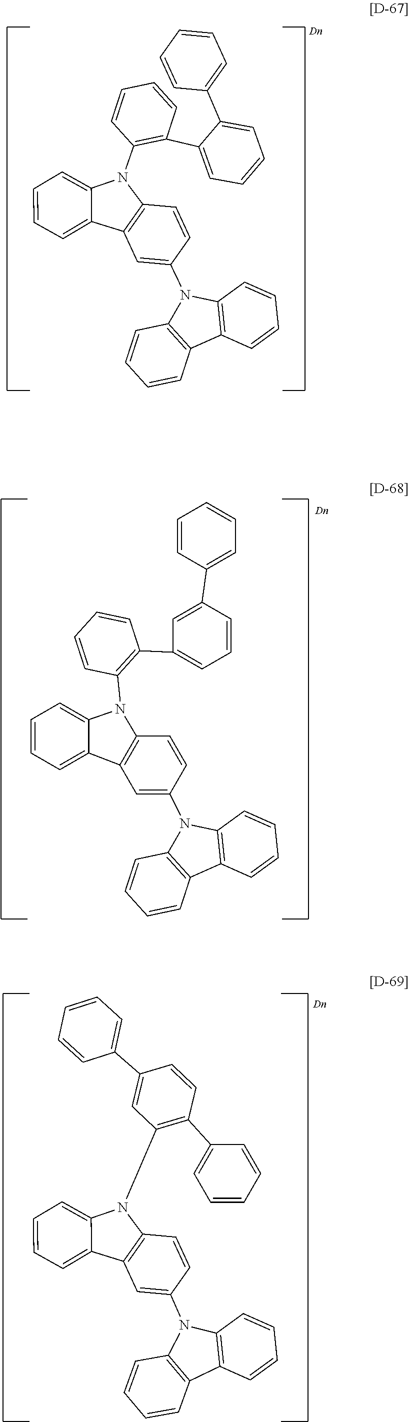

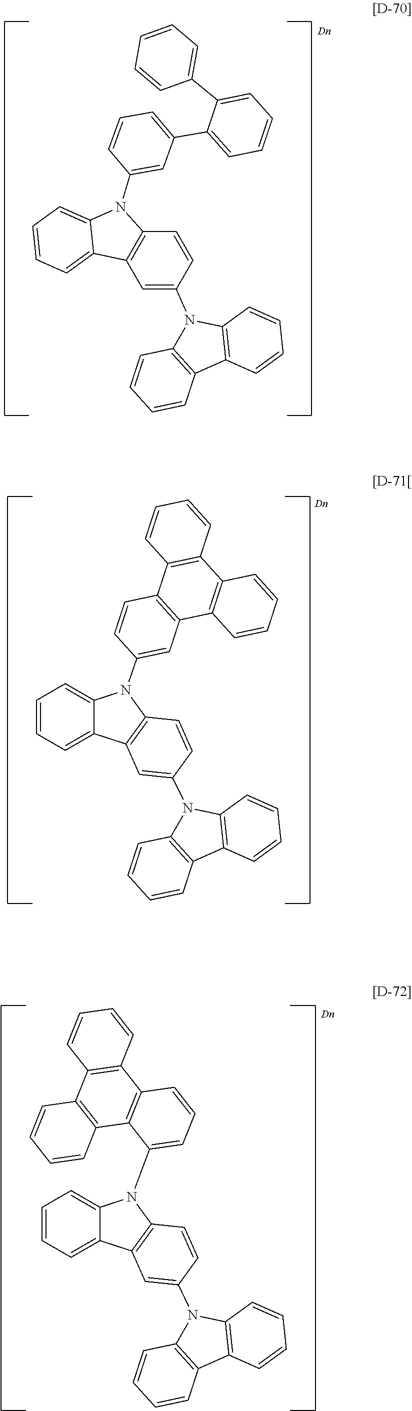

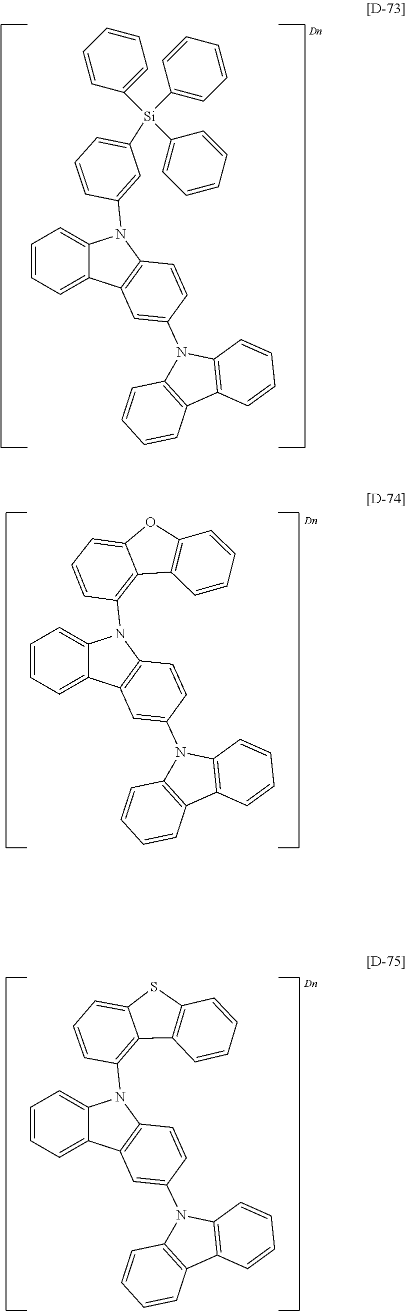

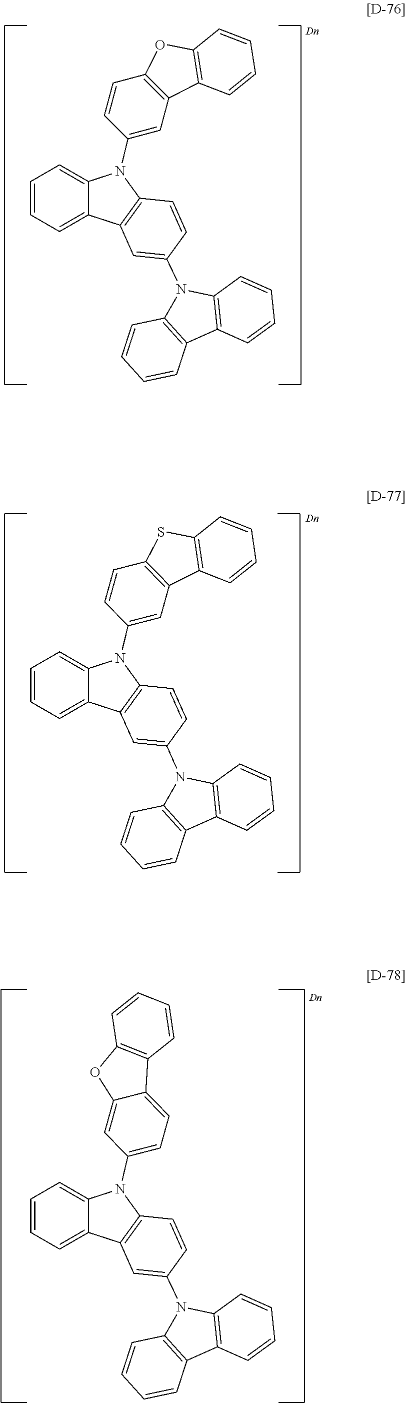

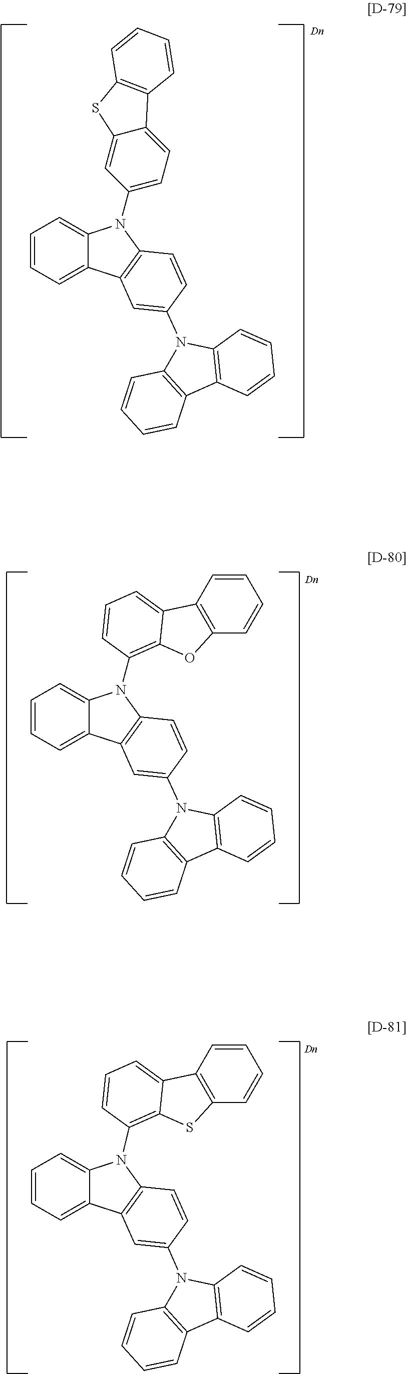

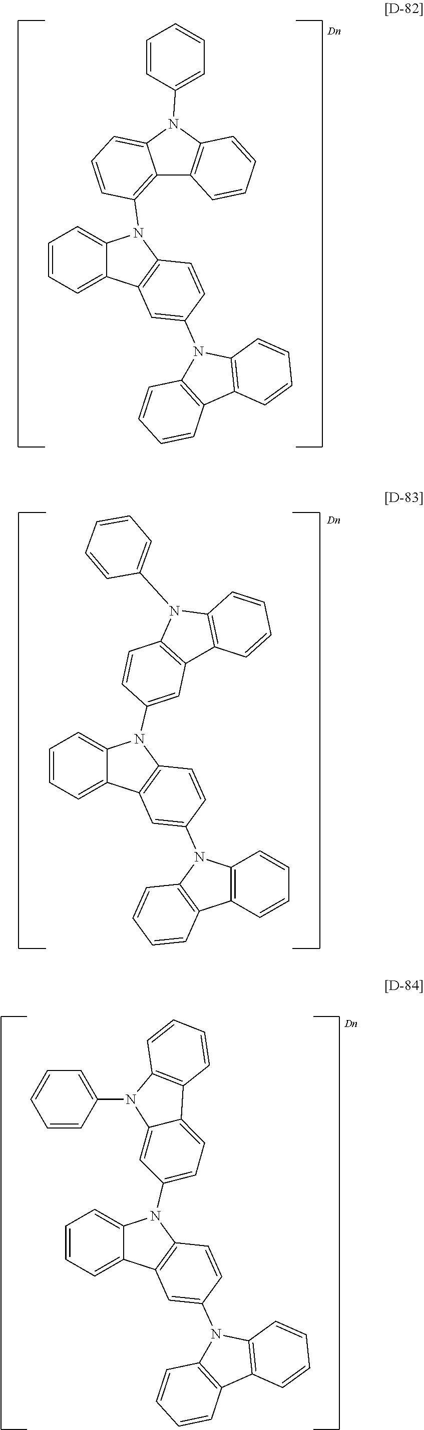

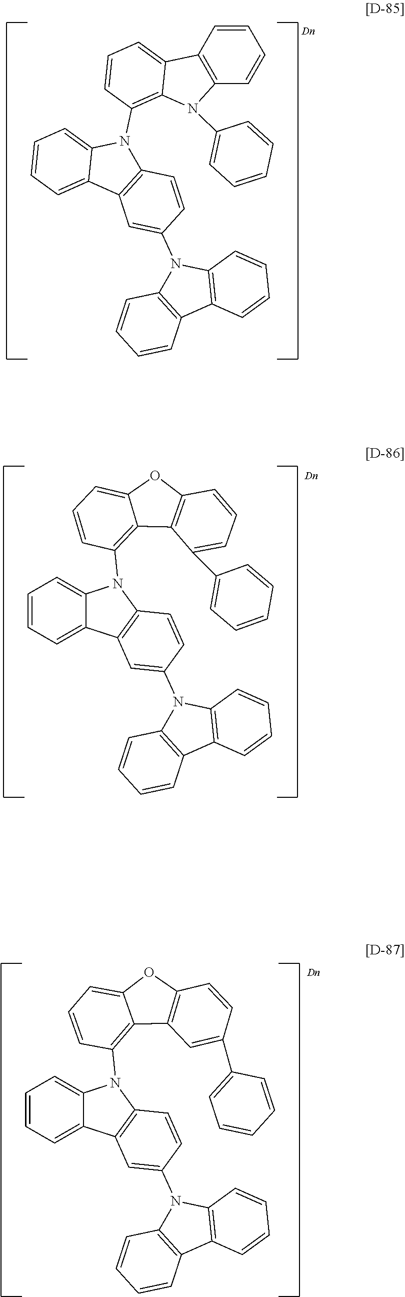

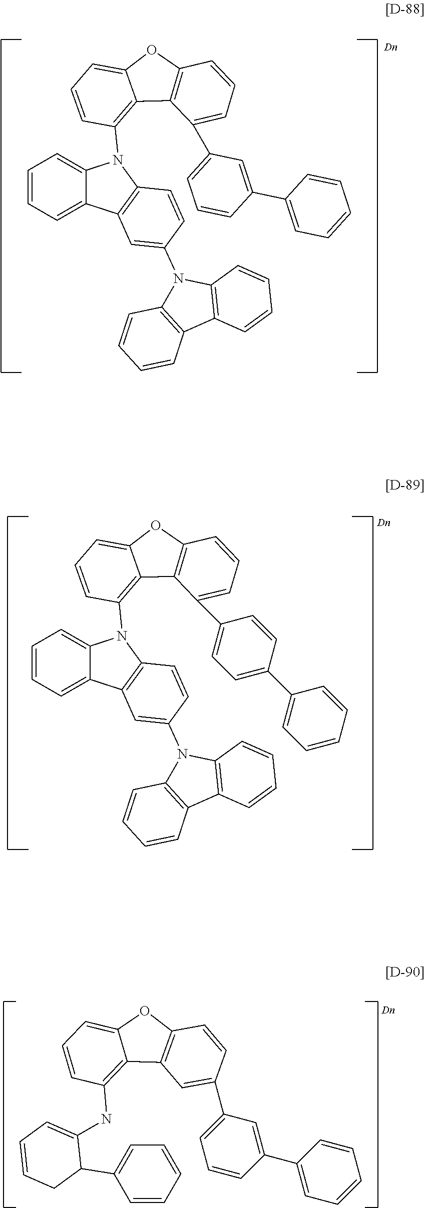

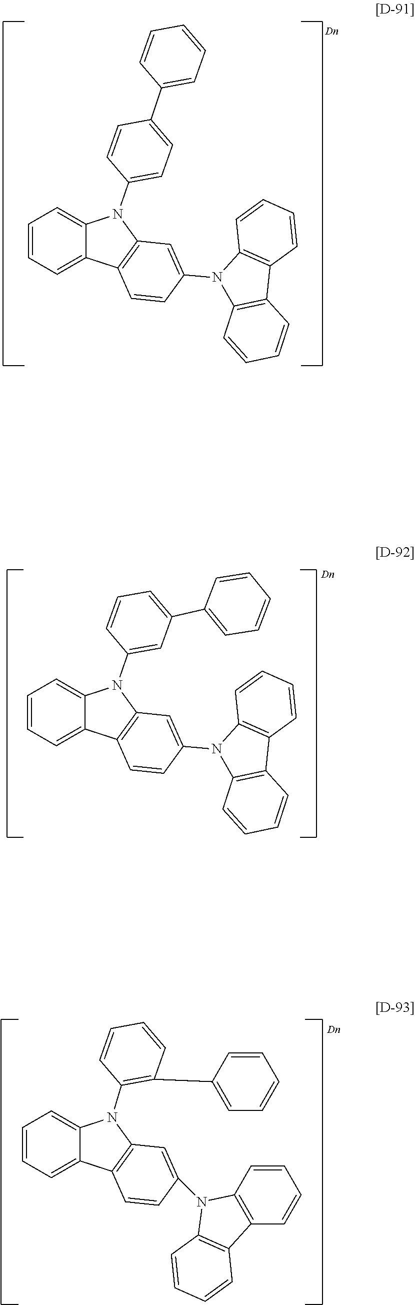

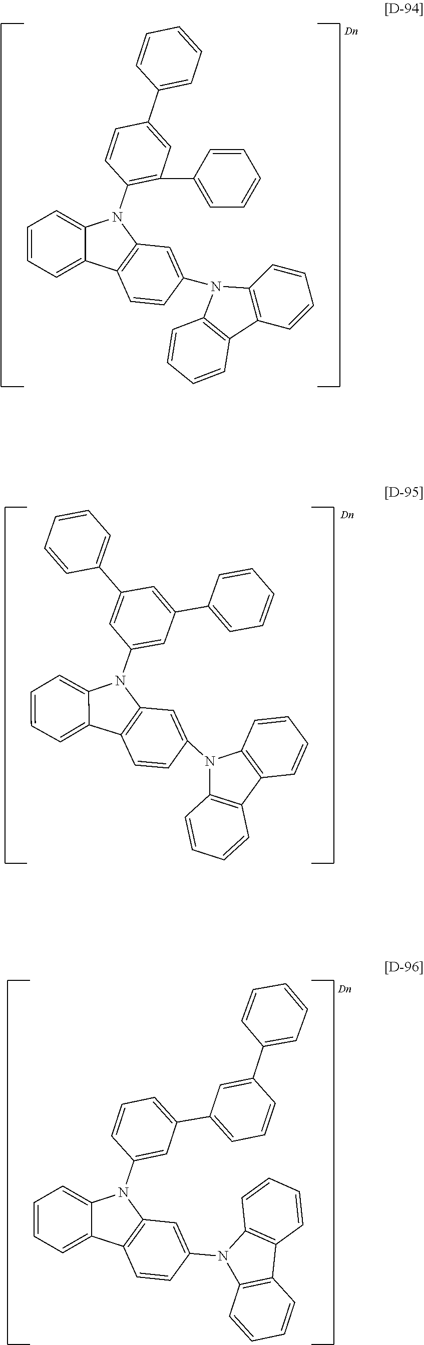

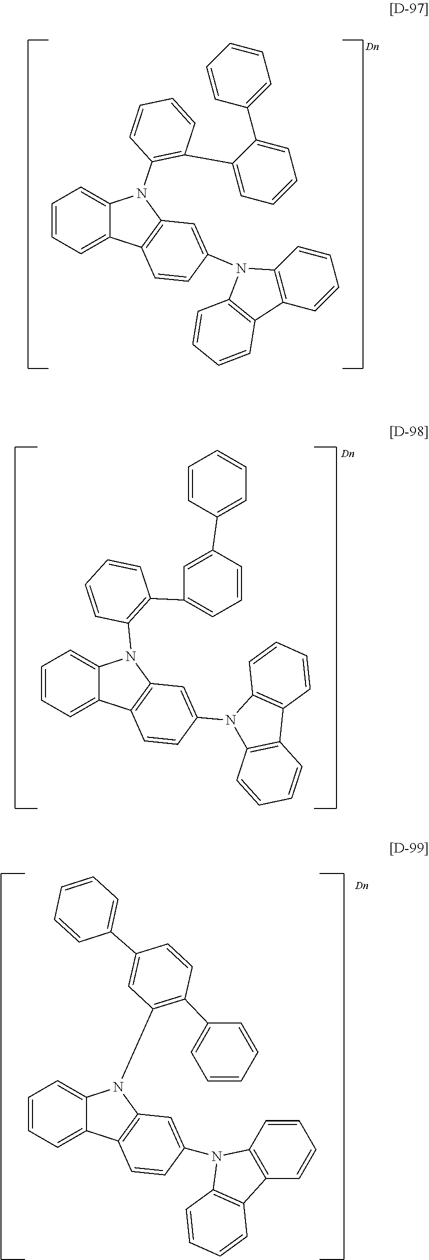

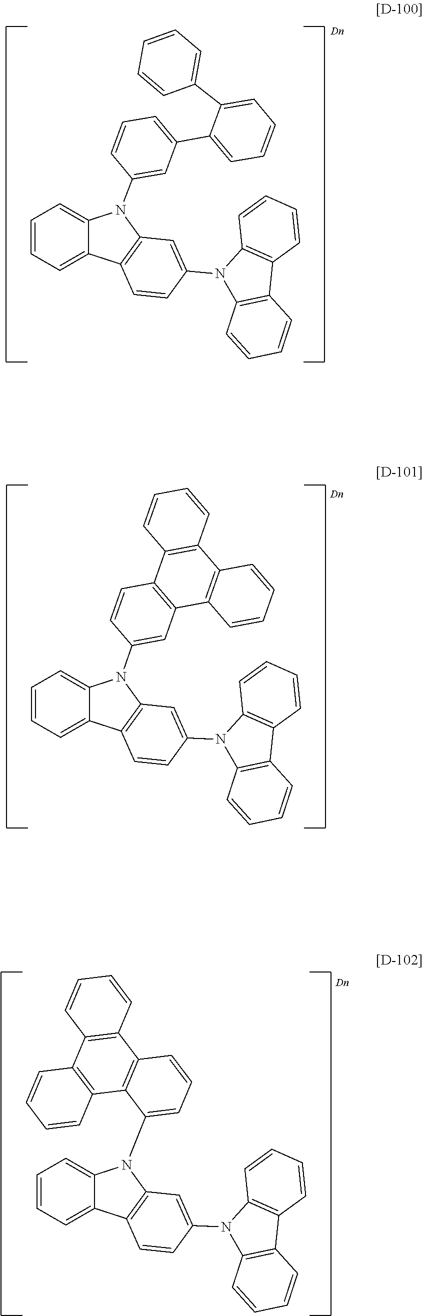

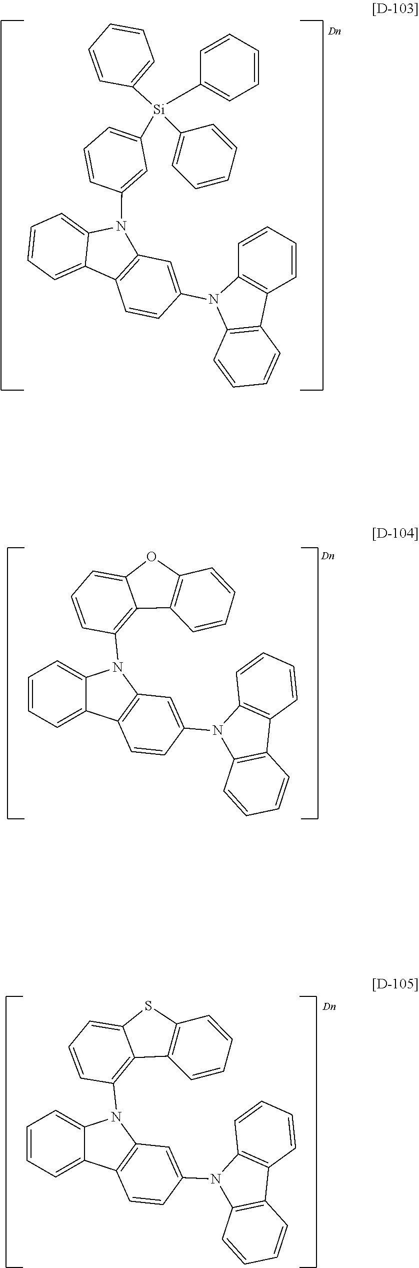

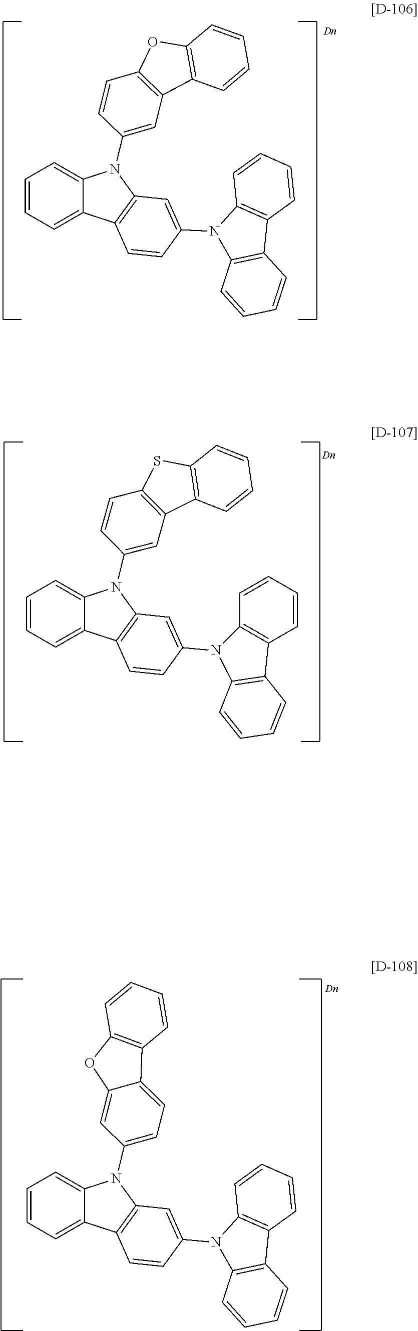

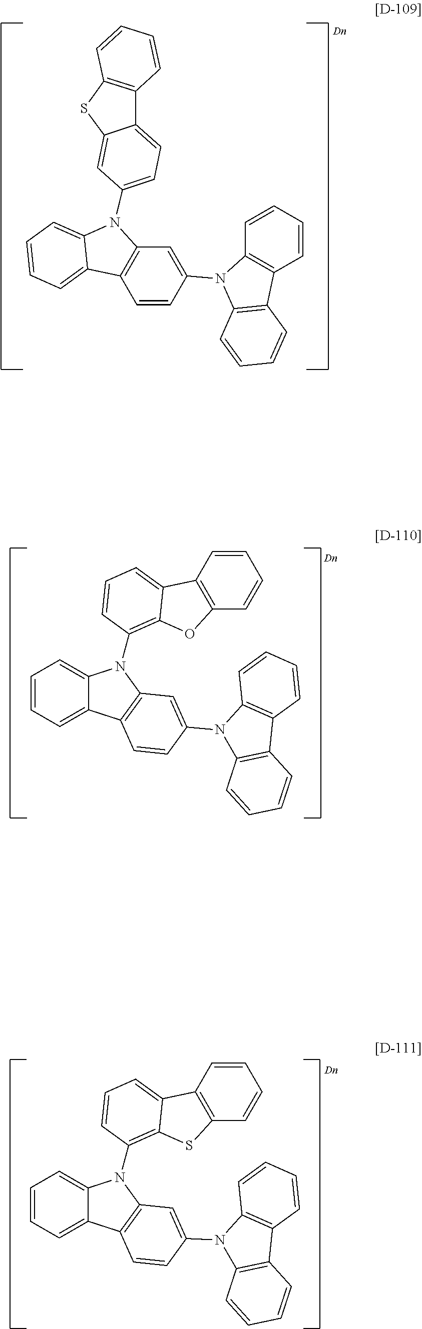

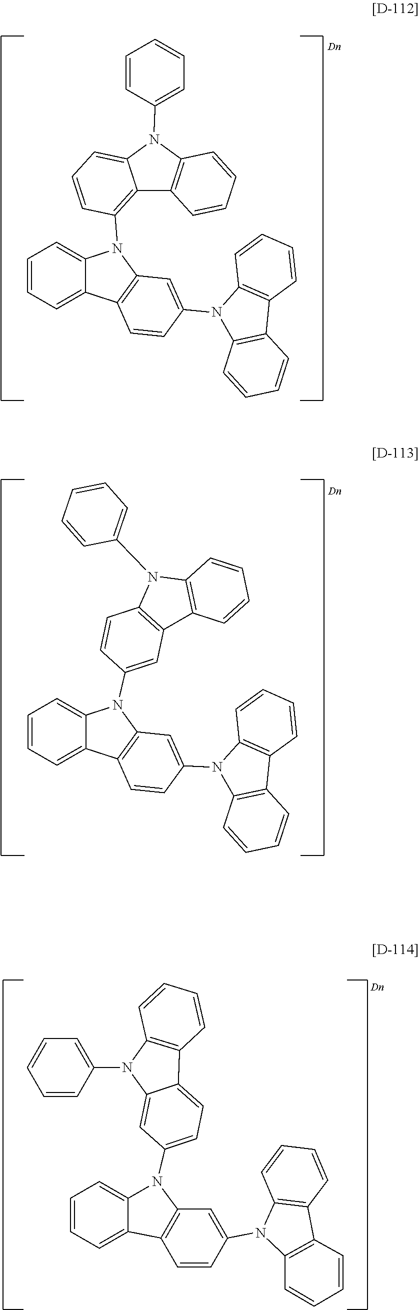

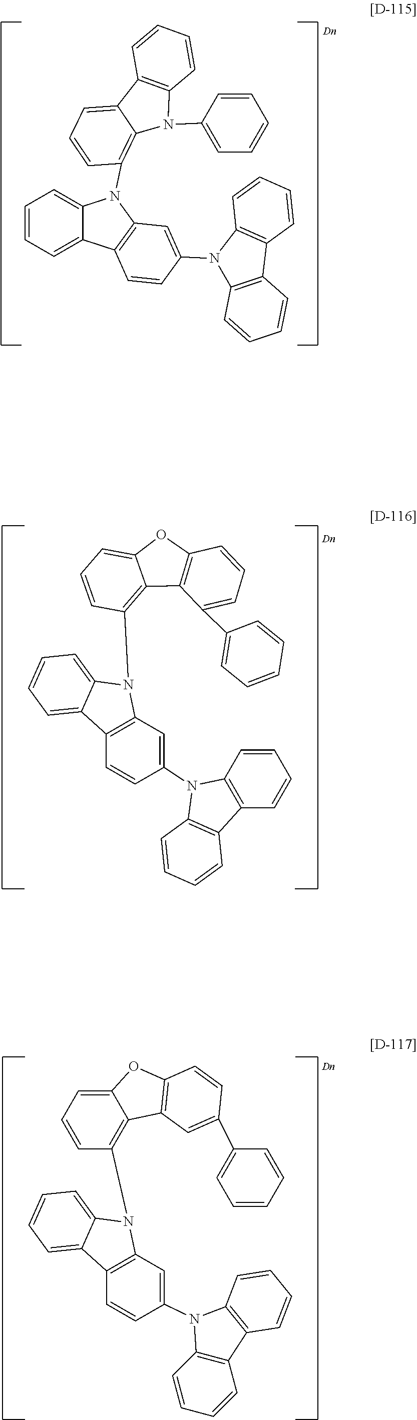

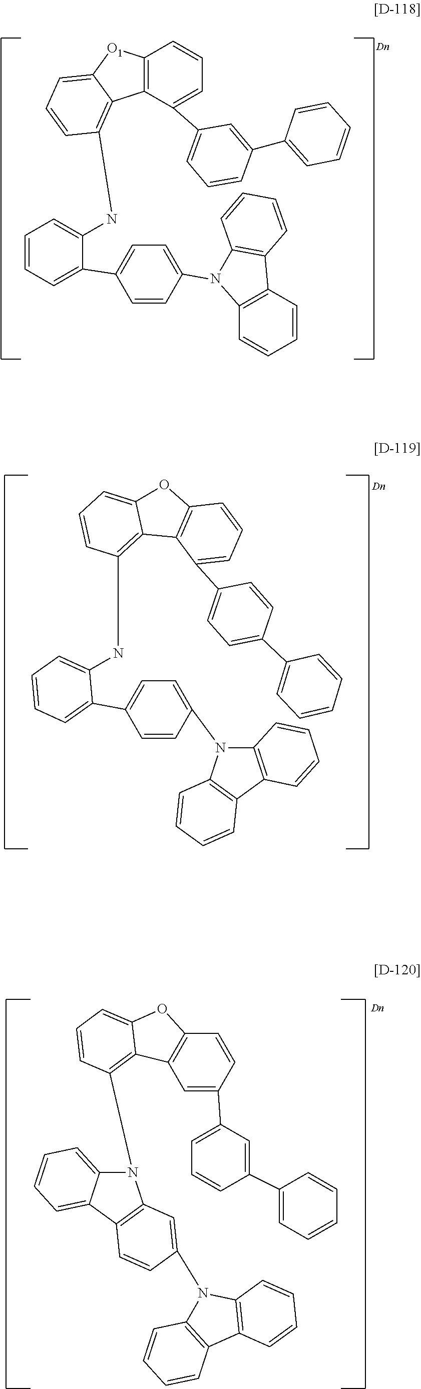

(Dn refers to the number of deuterium substitutions and indicates a structure substituted with one or more deuterium atoms)

The deuterium substitution position and deuterium substitution ratio may include all changeable ranges within the range of Compound D-1 to Compound D-120 (e.g., any hydrogen any compound may be a protium or a deuterium).

In an implementation, the first compound may be represented by Chemical Formula 1 and the second compound may be represented by Chemical Formula 2-8.

The first compound and the second compound may be included in a weight ratio of, e.g., about 1:99 to about 99:1. By being included in the above range, efficiency and life-span may be improved by implementing bipolar characteristics by adjusting the appropriate weight ratio using the electron transport capability of the first compound and the hole transport capability of the second compound. Within the above range, they may be included in a weight ratio of, e.g., about 10:90 to about 90:10, about 20:80 to about 80:20, e.g., about 20:80 to about 70:30, about 20:80 to about 60:40, and about 30:70 to about 60:40. In an implementation, they may be included in a weight ratio of about 40:60, about 50:50, or about 60:40.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the drawing.

The FIGURE is a cross-sectional view showing an organic light emitting diode according to some example embodiments.

Referring to the FIGURE, an organic light emitting diode 100 according to some example embodiments may include an anode 120 and a cathode 110 facing each other and an organic layer 105 between the anode 120 and cathode 110.

The anode 120 may be made of a conductor having a large work function to help hole injection, and may be, e.g., a metal, a metal oxide or a conductive polymer. The anode 120 may be, e.g., a metal such as nickel, platinum, vanadium, chromium, copper, zinc, gold, or the like or an alloy thereof, a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum, silver, tin, lead, cesium, barium, or the like, or an alloy thereof, a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include a light emitting layer 130, and the light emitting layer 130 may include a host and a dopant, the host may include the aforementioned compound for an organic optoelectronic device or composition for an organic optoelectronic device, and the dopant may be, e.g., a phosphorescent dopant, e.g., a red, green or blue phosphorescent dopant, e.g., a red or green phosphorescent dopant.

The dopant may be a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

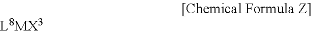

In Chemical Formula Z, M may be, e.g., a metal and L⁸ and X³ may be the same or different, and may each independently be, e.g., a ligand forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁸ and X³ may be, e.g., a bidentate ligand.

An example of a bidentate ligand may be represented by Chemical Formula Z-1 or Chemical Formula Z-2.

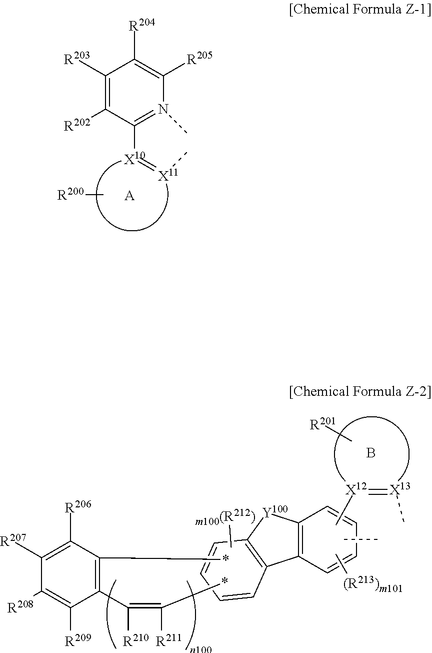

In Chemical Formula Z-1 and Chemical Formula Z-2, ring A and ring B may each independently be, e.g., a monocyclic ring or a polycyclic fused ring, wherein each ring among the monocyclic ring and polycyclic fused ring may be, e.g., a 5- or 6-membered carbocyclic or heterocyclic ring.

R²⁰⁰ and R²⁰¹ may each independently represent, e.g., one to a maximum number of monovalent substituents.

In an implementation, R²⁰⁰ and R²⁰¹ may each be two or more, and each R²⁰⁰ and R²⁰¹ may be the same or different from each other.

R²⁰² to R²¹³ may each independently be, e.g., hydrogen, deuterium, halogen, a substituted or unsubstituted C1 to C10 alkyl group, a substituted or unsubstituted C6 to C20 aryl group, —SiR²¹⁴R²¹⁵R²¹⁶, —GeR²¹⁴R²¹⁵R²¹⁶, or a combination thereof.

R²¹⁴ to R²¹⁶ may each independently be, e.g., a substituted or unsubstituted C1 to C6 alkyl group.

X¹⁰, X¹¹, X¹², and X¹³ may each independently be, e.g., carbon or nitrogen.

Y¹⁰⁰ may be O or S.

m100 may be, e.g., an integer of 1 to 3.

m101 may be, e.g., an integer of 1 to 2.

n100 may be, e.g., an integer of 0 or 1.

* is a linking point.

In an implementation, n100 is 0, and it may be formed with a monovalent substituent.

In an implementation, n100 is 1, and a fusion ring may be formed.

Examples of the ligands represented by L⁸ and X³ may be, e.g., a chemical formula of Group A.

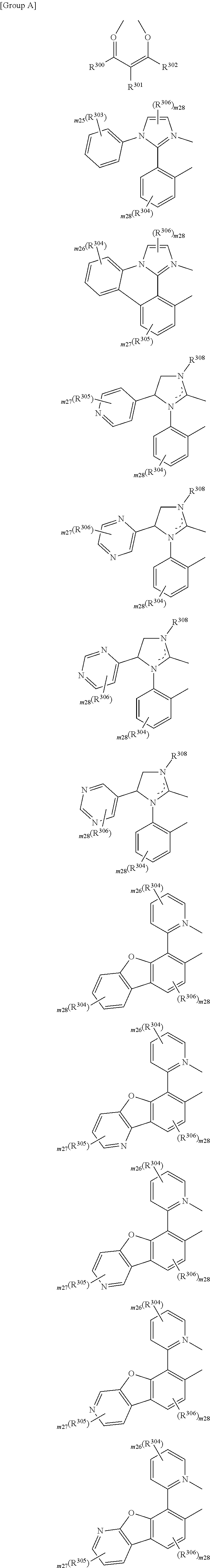

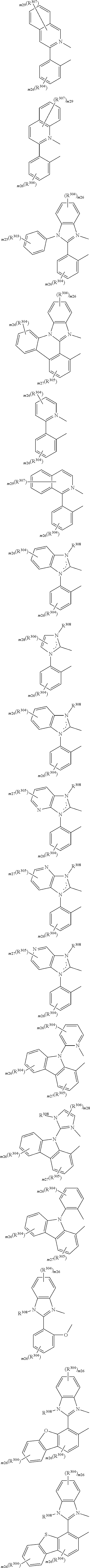

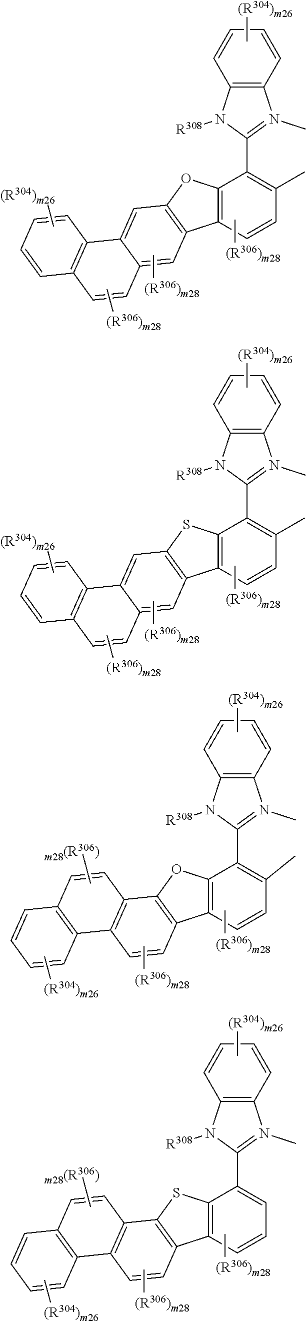

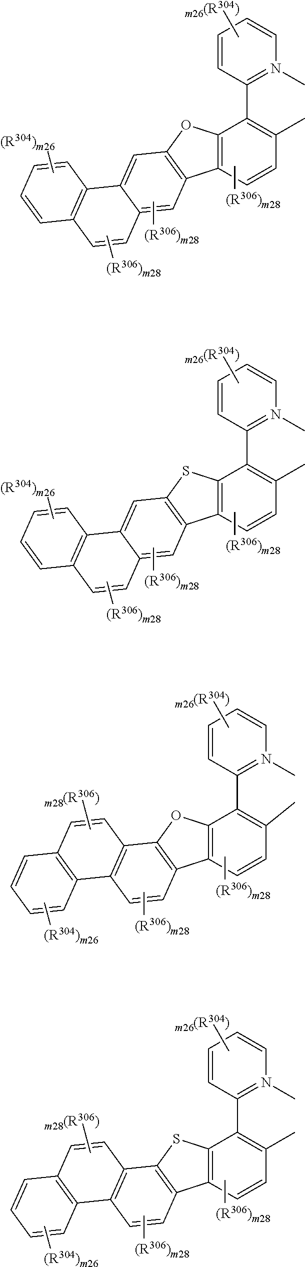

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³⁰⁸ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group or a substituted or unsubstituted C6 to C30 aryl group, or a triarylsilyl group having a substituted or unsubstituted C6 to C30 aryl group.

m25 may be, e.g., an integer of 1 to 5.

m26 may be, e.g., an integer of 1 to 4.

m27 may be, e.g., an integer of 1 to 3.

m28 may be, e.g., an integer of 1 or 2.

m29 may be, e.g., an integer of 1 to 6.

In an implementation, m25 to m29 may be 2 or more, and each R³⁰³ to R³⁰⁷ may be the same or different from each other.

The dopant according to some example embodiments may be an iridium complex, and may be represented, e.g., by Chemical Formula 6-1 to Chemical Formula 6-5.

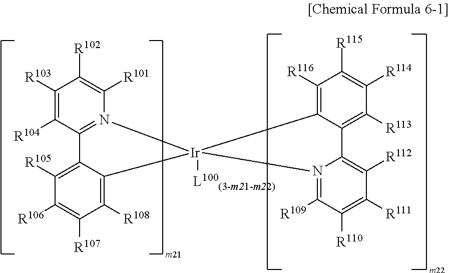

In Chemical Formula 6-1, R¹⁰¹ to R¹¹⁶ may each independently be, e.g., hydrogen, deuterium, halogen, a substituted or unsubstituted C1 to C10 alkyl group, a substituted or unsubstituted C6 to C20 aryl group, —SiR¹³²R¹³³R¹³⁴, or —GeR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be, e.g., a functional group represented by Chemical Formula V-1.

L¹⁰⁰ may be, e.g., a bidentate ligand of a monovalent anion and may be, e.g., a ligand that coordinates to iridium through a lone pair of carbons or heteroatoms.

m21 and m22 may each independently be, e.g., an integer of 0 to 3 and m21+m22 may be, e.g., an integer of 1 to 3.

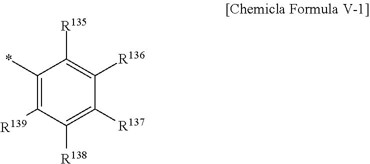

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* refers to a portion linked to a carbon atom.

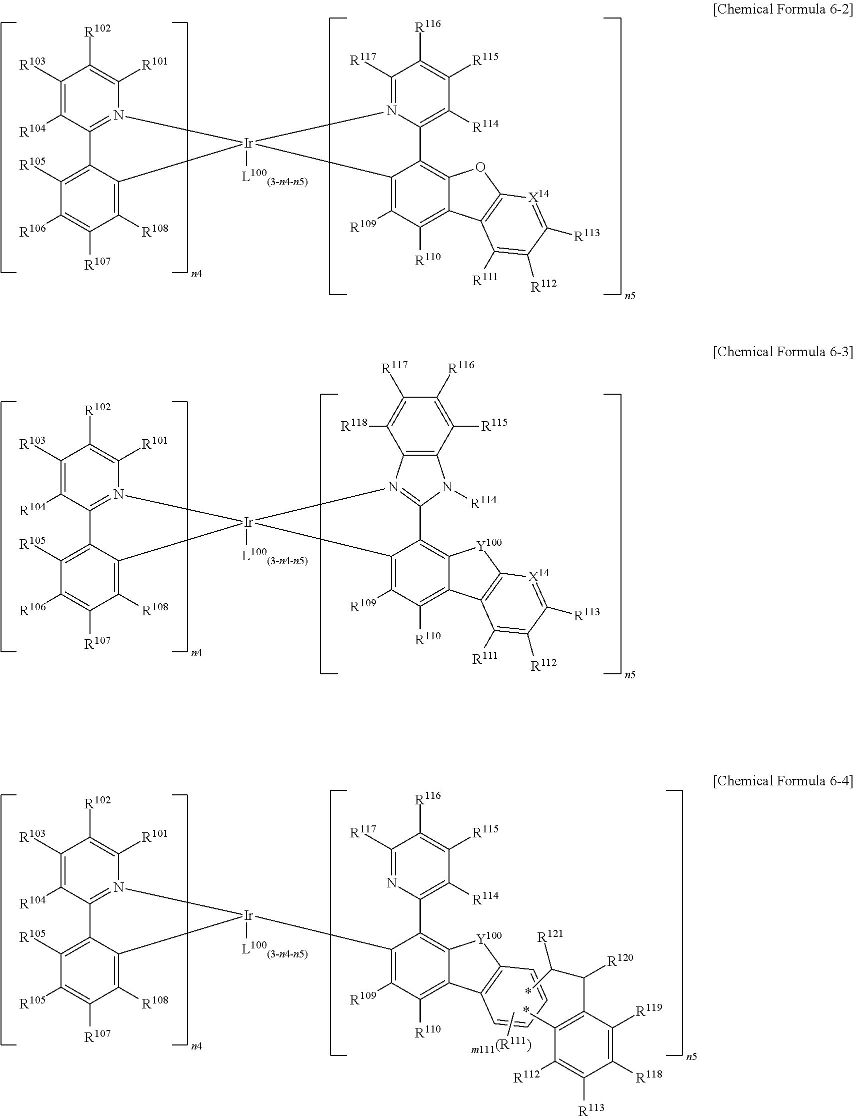

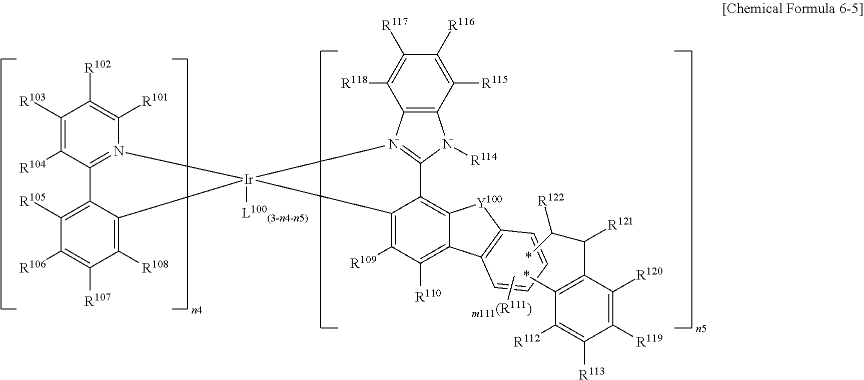

In Chemical Formula 6-2 to Chemical Formula 6-5, X¹⁴ may be, e.g., carbon or nitrogen.

Y¹⁰⁰ may be, e.g., O or S.

R¹⁰¹ to R¹²² may each independently be, e.g., hydrogen, deuterium, halogen, a substituted or unsubstituted C1 to C10 alkyl group, a substituted or unsubstituted C6 to C20 aryl group, —SiR¹³³R¹³⁴R¹³⁵, or —GeR¹³³R¹³⁴R¹³⁵.

R¹³³ to R¹³⁵ may each independently be, e.g., a substituted or unsubstituted C1 to C6 alkyl group.

L¹⁰⁰ may be a bidentate ligand of a monovalent anion, and may be a ligand that coordinates to iridium through a lone pair of carbons or heteroatoms.

m11 may be, e.g., an integer of 1 to 2.

n4 and n5 may each independently be, e.g., an integer of 0 to 3, and n4+n5 may be, e.g., an integer of 1 to 3.

The dopant according to some example embodiments may be a platinum complex, and may be represented, e.g., by Chemical Formula Z-3.

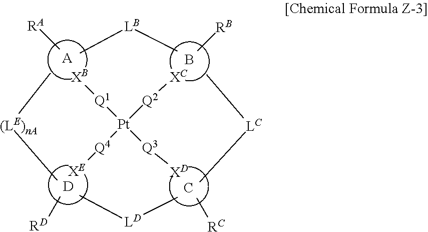

In Chemical Formula Z-3, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, Lc, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be a direct bond, BR, NR, PR, O, S, Se, C═O, S═O, SO₂, CRR′, SiRR′, GeRR′, or a combination thereof. In an implementation, nA may be 0, and L^{E} may not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The platinum complex may be represented, e.g., by Chemical Formula 7-1 or Chemical Formula 7-2.

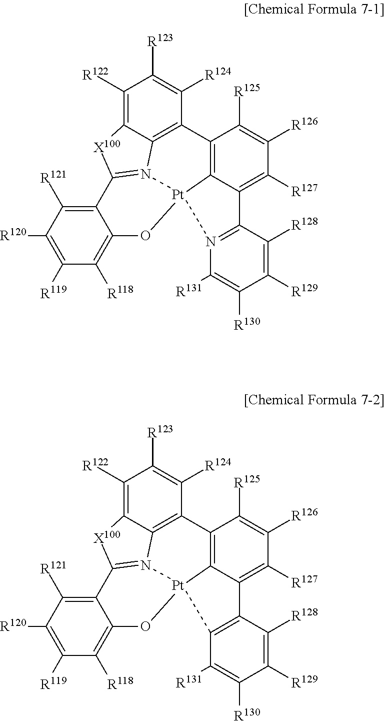

In Chemical Formula 7-1 and Chemical Formula 7-2, X¹⁰⁰ may be, e.g., O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be, e.g., —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., the hole transport region 140.

The hole transport region 140 may help further increase hole injection or hole mobility between the anode 120 and the light emitting layer 130 and block electrons.

In an implementation, the hole transport region 140 may include, e.g., a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

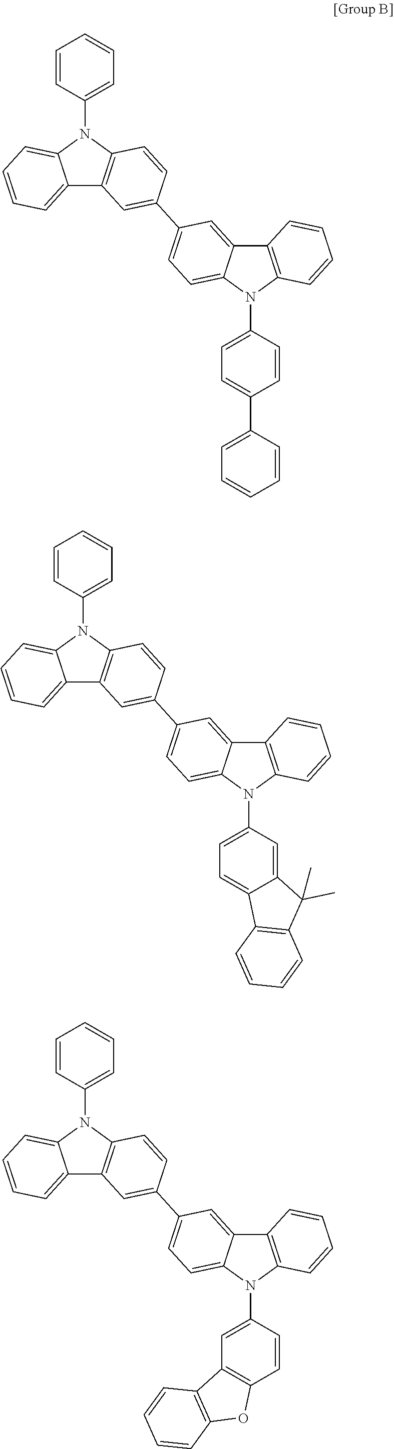 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

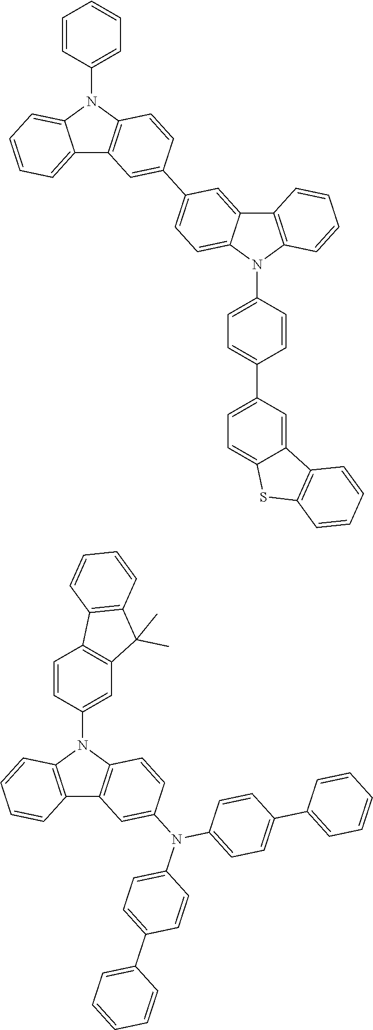 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

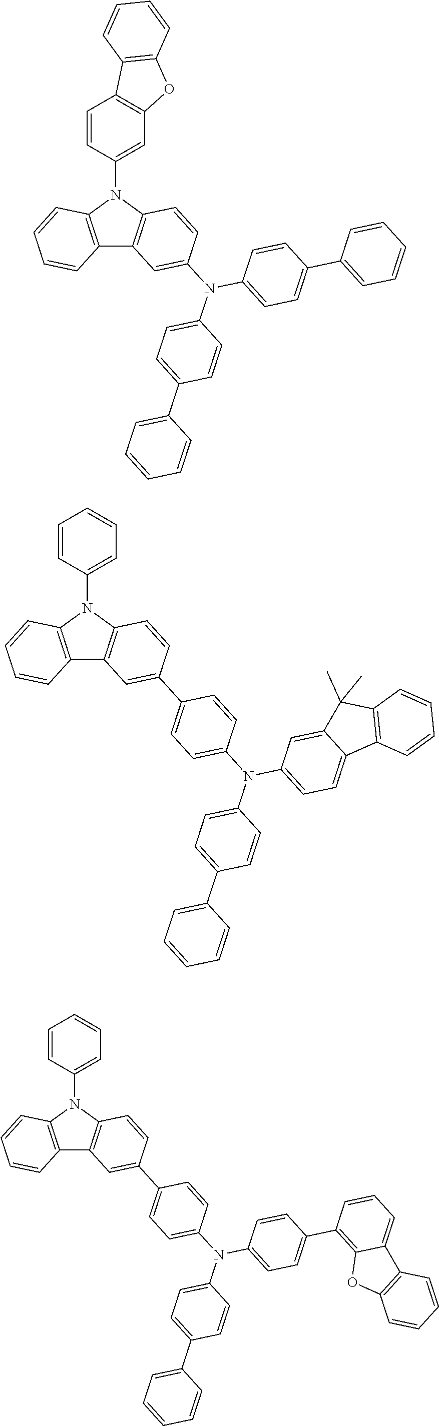 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

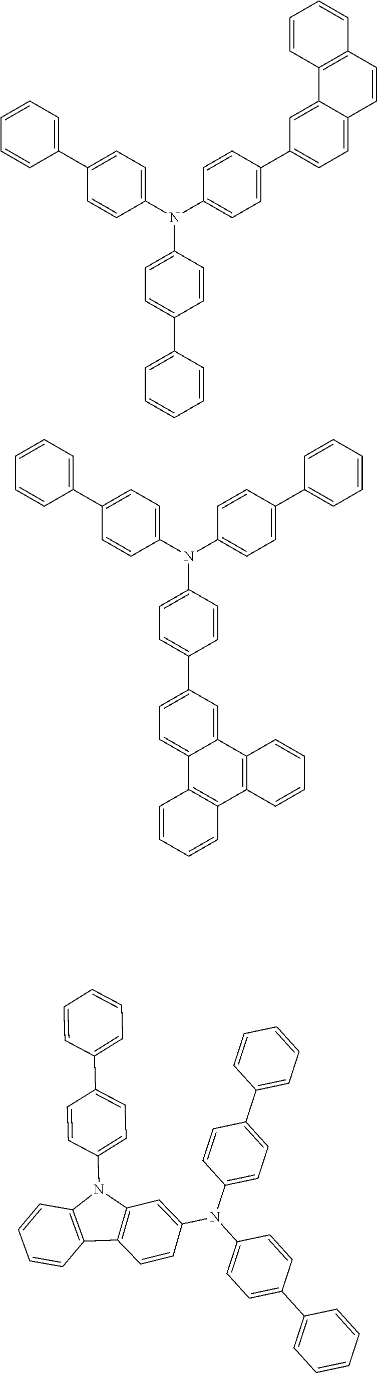 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

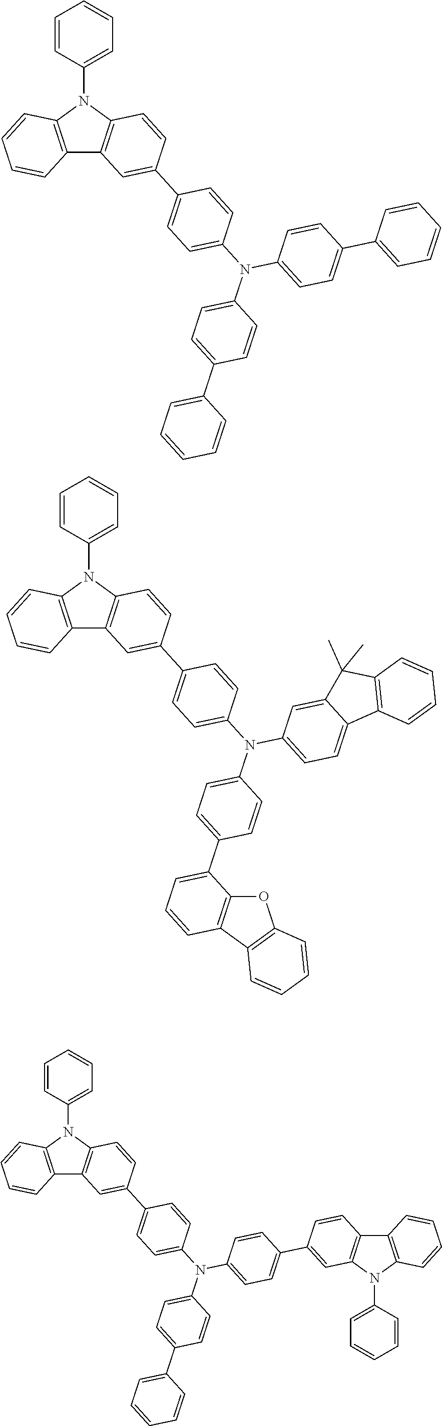 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

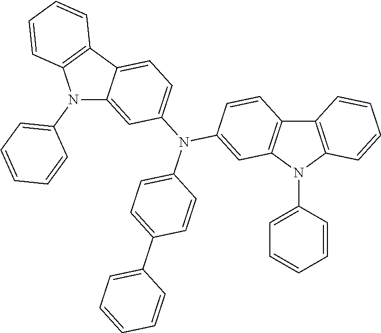 [SMILES: c1ccc(-c2ccc(N(c3ccc4c5ccccc5n(-c5ccccc5)c4c3)c3ccc4c5ccccc5n(-c5ccccc5)c4c3)cc2)cc1]

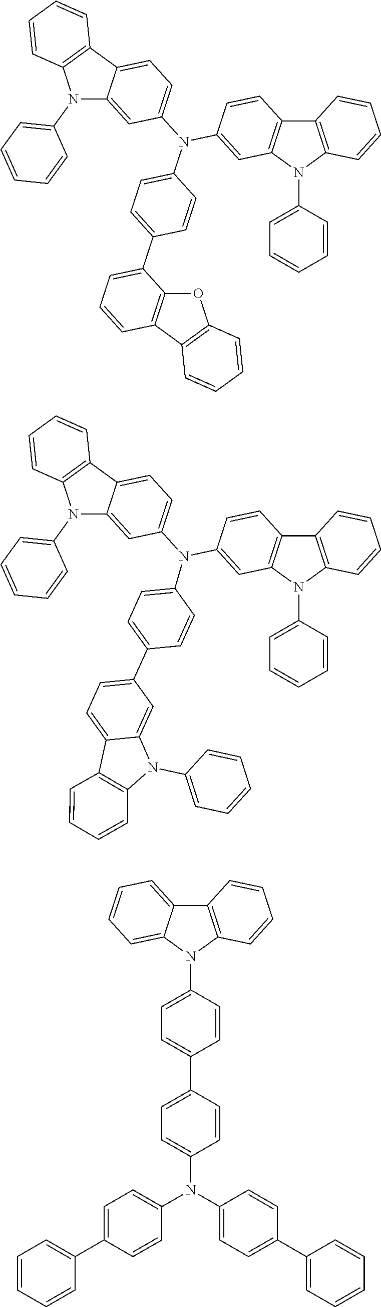 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

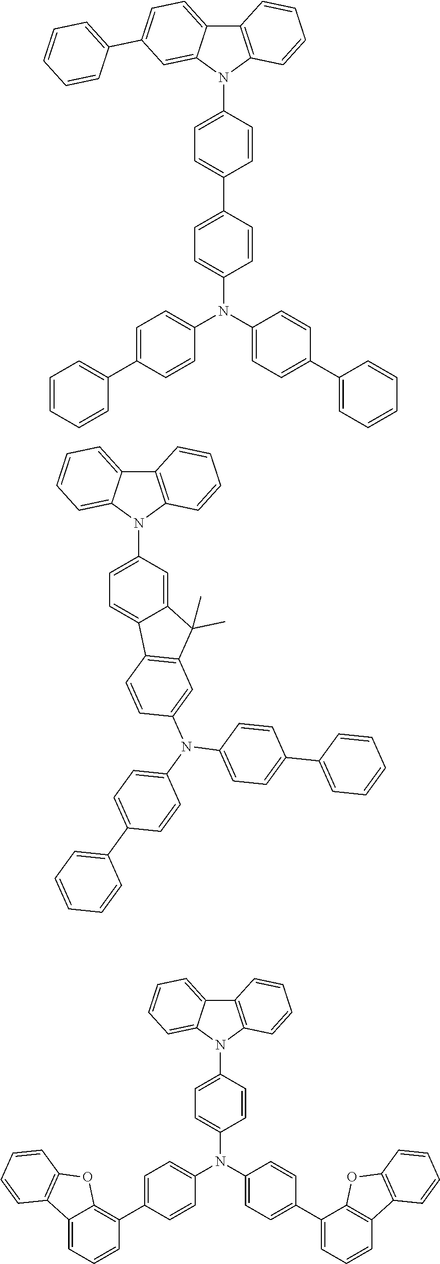

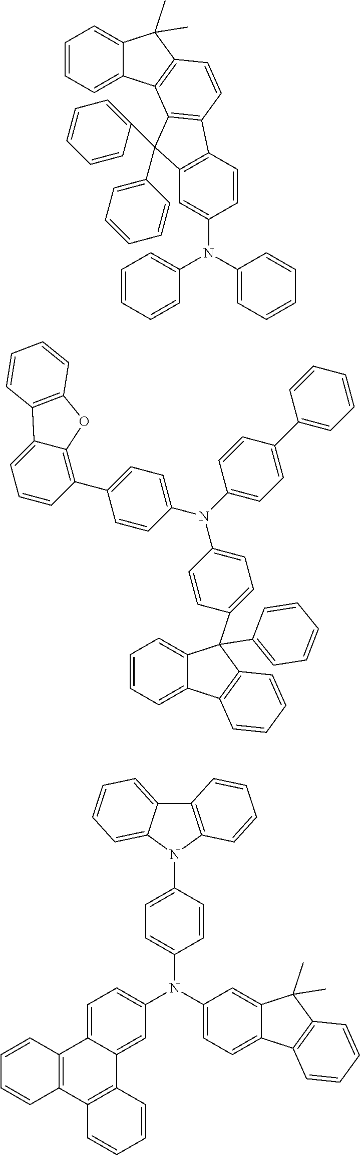 [SMILES: CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

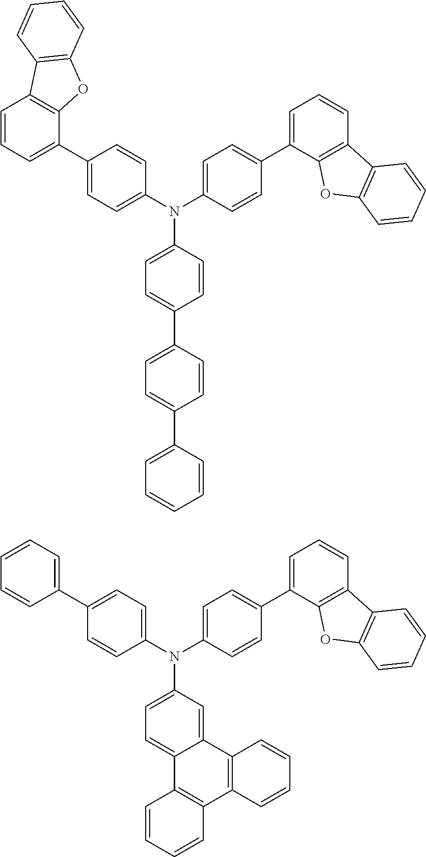 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

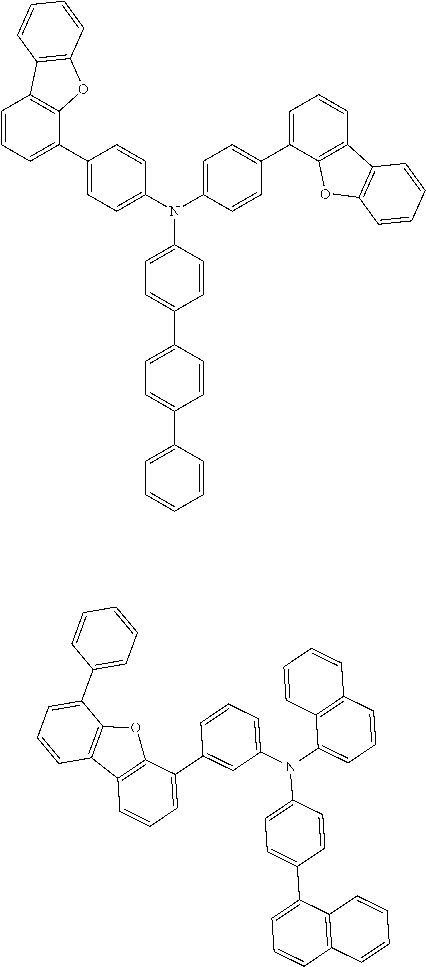 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cccc3c2oc2c(-c4cccc(N(c5ccc(-c6cccc7ccccc67)cc5)c5cccc6ccccc56)c4)cccc23)cc1]

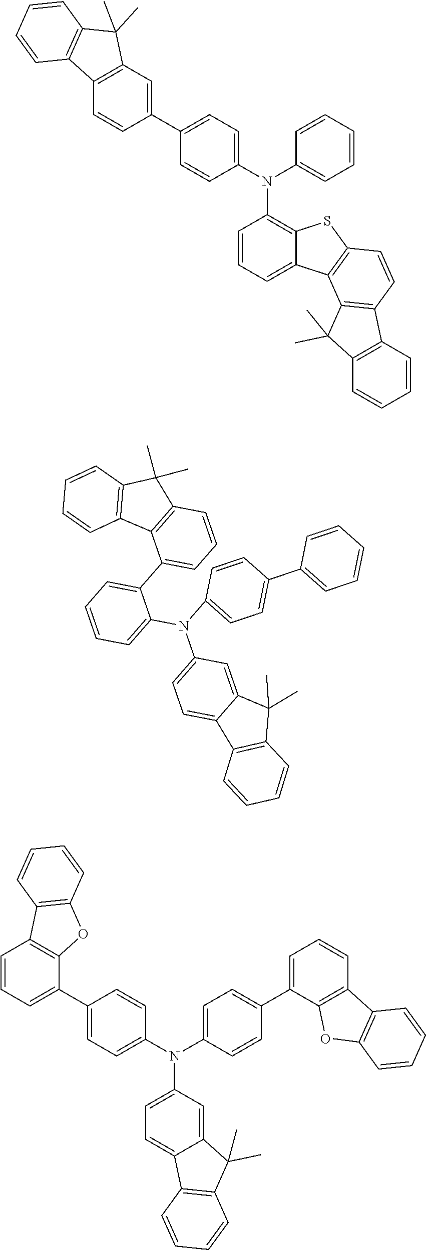 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

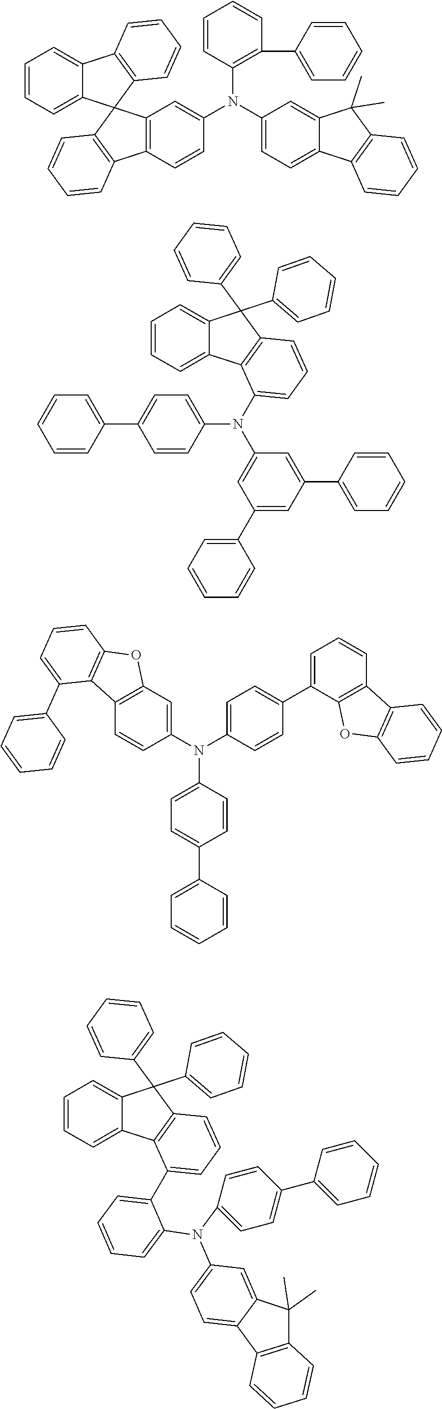 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

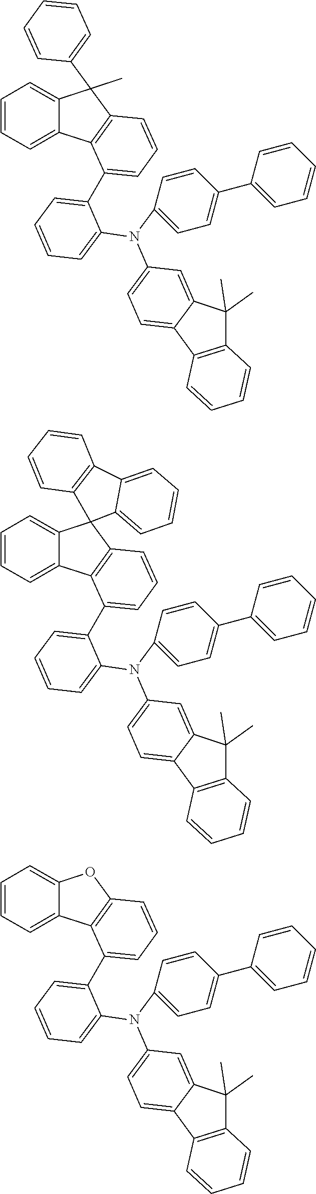 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

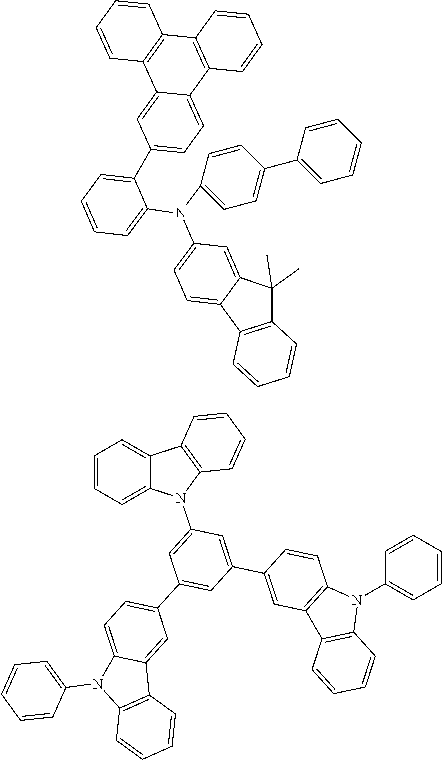

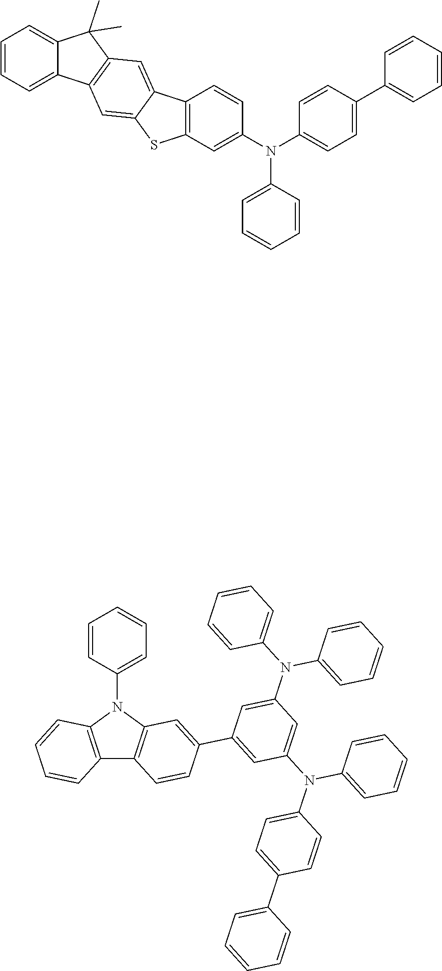 [SMILES: CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

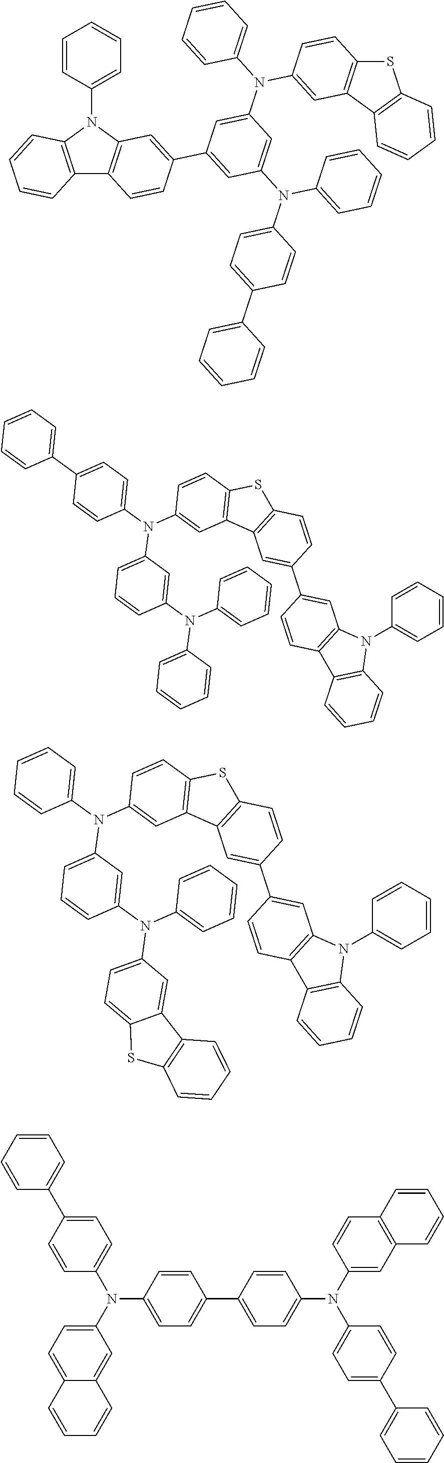 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

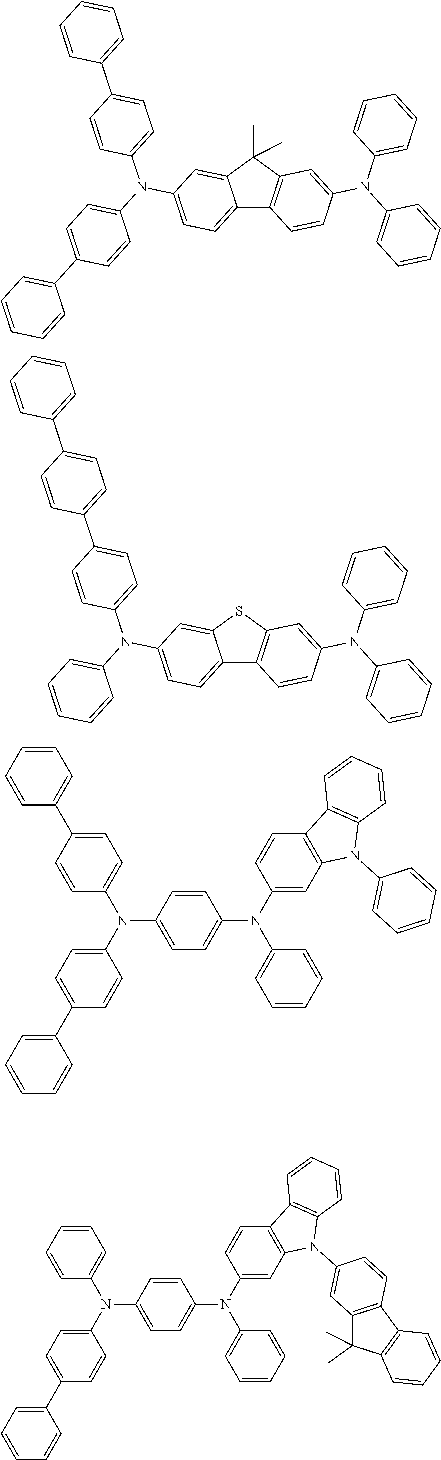

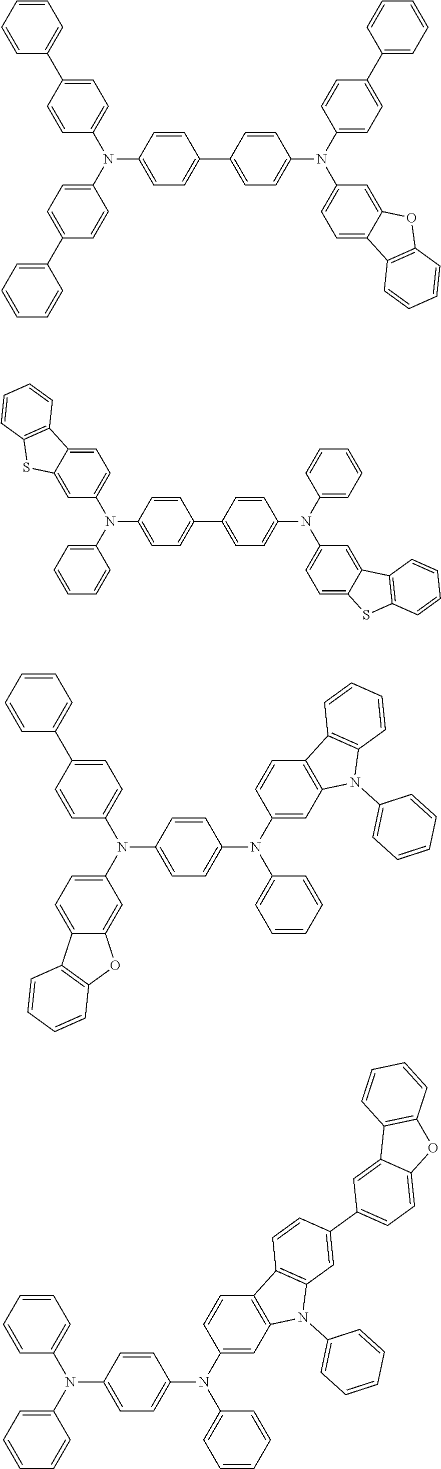 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

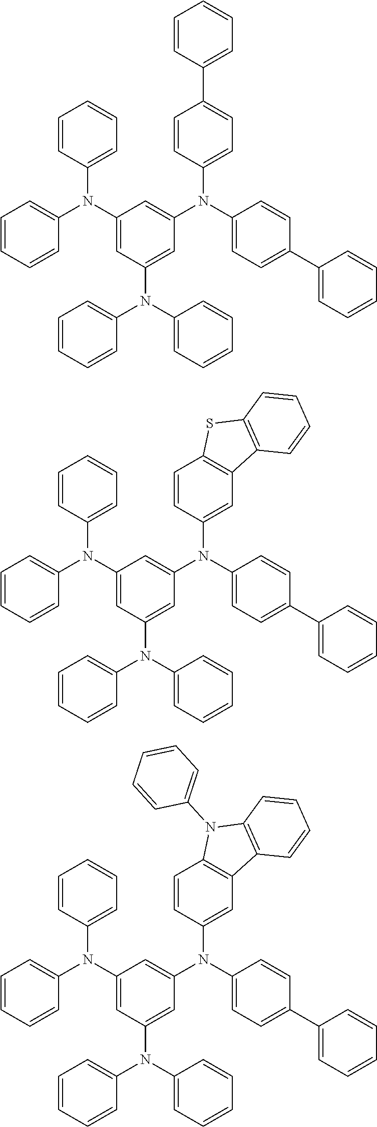

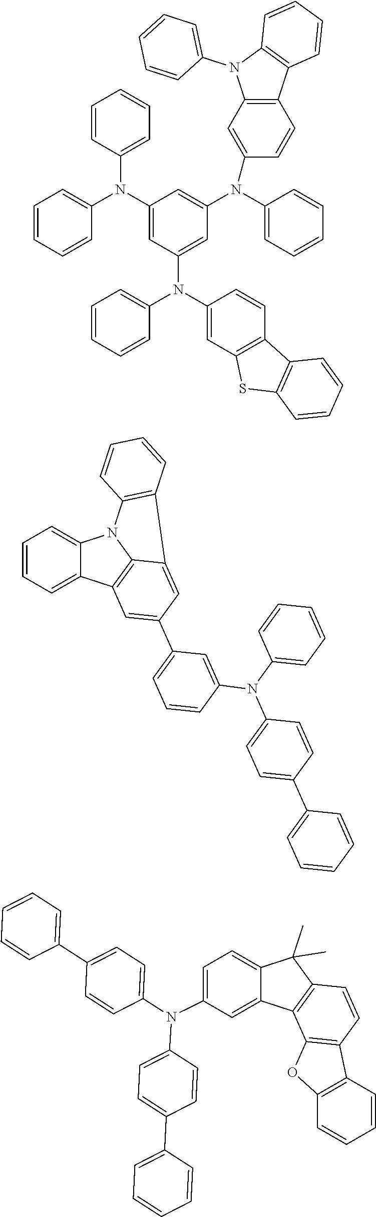 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

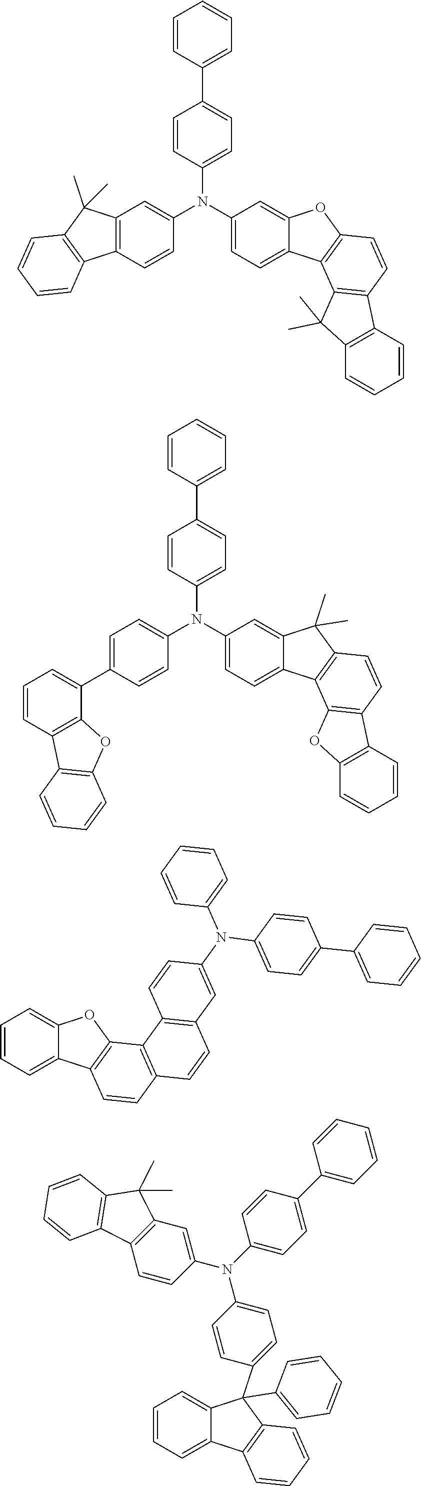 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

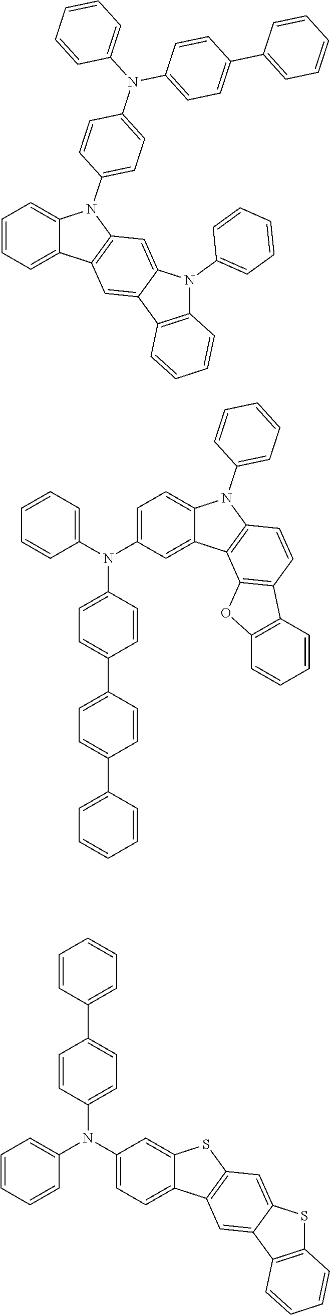

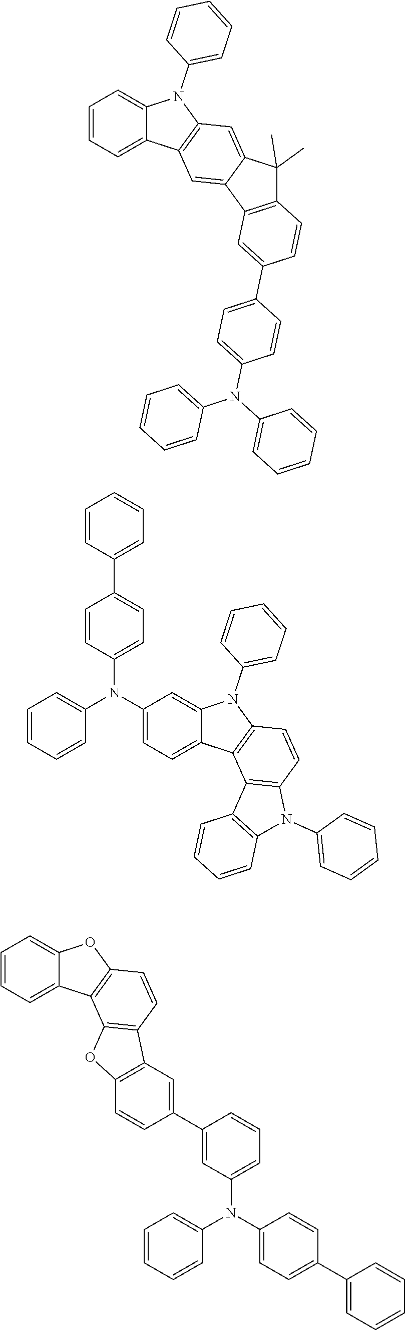 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

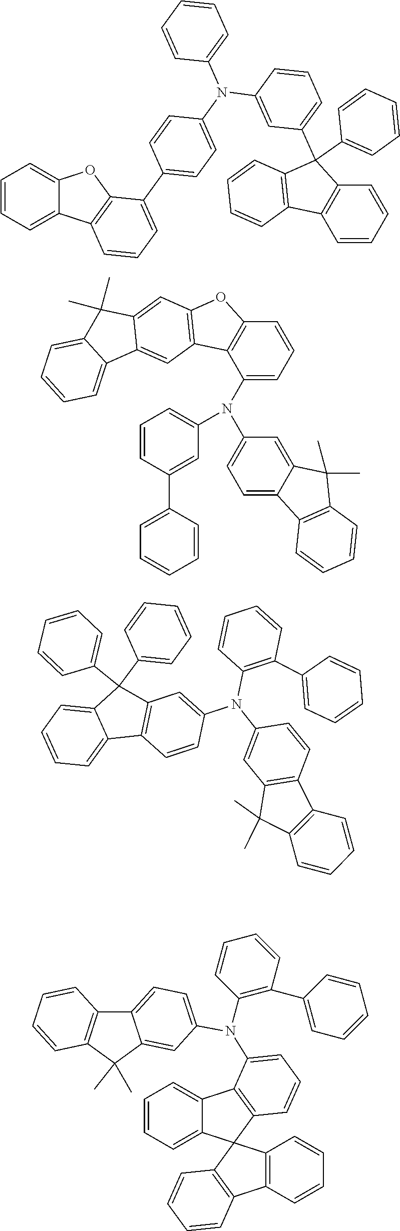

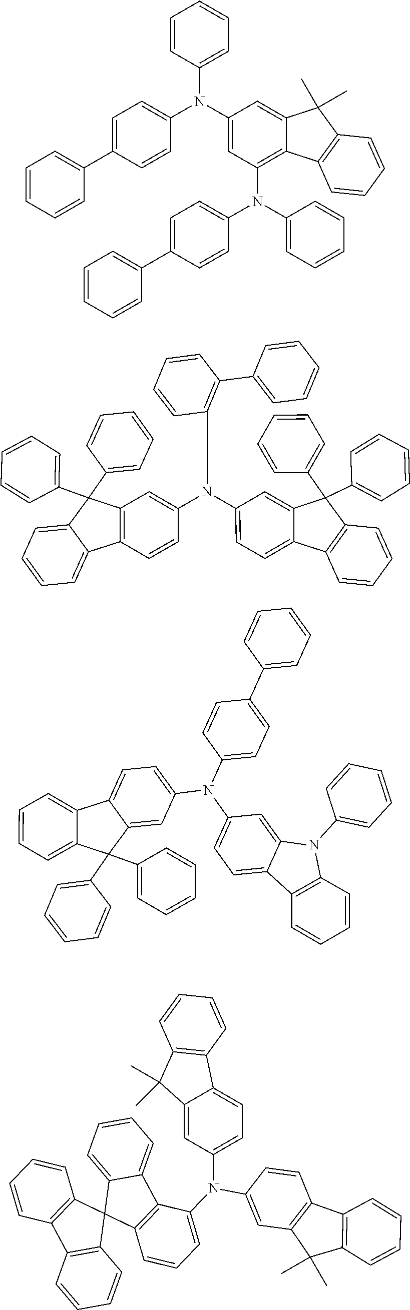 [SMILES: CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21]

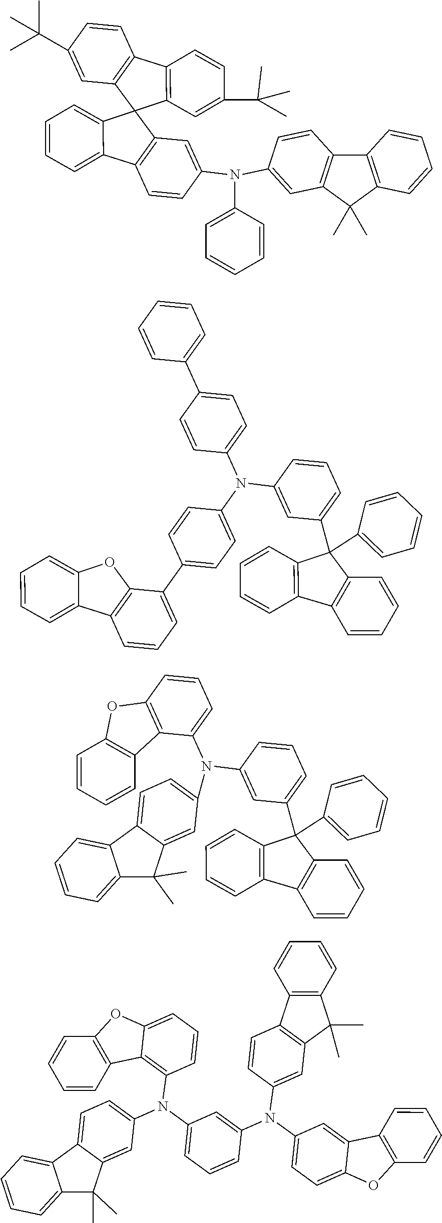

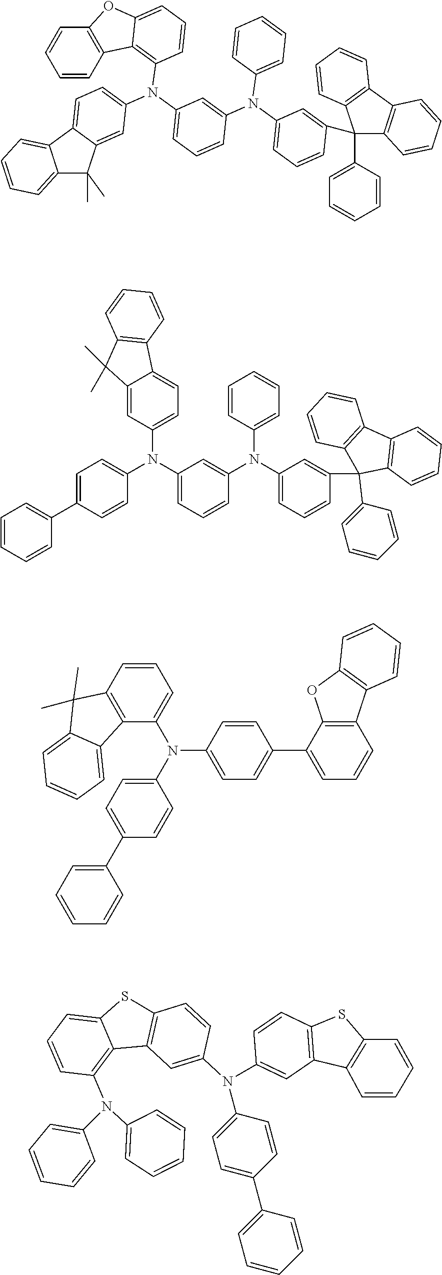 [SMILES: CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1]

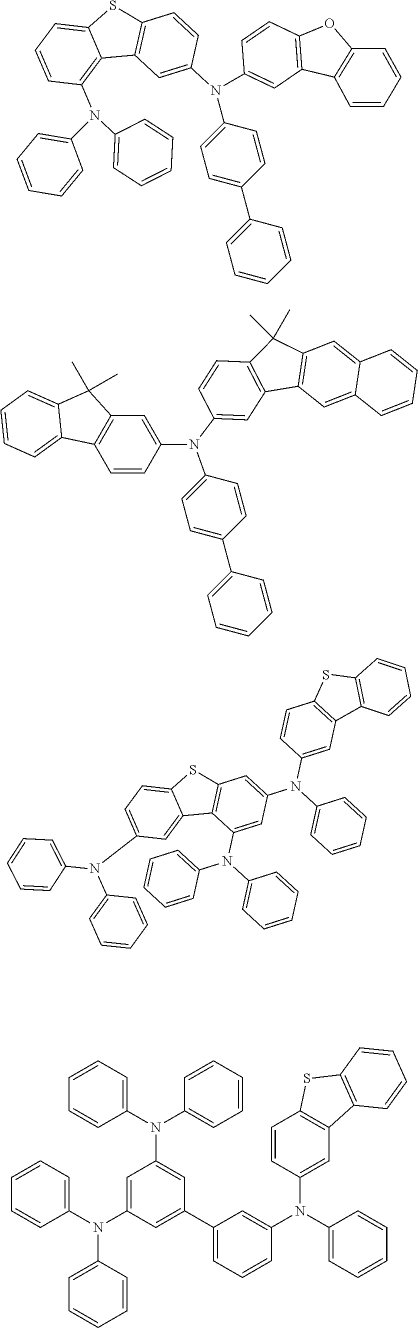

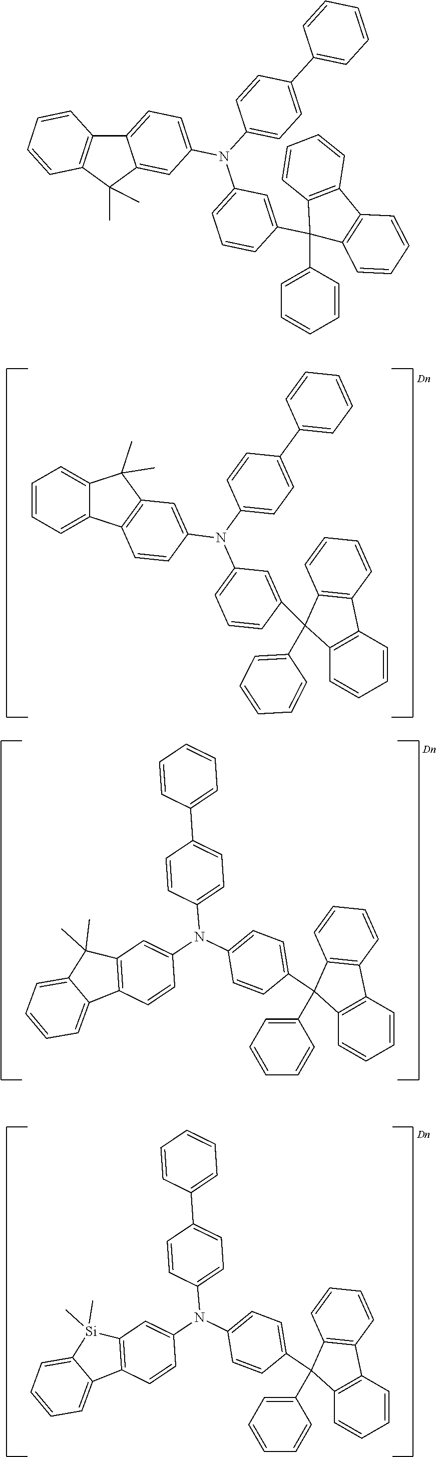

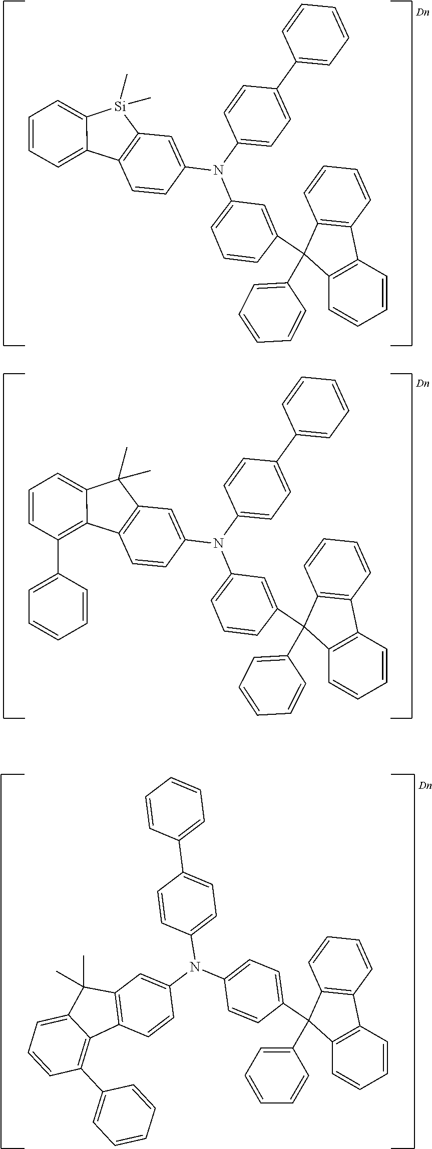

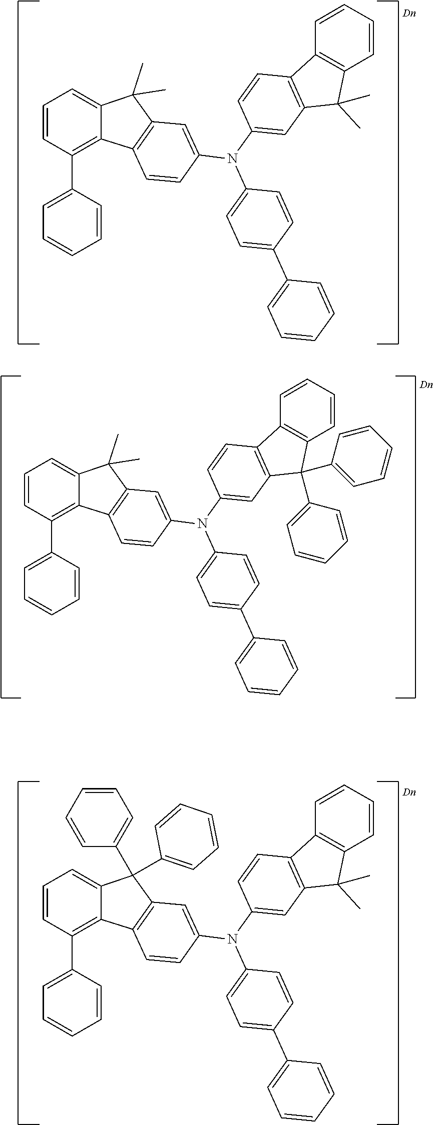

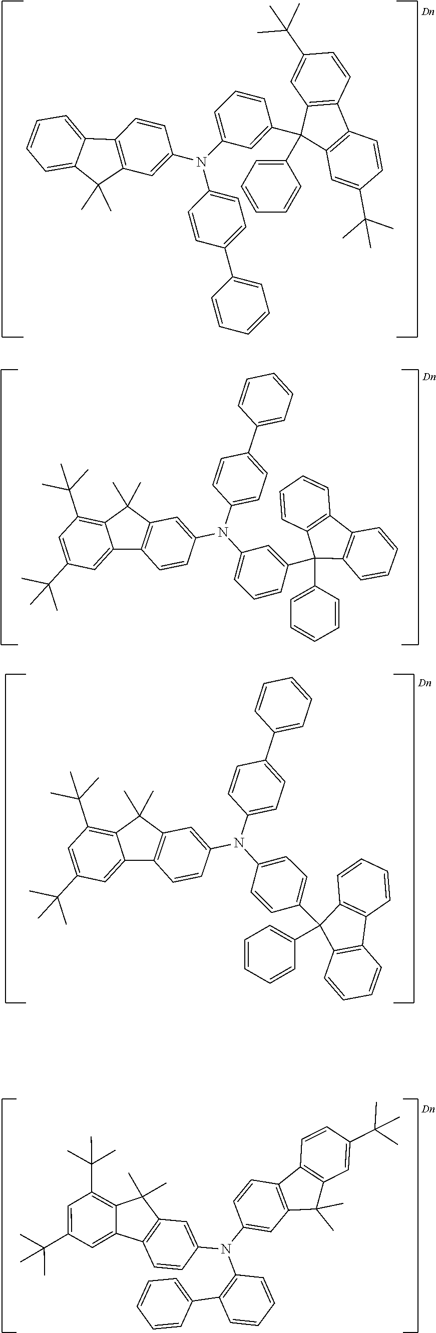

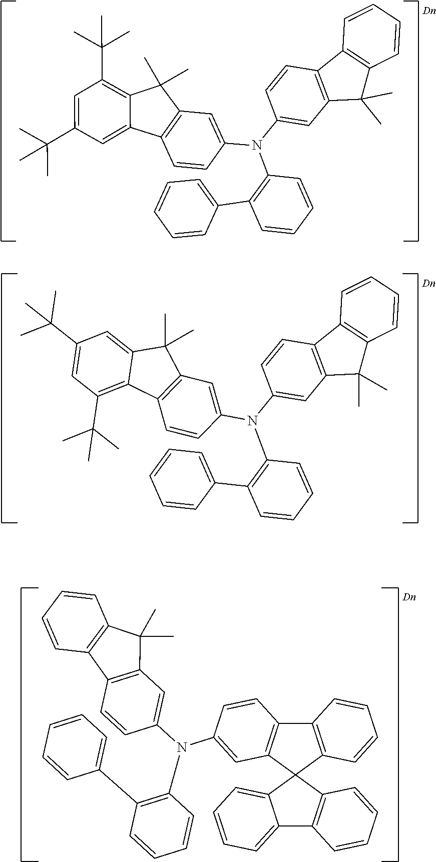

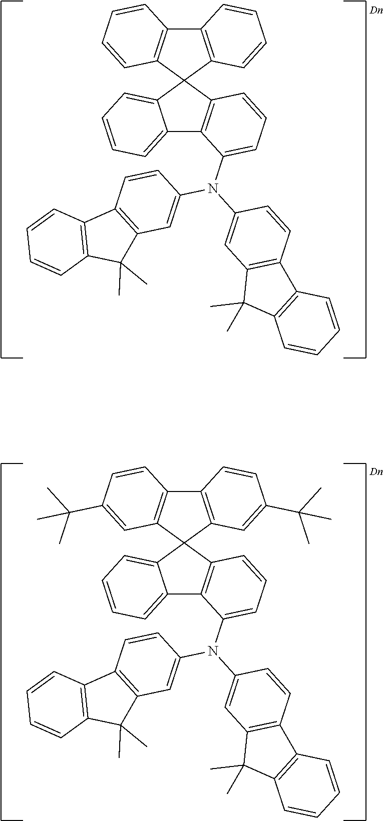

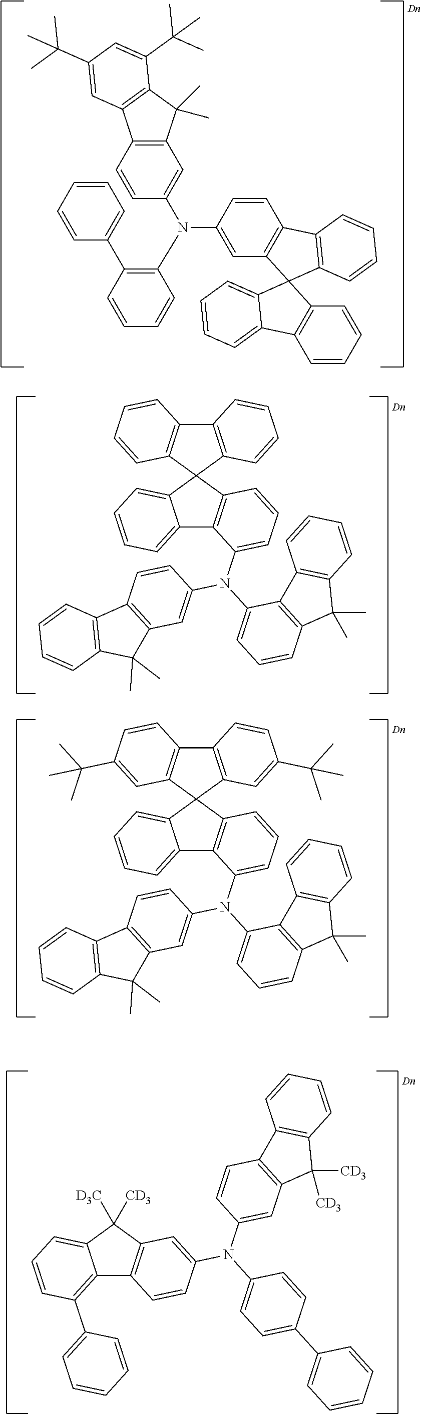

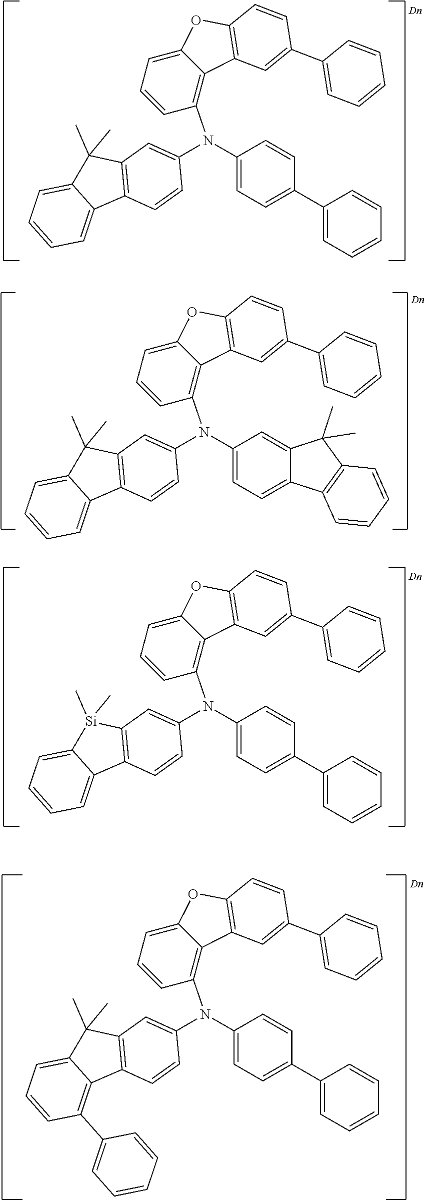

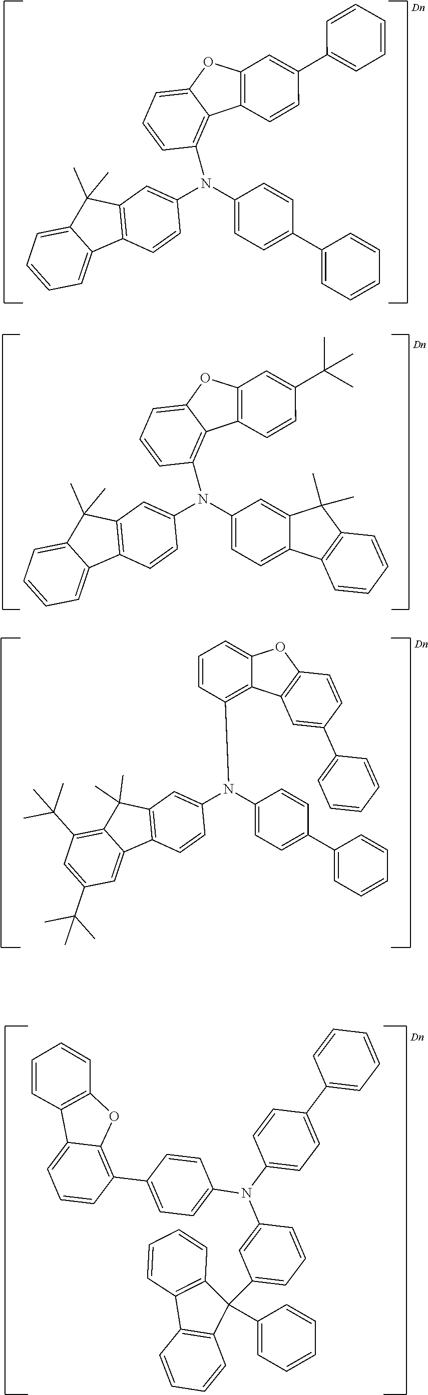

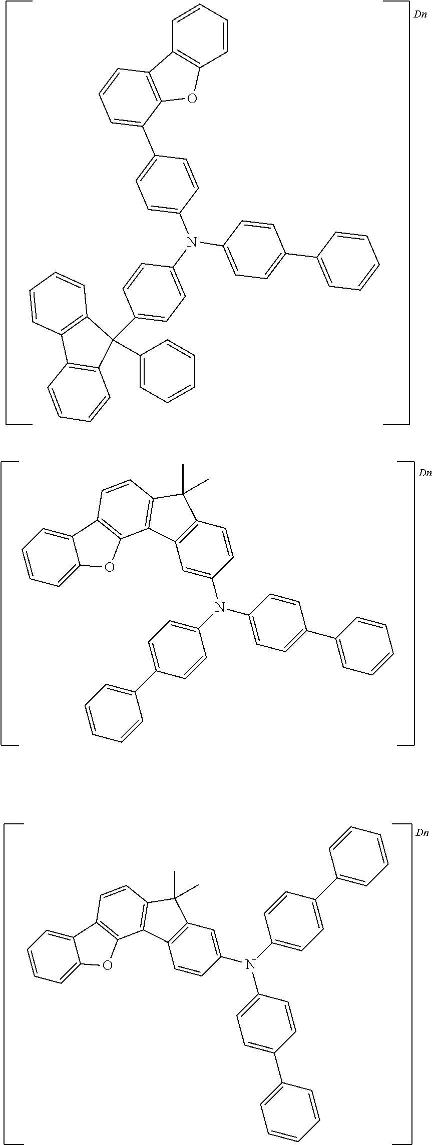

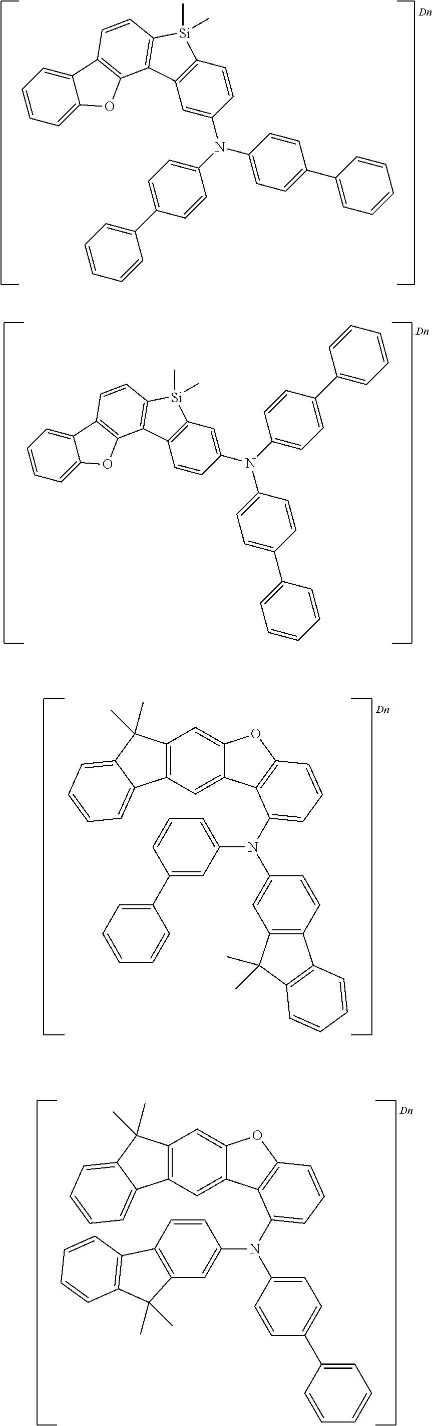

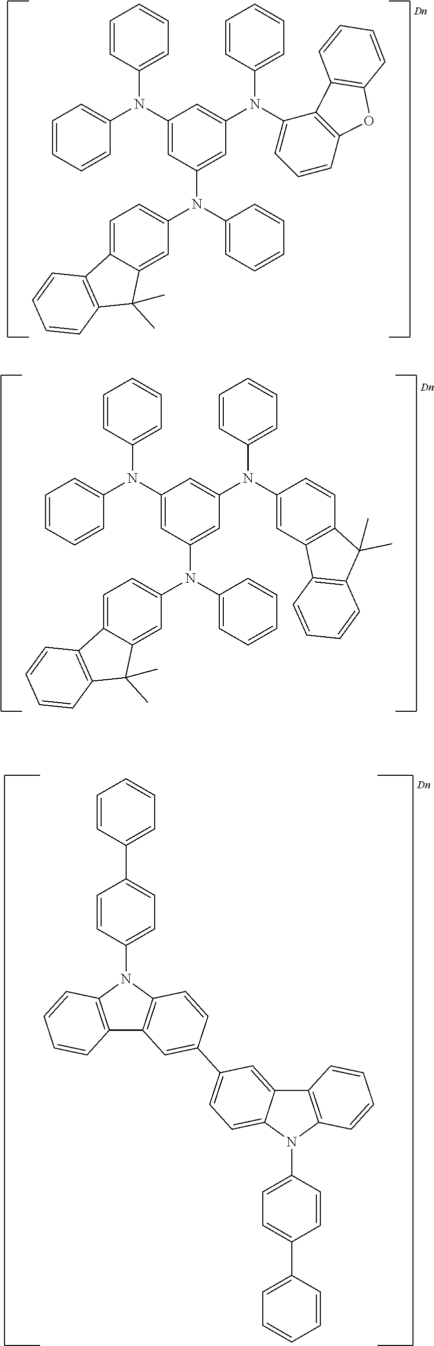

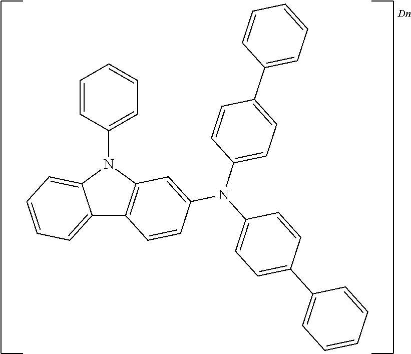

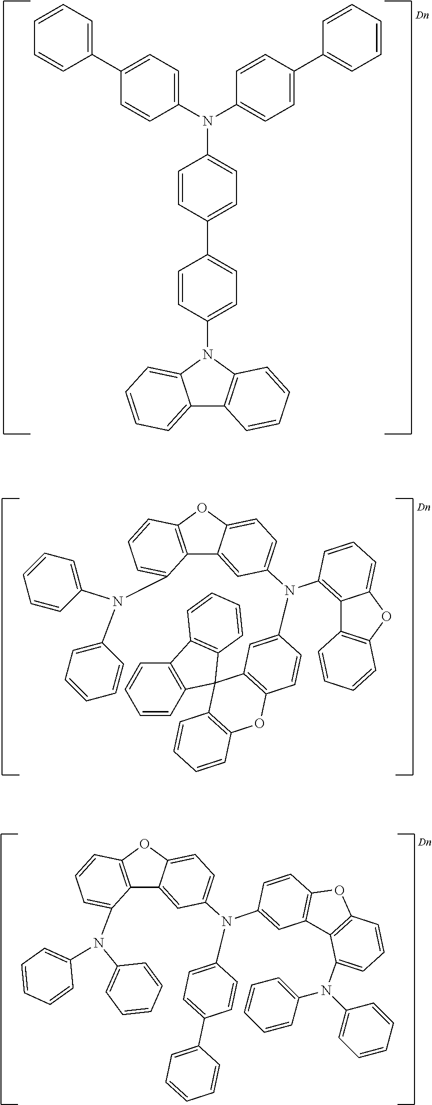

(Dn refers to the number of deuterium substitutions and indicates a structure substituted with one or more deuterium atoms)

In the hole transport region 140, in addition to the compounds described above, other suitable compounds having a similar structure may also be used.

Also, the charge transport region may be, e.g., the electron transport region 150.

The electron transport region 150 may further increase electron injection or electron mobility and block holes between the cathode 110 and the light emitting layer 130.

In an implementation, the electron transport region 150 may include, e.g., an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

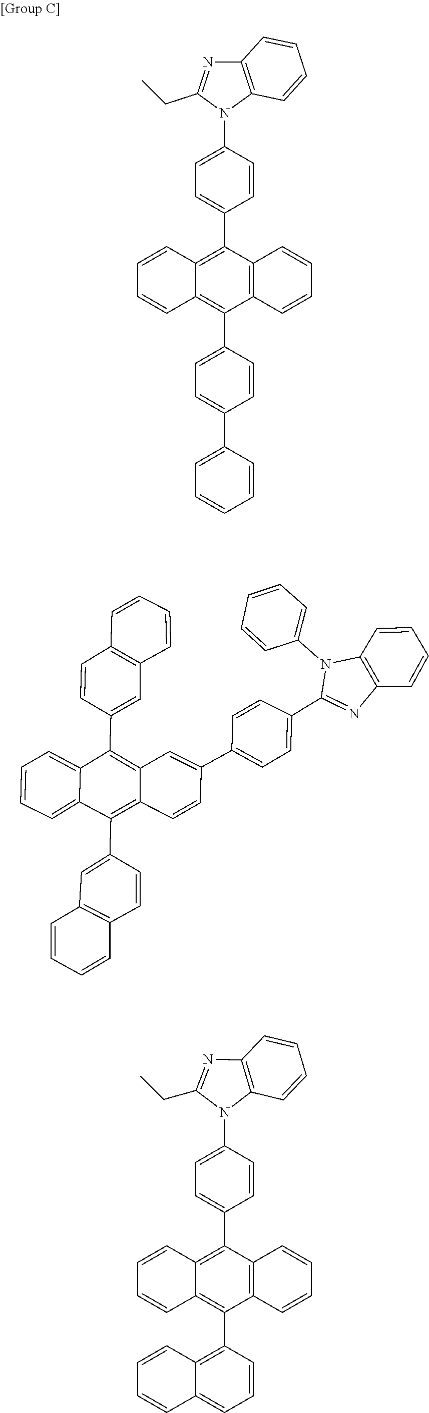

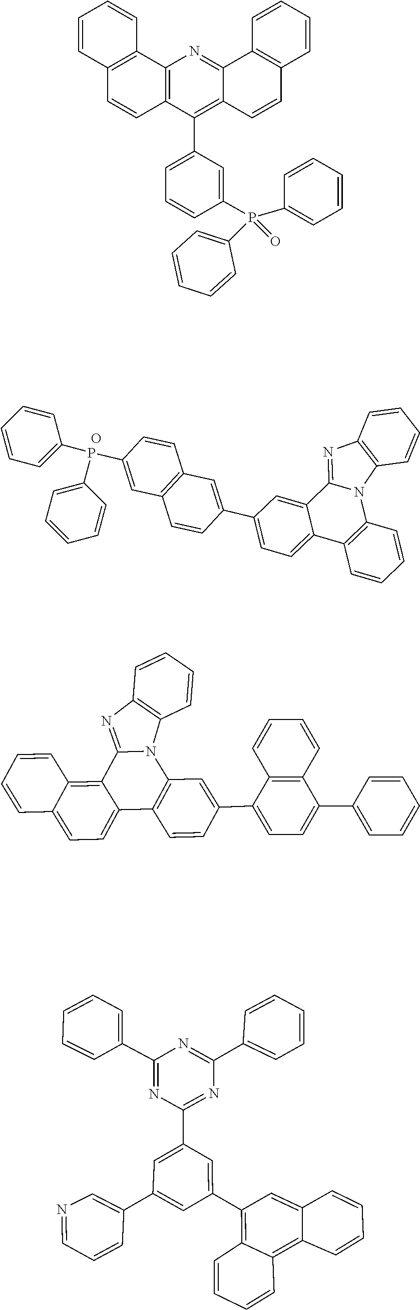

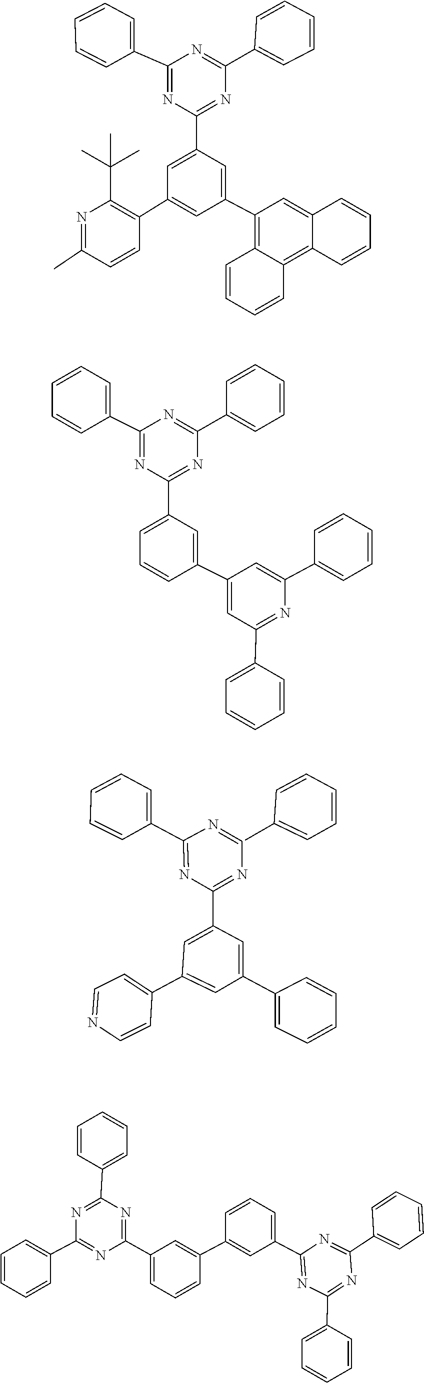

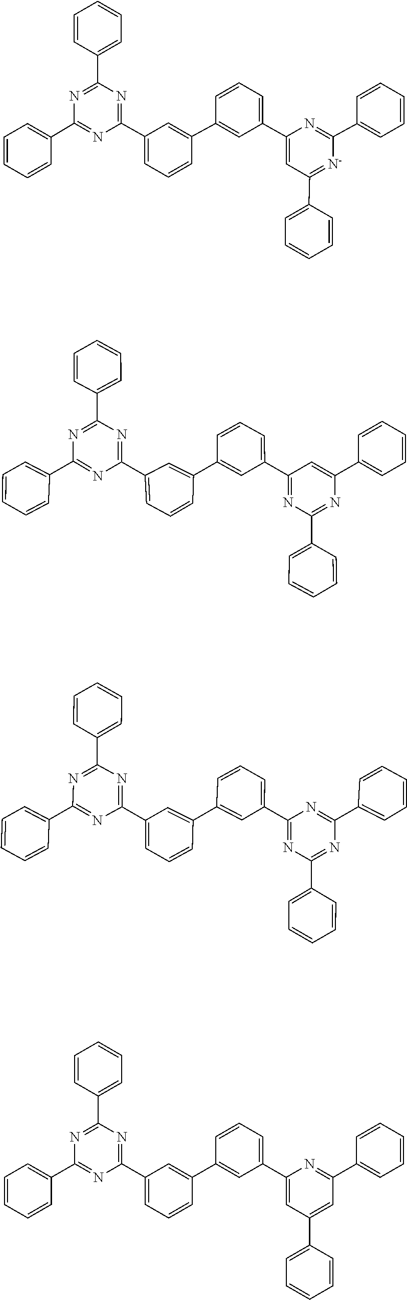

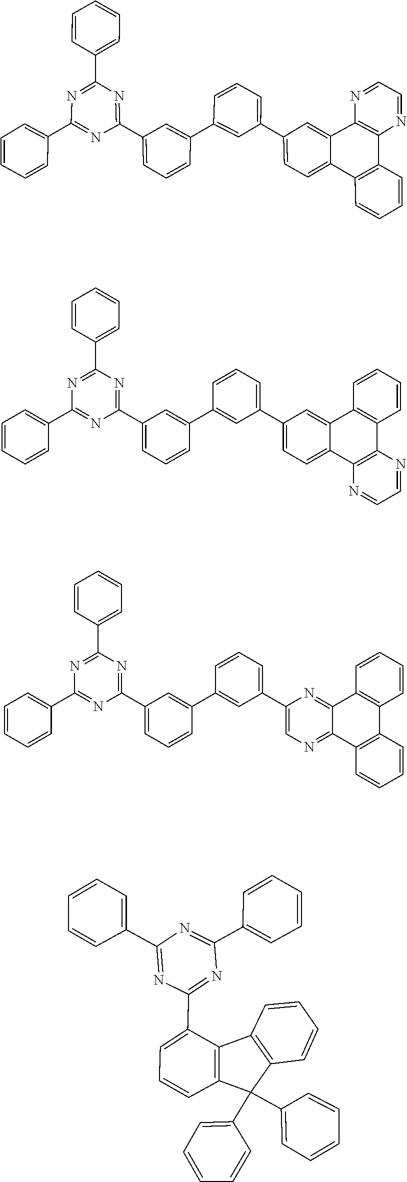 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1]

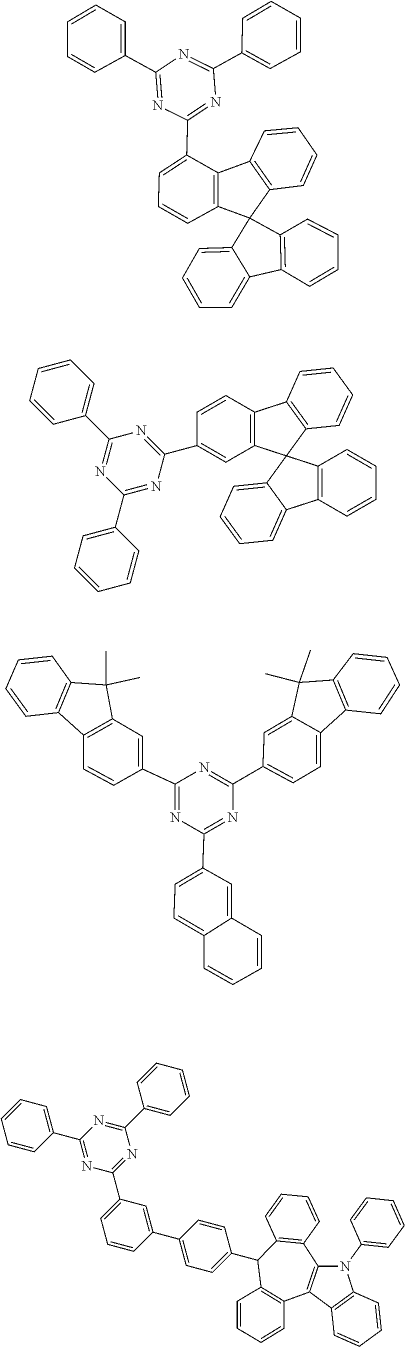 [SMILES: c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(C5c6ccccc6-c6c(n(-c7ccccc7)c7ccccc67)-c6ccccc65)cc4)c3)n2)cc1]

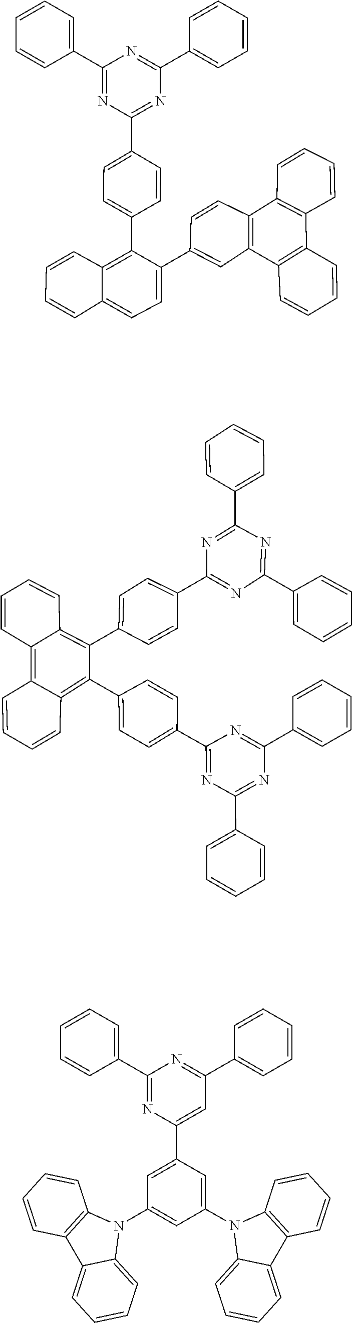 [SMILES: c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1]

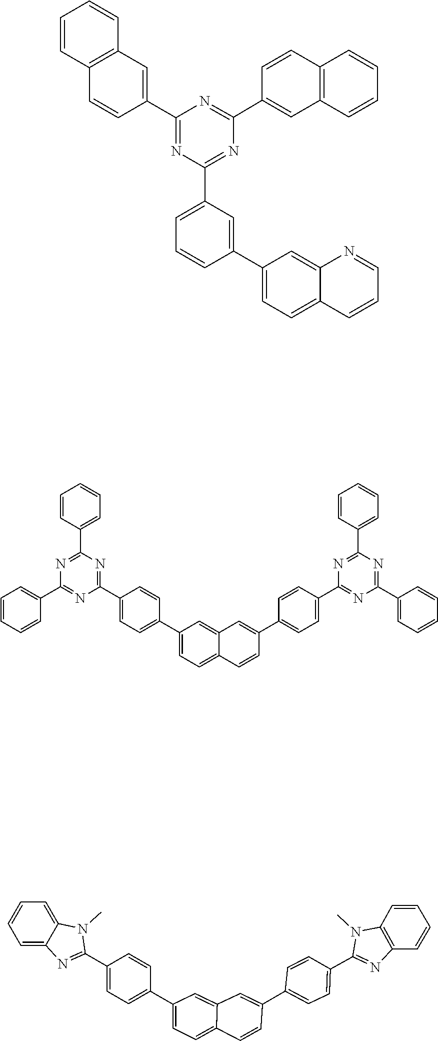 [SMILES: c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21]

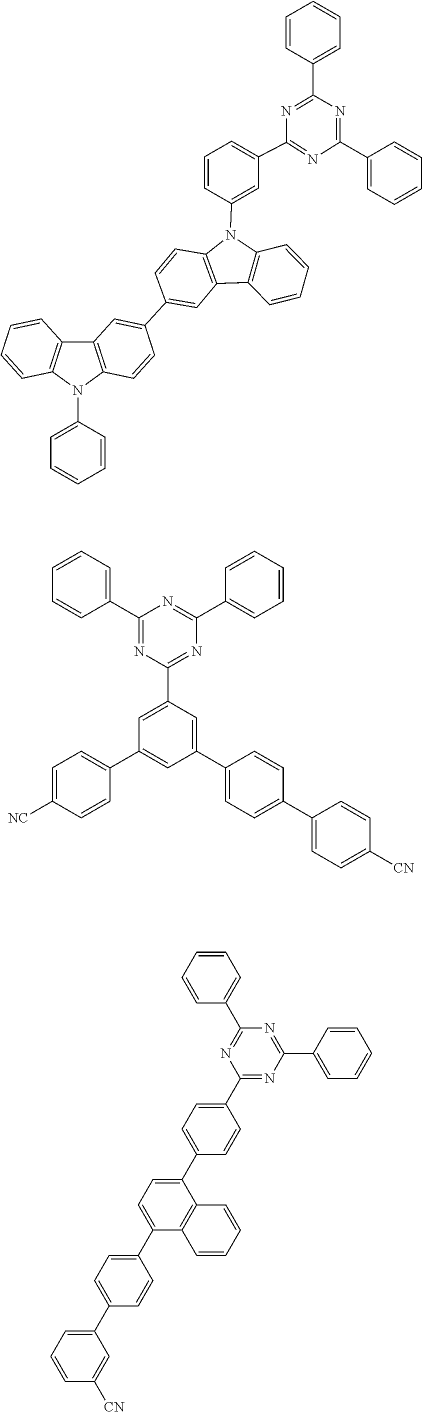

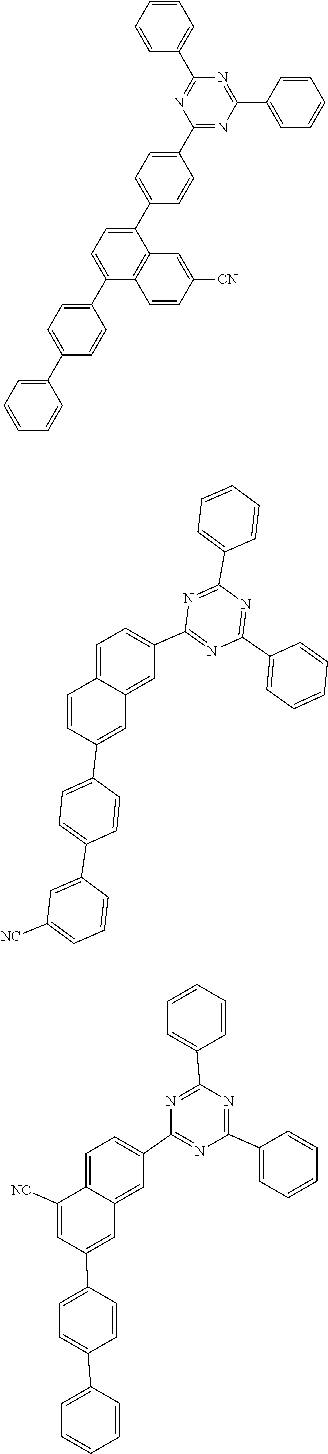 [SMILES: N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

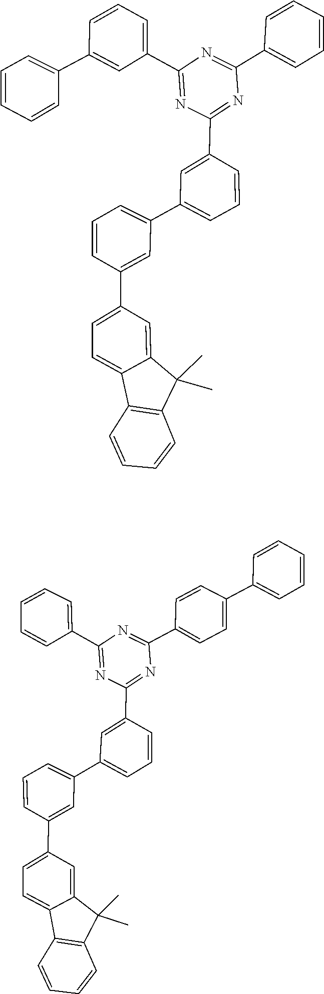 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

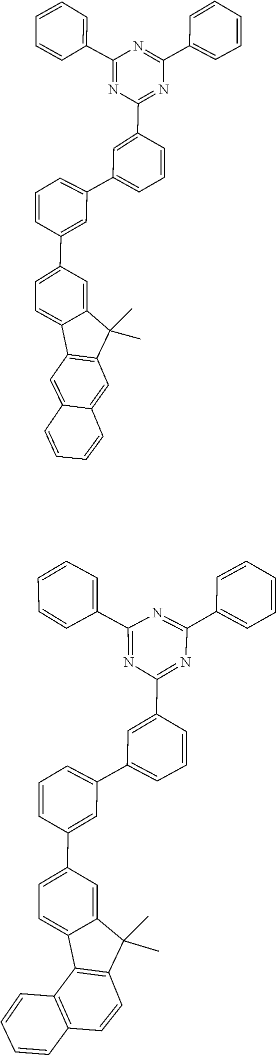 [SMILES: CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21]

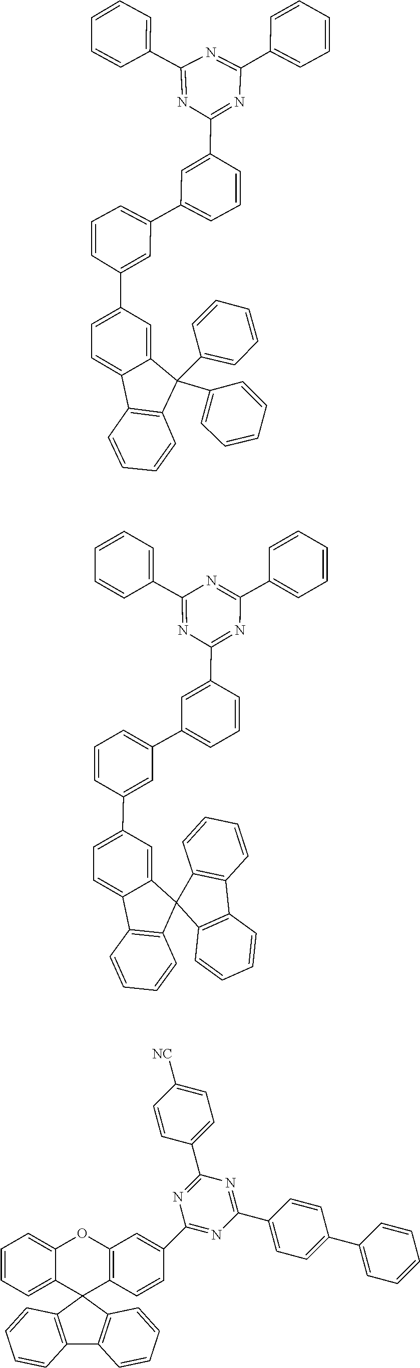 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; N#Cc1ccc(-c2nc(-c3ccc(-c4ccccc4)cc3)nc(-c3ccc4c(c3)Oc3ccccc3C43c4ccccc4-c4ccccc43)n2)cc1]

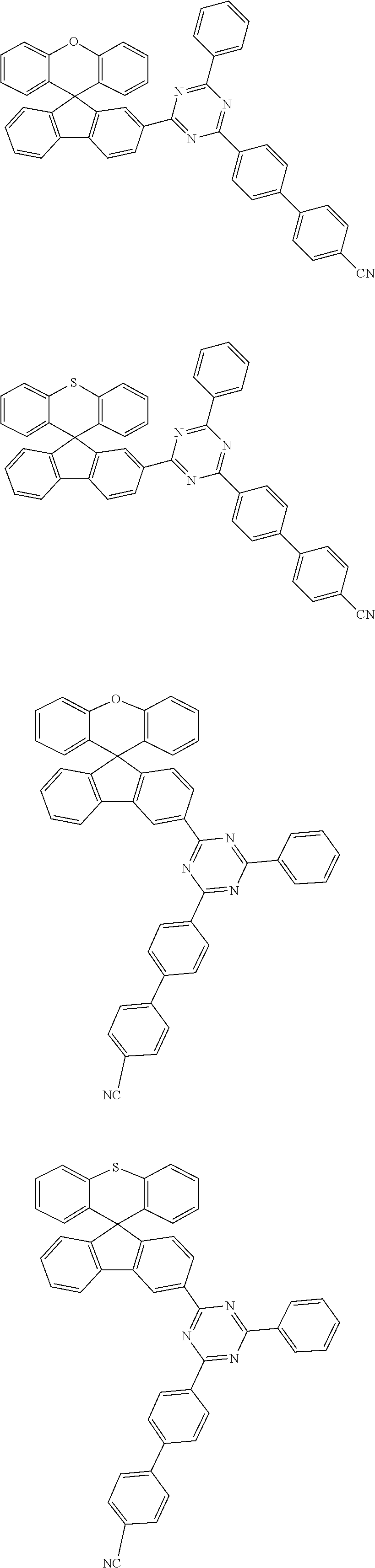 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1]

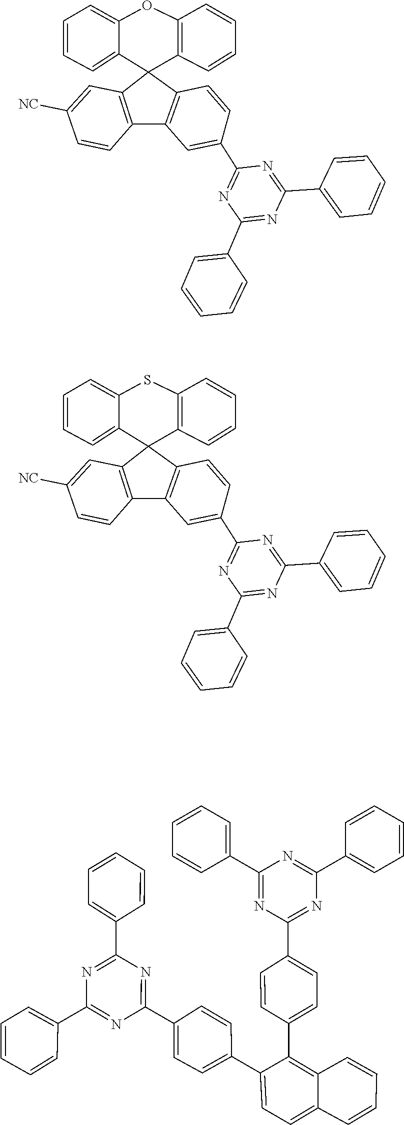 [SMILES: N#Cc1ccc2c(c1)C1(c3ccccc3Oc3ccccc31)c1ccc(-c3nc(-c4ccccc4)nc(-c4ccccc4)n3)cc1-2 ; N#Cc1ccc2c(c1)C1(c3ccccc3Sc3ccccc31)c1ccc(-c3nc(-c4ccccc4)nc(-c4ccccc4)n3)cc1-2 ; c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1]

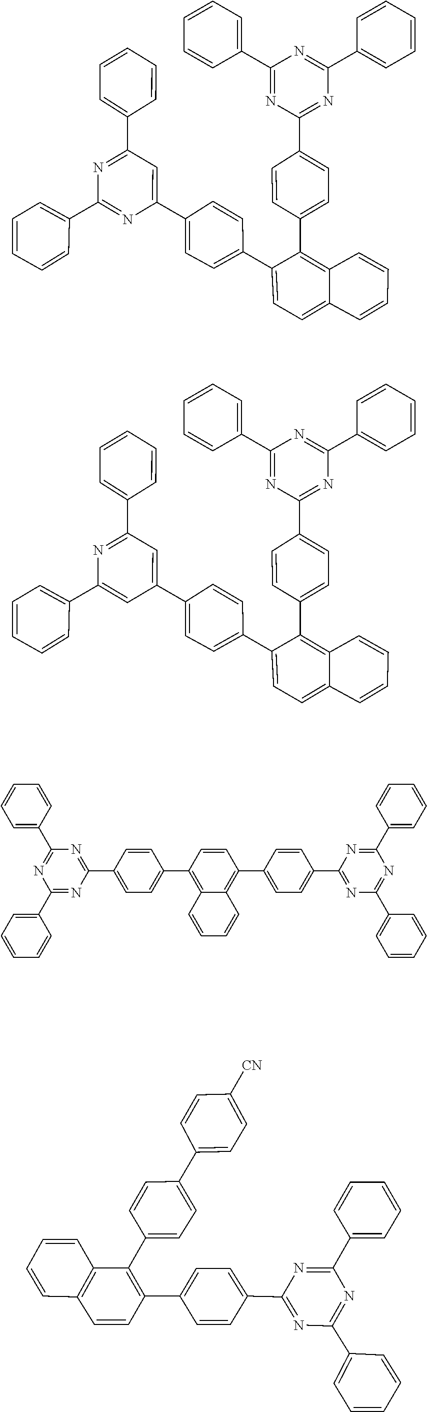 [SMILES: c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1]

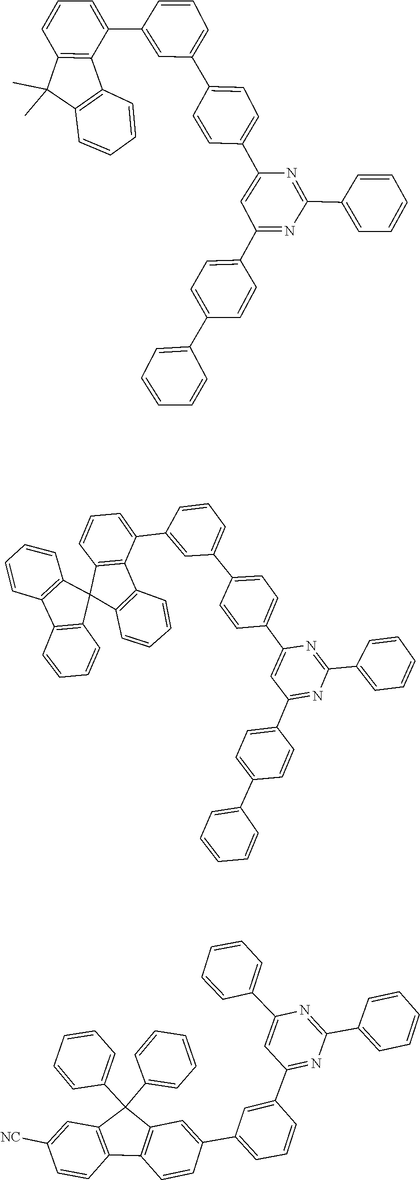 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

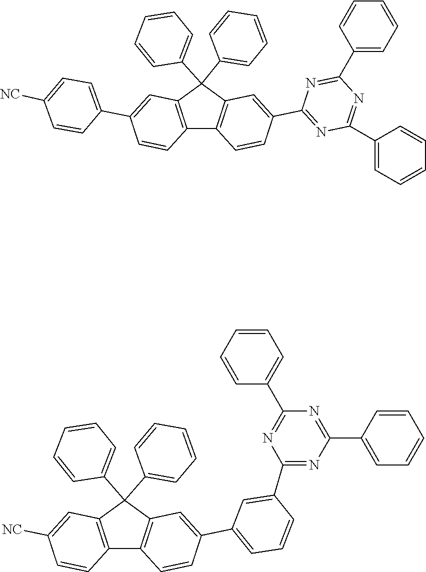

Some example embodiments may be an organic light emitting diode including the light emitting layer as the organic layer.

Some example embodiments may be an organic light emitting diode including a light emitting layer and a hole transport region as the organic layer.

Some example embodiments may be an organic light emitting diode including a light emitting layer and an electron transport region as the organic layer.

An organic light emitting diode according to an implementation includes a hole transport region 140 and an electron transport region 150 in addition to the light emitting layer 130 as the organic layer 105, as shown in the FIGURE.

In an implementation, an organic light emitting diode may further include an electron injection layer a hole injection layer, or the like, in addition to the light emitting layer as the organic layer.

The organic light emitting diodes 100 may be manufactured by forming an anode or a cathode on a substrate, and then forming an organic layer by a dry film method, e.g., vacuum deposition, sputtering, plasma plating, or ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by suitable methods.

### Synthesis of Compound for Organic Optoelectronic Device

### Synthesis Example 1: Synthesis of Compound 1

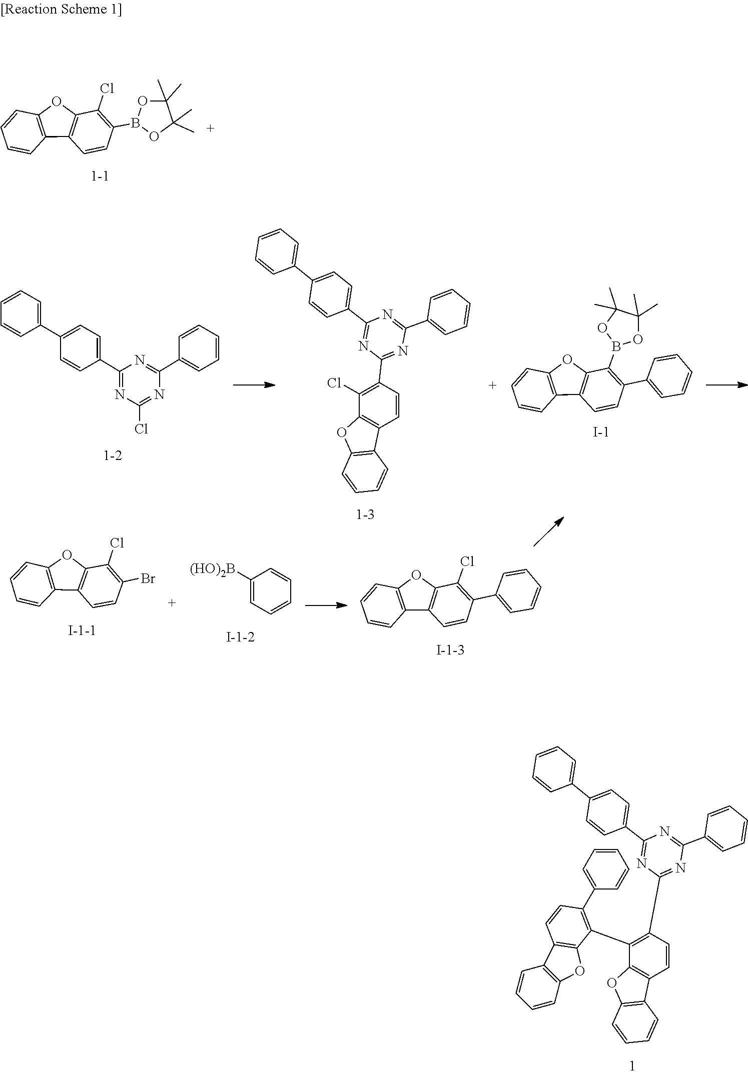

### 1st Step: Synthesis of Intermediate 1-3

1 equivalent of 2-(4-chlorodibenzo[b,d]furan-3-yl)-4,4,5,5-tetramethyl-1,3,2-dioxaborolane (50.0 g, 152.2 mmol, Intermediate 1-1) was added to 500 ml of THF (tetrahydrofuran) and 250 ml of distilled water, and 1.2 equivalents of 2-([1,1′-biphenyl]-4-yl)-4-chloro-6-phenyl-1,3,5-triazine (Intermediate 1-2), 0.03 equivalents of Pd(PPh₃)₄, and 2 equivalents of K₂CO₃ were added, and the mixture was stirred at 80° C. for 8 hours under a nitrogen atmosphere. After removing the water layer, the organic layer was dried under reduced pressure. After washing the obtained solid with water and hexane, the solid was recrystallized with 600 mL of xylene to obtain Intermediate 1-3 in a yield of 60%.

### 2nd Step: Synthesis of Intermediate I-1-3

50.0 g (177.6 mmol) of 1 equivalent of 3-bromo-4-chlorodibenzo[b,d]furan (Intermediate I-1-1) was added to 500 ml of THF and 250 ml of distilled water, and 1.2 equivalent of phenylboronic acid (Intermediate I-1-2), 0.03 equivalent of Pd(PPh₃)₄ and 2 equivalents of K₂CO₃ were added thereto and then, stirred at 80° C. for 8 hours under a nitrogen atmosphere. After removing an aqueous layer, Intermediate I-1-3 was obtained at a yield of 70% through column chromatography (hexane/dichloromethane=7:3).

### 3rd Step: Synthesis of Intermediate I-1

34.6 g (124.3 mmol) of 1 equivalent of Intermediate I-1-3 was added to 300 mL of xylene, and 0.03 equivalent of Pd₂(dba)₃, 0.12 equivalent of tricyclohexylphosphine, 1.2 equivalent of bis(pinacolato)diboron, and 2 equivalents of KOAc were added thereto and then, heated under reflux for 8 hours under a nitrogen atmosphere. Subsequently, after adding 200 mL of distilled water to the reaction solution and then, removing an aqueous layer, Intermediate I-1 was obtained at a yield of 55% through column chromatography (hexane/dichloromethane=5:5).

4th step: Synthesis of Compound 1

34.9 g (152.2 mmol) of 1 equivalent of Intermediate 1-3 was added to 300 ml of dioxane and 150 ml of distilled water, and 1 equivalent of Intermediate I-1, 0.02 equivalent of Pd₂(dba)₃, 0.1 equivalent of P(t-Bu)₃, and 2.5 equivalents of Cs₂CO₃ were added thereto and then, stirred for 8 hours at 100° C. under a nitrogen atmosphere. After removing a water layer, an organic layer therefrom was dried under a reduced pressure. A solid obtained therefrom was washed with water and hexane and then, recrystallized with 400 mL of xylene, obtaining Compound 1 at a yield of 50%.

### Synthesis Example 2: Synthesis of Compound 10

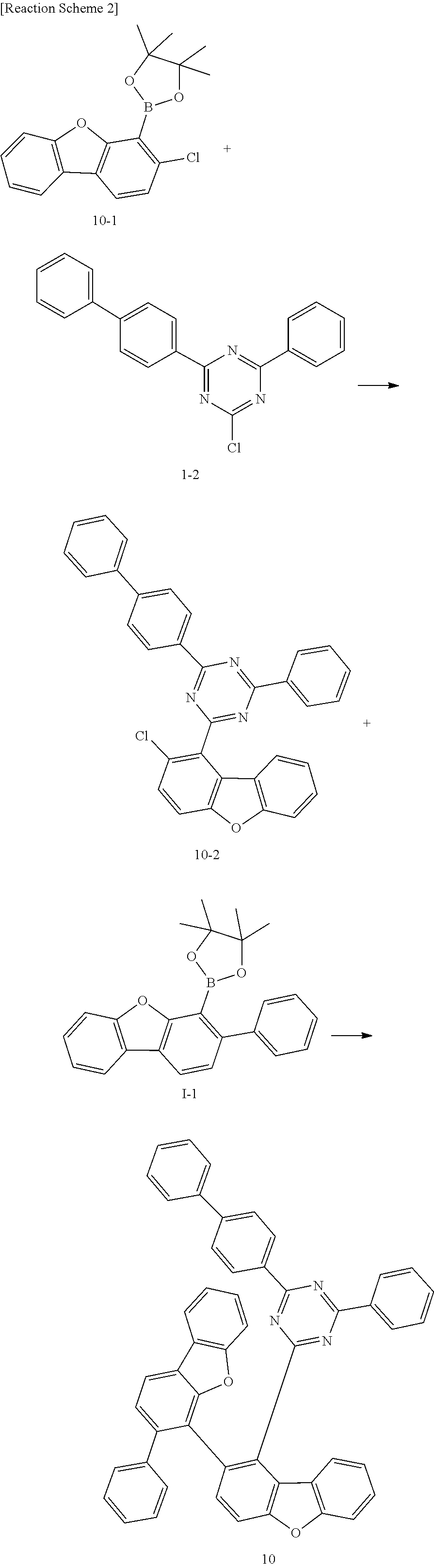 [SMILES: CC1(C)OB(c2c(Cl)ccc3c2oc2ccccc23)OC1(C)C ; Clc1nc(-c2ccccc2)nc(-c2ccc(-c3ccccc3)cc2)n1 ; Clc1ccc2oc3ccccc3c2c1-c1nc(-c2ccccc2)nc(-c2ccc(-c3ccccc3)cc2)n1 ; CC1(C)OB(c2c(-c3ccccc3)ccc3c2oc2ccccc23)OC1(C)C ; [I-] ; c1ccc(-c2ccc(-c3nc(-c4ccccc4)nc(-c4c(-c5c(-c6ccccc6)ccc6c5oc5ccccc56)ccc5oc6ccccc6c45)n3)cc2)cc1]

### 1st Step: Synthesis of Intermediate 10-2

Intermediate 10-2 was obtained at a yield of 55% in the same manner as in the 1st step of Synthesis Example 1 except that 50.0 g (152.2 mmol) of 1 equivalent of 2-(3-chlorodibenzo[b,d]furan-4-yl)-4,4,5,5-tetramethyl-1,3,2-dioxaborolane (Intermediate 10-1) and 1.2 equivalent of 2-([1,1′-biphenyl]-4-yl)-4-chloro-6-phenyl-1,3,5-triazine (Intermediate 1-2) were used.

### 2nd Step: Synthesis of Compound 10

Compound 10 was obtained at a yield of 48% in the same manner as in the 4th step of Synthesis Example 1 by using 1 equivalent of Intermediate 10-2 and 1 equivalent of Intermediate I-1.

### Synthesis Example 3: Synthesis of Compound 16

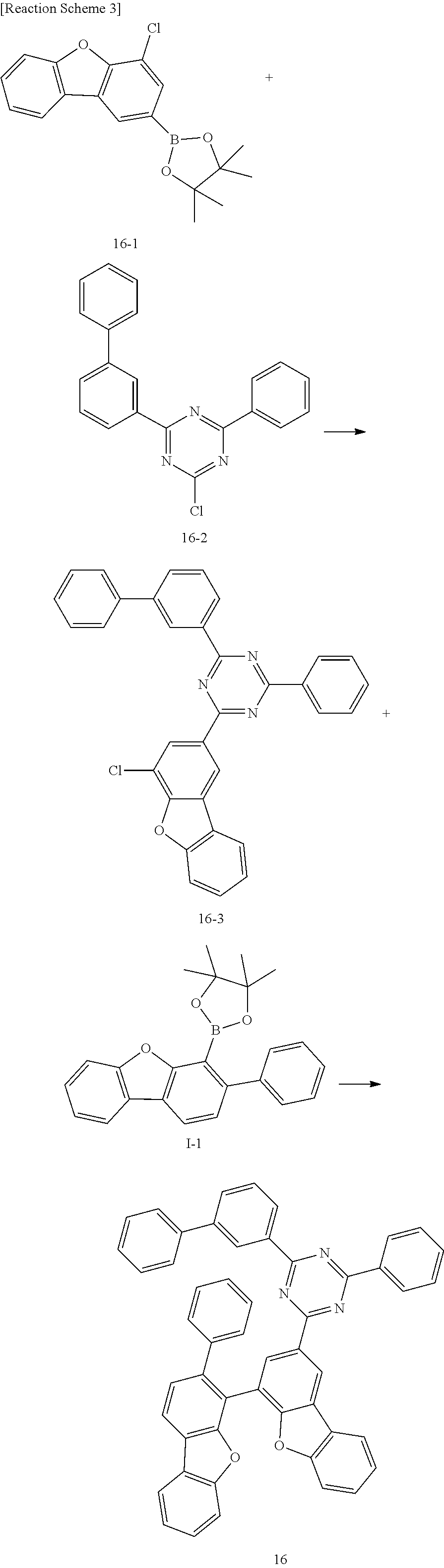 [SMILES: CC1(C)OB(c2cc(Cl)c3oc4ccccc4c3c2)OC1(C)C ; Clc1nc(-c2ccccc2)nc(-c2cccc(-c3ccccc3)c2)n1 ; Clc1cc(-c2nc(-c3ccccc3)nc(-c3cccc(-c4ccccc4)c3)n2)cc2c1oc1ccccc12 ; CC1(C)OB(c2c(-c3ccccc3)ccc3c2oc2ccccc23)OC1(C)C ; [I-] ; c1ccc(-c2cccc(-c3nc(-c4ccccc4)nc(-c4cc(-c5c(-c6ccccc6)ccc6c5oc5ccccc56)c5oc6ccccc6c5c4)n3)c2)cc1]

### 1st Step: Synthesis of Intermediate 16-3

Intermediate 16-3 was obtained at a yield of 63% in the same manner as in the 1st step of Synthesis Example 1 by using 50.0 g (152.2 mmol) of 1 equivalent of 2-(4-chlorodibenzo[b,d]furan-2-yl)-4,4,5,5-tetramethyl-1,3,2-dioxaborolane (Intermediate 16-1) and 1.2 equivalent of 2-([1,1′-biphenyl]-3-yl)-4-chloro-6-phenyl-1,3,5-triazine (Intermediate 16-2).

### 2nd Step: Synthesis of Compound 16

Compound 16 was obtained at a yield of 50% in the same manner as in the 4^{th} step of Synthesis Example 1 by using 1 equivalent of Intermediate 16-3 and 1 equivalent of Intermediate I-1.

### Synthesis Example 4: Synthesis of Compound 26

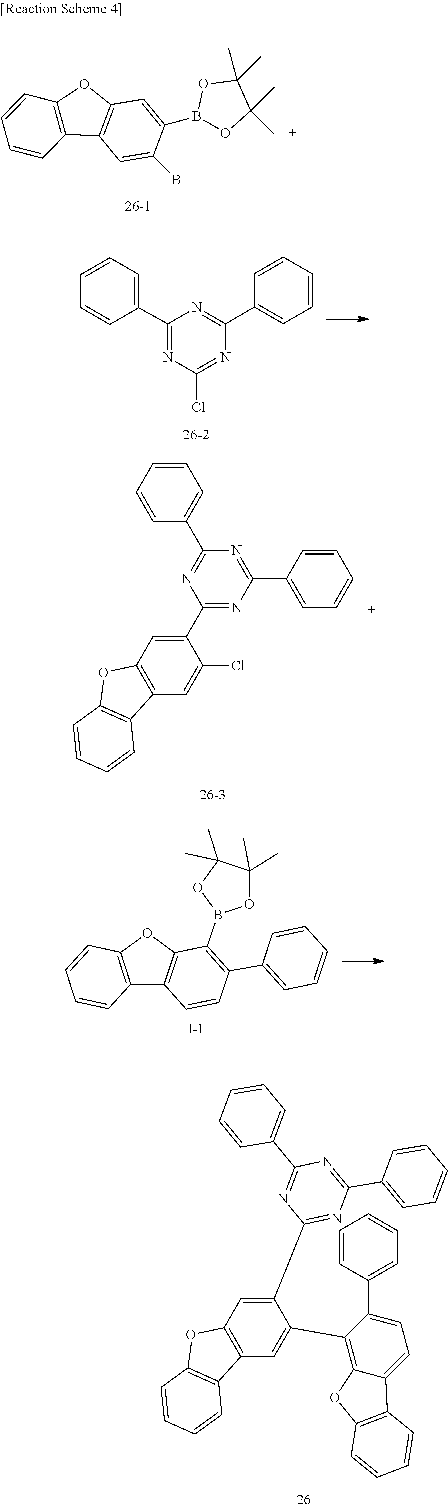

### 1st Step: Synthesis of Intermediate 26-3

Intermediate 26-3 was obtained at a yield of 60% in the same manner as in the 1st step of Synthesis Example 1 by using 50.0 g (152.2 mmol) of 1 equivalent of 2-(2-chlorodibenzo[b,d]furan-3-yl)-4,4,5,5-tetramethyl-1,3,2-dioxaborolane (Intermediate 26-1) and 1.2 equivalent of 2-chloro-4,6-diphenyl-1,3,5-triazine (Intermediate 26-2).

### 2nd Step: Synthesis of Compound 26

Compound 26 was obtained at a yield of 55% in the same manner as in the 4^{th} step of Synthesis Example 1 by using 1 equivalent of Intermediate 26-3 and 1 equivalent of Intermediate I-1.

### Comparative Synthesis Example 1: Synthesis of Compound R-1

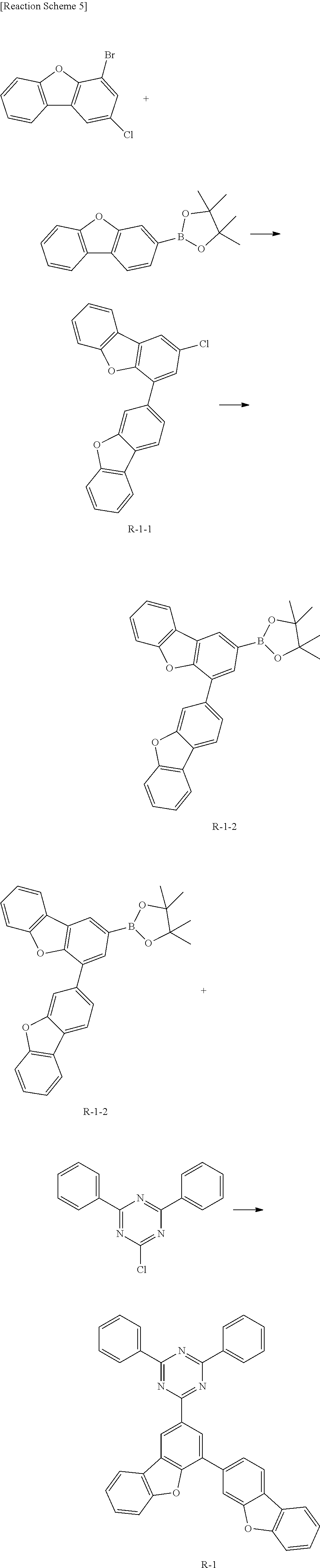

### 1st Step: Synthesis of Intermediate R-1-1

30 g (106.6 mmol) of 1 equivalent of 4-bromo-2-chloro-dibenzofuran was added to 200 ml of THF and 100 ml of distilled water, and 1 equivalent of 2-(dibenzo[b,d]furan-3-yl)-4,4,5,5-tetramethyl-1,3,2-dioxaborolane, 0.03 equivalent of Pd(PPh₃)₄, and 2 equivalents of K₂CO₃ were added thereto and then, stirred at 80° C. for 8 hours under a nitrogen atmosphere. After removing an aqueous layer, an organic layer therefrom was dried under reduced pressure. A solid obtained therefrom was washed with water and hexane and then, recrystallized with 400 mL of xylene, obtaining Intermediate R-1-1 at a yield of 55%.

### 2nd Step: Synthesis of Intermediate R-1-2

21.6 g (58.6 mmol) of 1 equivalent of Intermediate R-1-1 was added to 300 mL of xylene, 0.03 equivalent of Pd₂(dba)₃, 0.12 equivalent of tricyclohexylphosphine, 1.2 equivalent of bis(pinacolato)diboron, and 2 equivalents of KOAc were added thereto and then, heated under reflux for 8 hours under a nitrogen atmosphere. After adding 300 mL of distilled water to the reaction solution and then, removing an aqueous layer, Intermediate R-1-2 was obtained at a yield of 55% through column chromatography (hexane/dichloromethane=5:5).

### 3rd Step: Synthesis of Compound R-1

Compound R-1 was synthesized at a yield of 57% in the same manner as in the 1st step of Comparative Synthesis Example 1 by using 1 equivalent of Intermediate R-1-2 and 1 equivalent of 2-chloro-4,6-diphenyl-1,3,5-triazine.

### Comparative Synthesis Example 2: Synthesis of Compound R-2

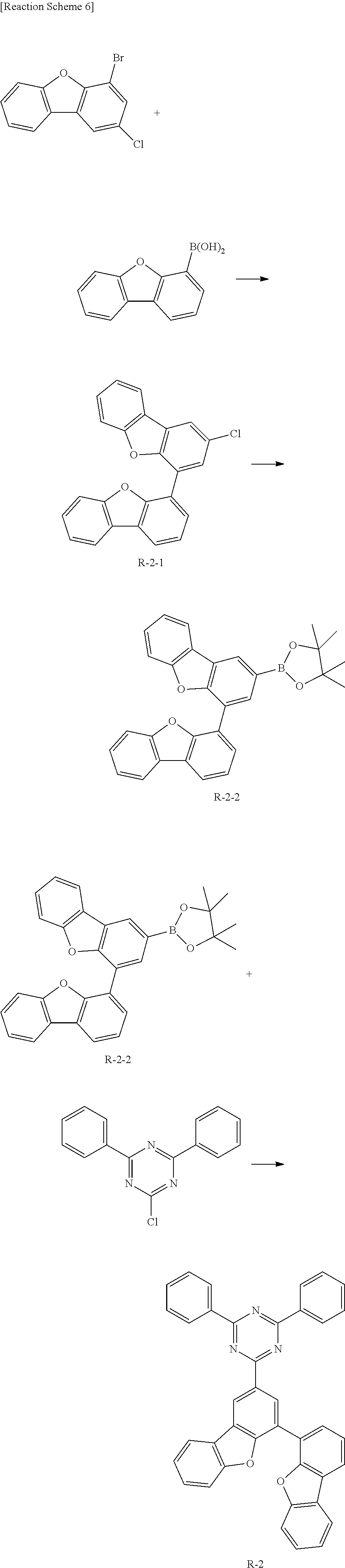

### 1st Step: Synthesis of Intermediate R-2-1

30 g (106.6 mmol) of 1 equivalent of 4-bromo-2-chloro-dibenzofuran was added to 200 ml of THF and 100 ml of distilled water, and 1 equivalent of dibenzofuran-4-yl-boronic acid, 0.03 equivalent of Pd(PPh₃)₄ and 2 equivalents of K₂CO₃ were added thereto and then, stirred at 80° C. for 8 hours under a nitrogen atmosphere. After removing an aqueous layer, an organic layer therefrom was dried under reduced pressure. The obtained solid was washed with water and hexane and then recrystallized with 400 mL of xylene, obtaining Intermediate R-2-1 at a yield of 62%.

### 2nd Step: Synthesis of Intermediate R-2-2

24.4 g (66.1 mmol) of 1 equivalent of Intermediate R-2-1 was added to 300 mL of xylene, and 0.03 equivalent of Pd₂(dba)₃, 0.12 equivalent of tricyclohexylphosphine, 1.2 equivalent of bis(pinacolato)diboron, and 2 equivalents of KOAc were added thereto and then, heated under reflux for 8 hours under a nitrogen atmosphere. After adding 300 mL of distilled water to the reaction solution and then, removing an aqueous layer, Intermediate R-2-2 was obtained at a yield of 55% through column chromatography (hexane/dichloromethane=5:5).

### 3rd Step: Synthesis of Compound R-2

Compound R-2 was synthesized at a yield of 60% in the same manner as in the 1st step of Comparative Synthesis Example 2 by using 1 equivalent of Intermediate R-2-2 and 1 equivalent of 2-chloro-4,6-diphenyl-1,3,5-triazine.

### Comparative Synthesis Example 3: Synthesis of Compound R-3

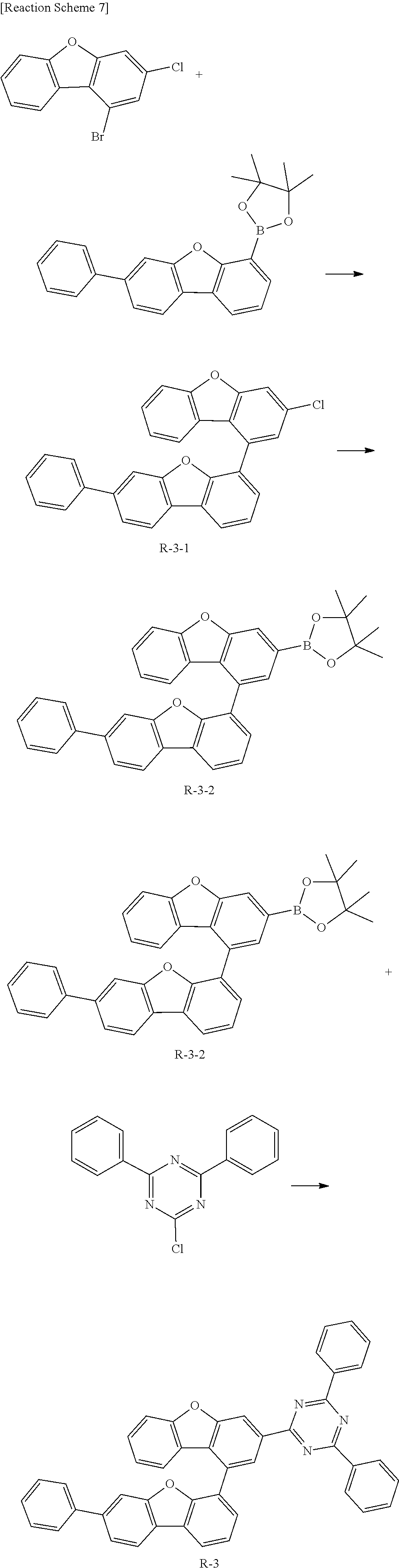

### 1st Step: Synthesis of Intermediate R-3-1

30 g (106.6 mmol) of 1 equivalent of 1-bromo-3-chlorodibenzofuran was added to 200 ml of dioxane and 100 ml of distilled water, and 1 equivalent of 4,4,5,5-tetramethyl-2-(7-phenyldibenzofuran-4-yl)-1,3,2-dioxaborolane, 0.03 equivalent of Pd(PPh₃)₄, and 2 equivalents of K₂CO₃ were added thereto and then, stirred at 100° C. for 8 hours under a nitrogen atmosphere. After removing an aqueous layer, an organic layer therefrom was dried under a reduced pressure. A solid obtained therefrom was washed with water and hexane and then, recrystallized with 400 mL of xylene, obtaining Intermediate R-3-1 at a yield of 48%.

### 2nd Step: Synthesis of Intermediate R-3-2

22.8 g (51.2 mmol) of 1 equivalent of Intermediate R-3-1 was added to 300 mL of xylene, and 0.03 equivalent of Pd₂(dba)₃, 0.12 equivalent of tricyclohexylphosphine, 1.2 equivalent of bis(pinacolato)diboron, and 2 equivalents of KOAc were added thereto and then, heated under reflux for 8 hours under a nitrogen atmosphere. After adding 300 mL of distilled water to the reaction solution and then, removing an aqueous layer, Intermediate R-3-2 was obtained at a yield of 45% through column chromatography (hexane/dichloromethane=5:5).

### 3rd Step: Synthesis of Compound R-3

Compound R-3 was synthesized at a yield of 62% in the same manner as in the 1st step of Comparative Synthesis Example 3 by using 1 equivalent of Intermediate R-3-2 and 1 equivalent of 2-chloro-4,6-diphenyl-1,3,5-triazine.

### Synthesis of Second Compound

### Synthesis Example 5: Synthesis of Compound B-136

Compound B-136 was synthesized by using the same method as in Korean Publication No. 2016-0049842.

### Example 1: Manufacturing of Green Organic Light Emitting Diode (Single Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound B was deposited on the hole transport layer to a thickness of 350 Å to form a hole transport auxiliary layer. On the hole transport auxiliary layer, Compound 1 was used as a host and PhGD was doped at 7 wt % as a dopant to form a 400 Å-thick light emitting layer by vacuum deposition. Then, Compound C was deposited on the light emitting layer to a thickness of 50 Å to form an electron transport auxiliary layer, and Compound D and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured to have a structure of ITO/Compound A (3% NDP-9 doping, 100 Å)/Compound A (1,350 Å)/Compound B (350 Å)/EML [Host (Compound 1):PhGD=93 wt %:7 wt %](400 Å)/Compound C (50 Å)/Compound D:LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-dibenzofuranyl)phenyl]-N-[4-(9-phenyl-9H-fluoren-9-yl)phenyl][1,1′-biphenyl]-4-amine
Compound C: 2,4-diphenyl-6-(4′,5′,6′-triphenyl[,1′: 2′,1″: 3″,1′″:3′″, 1″″-quinquephenyl]-3″″-yl)-1,3,5-triazine
Compound D: 2-(1,1′-biphenyl-4-yl)-4-(9,9-diphenylfluoren-4-yl)-6-phenyl-1,3,5-triazine

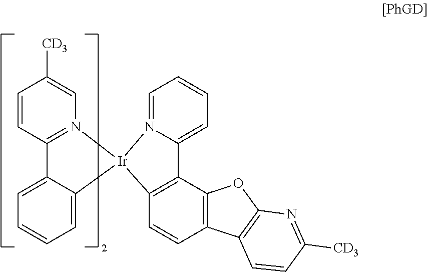

### Examples 2 to 4 and Comparative Examples 1 to 3

Each organic light emitting diode was manufactured in the same manner as Example 1, except that the compositions were changed to those shown in Table 1.

### Example 5: Manufacturing of Green Organic Light Emitting Diode (Mixed Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound E was deposited on the hole injection layer to a thickness of 1,350 Å to form a hole transport layer. Compound F was deposited on the hole transport layer to a thickness of 320 Å to form a hole transport auxiliary layer. On the hole transport auxiliary layer, Compound 1 and Compound B-136 were simultaneously used as hosts in a weight ratio of 4:6, and PhGD was doped at 10 wt % as a dopant to form a 380 Å-thick light emitting layer by vacuum deposition. Subsequently, Compound G was deposited on the light emitting layer to a thickness of 50 Å to form an electron transport auxiliary layer, and Compound H and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured to have a structure of ITO/Compound A (3% NDP-9 doping, 100 Å)/Compound E (1,350 Å)/Compound F (320 Å)/EML [Host (Compound 1:Compound B-136=4: 6 wt %/wt %):PhGD=90 wt %:10 wt %](380 Å)/Compound G (50 Å)/Compound H:LiQ (300 Å)/LiQ (15 Å)/Al (1,200 Å).
Compound E: N-(9,9-diphenyl-9H-fluoren-2-yl)-N,9-diphenyl-9H-carbazol-2-amine
Compound F: 9,9-dimethyl-N-[3-(9-phenyl-9H-fluoren-9-yl)phenyl]-4-(4-phenylphenyl)-9H-fluoren-2-amine
Compound G: 4-{4-[4-(9,9-dimethyl-9H-fluoren-4-yl)phenyl]phenyl}-2-phenyl-6-(4-phenylphenyl)pyrimidine
Compound H: 2-(4-{1-[4-(diphenyl-1,3,5-triazin-2-yl)phenyl]naphthalene-2-yl}-4,6-diphenyl-1,3,5-triazine

### Example 6 and Comparative Example 4

Each organic light emitting diode was manufactured in the same manner as Example 5, except that the compositions were changed to those shown in Table 2.

### Evaluation

The power efficiency of the organic light emitting diodes according to Examples 1 to 6 and Comparative Examples 1 to 4 were evaluated.

The specific measurement method is as follows, and the results are as shown in Tables 1 to 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000 Å), while the voltage of the organic light emitting diodes was increased from 0 V to 10 V.

### (3) Measurement of Current Efficiency

Current efficiency (cd/A) at the same current density (10 mA/cm²) were calculated by using the luminance and current density from (1) and (2) above and voltage.

### (4) Measurement of Driving Voltage

The driving voltage of each diode at 15 mA/cm² using a current-voltage meter (Keithley 2400) were measured to obtain the results.

### (5) Measurement of Power Efficiency

The power efficiency value was calculated from Equation 1 below, and the relative value based on the power efficiency of Comparative Example 1 was calculated and shown in Table 1.

$$Power efficiency ( l m / W ) = [ current efficiency ( cd / A ) / driving voltage ( V ) ] * Π ( Π refers to pi ) \qquad [ Equation 1 ]$$

The power efficiency values of Examples 1 to 4 and Comparative Examples 1 to 3 were calculated as relative values based on Comparative Example 1 and are listed in Table 1.

The power efficiency values of Examples 5 to 6 and Comparative Example 4 were calculated as relative values based on Comparative Example 4 and are listed in Table 2.

TABLE 1

| No. | Compound | Power efficiency (%) |
|---|---|---|
| Example 1 | 1 | 111 |
| Example 2 | 10 | 109 |
| Example 3 | 16 | 108 |
| Example 4 | 26 | 106 |
| Comparative | R-1 | 100 |
| Example 1 | | |
| Comparative | R-2 | 100 |
| Example 2 | | |
| Comparative | R-3 | 99 |
| Example 3 | | |

TABLE 2

| | First | Second | Power |
|---|---|---|---|
| No. | compound | compound | efficiency (%) |
| Example 5 | 1 | B-136 | 108 |
| Example 6 | 10 | B-136 | 106 |
| Comparative | R-1 | B-136 | 100 |
| Example 4 | | | |

Referring to Tables 1 and 2, power efficiency of the organic light emitting diodes according to Examples 1 to 6 are significantly improved compared to the organic light emitting diodes according to Comparative Examples 1 to 4.

By way of summation and review, some example embodiments provide a compound for an organic optoelectronic device that can lower the driving voltage and realize an organic optoelectronic device with high efficiency and long life-span.

Some example embodiments provide a composition for an organic optoelectronic device including the compound for an organic optoelectronic device.

Some example embodiments provide an organic optoelectronic device including the compound for an organic optoelectronic device or the composition for an organic optoelectronic device.

Some example embodiments provide a display device including the organic optoelectronic device.

A high-efficiency and long life-span organic optoelectronic device can be realized while lowering the operating voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound represented by Chemical Formula 1: 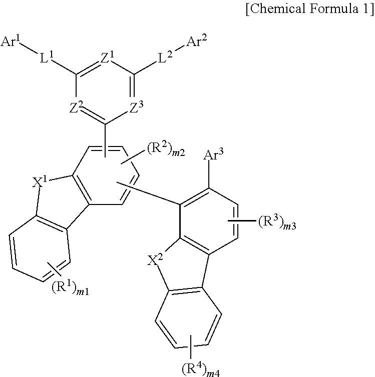
wherein, in Chemical Formula 1,
Z¹ to Z³ are each independently N or CR^{a},
at least two of Z¹ to Z³ are N,
X¹ and X² are each independently O, S, or SiR^{b}R^{c},
at least one of X¹ and X² is O or S,
Ar¹ to Ar³ are each independently a substituted or unsubstituted C6 to C30 aryl group or a substituted or unsubstituted C2 to C30 heterocyclic group,
L¹ and L² are each independently a single bond or a substituted or unsubstituted C6 to C30 arylene group,
R^{a} and R¹ to R⁴ are each independently hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C12 aryl group,
R^{b} and R^{c} are each independently a substituted or unsubstituted C1 to C20 alkyl group or a substituted or unsubstituted C6 to C20 aryl group,
m1 and m4 are each independently an integer of 1 to 4, and
m2 and m3 are each independently an integer of 1 or 2.

2. The compound for an organic optoelectronic device as claimed in claim 1, wherein:
Chemical Formula 1 is represented by one of Chemical Formula 1-1 to Chemical Formula 1-4: 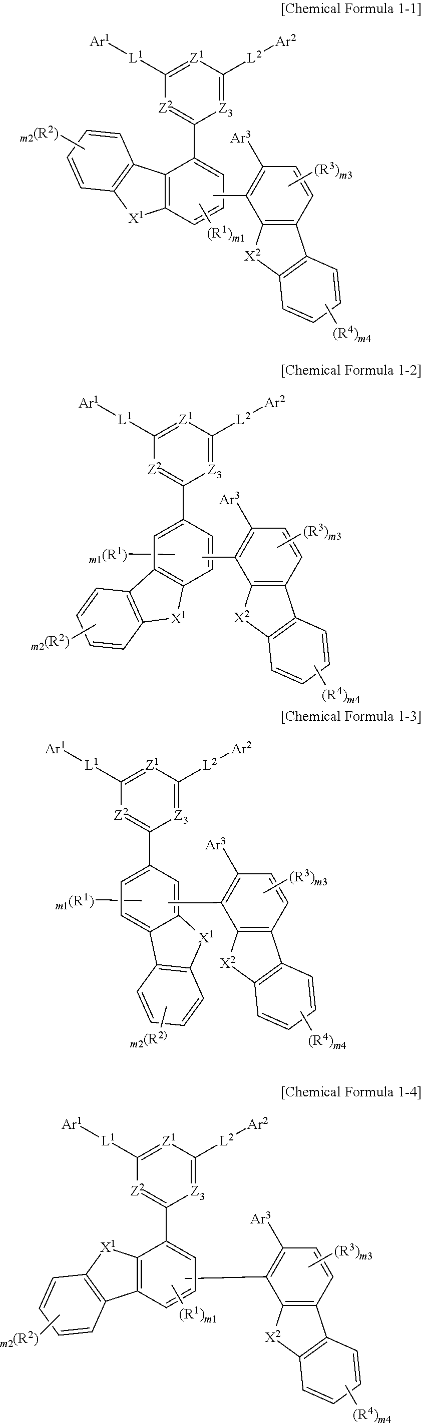
in Chemical Formula 1-1 to Chemical Formula 1-4, Z¹ to Z³, X¹ and X², Ar¹ to Ar³, L¹ and L², R¹ to R⁴ and m1 to m4 are defined the same as those of Chemical Formula 1.

3. The compound for an organic optoelectronic device as claimed in claim 1, wherein:
Chemical Formula 1 is represented by one of Chemical Formula 1-1a, Chemical Formula 1-2c, Chemical Formula 1-3a, or Chemical Formula 1-3c: 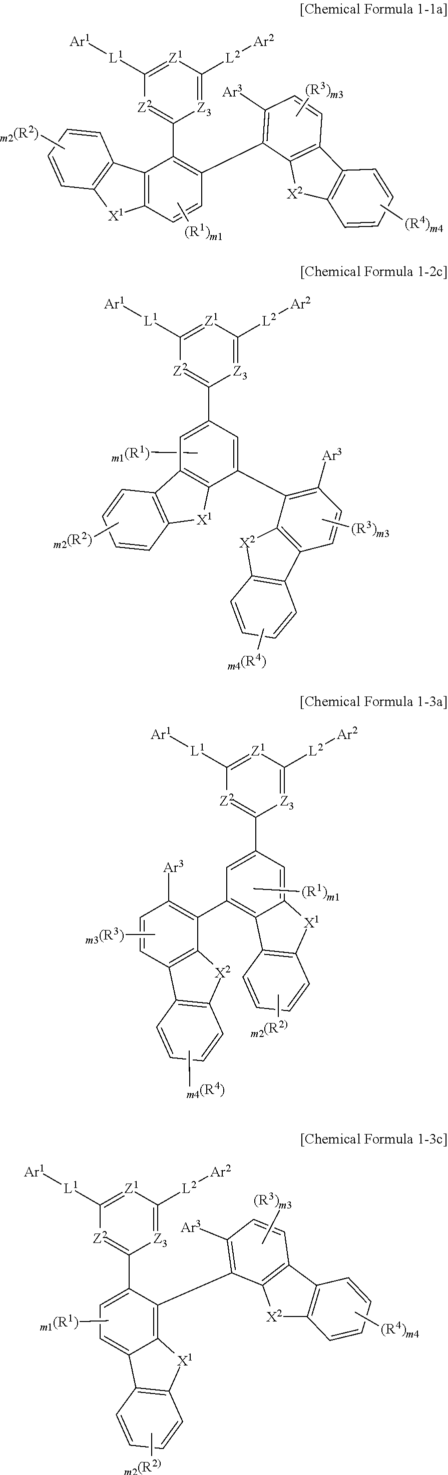
in Chemical Formula 1-1a, Chemical Formula 1-2c, Chemical Formula 1-3a, and Chemical Formula 1-3c, Z¹ to Z³, X¹ and X², Ar¹ to Ar³, L¹ and L², R¹ to R⁴, and m1 to m4 are defined the same as those of Chemical Formula 1.

4. The compound for an organic optoelectronic device as claimed in claim 1, wherein Ar¹ and Ar² are each independently a substituted or unsubstituted phenyl group, a substituted or unsubstituted biphenol group, or a substituted or unsubstituted carbazolyl group.

5. The compound for an organic optoelectronic device as claimed in claim 1, wherein Ar³ is a substituted or unsubstituted phenyl group, a substituted or unsubstituted biphenyl group, or a substituted or unsubstituted naphthyl group.

6. The compound for an organic optoelectronic device as claimed in claim 1, wherein R¹ to R⁴ are each independently hydrogen, deuterium, a cyano group, a substituted or unsubstituted C1 to C5 alkyl group, a substituted or unsubstituted C1 to C5 alkylsilyl group, a substituted or unsubstituted C3 to C10 cycloalkyl group, or a substituted or unsubstituted C6 to C12 aryl group.

7. The compound for an organic optoelectronic device as claimed in claim 1, wherein R¹ to R⁴ are each independently hydrogen, deuterium, a cyano group, a tert-butyl group, a trimethylsilyl group, a substituted or unsubstituted phenyl group, or a substituted or unsubstituted biphenyl group.

8. The compound for an organic optoelectronic device as claimed in claim 1, wherein:
the compound is a compound of Group 1: 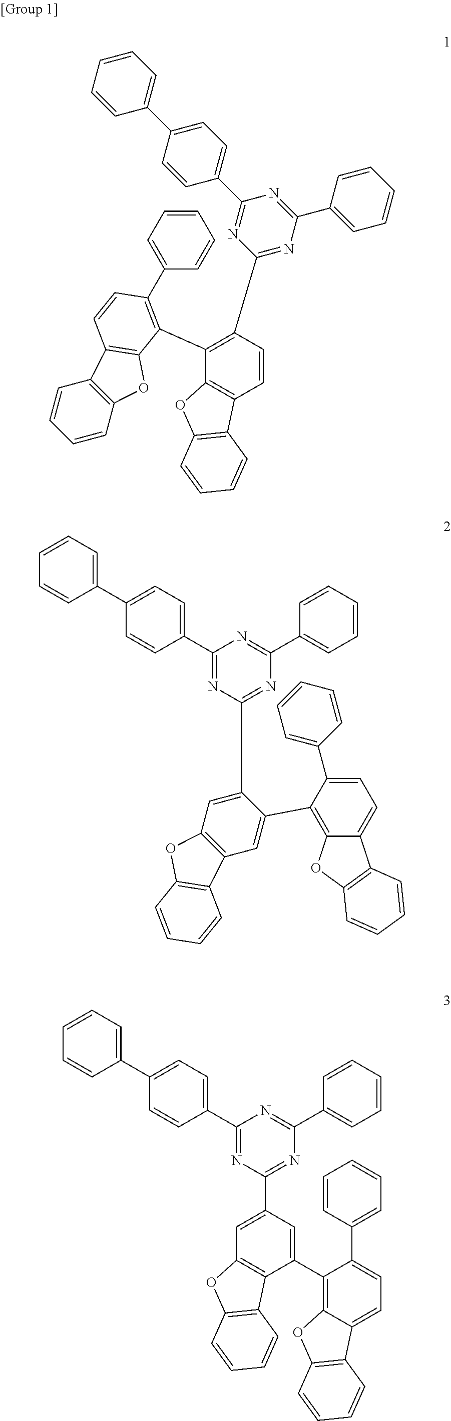 [SMILES: c1ccc(-c2ccc(-c3nc(-c4ccccc4)nc(-c4ccc5c(oc6ccccc65)c4-c4c(-c5ccccc5)ccc5c4oc4ccccc45)n3)cc2)cc1 ; c1ccc(-c2ccc(-c3nc(-c4ccccc4)nc(-c4cc5oc6ccccc6c5cc4-c4c(-c5ccccc5)ccc5c4oc4ccccc45)n3)cc2)cc1 ; c1ccc(-c2ccc(-c3nc(-c4ccccc4)nc(-c4cc(-c5c(-c6ccccc6)ccc6c5oc5ccccc56)c5c(c4)oc4ccccc45)n3)cc2)cc1] 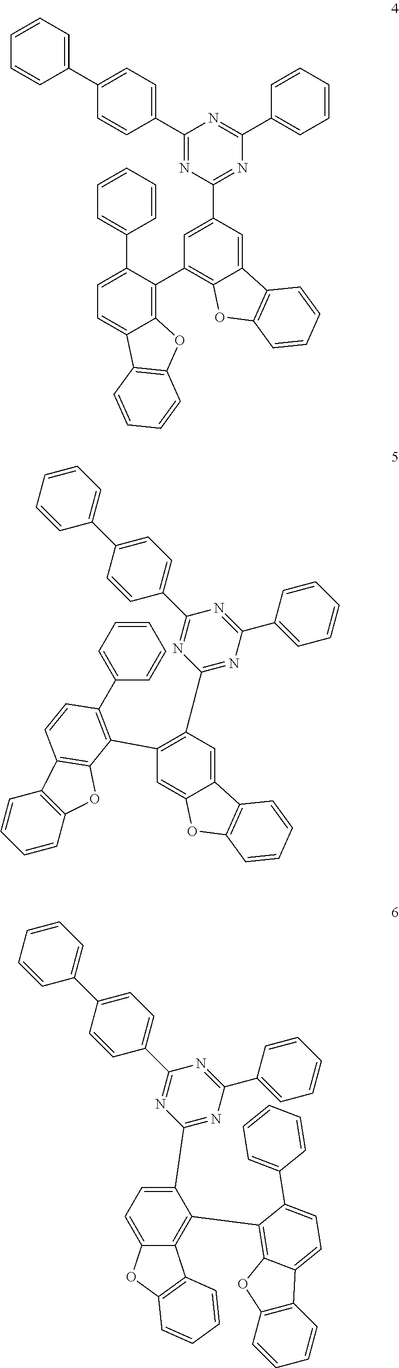 [SMILES: c1ccc(-c2ccc(-c3nc(-c4ccccc4)nc(-c4cc(-c5c(-c6ccccc6)ccc6c5oc5ccccc56)c5oc6ccccc6c5c4)n3)cc2)cc1 ; c1ccc(-c2ccc(-c3nc(-c4ccccc4)nc(-c4cc5c(cc4-c4c(-c6ccccc6)ccc6c4oc4ccccc46)oc4ccccc45)n3)cc2)cc1 ; c1ccc(-c2ccc(-c3nc(-c4ccccc4)nc(-c4ccc5oc6ccccc6c5c4-c4c(-c5ccccc5)ccc5c4oc4ccccc45)n3)cc2)cc1] 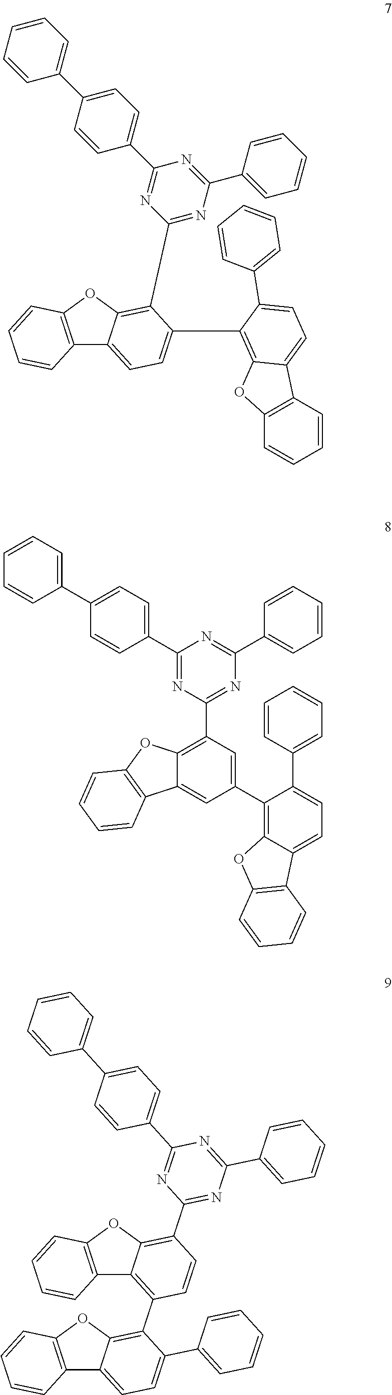 [SMILES: c1ccc(-c2ccc(-c3nc(-c4ccccc4)nc(-c4c(-c5c(-c6ccccc6)ccc6c5oc5ccccc56)ccc5c4oc4ccccc45)n3)cc2)cc1 ; c1ccc(-c2ccc(-c3nc(-c4ccccc4)nc(-c4cc(-c5c(-c6ccccc6)ccc6c5oc5ccccc56)cc5c4oc4ccccc45)n3)cc2)cc1 ; c1ccc(-c2ccc(-c3nc(-c4ccccc4)nc(-c4ccc(-c5c(-c6ccccc6)ccc6c5oc5ccccc56)c5c4oc4ccccc45)n3)cc2)cc1] 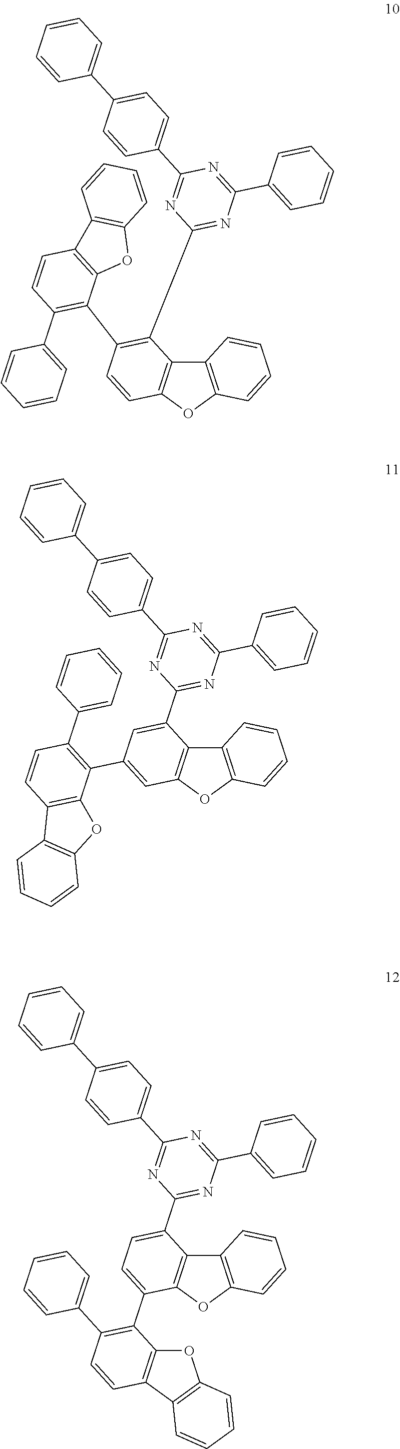 [SMILES: c1ccc(-c2ccc(-c3nc(-c4ccccc4)nc(-c4c(-c5c(-c6ccccc6)ccc6c5oc5ccccc56)ccc5oc6ccccc6c45)n3)cc2)cc1 ; c1ccc(-c2ccc(-c3nc(-c4ccccc4)nc(-c4cc(-c5c(-c6ccccc6)ccc6c5oc5ccccc56)cc5oc6ccccc6c45)n3)cc2)cc1 ; c1ccc(-c2ccc(-c3nc(-c4ccccc4)nc(-c4ccc(-c5c(-c6ccccc6)ccc6c5oc5ccccc56)c5oc6ccccc6c45)n3)cc2)cc1] 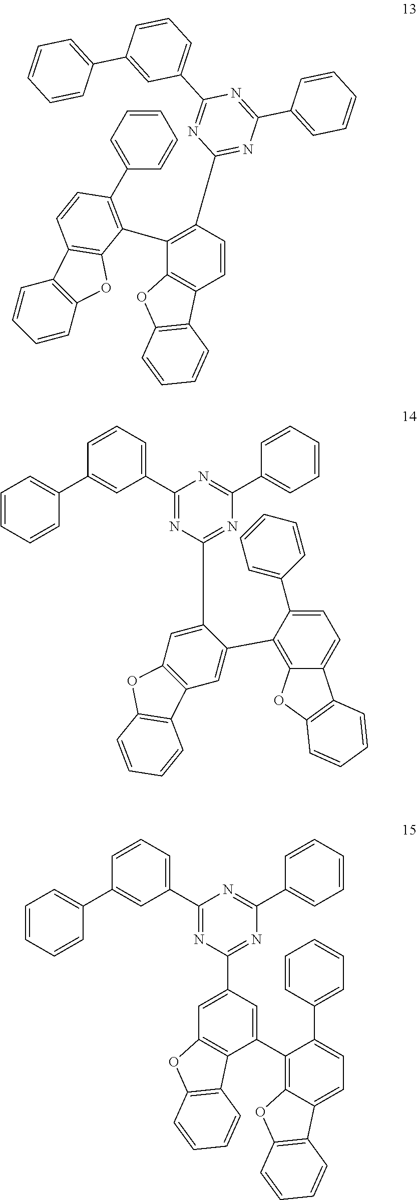 [SMILES: c1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; c1ccc(-c2cccc(-c3nc(-c4ccccc4)nc(-c4cc5oc6ccccc6c5cc4-c4c(-c5ccccc5)ccc5c4oc4ccccc45)n3)c2)cc1 ; c1ccc(-c2cccc(-c3nc(-c4ccccc4)nc(-c4cc(-c5c(-c6ccccc6)ccc6c5oc5ccccc56)c5c(c4)oc4ccccc45)n3)c2)cc1] 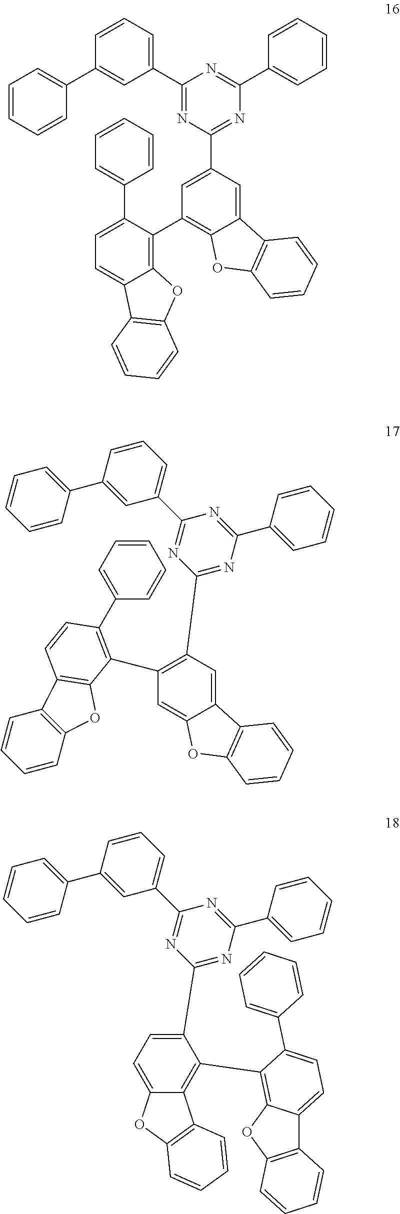 [SMILES: c1ccc(-c2cccc(-c3nc(-c4ccccc4)nc(-c4cc(-c5c(-c6ccccc6)ccc6c5oc5ccccc56)c5oc6ccccc6c5c4)n3)c2)cc1 ; c1ccc(-c2cccc(-c3nc(-c4ccccc4)nc(-c4cc5c(cc4-c4c(-c6ccccc6)ccc6c4oc4ccccc46)oc4ccccc45)n3)c2)cc1 ; c1ccc(-c2cccc(-c3nc(-c4ccccc4)nc(-c4ccc5oc6ccccc6c5c4-c4c(-c5ccccc5)ccc5c4oc4ccccc45)n3)c2)cc1] 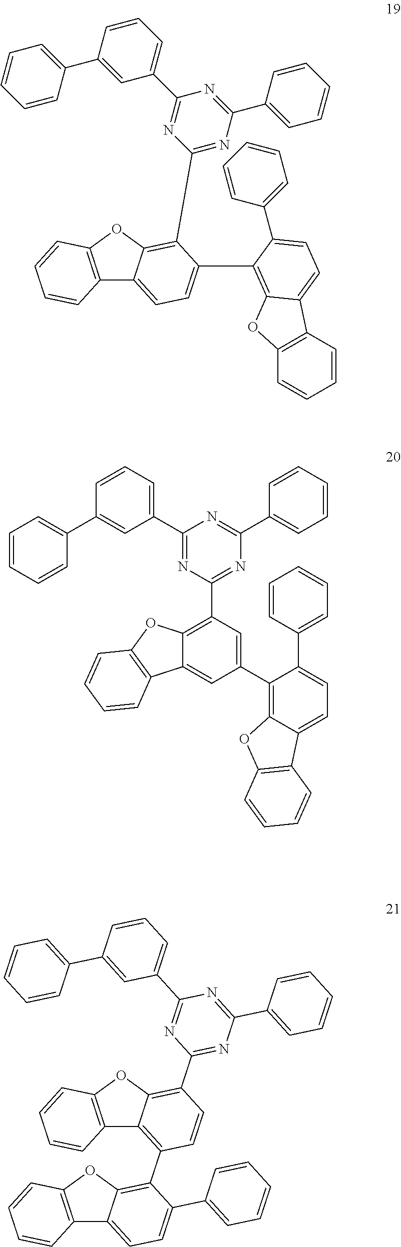 [SMILES: c1ccc(-c2cccc(-c3nc(-c4ccccc4)nc(-c4c(-c5c(-c6ccccc6)ccc6c5oc5ccccc56)ccc5c4oc4ccccc45)n3)c2)cc1 ; c1ccc(-c2cccc(-c3nc(-c4ccccc4)nc(-c4cc(-c5c(-c6ccccc6)ccc6c5oc5ccccc56)cc5c4oc4ccccc45)n3)c2)cc1 ; c1ccc(-c2cccc(-c3nc(-c4ccccc4)nc(-c4ccc(-c5c(-c6ccccc6)ccc6c5oc5ccccc56)c5c4oc4ccccc45)n3)c2)cc1] 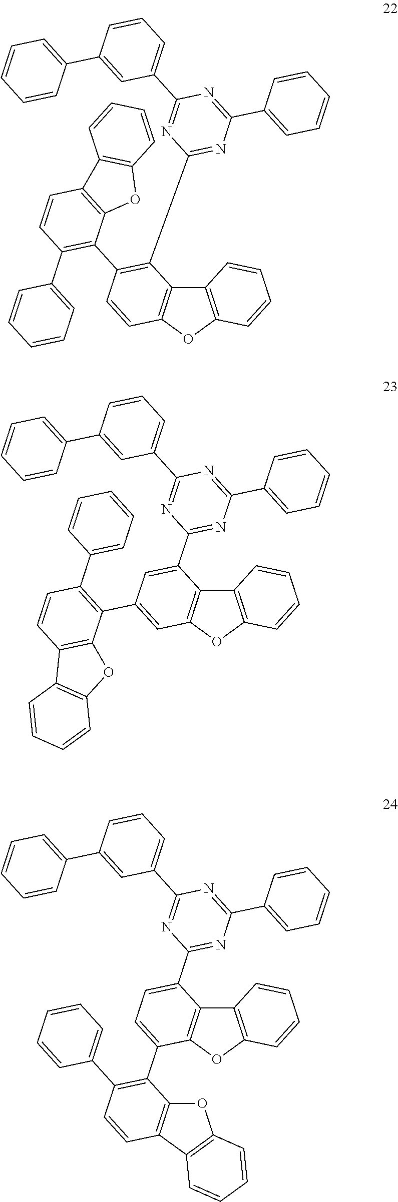 [SMILES: c1ccc(-c2cccc(-c3nc(-c4ccccc4)nc(-c4c(-c5c(-c6ccccc6)ccc6c5oc5ccccc56)ccc5oc6ccccc6c45)n3)c2)cc1 ; c1ccc(-c2cccc(-c3nc(-c4ccccc4)nc(-c4cc(-c5c(-c6ccccc6)ccc6c5oc5ccccc56)cc5oc6ccccc6c45)n3)c2)cc1 ; c1ccc(-c2cccc(-c3nc(-c4ccccc4)nc(-c4ccc(-c5c(-c6ccccc6)ccc6c5oc5ccccc56)c5oc6ccccc6c45)n3)c2)cc1] 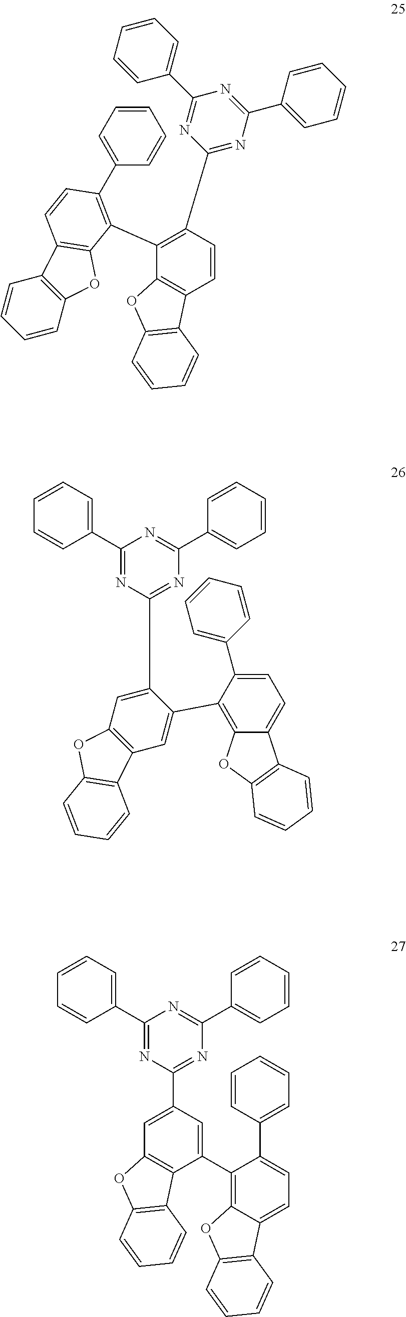 [SMILES: c1ccc(-c2nc(-c3ccccc3)nc(-c3ccc4c(oc5ccccc54)c3-c3c(-c4ccccc4)ccc4c3oc3ccccc34)n2)cc1 ; c1ccc(-c2nc(-c3ccccc3)nc(-c3cc4oc5ccccc5c4cc3-c3c(-c4ccccc4)ccc4c3oc3ccccc34)n2)cc1 ; c1ccc(-c2nc(-c3ccccc3)nc(-c3cc(-c4c(-c5ccccc5)ccc5c4oc4ccccc45)c4c(c3)oc3ccccc34)n2)cc1] 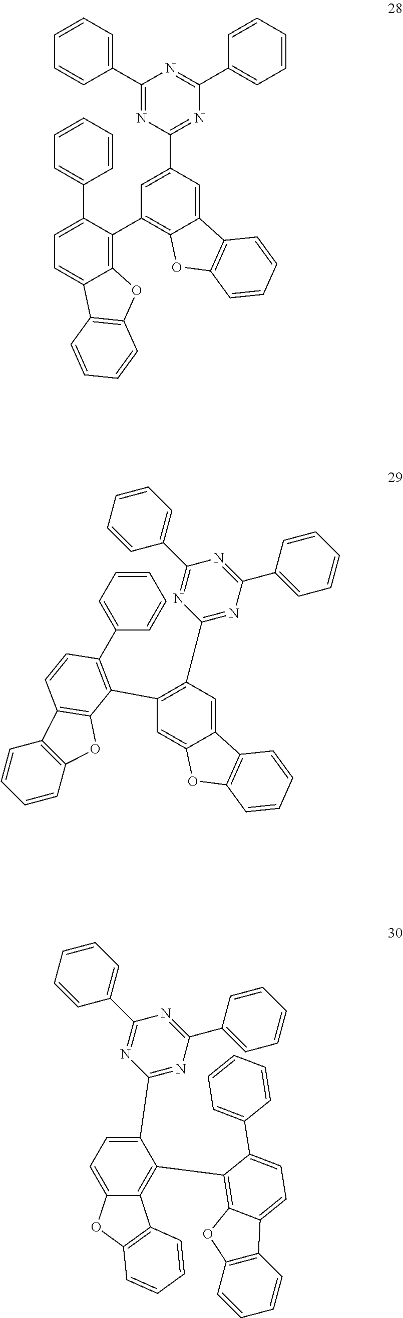 [SMILES: c1ccc(-c2nc(-c3ccccc3)nc(-c3cc(-c4c(-c5ccccc5)ccc5c4oc4ccccc45)c4oc5ccccc5c4c3)n2)cc1 ; c1ccc(-c2nc(-c3ccccc3)nc(-c3cc4c(cc3-c3c(-c5ccccc5)ccc5c3oc3ccccc35)oc3ccccc34)n2)cc1 ; c1ccc(-c2nc(-c3ccccc3)nc(-c3ccc4oc5ccccc5c4c3-c3c(-c4ccccc4)ccc4c3oc3ccccc34)n2)cc1] 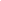 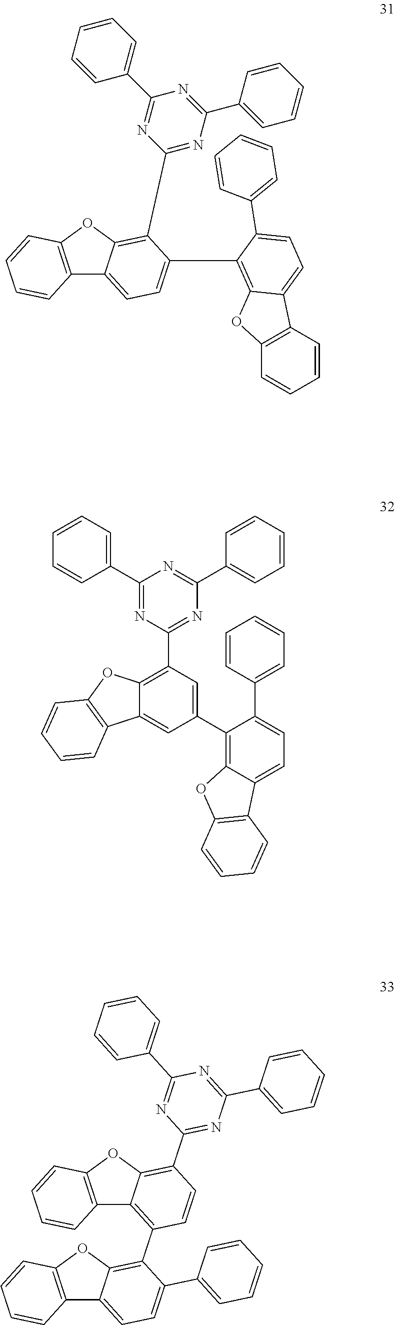 [SMILES: c1ccc(-c2nc(-c3ccccc3)nc(-c3c(-c4c(-c5ccccc5)ccc5c4oc4ccccc45)ccc4c3oc3ccccc34)n2)cc1 ; c1ccc(-c2nc(-c3ccccc3)nc(-c3cc(-c4c(-c5ccccc5)ccc5c4oc4ccccc45)cc4c3oc3ccccc34)n2)cc1 ; c1ccc(-c2nc(-c3ccccc3)nc(-c3ccc(-c4c(-c5ccccc5)ccc5c4oc4ccccc45)c4c3oc3ccccc34)n2)cc1] 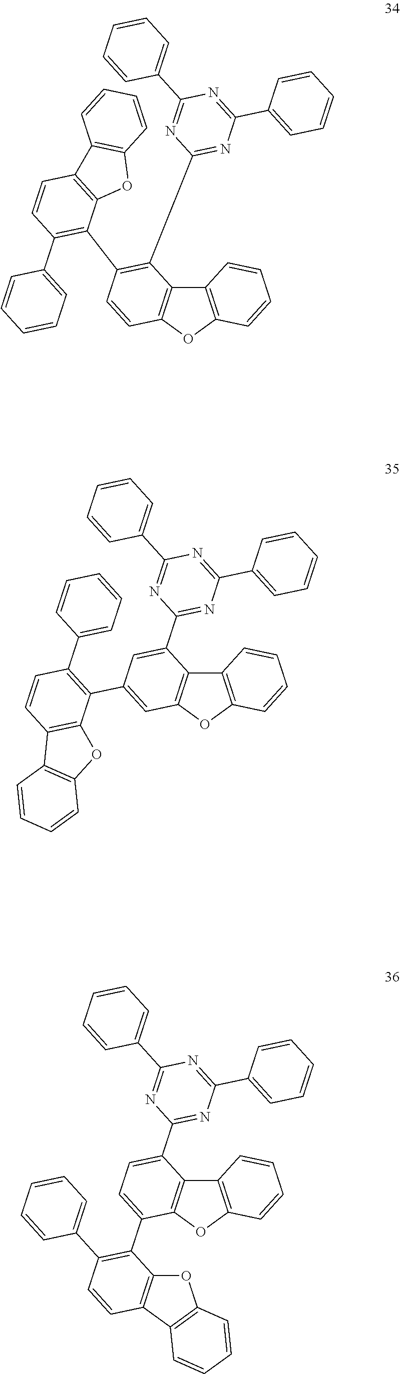 [SMILES: c1ccc(-c2nc(-c3ccccc3)nc(-c3c(-c4c(-c5ccccc5)ccc5c4oc4ccccc45)ccc4oc5ccccc5c34)n2)cc1 ; c1ccc(-c2nc(-c3ccccc3)nc(-c3cc(-c4c(-c5ccccc5)ccc5c4oc4ccccc45)cc4oc5ccccc5c34)n2)cc1 ; c1ccc(-c2nc(-c3ccccc3)nc(-c3ccc(-c4c(-c5ccccc5)ccc5c4oc4ccccc45)c4oc5ccccc5c34)n2)cc1] 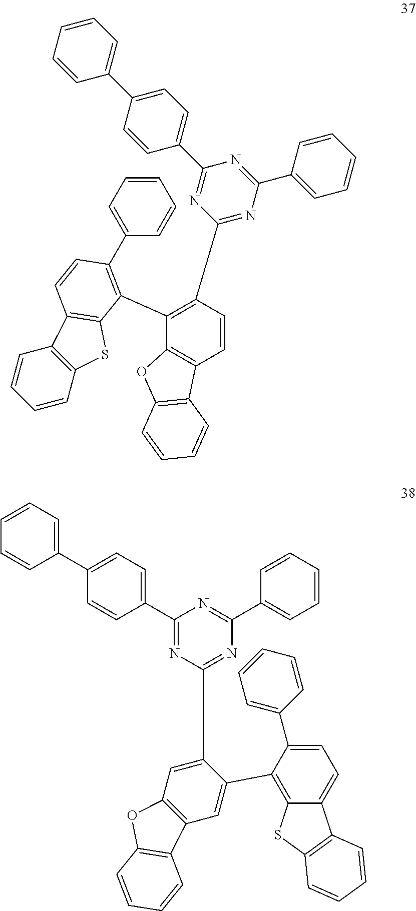 [SMILES: c1ccc(-c2ccc(-c3nc(-c4ccccc4)nc(-c4ccc5c(oc6ccccc65)c4-c4c(-c5ccccc5)ccc5c4sc4ccccc45)n3)cc2)cc1 ; c1ccc(-c2ccc(-c3nc(-c4ccccc4)nc(-c4cc5oc6ccccc6c5cc4-c4c(-c5ccccc5)ccc5c4sc4ccccc45)n3)cc2)cc1] 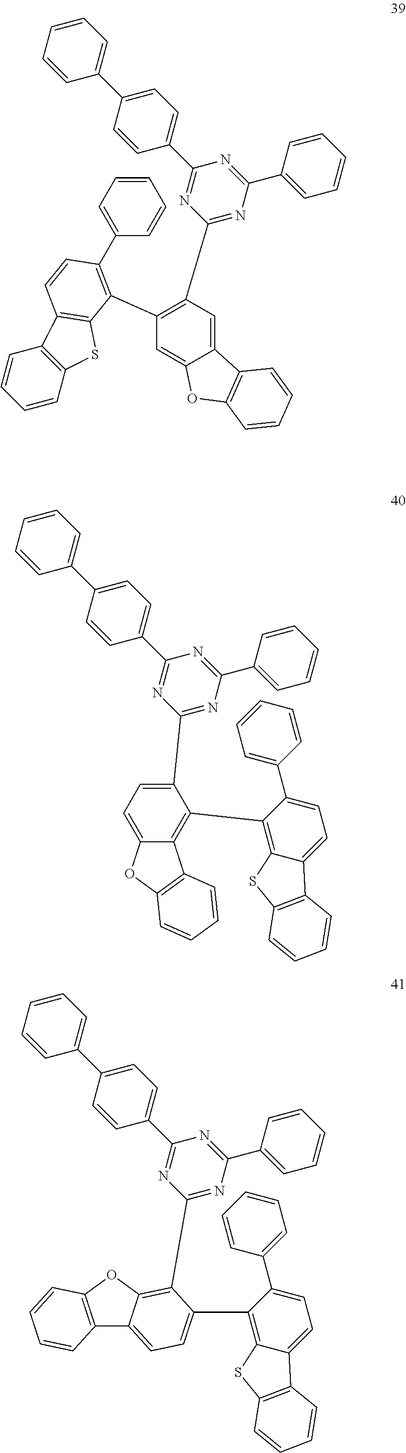 [SMILES: c1ccc(-c2ccc(-c3nc(-c4ccccc4)nc(-c4cc5c(cc4-c4c(-c6ccccc6)ccc6c4sc4ccccc46)oc4ccccc45)n3)cc2)cc1 ; c1ccc(-c2ccc(-c3nc(-c4ccccc4)nc(-c4ccc5oc6ccccc6c5c4-c4c(-c5ccccc5)ccc5c4sc4ccccc45)n3)cc2)cc1 ; c1ccc(-c2ccc(-c3nc(-c4ccccc4)nc(-c4c(-c5c(-c6ccccc6)ccc6c5sc5ccccc56)ccc5c4oc4ccccc45)n3)cc2)cc1] 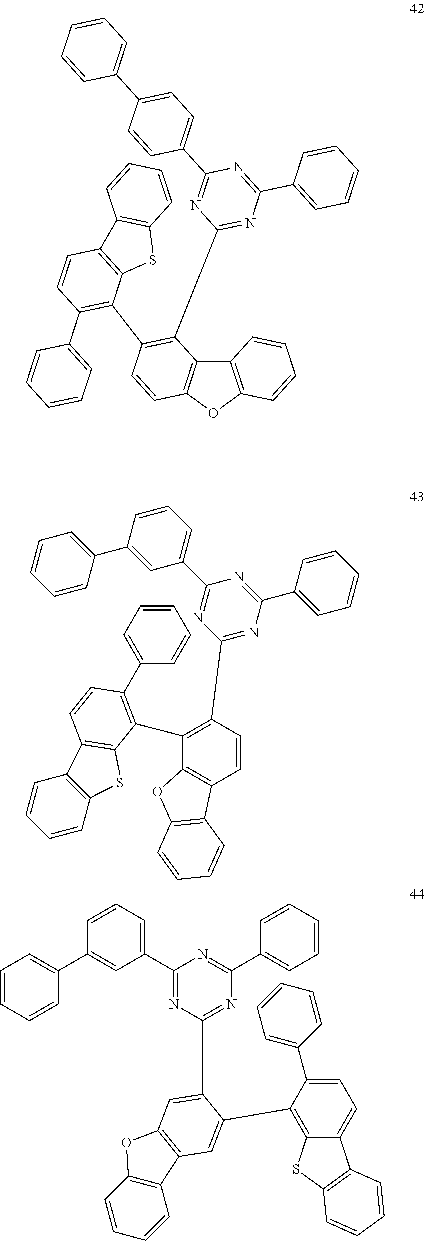 [SMILES: c1ccc(-c2ccc(-c3nc(-c4ccccc4)nc(-c4c(-c5c(-c6ccccc6)ccc6c5sc5ccccc56)ccc5oc6ccccc6c45)n3)cc2)cc1 ; c1ccc(-c2cccc(-c3nc(-c4ccccc4)nc(-c4ccc5c(oc6ccccc65)c4-c4c(-c5ccccc5)ccc5c4sc4ccccc45)n3)c2)cc1 ; c1ccc(-c2cccc(-c3nc(-c4ccccc4)nc(-c4cc5oc6ccccc6c5cc4-c4c(-c5ccccc5)ccc5c4sc4ccccc45)n3)c2)cc1] 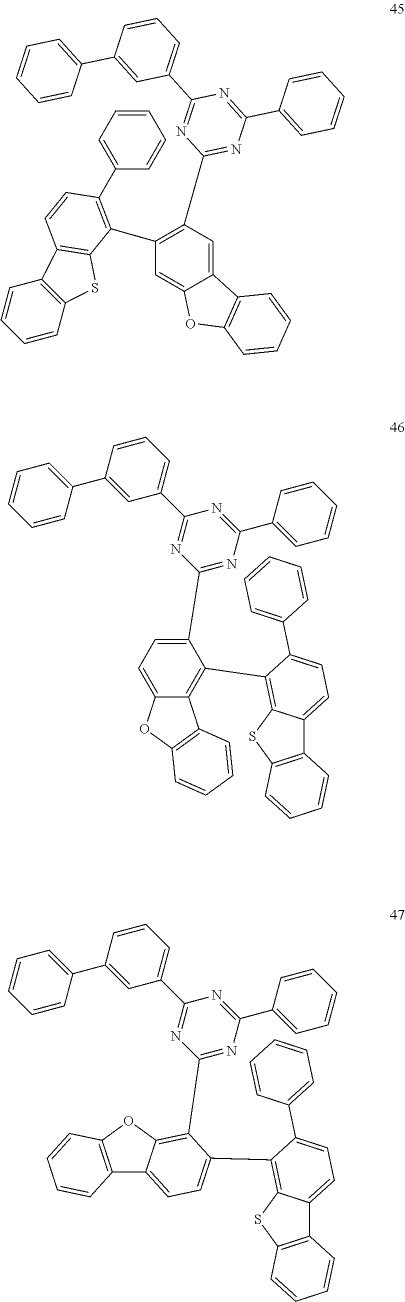 [SMILES: c1ccc(-c2cccc(-c3nc(-c4ccccc4)nc(-c4cc5c(cc4-c4c(-c6ccccc6)ccc6c4sc4ccccc46)oc4ccccc45)n3)c2)cc1 ; c1ccc(-c2cccc(-c3nc(-c4ccccc4)nc(-c4ccc5oc6ccccc6c5c4-c4c(-c5ccccc5)ccc5c4sc4ccccc45)n3)c2)cc1 ; c1ccc(-c2cccc(-c3nc(-c4ccccc4)nc(-c4c(-c5c(-c6ccccc6)ccc6c5sc5ccccc56)ccc5c4oc4ccccc45)n3)c2)cc1] 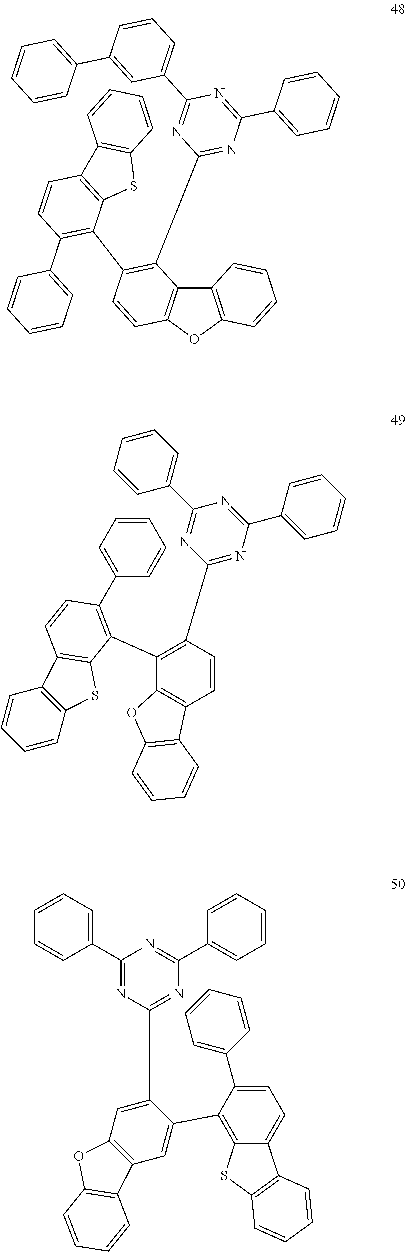 [SMILES: c1ccc(-c2cccc(-c3nc(-c4ccccc4)nc(-c4c(-c5c(-c6ccccc6)ccc6c5sc5ccccc56)ccc5oc6ccccc6c45)n3)c2)cc1 ; c1ccc(-c2nc(-c3ccccc3)nc(-c3ccc4c(oc5ccccc54)c3-c3c(-c4ccccc4)ccc4c3sc3ccccc34)n2)cc1 ; c1ccc(-c2nc(-c3ccccc3)nc(-c3cc4oc5ccccc5c4cc3-c3c(-c4ccccc4)ccc4c3sc3ccccc34)n2)cc1] 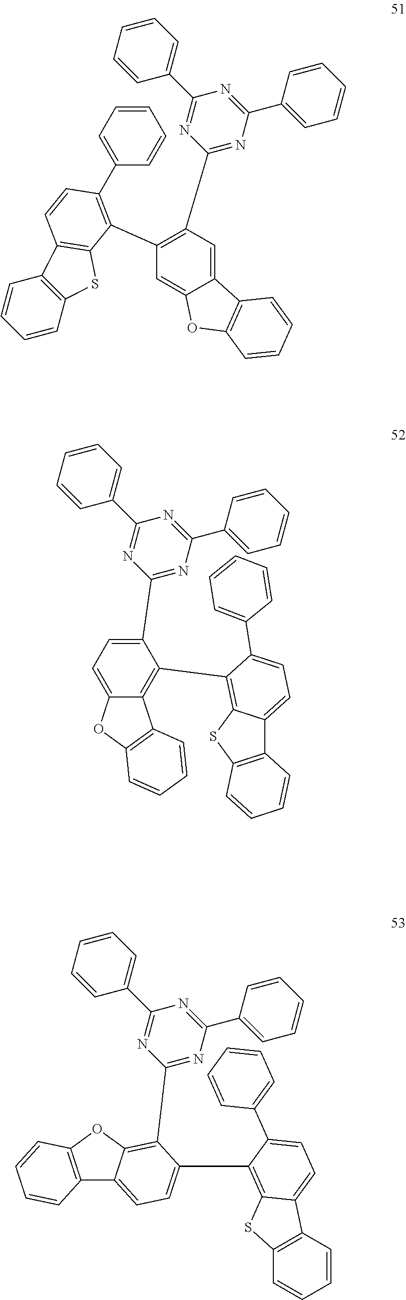 [SMILES: c1ccc(-c2nc(-c3ccccc3)nc(-c3cc4c(cc3-c3c(-c5ccccc5)ccc5c3sc3ccccc35)oc3ccccc34)n2)cc1 ; c1ccc(-c2nc(-c3ccccc3)nc(-c3ccc4oc5ccccc5c4c3-c3c(-c4ccccc4)ccc4c3sc3ccccc34)n2)cc1 ; c1ccc(-c2nc(-c3ccccc3)nc(-c3c(-c4c(-c5ccccc5)ccc5c4sc4ccccc45)ccc4c3oc3ccccc34)n2)cc1] 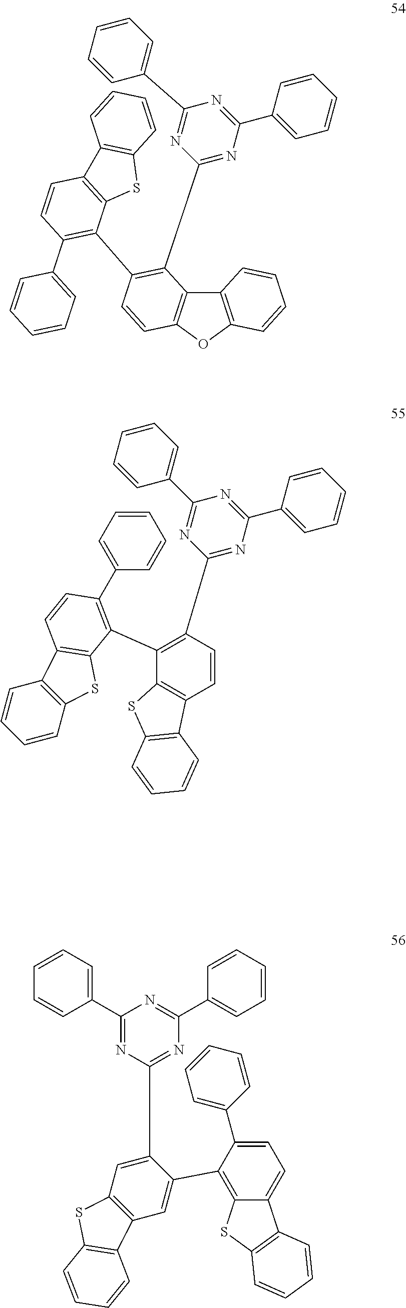 [SMILES: c1ccc(-c2nc(-c3ccccc3)nc(-c3c(-c4c(-c5ccccc5)ccc5c4sc4ccccc45)ccc4oc5ccccc5c34)n2)cc1 ; c1ccc(-c2nc(-c3ccccc3)nc(-c3ccc4c(sc5ccccc54)c3-c3c(-c4ccccc4)ccc4c3sc3ccccc34)n2)cc1 ; c1ccc(-c2nc(-c3ccccc3)nc(-c3cc4sc5ccccc5c4cc3-c3c(-c4ccccc4)ccc4c3sc3ccccc34)n2)cc1] 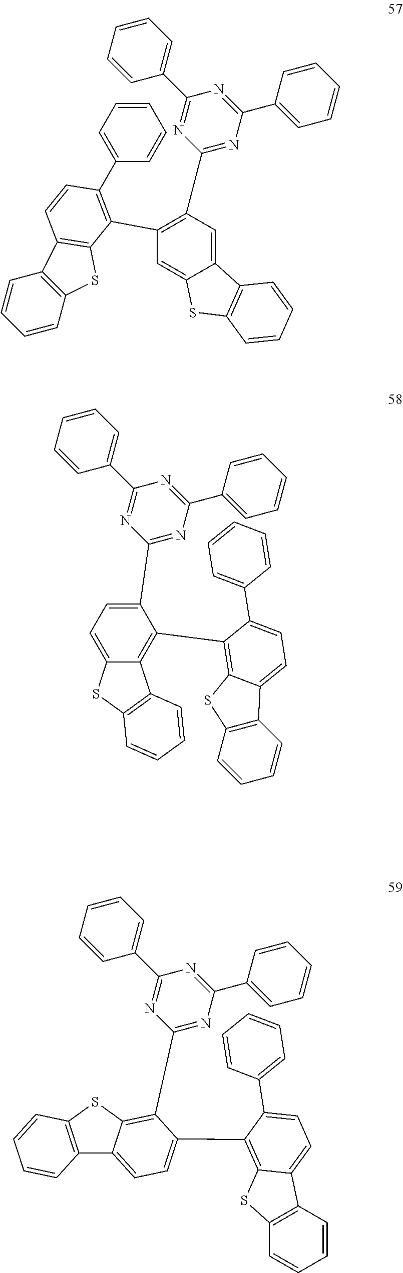 [SMILES: c1ccc(-c2nc(-c3ccccc3)nc(-c3cc4c(cc3-c3c(-c5ccccc5)ccc5c3sc3ccccc35)sc3ccccc34)n2)cc1 ; c1ccc(-c2nc(-c3ccccc3)nc(-c3ccc4sc5ccccc5c4c3-c3c(-c4ccccc4)ccc4c3sc3ccccc34)n2)cc1 ; c1ccc(-c2nc(-c3ccccc3)nc(-c3c(-c4c(-c5ccccc5)ccc5c4sc4ccccc45)ccc4c3sc3ccccc34)n2)cc1] 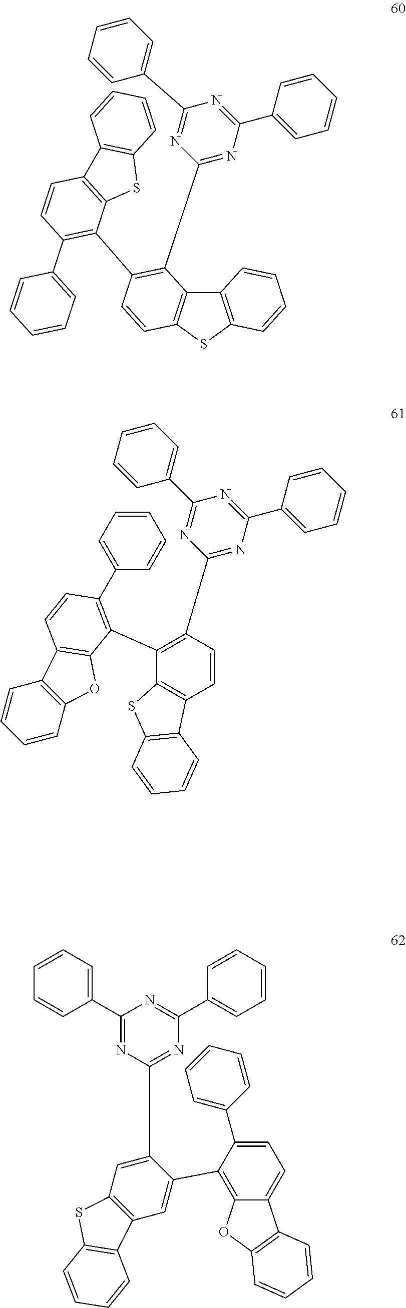 [SMILES: c1ccc(-c2nc(-c3ccccc3)nc(-c3c(-c4c(-c5ccccc5)ccc5c4sc4ccccc45)ccc4sc5ccccc5c34)n2)cc1 ; c1ccc(-c2nc(-c3ccccc3)nc(-c3ccc4c(sc5ccccc54)c3-c3c(-c4ccccc4)ccc4c3oc3ccccc34)n2)cc1 ; c1ccc(-c2nc(-c3ccccc3)nc(-c3cc4sc5ccccc5c4cc3-c3c(-c4ccccc4)ccc4c3oc3ccccc34)n2)cc1] 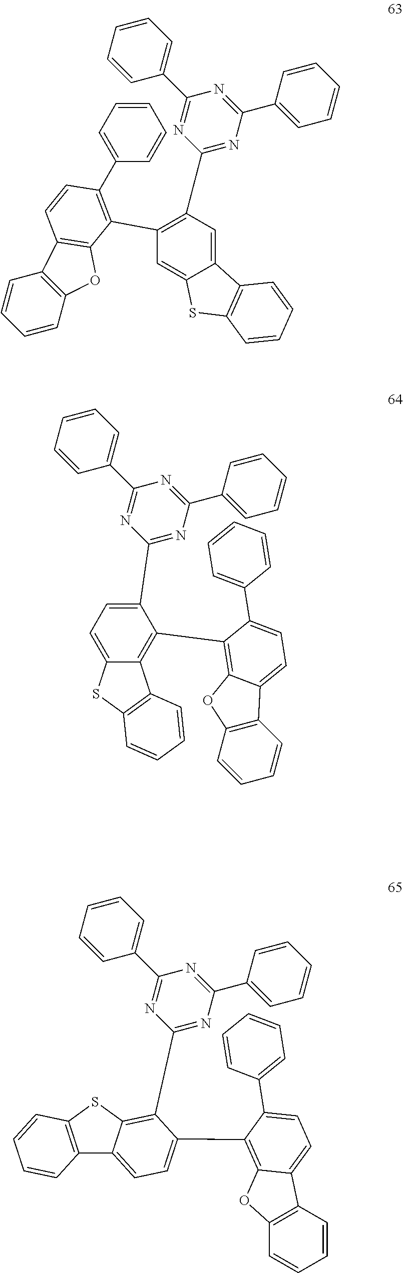 [SMILES: c1ccc(-c2nc(-c3ccccc3)nc(-c3cc4c(cc3-c3c(-c5ccccc5)ccc5c3oc3ccccc35)sc3ccccc34)n2)cc1 ; c1ccc(-c2nc(-c3ccccc3)nc(-c3ccc4sc5ccccc5c4c3-c3c(-c4ccccc4)ccc4c3oc3ccccc34)n2)cc1 ; c1ccc(-c2nc(-c3ccccc3)nc(-c3c(-c4c(-c5ccccc5)ccc5c4oc4ccccc45)ccc4c3sc3ccccc34)n2)cc1] 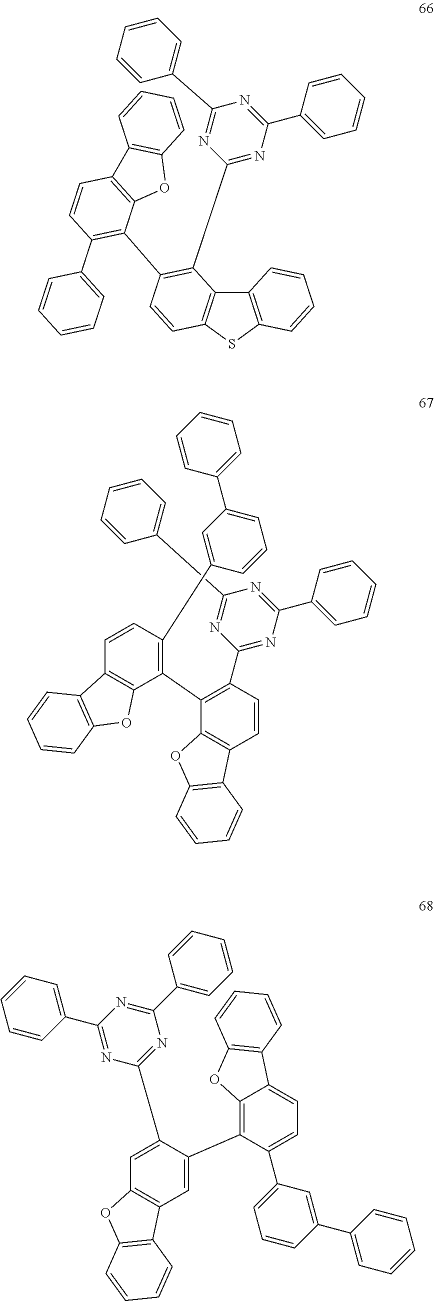 [SMILES: c1ccc(-c2nc(-c3ccccc3)nc(-c3c(-c4c(-c5ccccc5)ccc5c4oc4ccccc45)ccc4sc5ccccc5c34)n2)cc1 ; c1ccc(-c2cccc(-c3ccc4c(oc5ccccc54)c3-c3c(-c4nc(-c5ccccc5)nc(-c5ccccc5)n4)ccc4c3oc3ccccc34)c2)cc1 ; c1ccc(-c2cccc(-c3ccc4c(oc5ccccc54)c3-c3cc4c(cc3-c3nc(-c5ccccc5)nc(-c5ccccc5)n3)oc3ccccc34)c2)cc1] 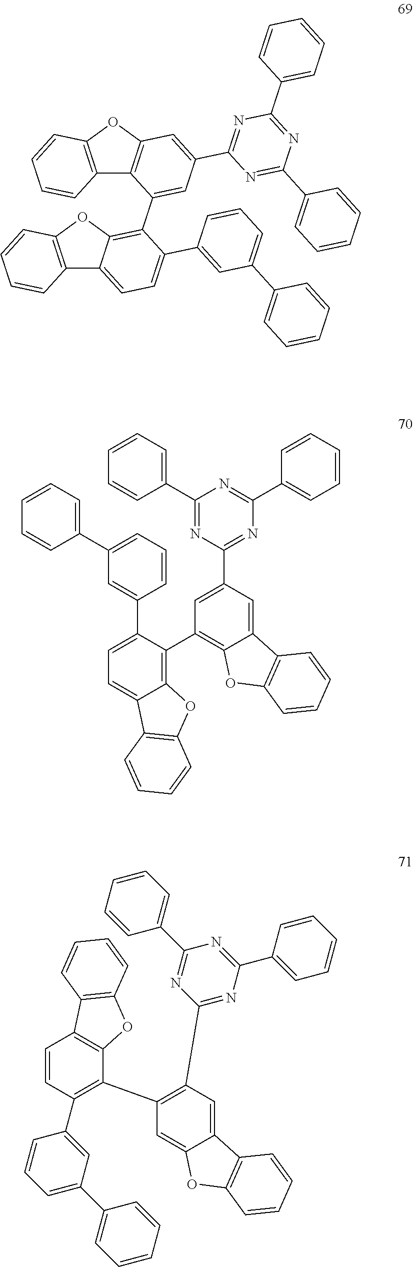 [SMILES: c1ccc(-c2cccc(-c3ccc4c(oc5ccccc54)c3-c3cc(-c4nc(-c5ccccc5)nc(-c5ccccc5)n4)cc4oc5ccccc5c34)c2)cc1 ; c1ccc(-c2cccc(-c3ccc4c(oc5ccccc54)c3-c3cc(-c4nc(-c5ccccc5)nc(-c5ccccc5)n4)cc4c3oc3ccccc34)c2)cc1 ; c1ccc(-c2cccc(-c3ccc4c(oc5ccccc54)c3-c3cc4oc5ccccc5c4cc3-c3nc(-c4ccccc4)nc(-c4ccccc4)n3)c2)cc1] 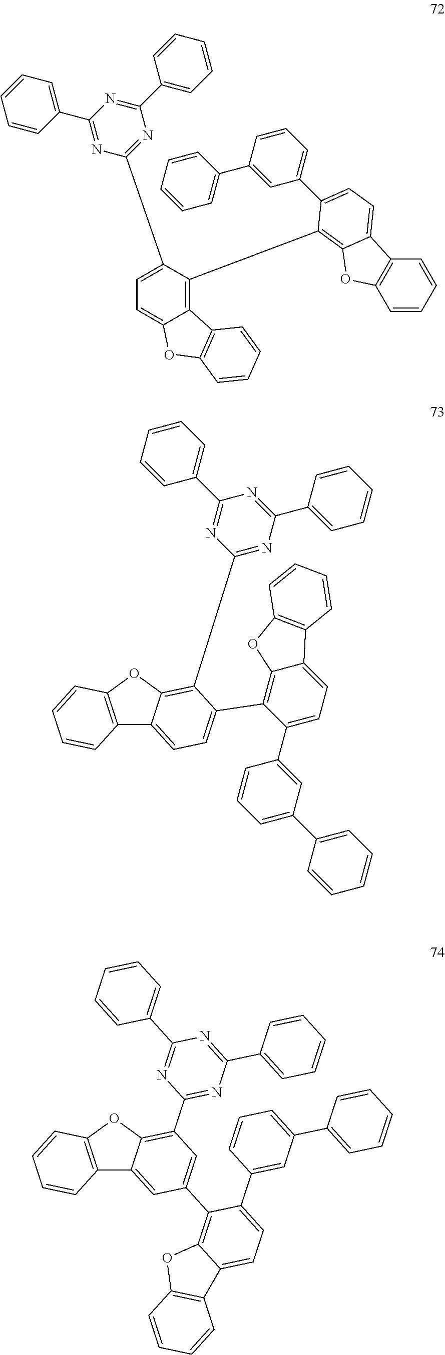 [SMILES: c1ccc(-c2cccc(-c3ccc4c(oc5ccccc54)c3-c3c(-c4nc(-c5ccccc5)nc(-c5ccccc5)n4)ccc4oc5ccccc5c34)c2)cc1 ; c1ccc(-c2cccc(-c3ccc4c(oc5ccccc54)c3-c3ccc4c(oc5ccccc54)c3-c3nc(-c4ccccc4)nc(-c4ccccc4)n3)c2)cc1 ; c1ccc(-c2cccc(-c3ccc4c(oc5ccccc54)c3-c3cc(-c4nc(-c5ccccc5)nc(-c5ccccc5)n4)c4oc5ccccc5c4c3)c2)cc1] 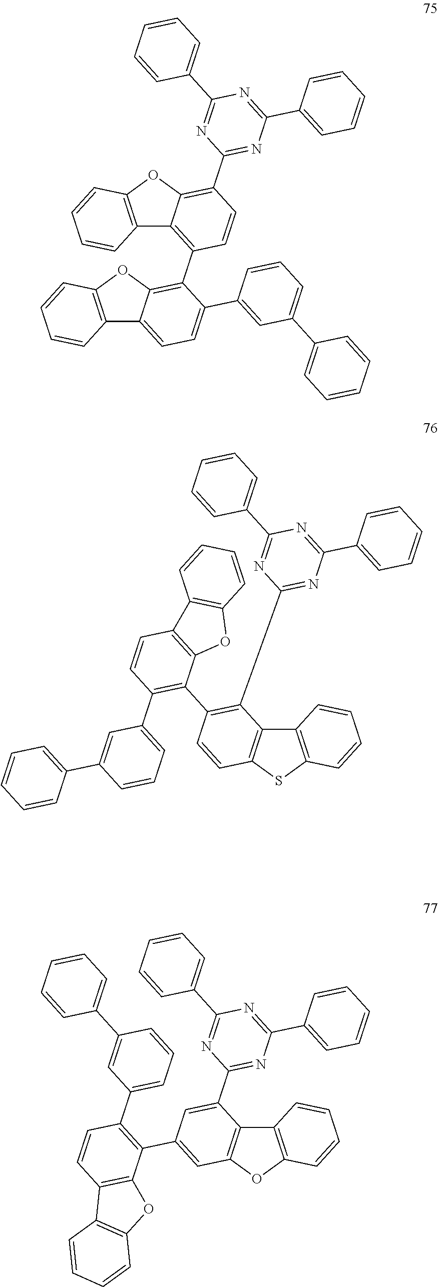 [SMILES: c1ccc(-c2cccc(-c3ccc4c(oc5ccccc54)c3-c3ccc(-c4nc(-c5ccccc5)nc(-c5ccccc5)n4)c4oc5ccccc5c34)c2)cc1 ; c1ccc(-c2cccc(-c3ccc4c(oc5ccccc54)c3-c3ccc4sc5ccccc5c4c3-c3nc(-c4ccccc4)nc(-c4ccccc4)n3)c2)cc1 ; c1ccc(-c2cccc(-c3ccc4c(oc5ccccc54)c3-c3cc(-c4nc(-c5ccccc5)nc(-c5ccccc5)n4)c4c(c3)oc3ccccc34)c2)cc1] 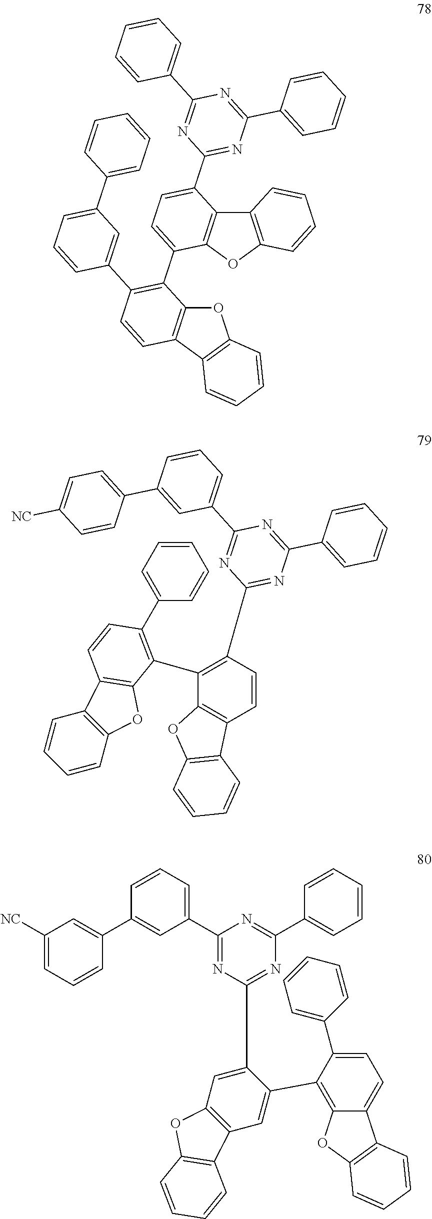 [SMILES: c1ccc(-c2cccc(-c3ccc4c(oc5ccccc54)c3-c3ccc(-c4nc(-c5ccccc5)nc(-c5ccccc5)n4)c4c3oc3ccccc34)c2)cc1 ; N#Cc1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; N#Cc1cccc(-c2cccc(-c3nc(-c4ccccc4)nc(-c4cc5oc6ccccc6c5cc4-c4c(-c5ccccc5)ccc5c4oc4ccccc45)n3)c2)c1] 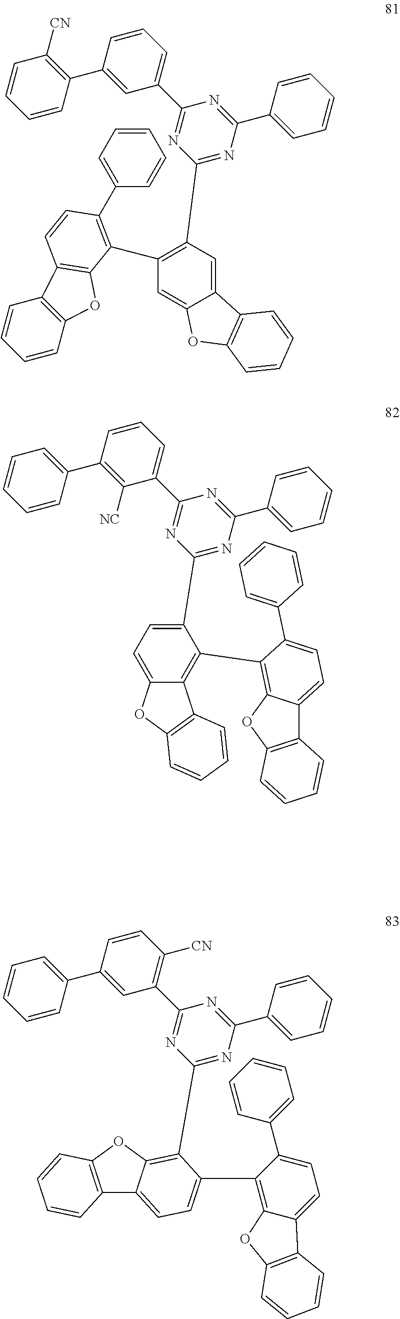 [SMILES: N#Cc1ccccc1-c1cccc(-c2nc(-c3ccccc3)nc(-c3cc4c(cc3-c3c(-c5ccccc5)ccc5c3oc3ccccc35)oc3ccccc34)n2)c1 ; N#Cc1c(-c2ccccc2)cccc1-c1nc(-c2ccccc2)nc(-c2ccc3oc4ccccc4c3c2-c2c(-c3ccccc3)ccc3c2oc2ccccc23)n1 ; N#Cc1ccc(-c2ccccc2)cc1-c1nc(-c2ccccc2)nc(-c2c(-c3c(-c4ccccc4)ccc4c3oc3ccccc34)ccc3c2oc2ccccc23)n1] 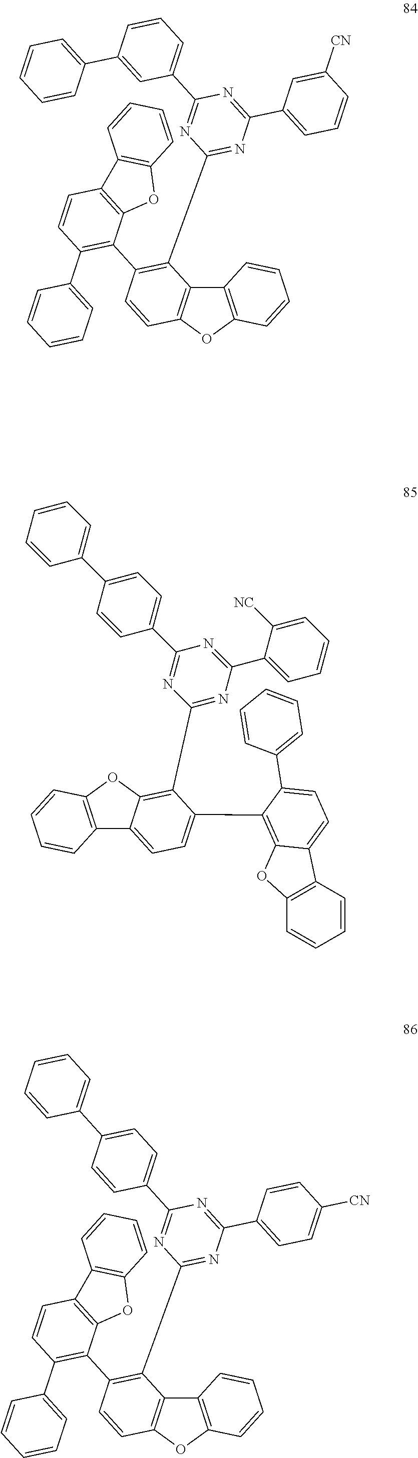 [SMILES: N#Cc1cccc(-c2nc(-c3cccc(-c4ccccc4)c3)nc(-c3c(-c4c(-c5ccccc5)ccc5c4oc4ccccc45)ccc4oc5ccccc5c34)n2)c1 ; N#Cc1ccccc1-c1nc(-c2ccc(-c3ccccc3)cc2)nc(-c2c(-c3c(-c4ccccc4)ccc4c3oc3ccccc34)ccc3c2oc2ccccc23)n1 ; N#Cc1ccc(-c2nc(-c3ccc(-c4ccccc4)cc3)nc(-c3c(-c4c(-c5ccccc5)ccc5c4oc4ccccc45)ccc4oc5ccccc5c34)n2)cc1] 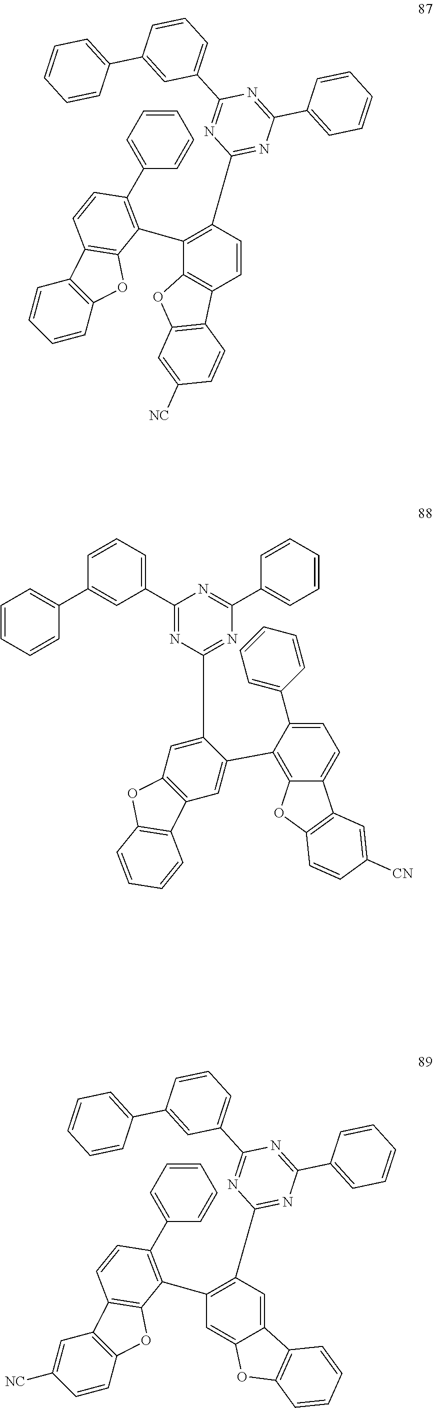 [SMILES: N#Cc1ccc2c(c1)oc1c(-c3c(-c4ccccc4)ccc4c3oc3ccccc34)c(-c3nc(-c4ccccc4)nc(-c4cccc(-c5ccccc5)c4)n3)ccc12 ; N#Cc1ccc2oc3c(-c4cc5c(cc4-c4nc(-c6ccccc6)nc(-c6cccc(-c7ccccc7)c6)n4)oc4ccccc45)c(-c4ccccc4)ccc3c2c1 ; N#Cc1ccc2oc3c(-c4cc5oc6ccccc6c5cc4-c4nc(-c5ccccc5)nc(-c5cccc(-c6ccccc6)c5)n4)c(-c4ccccc4)ccc3c2c1] 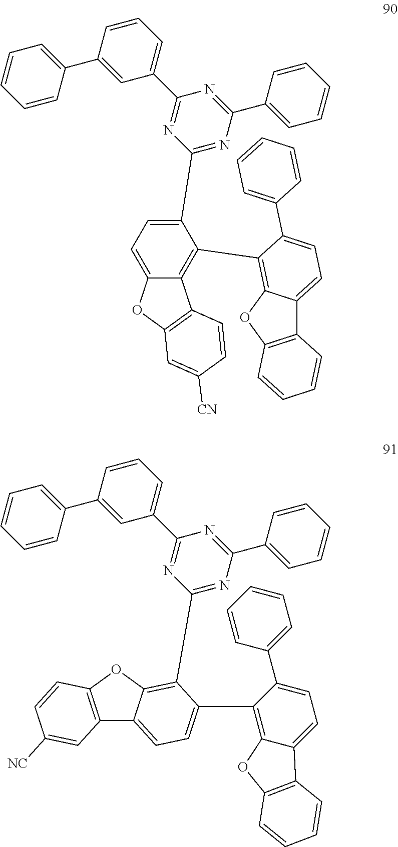 [SMILES: N#Cc1ccc2c(c1)oc1ccc(-c3nc(-c4ccccc4)nc(-c4cccc(-c5ccccc5)c4)n3)c(-c3c(-c4ccccc4)ccc4c3oc3ccccc34)c12 ; N#Cc1ccc2oc3c(-c4nc(-c5ccccc5)nc(-c5cccc(-c6ccccc6)c5)n4)c(-c4c(-c5ccccc5)ccc5c4oc4ccccc45)ccc3c2c1] 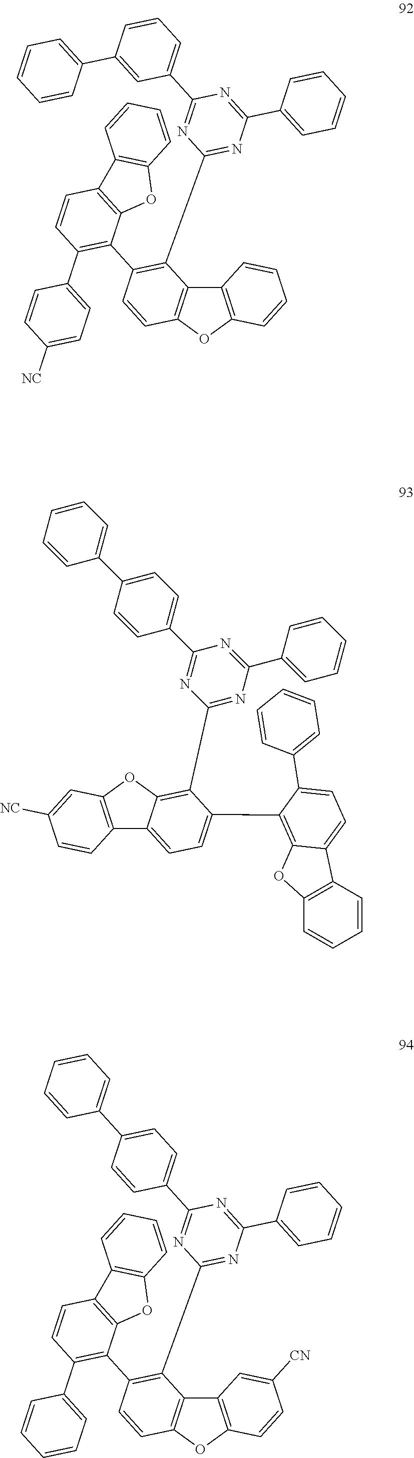 [SMILES: N#Cc1ccc(-c2ccc3c(oc4ccccc43)c2-c2ccc3oc4ccccc4c3c2-c2nc(-c3ccccc3)nc(-c3cccc(-c4ccccc4)c3)n2)cc1 ; N#Cc1ccc2c(c1)oc1c(-c3nc(-c4ccccc4)nc(-c4ccc(-c5ccccc5)cc4)n3)c(-c3c(-c4ccccc4)ccc4c3oc3ccccc34)ccc12 ; N#Cc1ccc2oc3ccc(-c4c(-c5ccccc5)ccc5c4oc4ccccc45)c(-c4nc(-c5ccccc5)nc(-c5ccc(-c6ccccc6)cc5)n4)c3c2c1] 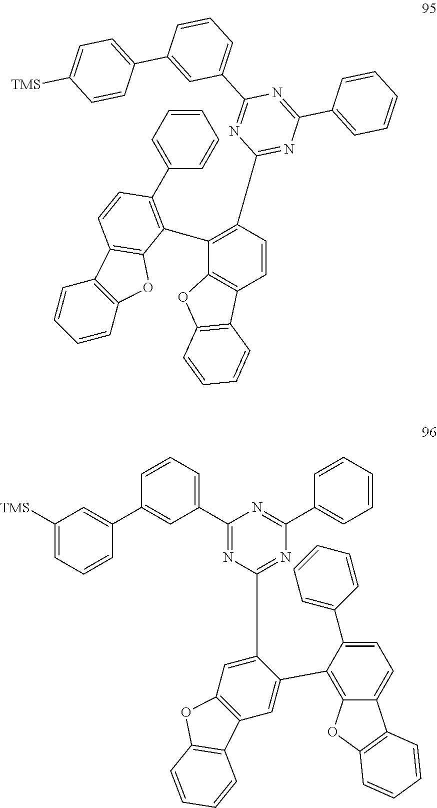 [SMILES: C[Si](C)(C)c1ccc(-c2cccc(-c3nc(-c4ccccc4)nc(-c4ccc5c(oc6ccccc65)c4-c4c(-c5ccccc5)ccc5c4oc4ccccc45)n3)c2)cc1 ; C[Si](C)(C)c1cccc(-c2cccc(-c3nc(-c4ccccc4)nc(-c4cc5oc6ccccc6c5cc4-c4c(-c5ccccc5)ccc5c4oc4ccccc45)n3)c2)c1] 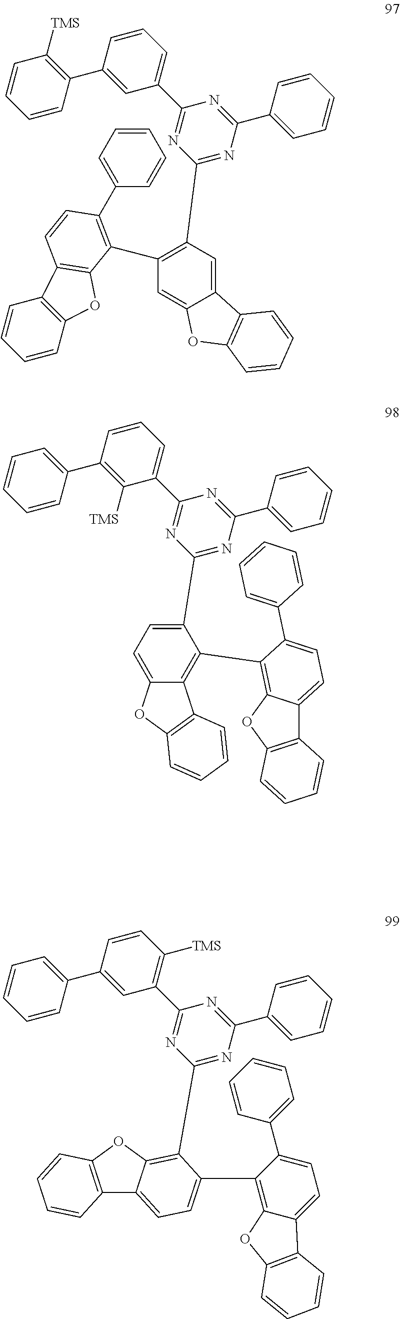 [SMILES: C[Si](C)(C)c1ccccc1-c1cccc(-c2nc(-c3ccccc3)nc(-c3cc4c(cc3-c3c(-c5ccccc5)ccc5c3oc3ccccc35)oc3ccccc34)n2)c1 ; C[Si](C)(C)c1c(-c2ccccc2)cccc1-c1nc(-c2ccccc2)nc(-c2ccc3oc4ccccc4c3c2-c2c(-c3ccccc3)ccc3c2oc2ccccc23)n1 ; C[Si](C)(C)c1ccc(-c2ccccc2)cc1-c1nc(-c2ccccc2)nc(-c2c(-c3c(-c4ccccc4)ccc4c3oc3ccccc34)ccc3c2oc2ccccc23)n1] 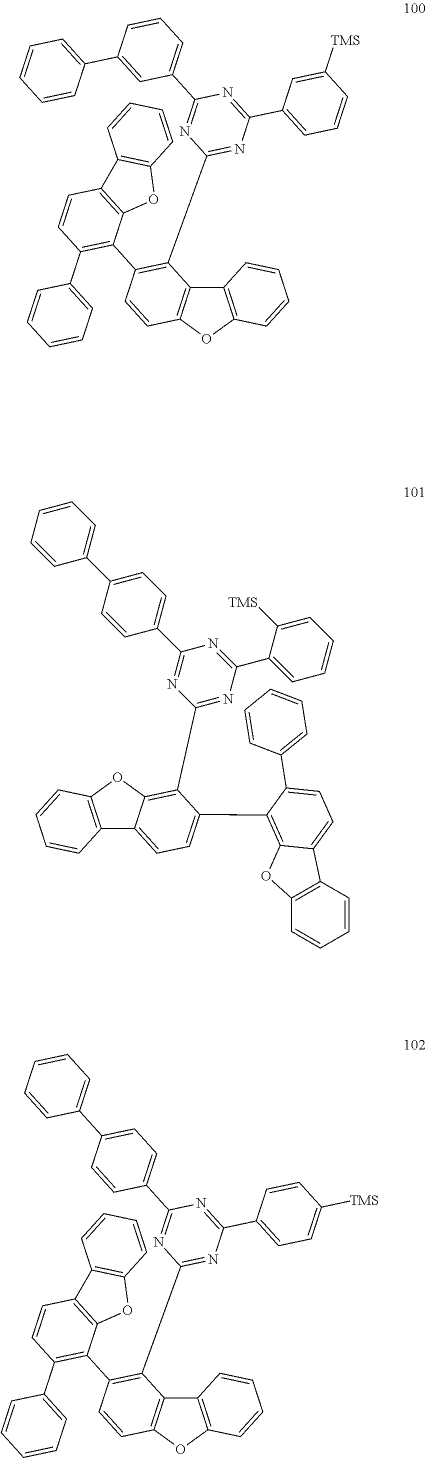 [SMILES: C[Si](C)(C)c1cccc(-c2nc(-c3cccc(-c4ccccc4)c3)nc(-c3c(-c4c(-c5ccccc5)ccc5c4oc4ccccc45)ccc4oc5ccccc5c34)n2)c1 ; C[Si](C)(C)c1ccccc1-c1nc(-c2ccc(-c3ccccc3)cc2)nc(-c2c(-c3c(-c4ccccc4)ccc4c3oc3ccccc34)ccc3c2oc2ccccc23)n1 ; C[Si](C)(C)c1ccc(-c2nc(-c3ccc(-c4ccccc4)cc3)nc(-c3c(-c4c(-c5ccccc5)ccc5c4oc4ccccc45)ccc4oc5ccccc5c34)n2)cc1] 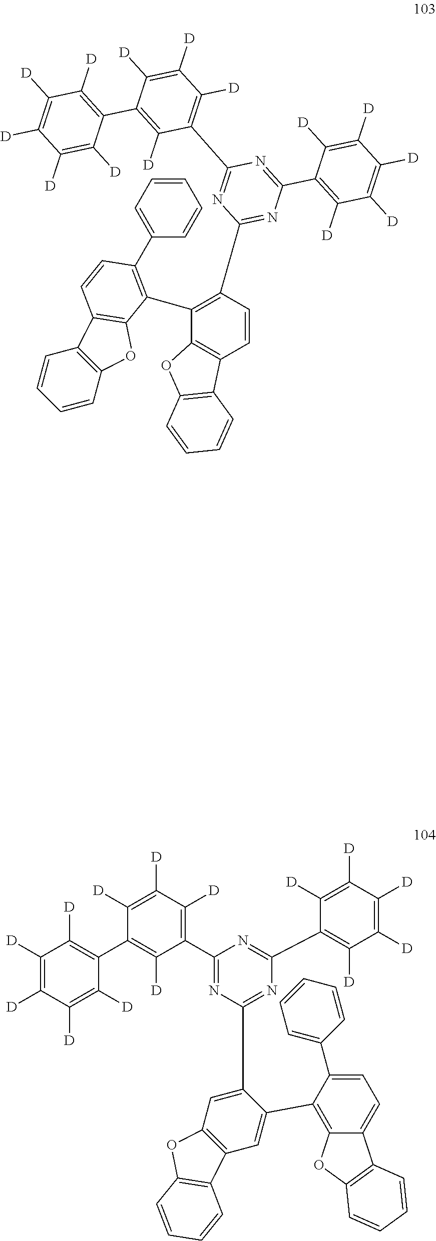 [SMILES: [2H]c1c([2H])c([2H])c(-c2nc(-c3ccc4c(oc5ccccc54)c3-c3c(-c4ccccc4)ccc4c3oc3ccccc34)nc(-c3c([2H])c([2H])c([2H])c(-c4c([2H])c([2H])c([2H])c([2H])c4[2H])c3[2H])n2)c([2H])c1[2H] ; [2H]c1c([2H])c([2H])c(-c2nc(-c3cc4oc5ccccc5c4cc3-c3c(-c4ccccc4)ccc4c3oc3ccccc34)nc(-c3c([2H])c([2H])c([2H])c(-c4c([2H])c([2H])c([2H])c([2H])c4[2H])c3[2H])n2)c([2H])c1[2H]] 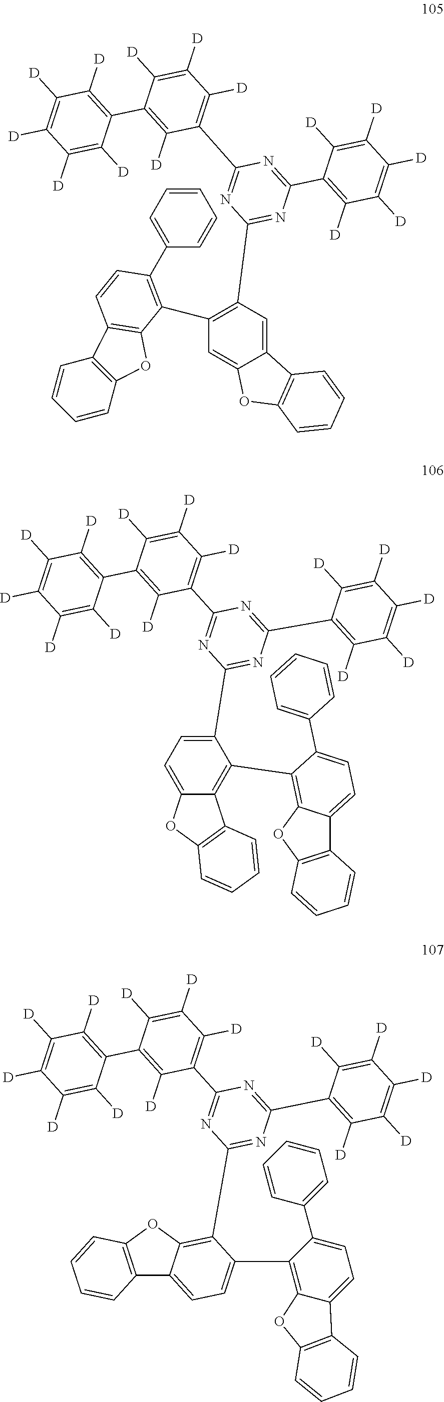 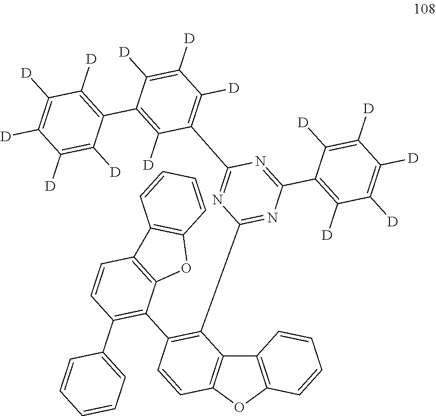 [SMILES: [2H]c1c([2H])c([2H])c(-c2nc(-c3c([2H])c([2H])c([2H])c(-c4c([2H])c([2H])c([2H])c([2H])c4[2H])c3[2H])nc(-c3c(-c4c(-c5ccccc5)ccc5c4oc4ccccc45)ccc4oc5ccccc5c34)n2)c([2H])c1[2H]] 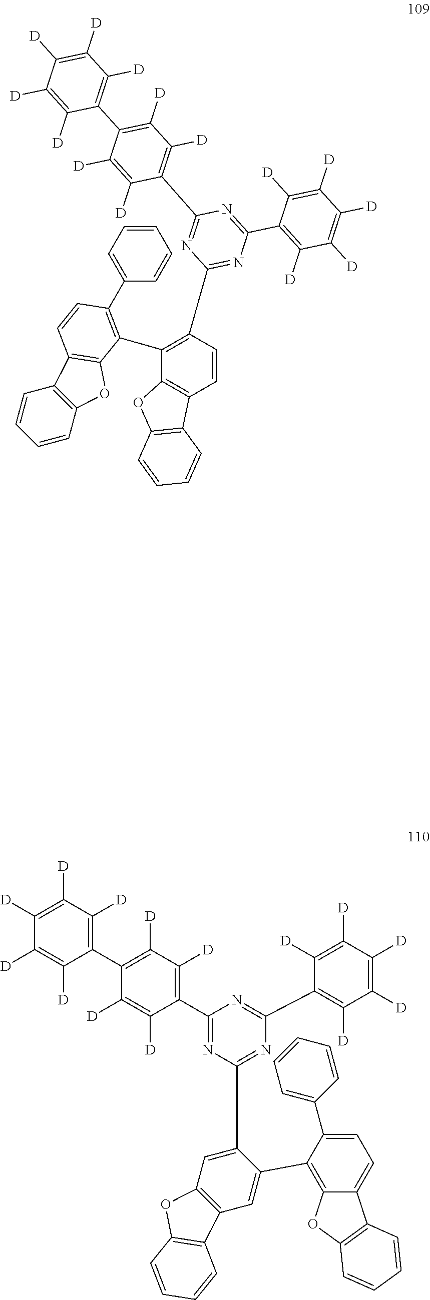 [SMILES: [2H]c1c([2H])c([2H])c(-c2nc(-c3ccc4c(oc5ccccc54)c3-c3c(-c4ccccc4)ccc4c3oc3ccccc34)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c(-c2nc(-c3cc4oc5ccccc5c4cc3-c3c(-c4ccccc4)ccc4c3oc3ccccc34)nc(-c3c([2H])c([2H])c(-c4c([2H])c([2H])c([2H])c([2H])c4[2H])c([2H])c3[2H])n2)c([2H])c1[2H]] 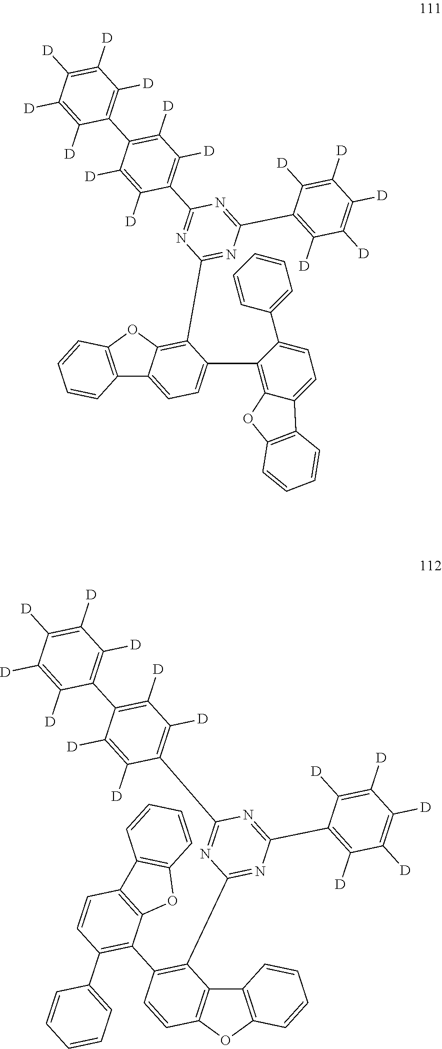 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c(-c4c(-c5ccccc5)ccc5c4oc4ccccc45)ccc4c3oc3ccccc34)n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c(-c4c(-c5ccccc5)ccc5c4oc4ccccc45)ccc4oc5ccccc5c34)n2)c([2H])c1[2H]] 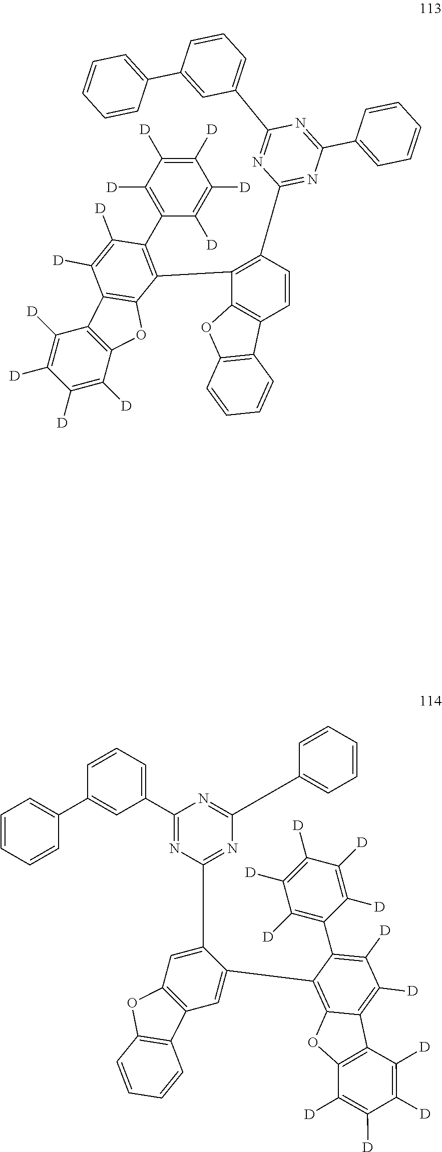 [SMILES: [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)ccc3c2oc2ccccc23)c([2H])c1[2H] ; [2H]c1c([2H])c([2H])c(-c2c([2H])c([2H])c3c(oc4c([2H])c([2H])c([2H])c([2H])c43)c2-c2cc3c(cc2-c2nc(-c4ccccc4)nc(-c4cccc(-c5ccccc5)c4)n2)oc2ccccc23)c([2H])c1[2H]] 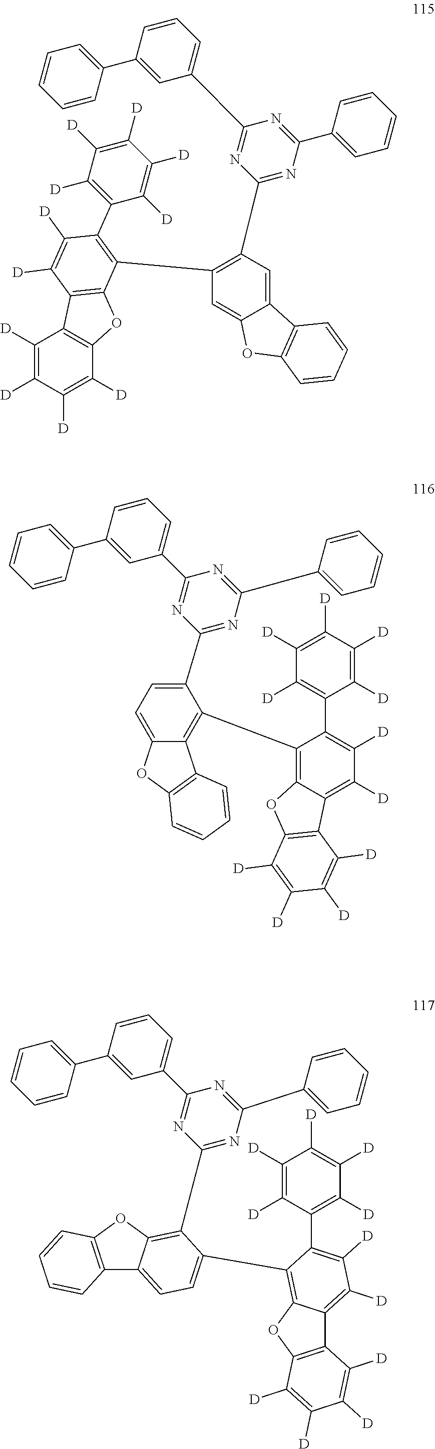 [SMILES: [2H]c1c([2H])c([2H])c(-c2c([2H])c([2H])c3c(oc4c([2H])c([2H])c([2H])c([2H])c43)c2-c2cc3oc4ccccc4c3cc2-c2nc(-c3ccccc3)nc(-c3cccc(-c4ccccc4)c3)n2)c([2H])c1[2H] ; [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)ccc3oc4ccccc4c23)c([2H])c1[2H] ; [2H]c1c([2H])c([2H])c(-c2c([2H])c([2H])c3c(oc4c([2H])c([2H])c([2H])c([2H])c43)c2-c2ccc3c(oc4ccccc43)c2-c2nc(-c3ccccc3)nc(-c3cccc(-c4ccccc4)c3)n2)c([2H])c1[2H]] 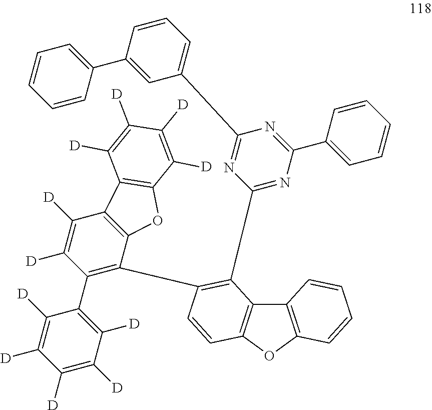 [SMILES: [2H]c1c([2H])c([2H])c(-c2c([2H])c([2H])c3c(oc4c([2H])c([2H])c([2H])c([2H])c43)c2-c2ccc3oc4ccccc4c3c2-c2nc(-c3ccccc3)nc(-c3cccc(-c4ccccc4)c3)n2)c([2H])c1[2H]] 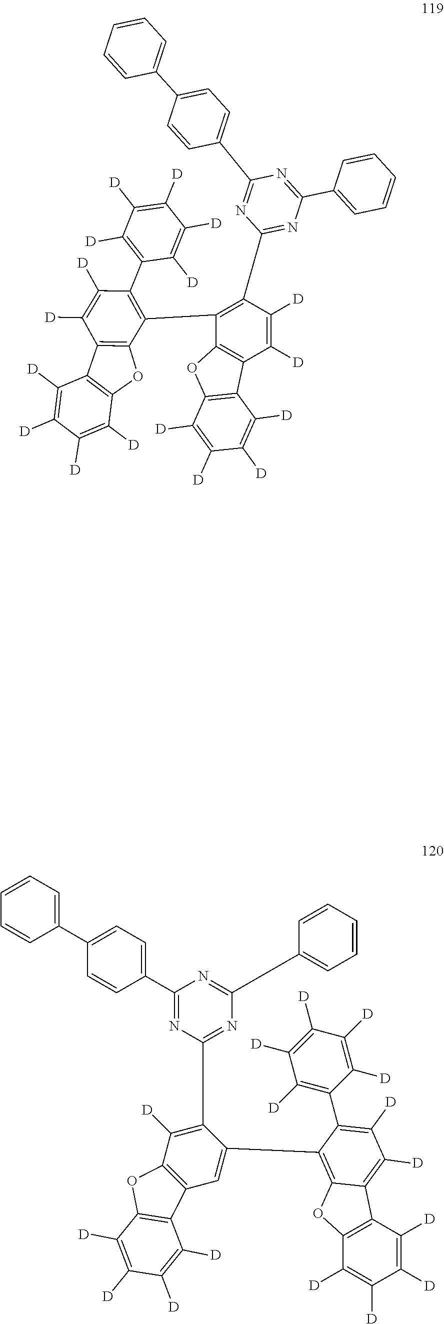 [SMILES: [2H]c1c([2H])c([2H])c(-c2c([2H])c([2H])c3c(oc4c([2H])c([2H])c([2H])c([2H])c43)c2-c2c(-c3nc(-c4ccccc4)nc(-c4ccc(-c5ccccc5)cc4)n3)c([2H])c([2H])c3c2oc2c([2H])c([2H])c([2H])c([2H])c23)c([2H])c1[2H] ; [2H]c1c([2H])c([2H])c(-c2c([2H])c([2H])c3c(oc4c([2H])c([2H])c([2H])c([2H])c43)c2-c2cc3c(oc4c([2H])c([2H])c([2H])c([2H])c43)c([2H])c2-c2nc(-c3ccccc3)nc(-c3ccc(-c4ccccc4)cc3)n2)c([2H])c1[2H]] 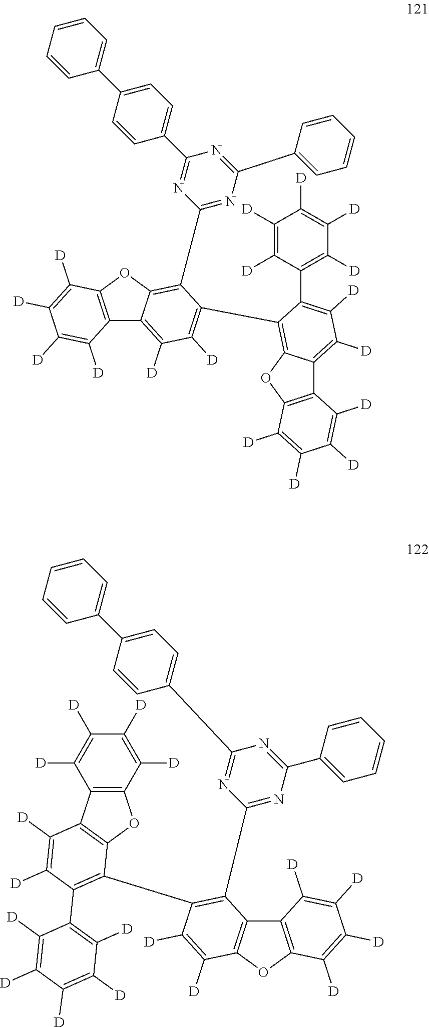 [SMILES: [2H]c1c([2H])c([2H])c(-c2c([2H])c([2H])c3c(oc4c([2H])c([2H])c([2H])c([2H])c43)c2-c2c([2H])c([2H])c3c(oc4c([2H])c([2H])c([2H])c([2H])c43)c2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(oc4c([2H])c([2H])c([2H])c([2H])c43)c2-c2c([2H])c([2H])c3oc4c([2H])c([2H])c([2H])c([2H])c4c3c2-c2nc(-c3ccccc3)nc(-c3ccc(-c4ccccc4)cc3)n2)c([2H])c1[2H]] 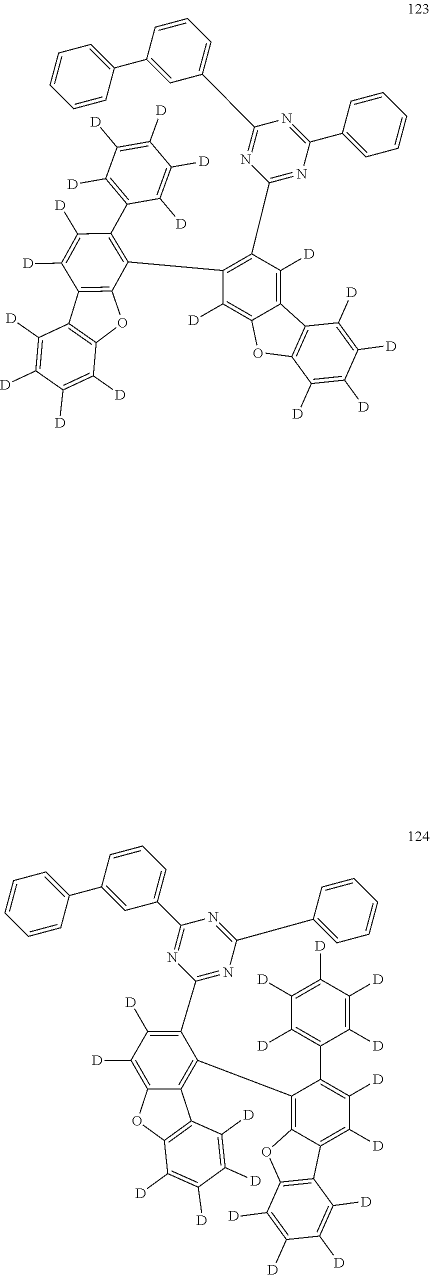 [SMILES: [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)c([2H])c3c(oc4c([2H])c([2H])c([2H])c([2H])c43)c2[2H])c([2H])c1[2H] ; [2H]c1c([2H])c([2H])c(-c2c([2H])c([2H])c3c(oc4c([2H])c([2H])c([2H])c([2H])c43)c2-c2c(-c3nc(-c4ccccc4)nc(-c4cccc(-c5ccccc5)c4)n3)c([2H])c([2H])c3oc4c([2H])c([2H])c([2H])c([2H])c4c23)c([2H])c1[2H]] 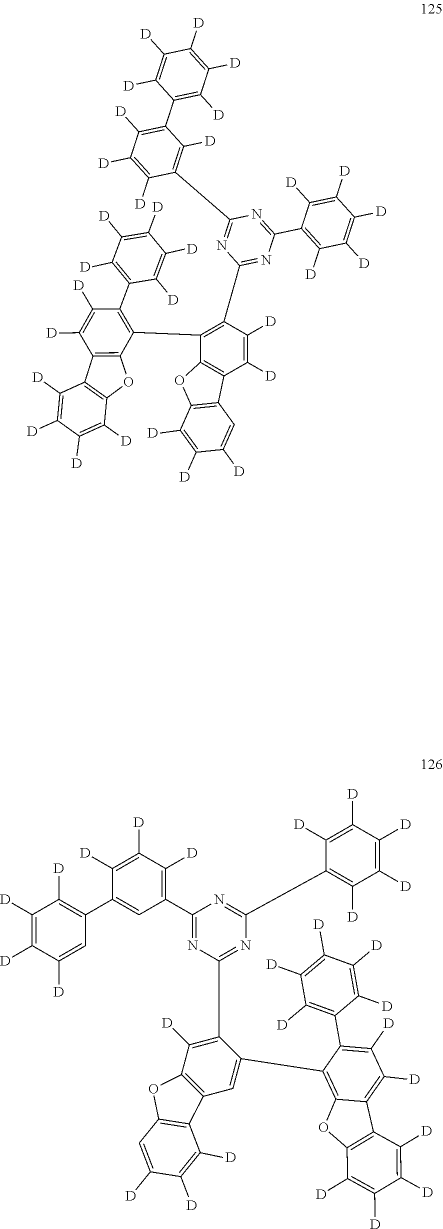 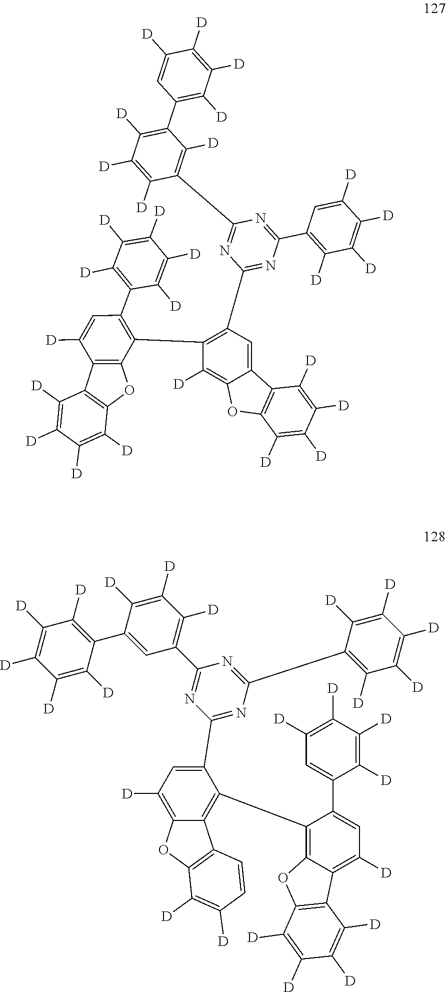 [SMILES: [2H]c1cc(-c2nc(-c3cc4c(oc5c([2H])c([2H])c([2H])c([2H])c54)c([2H])c3-c3c(-c4c([2H])c([2H])c([2H])c([2H])c4[2H])cc([2H])c4c3oc3c([2H])c([2H])c([2H])c([2H])c34)nc(-c3c([2H])c([2H])c([2H])c(-c4cc([2H])c([2H])c([2H])c4[2H])c3[2H])n2)c([2H])c([2H])c1[2H] ; [2H]c1cc(-c2cc([2H])c3c(oc4c([2H])c([2H])c([2H])c([2H])c43)c2-c2c(-c3nc(-c4cc(-c5c([2H])c([2H])c([2H])c([2H])c5[2H])c([2H])c([2H])c4[2H])nc(-c4c([2H])c([2H])c([2H])c([2H])c4[2H])n3)cc([2H])c3oc4c([2H])c([2H])ccc4c23)c([2H])c([2H])c1[2H]] 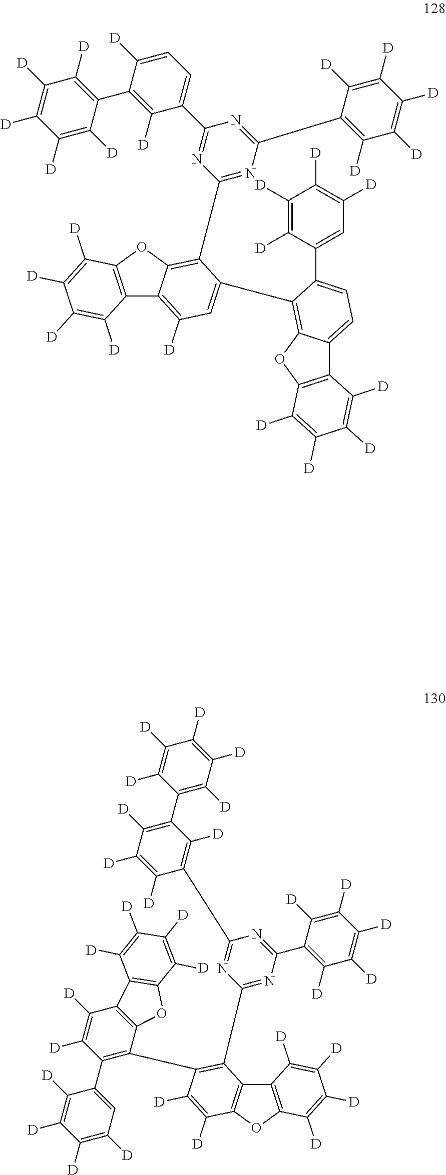 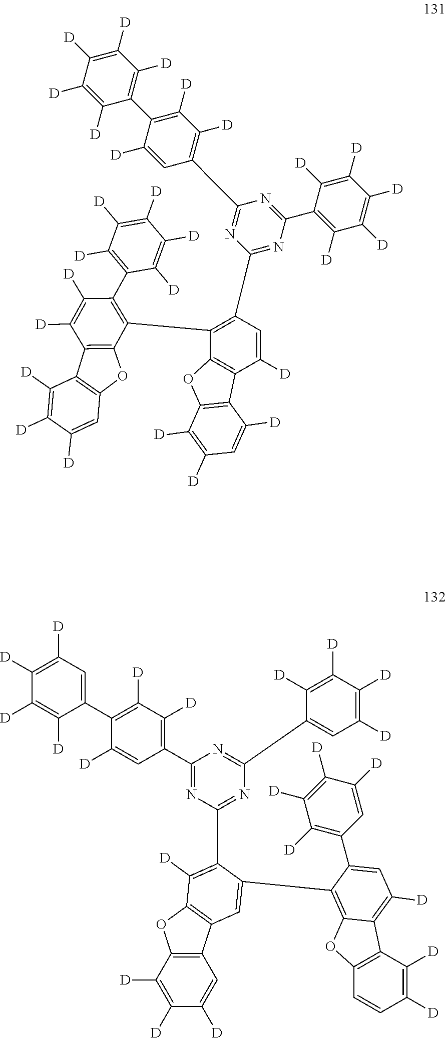 [SMILES: [2H]c1cc2oc3c(-c4c(-c5nc(-c6cc([2H])c(-c7c([2H])c([2H])c([2H])c([2H])c7[2H])c([2H])c6[2H])nc(-c6c([2H])c([2H])c([2H])c([2H])c6[2H])n5)cc([2H])c5c4oc4c([2H])c([2H])cc([2H])c45)c(-c4c([2H])c([2H])c([2H])c([2H])c4[2H])c([2H])c([2H])c3c2c([2H])c1[2H] ; [2H]c1cc(-c2nc(-c3cc([2H])c(-c4cc([2H])c([2H])c([2H])c4[2H])c([2H])c3[2H])nc(-c3c(-c4c(-c5cc([2H])c([2H])c([2H])c5[2H])cc([2H])c5c4oc4ccc([2H])c([2H])c45)cc4c(oc5c([2H])c([2H])c([2H])cc54)c3[2H])n2)c([2H])c([2H])c1[2H]] 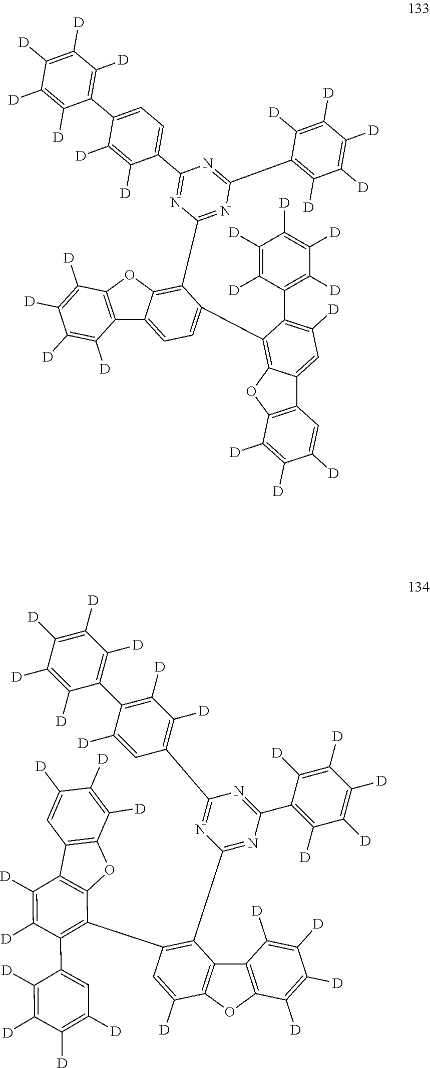 [SMILES: [2H]c1cc2c(oc3c(-c4ccc5c(oc6c([2H])c([2H])c([2H])c([2H])c65)c4-c4nc(-c5ccc(-c6c([2H])c([2H])c([2H])c([2H])c6[2H])c([2H])c5[2H])nc(-c5c([2H])c([2H])c([2H])c([2H])c5[2H])n4)c(-c4c([2H])c([2H])c([2H])c([2H])c4[2H])c([2H])cc32)c([2H])c1[2H] ; [2H]c1cc(-c2c([2H])c([2H])c3c(oc4c([2H])c([2H])c([2H])cc43)c2-c2cc([2H])c3oc4c([2H])c([2H])c([2H])c([2H])c4c3c2-c2nc(-c3cc([2H])c(-c4c([2H])c([2H])c([2H])c([2H])c4[2H])c([2H])c3[2H])nc(-c3c([2H])c([2H])c([2H])c([2H])c3[2H])n2)c([2H])c([2H])c1[2H]] 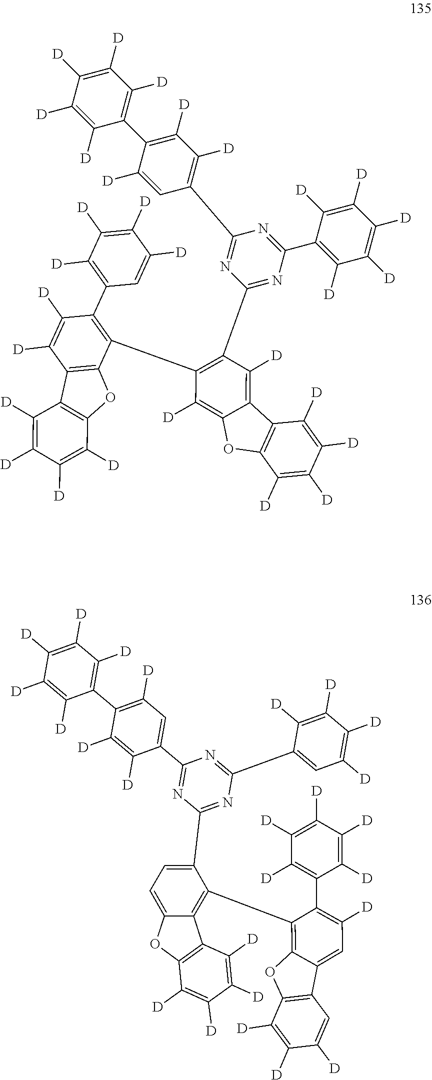 [SMILES: [2H]c1cc(-c2c([2H])c([2H])c3c(oc4c([2H])c([2H])c([2H])c([2H])c43)c2-c2c(-c3nc(-c4cc([2H])c(-c5c([2H])c([2H])c([2H])c([2H])c5[2H])c([2H])c4[2H])nc(-c4c([2H])c([2H])c([2H])c([2H])c4[2H])n3)c([2H])c3c(oc4c([2H])c([2H])c([2H])c([2H])c43)c2[2H])c([2H])c([2H])c1[2H] ; [2H]c1cc(-c2nc(-c3cc([2H])c(-c4c([2H])c([2H])c([2H])c([2H])c4[2H])c([2H])c3[2H])nc(-c3ccc4oc5c([2H])c([2H])c([2H])c([2H])c5c4c3-c3c(-c4c([2H])c([2H])c([2H])c([2H])c4[2H])c([2H])cc4c3oc3c([2H])c([2H])c([2H])cc34)n2)c([2H])c([2H])c1[2H]] 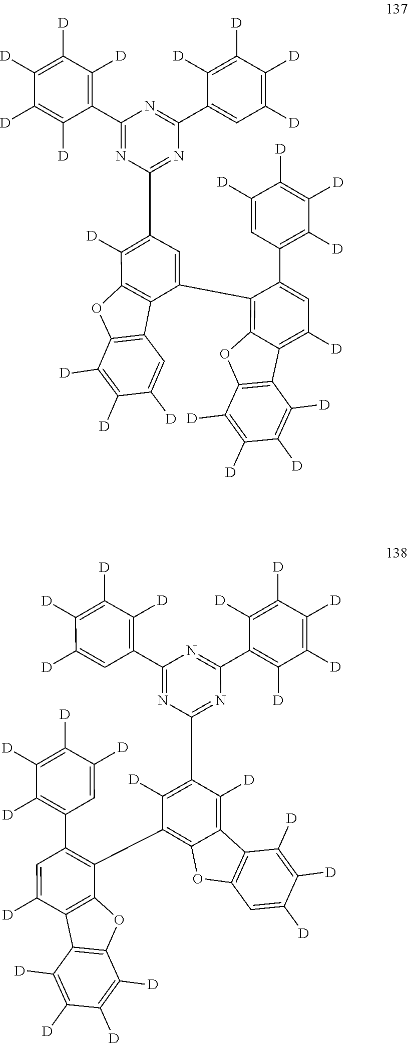 [SMILES: [2H]c1cc(-c2nc(-c3cc(-c4c(-c5cc([2H])c([2H])c([2H])c5[2H])cc([2H])c5c4oc4c([2H])c([2H])c([2H])c([2H])c45)c4c(oc5c([2H])c([2H])c([2H])cc54)c3[2H])nc(-c3c([2H])c([2H])c([2H])c([2H])c3[2H])n2)c([2H])c([2H])c1[2H] ; [2H]c1cc(-c2nc(-c3c([2H])c([2H])c([2H])c([2H])c3[2H])nc(-c3c([2H])c(-c4c(-c5cc([2H])c([2H])c([2H])c5[2H])cc([2H])c5c4oc4c([2H])c([2H])c([2H])c([2H])c45)c4oc5cc([2H])c([2H])c([2H])c5c4c3[2H])n2)c([2H])c([2H])c1[2H]] 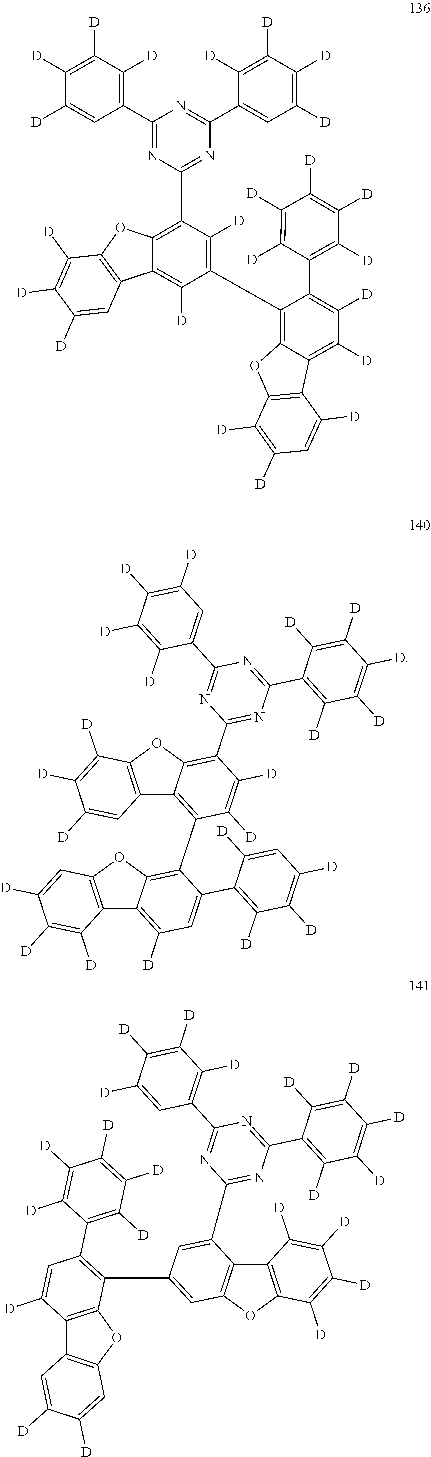 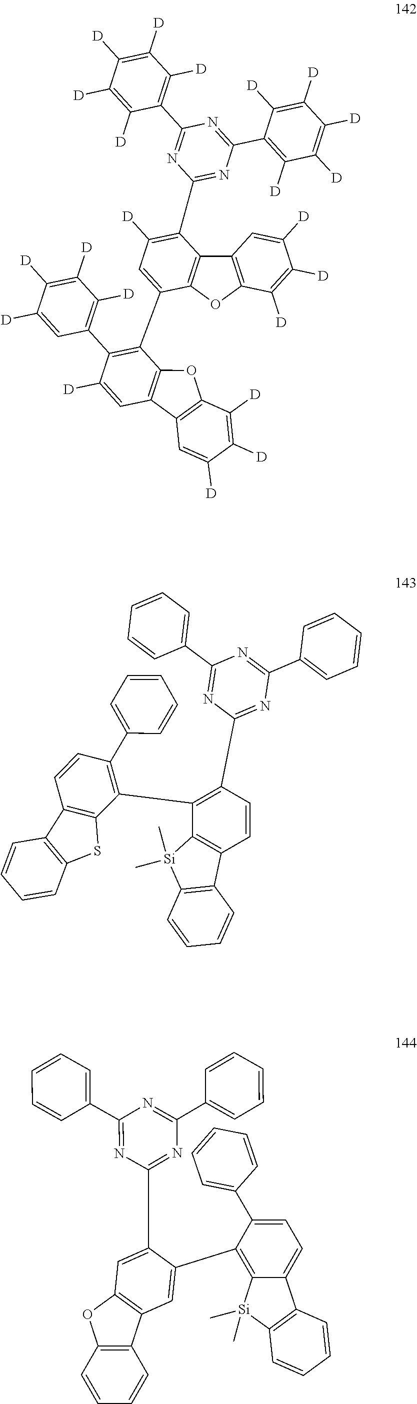 [SMILES: [2H]c1cc(-c2c([2H])cc3c(oc4c([2H])c([2H])c([2H])cc43)c2-c2cc([2H])c(-c3nc(-c4c([2H])c([2H])c([2H])c([2H])c4[2H])nc(-c4c([2H])c([2H])c([2H])c([2H])c4[2H])n3)c3c2oc2c([2H])c([2H])c([2H])cc23)c([2H])c([2H])c1[2H] ; C[Si]1(C)c2ccccc2-c2ccc(-c3nc(-c4ccccc4)nc(-c4ccccc4)n3)c(-c3c(-c4ccccc4)ccc4c3sc3ccccc34)c21 ; C[Si]1(C)c2ccccc2-c2ccc(-c3ccccc3)c(-c3cc4c(cc3-c3nc(-c5ccccc5)nc(-c5ccccc5)n3)oc3ccccc34)c21] 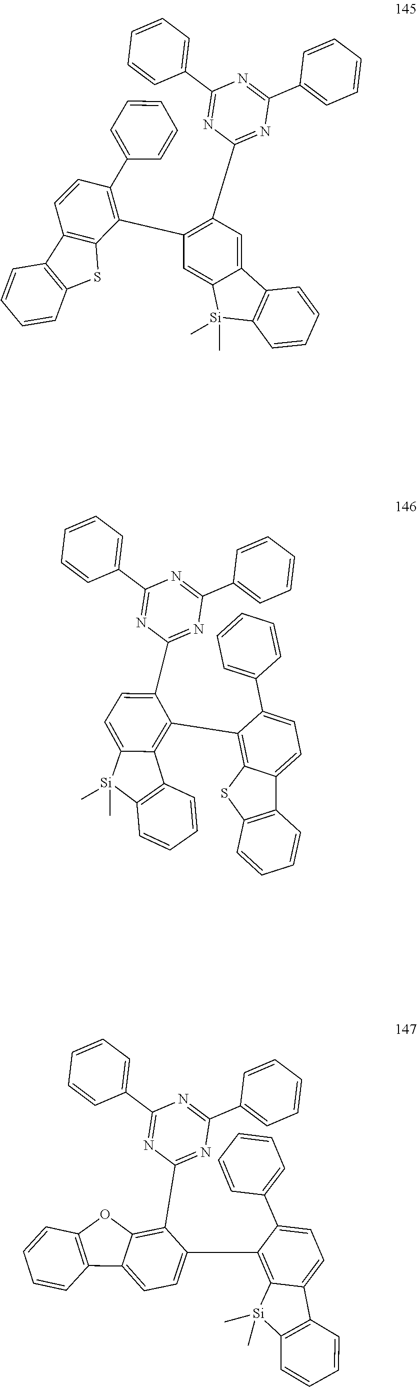 [SMILES: C[Si]1(C)c2ccccc2-c2cc(-c3nc(-c4ccccc4)nc(-c4ccccc4)n3)c(-c3c(-c4ccccc4)ccc4c3sc3ccccc34)cc21 ; C[Si]1(C)c2ccccc2-c2c1ccc(-c1nc(-c3ccccc3)nc(-c3ccccc3)n1)c2-c1c(-c2ccccc2)ccc2c1sc1ccccc12 ; C[Si]1(C)c2ccccc2-c2ccc(-c3ccccc3)c(-c3ccc4c(oc5ccccc54)c3-c3nc(-c4ccccc4)nc(-c4ccccc4)n3)c21] 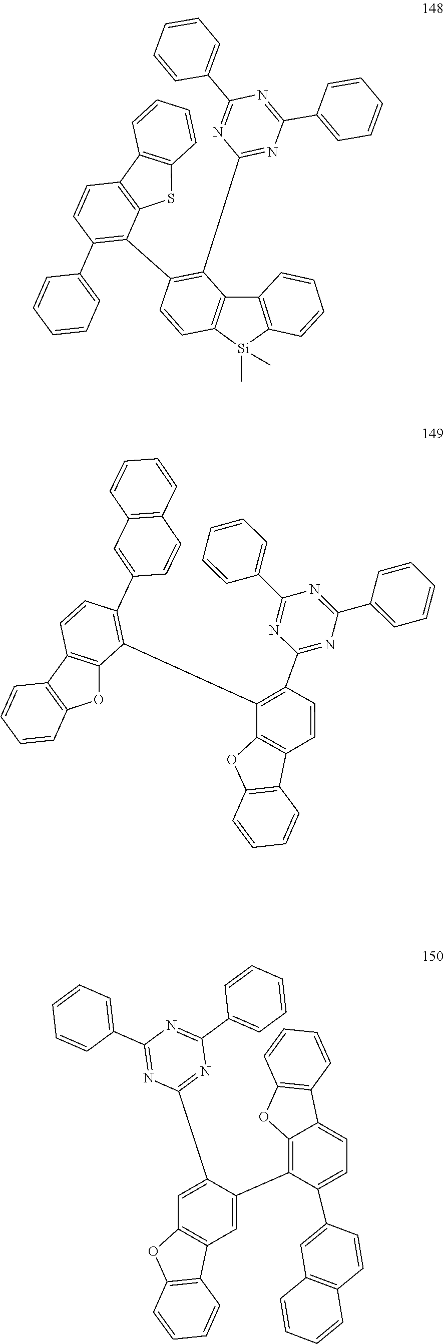 [SMILES: C[Si]1(C)c2ccccc2-c2c1ccc(-c1c(-c3ccccc3)ccc3c1sc1ccccc13)c2-c1nc(-c2ccccc2)nc(-c2ccccc2)n1 ; c1ccc(-c2nc(-c3ccccc3)nc(-c3ccc4c(oc5ccccc54)c3-c3c(-c4ccc5ccccc5c4)ccc4c3oc3ccccc34)n2)cc1 ; c1ccc(-c2nc(-c3ccccc3)nc(-c3cc4oc5ccccc5c4cc3-c3c(-c4ccc5ccccc5c4)ccc4c3oc3ccccc34)n2)cc1] 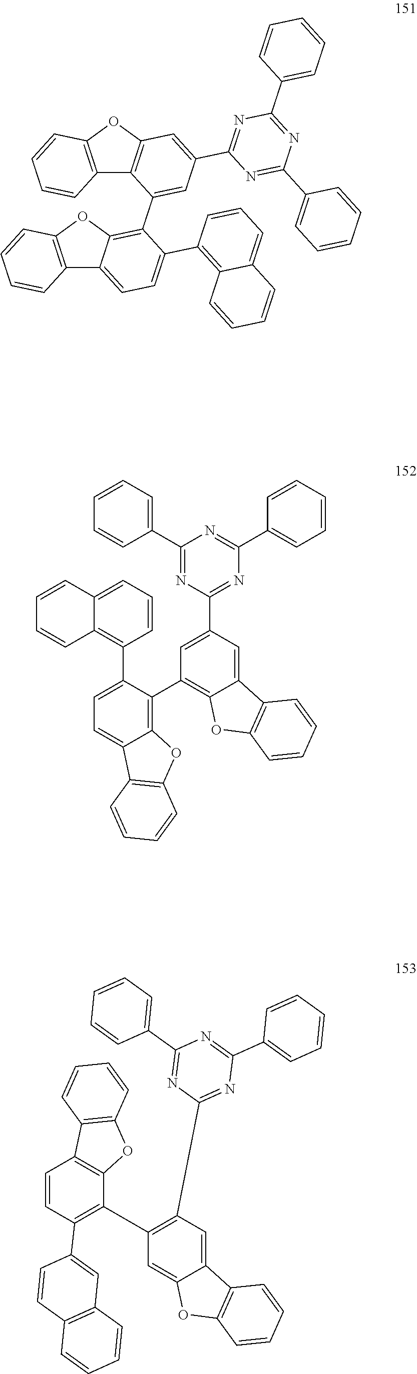 [SMILES: c1ccc(-c2nc(-c3ccccc3)nc(-c3cc(-c4c(-c5cccc6ccccc56)ccc5c4oc4ccccc45)c4c(c3)oc3ccccc34)n2)cc1 ; c1ccc(-c2nc(-c3ccccc3)nc(-c3cc(-c4c(-c5cccc6ccccc56)ccc5c4oc4ccccc45)c4oc5ccccc5c4c3)n2)cc1 ; c1ccc(-c2nc(-c3ccccc3)nc(-c3cc4c(cc3-c3c(-c5ccc6ccccc6c5)ccc5c3oc3ccccc35)oc3ccccc34)n2)cc1] 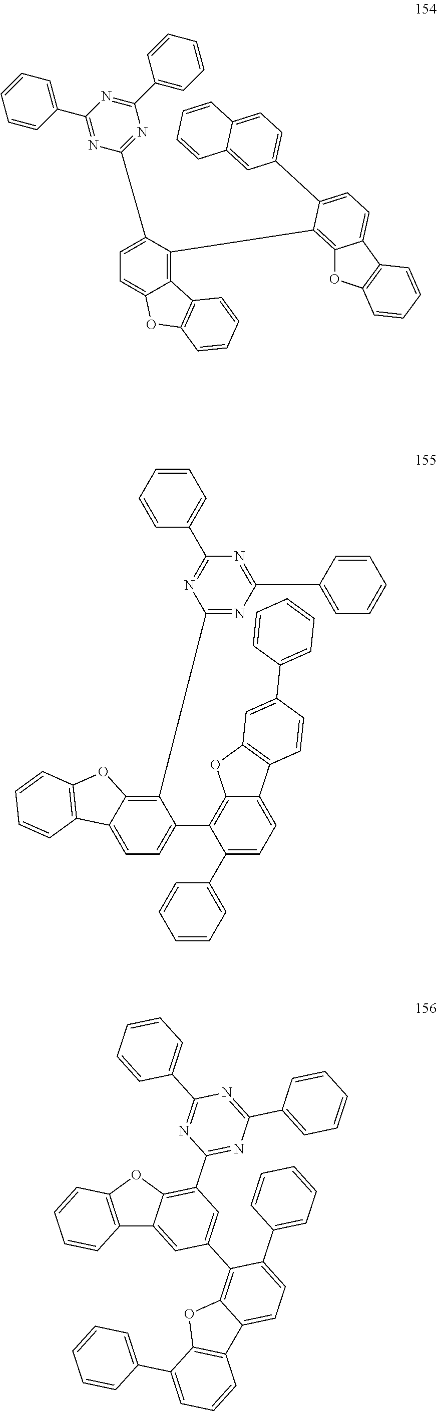 [SMILES: c1ccc(-c2nc(-c3ccccc3)nc(-c3ccc4oc5ccccc5c4c3-c3c(-c4ccc5ccccc5c4)ccc4c3oc3ccccc34)n2)cc1 ; c1ccc(-c2ccc3c(c2)oc2c(-c4ccc5c(oc6ccccc65)c4-c4nc(-c5ccccc5)nc(-c5ccccc5)n4)c(-c4ccccc4)ccc23)cc1 ; c1ccc(-c2nc(-c3ccccc3)nc(-c3cc(-c4c(-c5ccccc5)ccc5c4oc4c(-c6ccccc6)cccc45)cc4c3oc3ccccc34)n2)cc1] 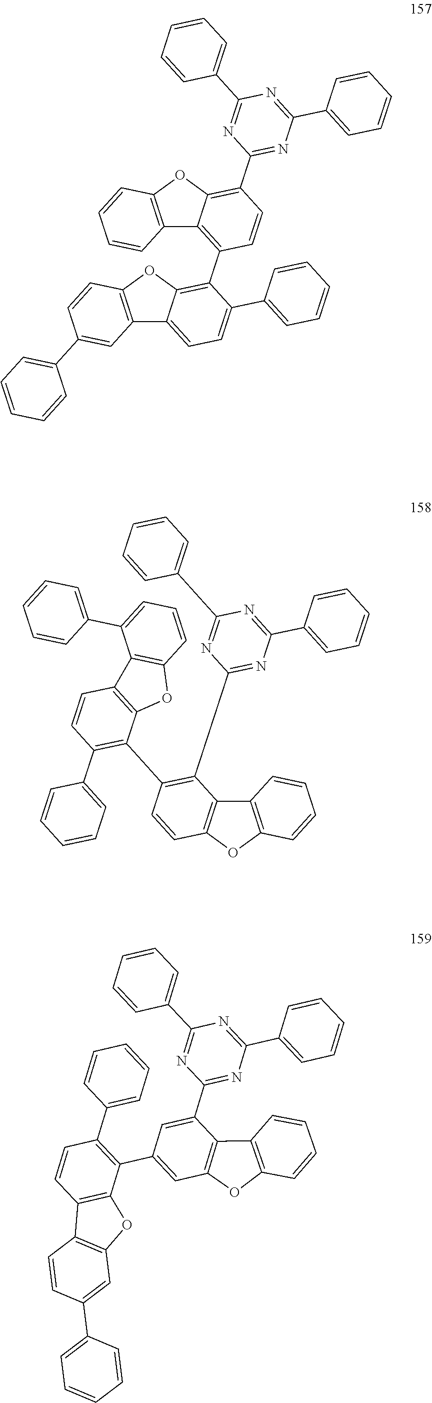 [SMILES: c1ccc(-c2ccc3oc4c(-c5ccc(-c6nc(-c7ccccc7)nc(-c7ccccc7)n6)c6oc7ccccc7c56)c(-c5ccccc5)ccc4c3c2)cc1 ; c1ccc(-c2nc(-c3ccccc3)nc(-c3c(-c4c(-c5ccccc5)ccc5c4oc4cccc(-c6ccccc6)c45)ccc4oc5ccccc5c34)n2)cc1 ; c1ccc(-c2ccc3c(c2)oc2c(-c4cc(-c5nc(-c6ccccc6)nc(-c6ccccc6)n5)c5c(c4)oc4ccccc45)c(-c4ccccc4)ccc23)cc1] 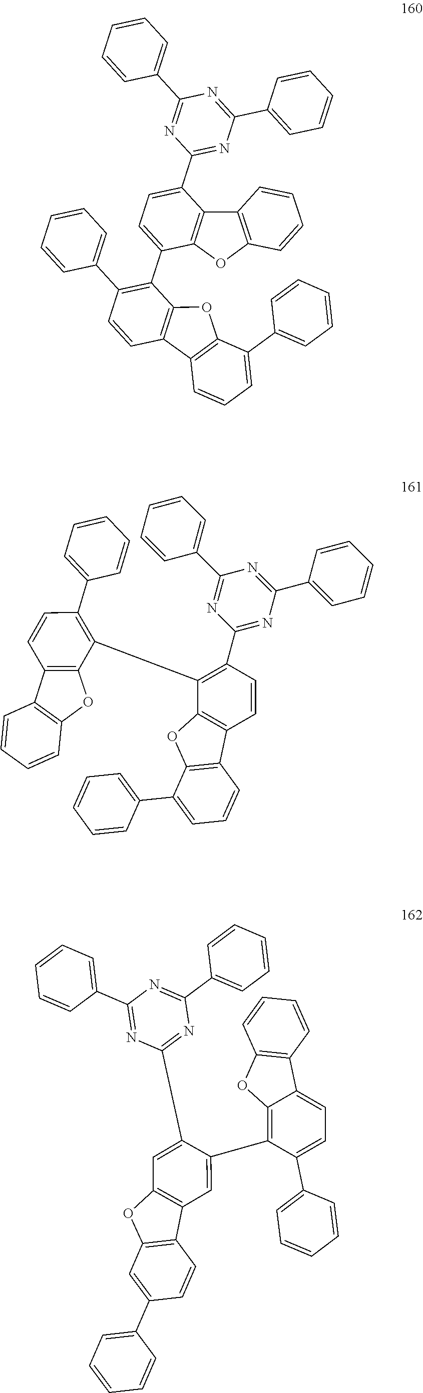 [SMILES: c1ccc(-c2nc(-c3ccccc3)nc(-c3ccc(-c4c(-c5ccccc5)ccc5c4oc4c(-c6ccccc6)cccc45)c4oc5ccccc5c34)n2)cc1 ; c1ccc(-c2nc(-c3ccccc3)nc(-c3ccc4c(oc5c(-c6ccccc6)cccc54)c3-c3c(-c4ccccc4)ccc4c3oc3ccccc34)n2)cc1 ; c1ccc(-c2ccc3c(c2)oc2cc(-c4nc(-c5ccccc5)nc(-c5ccccc5)n4)c(-c4c(-c5ccccc5)ccc5c4oc4ccccc45)cc23)cc1] 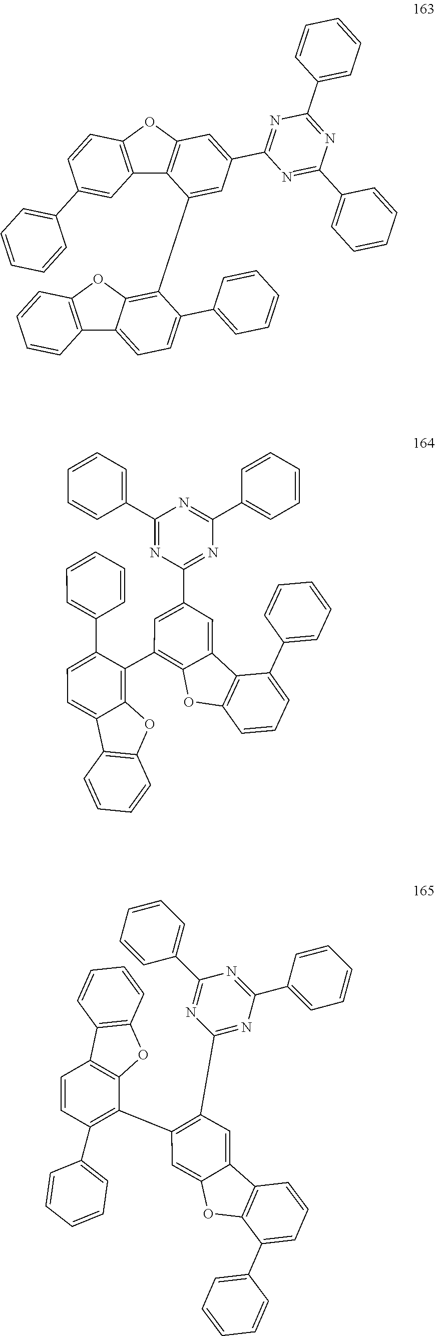 [SMILES: c1ccc(-c2ccc3oc4cc(-c5nc(-c6ccccc6)nc(-c6ccccc6)n5)cc(-c5c(-c6ccccc6)ccc6c5oc5ccccc56)c4c3c2)cc1 ; c1ccc(-c2nc(-c3ccccc3)nc(-c3cc(-c4c(-c5ccccc5)ccc5c4oc4ccccc45)c4oc5cccc(-c6ccccc6)c5c4c3)n2)cc1 ; c1ccc(-c2nc(-c3ccccc3)nc(-c3cc4c(cc3-c3c(-c5ccccc5)ccc5c3oc3ccccc35)oc3c(-c5ccccc5)cccc34)n2)cc1] 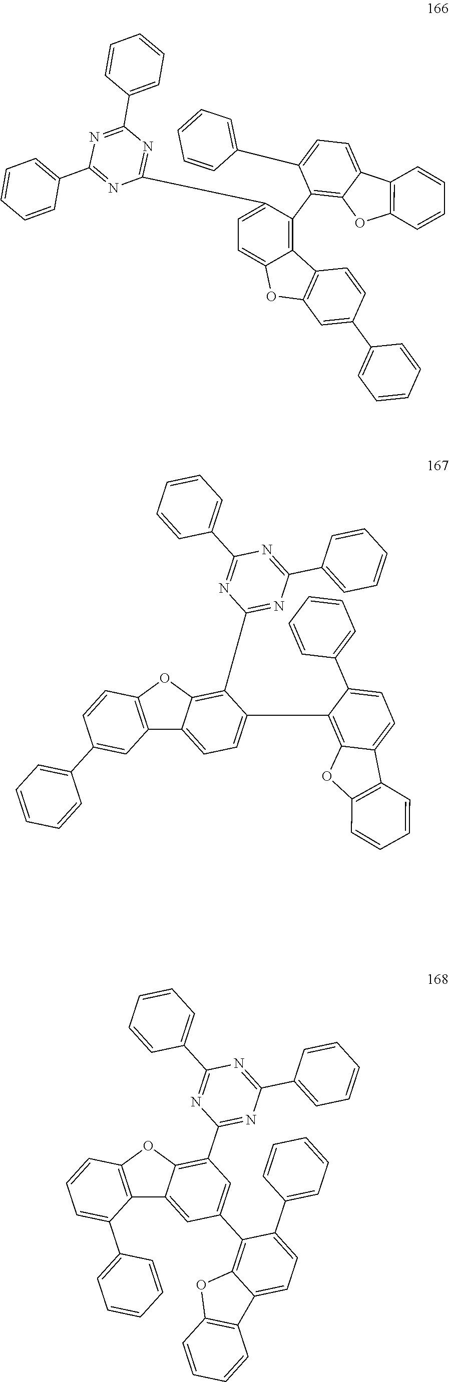 [SMILES: c1ccc(-c2ccc3c(c2)oc2ccc(-c4nc(-c5ccccc5)nc(-c5ccccc5)n4)c(-c4c(-c5ccccc5)ccc5c4oc4ccccc45)c23)cc1 ; c1ccc(-c2ccc3oc4c(-c5nc(-c6ccccc6)nc(-c6ccccc6)n5)c(-c5c(-c6ccccc6)ccc6c5oc5ccccc56)ccc4c3c2)cc1 ; c1ccc(-c2nc(-c3ccccc3)nc(-c3cc(-c4c(-c5ccccc5)ccc5c4oc4ccccc45)cc4c3oc3cccc(-c5ccccc5)c34)n2)cc1] 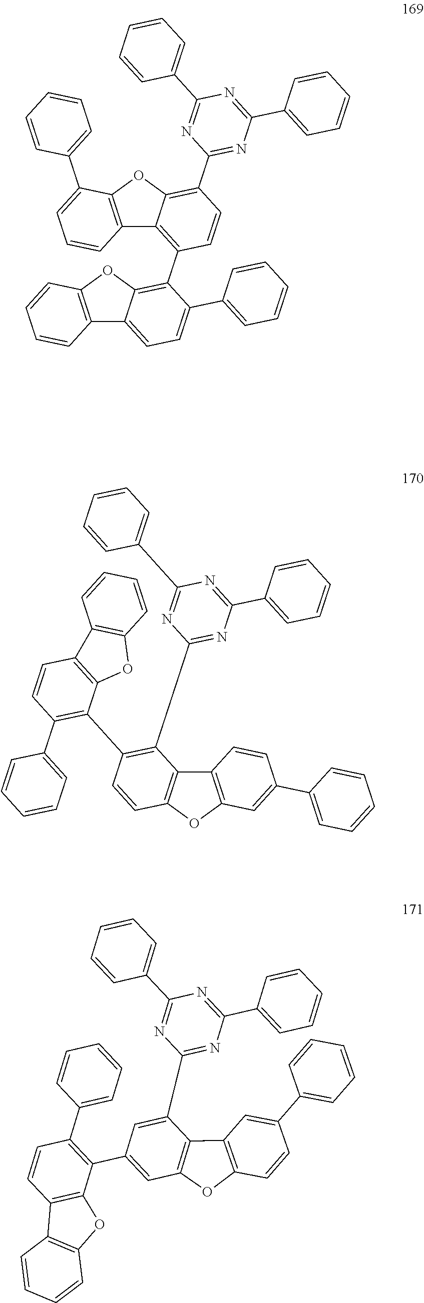 [SMILES: c1ccc(-c2nc(-c3ccccc3)nc(-c3ccc(-c4c(-c5ccccc5)ccc5c4oc4ccccc45)c4c3oc3c(-c5ccccc5)cccc34)n2)cc1 ; c1ccc(-c2ccc3c(c2)oc2ccc(-c4c(-c5ccccc5)ccc5c4oc4ccccc45)c(-c4nc(-c5ccccc5)nc(-c5ccccc5)n4)c23)cc1 ; c1ccc(-c2ccc3oc4cc(-c5c(-c6ccccc6)ccc6c5oc5ccccc56)cc(-c5nc(-c6ccccc6)nc(-c6ccccc6)n5)c4c3c2)cc1] 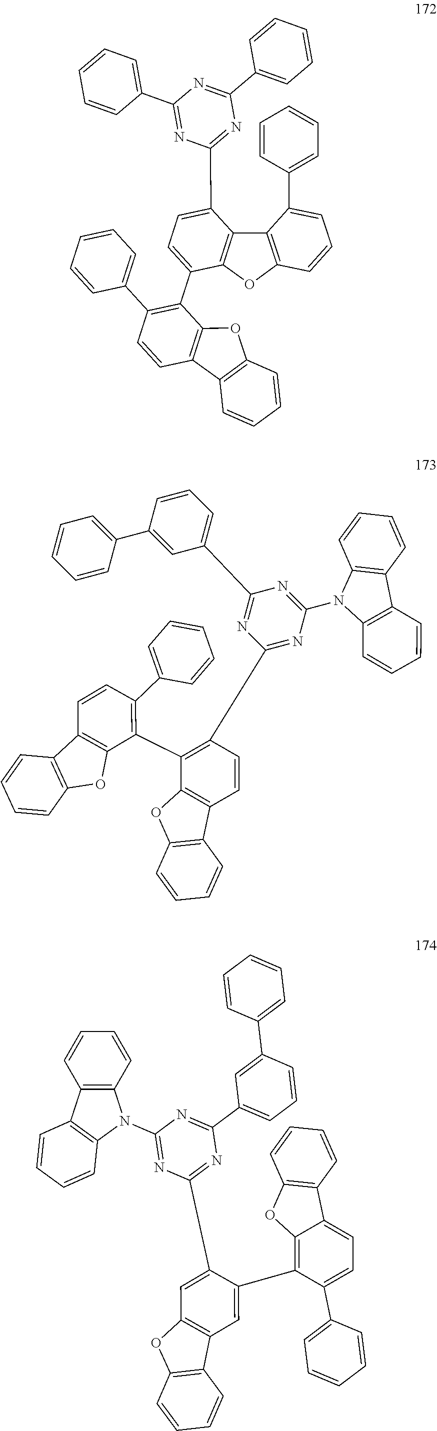 [SMILES: c1ccc(-c2nc(-c3ccccc3)nc(-c3ccc(-c4c(-c5ccccc5)ccc5c4oc4ccccc45)c4oc5cccc(-c6ccccc6)c5c34)n2)cc1 ; c1ccc(-c2cccc(-c3nc(-c4ccc5c(oc6ccccc65)c4-c4c(-c5ccccc5)ccc5c4oc4ccccc45)nc(-n4c5ccccc5c5ccccc54)n3)c2)cc1 ; c1ccc(-c2cccc(-c3nc(-c4cc5oc6ccccc6c5cc4-c4c(-c5ccccc5)ccc5c4oc4ccccc45)nc(-n4c5ccccc5c5ccccc54)n3)c2)cc1] 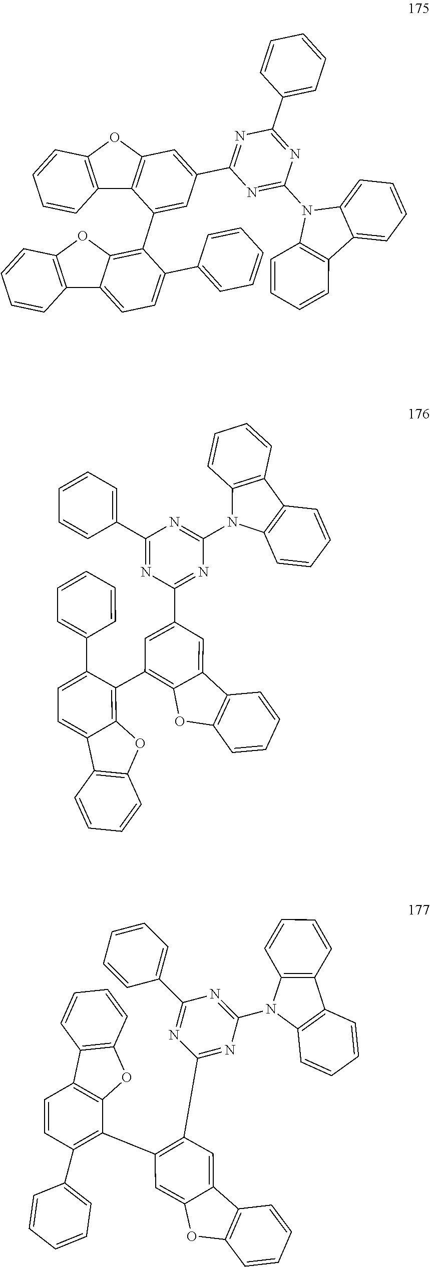 [SMILES: c1ccc(-c2nc(-c3cc(-c4c(-c5ccccc5)ccc5c4oc4ccccc45)c4c(c3)oc3ccccc34)nc(-n3c4ccccc4c4ccccc43)n2)cc1 ; c1ccc(-c2nc(-c3cc(-c4c(-c5ccccc5)ccc5c4oc4ccccc45)c4oc5ccccc5c4c3)nc(-n3c4ccccc4c4ccccc43)n2)cc1 ; c1ccc(-c2nc(-c3cc4c(cc3-c3c(-c5ccccc5)ccc5c3oc3ccccc35)oc3ccccc34)nc(-n3c4ccccc4c4ccccc43)n2)cc1] 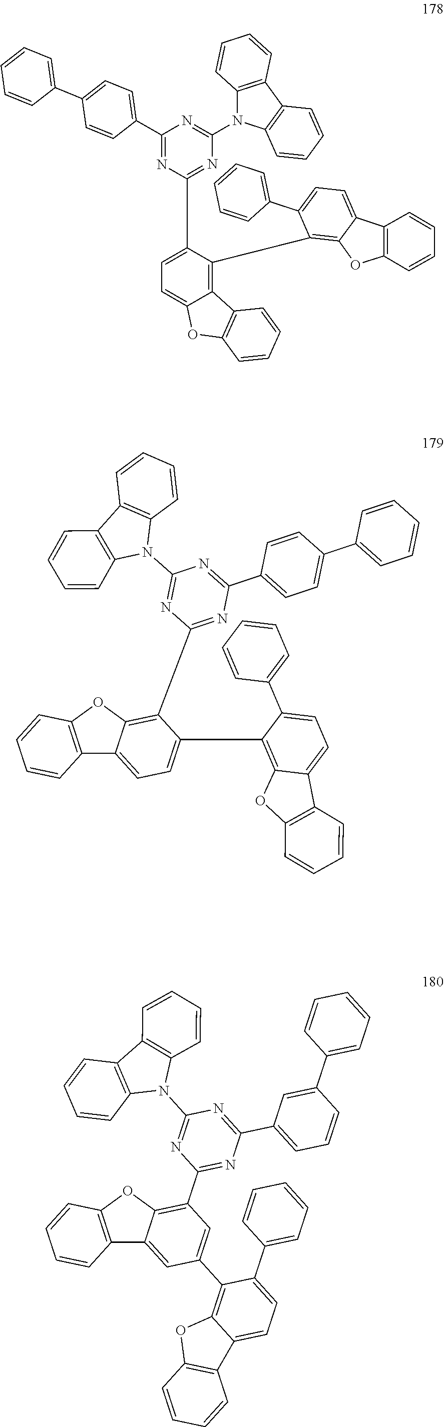 [SMILES: c1ccc(-c2ccc(-c3nc(-c4ccc5oc6ccccc6c5c4-c4c(-c5ccccc5)ccc5c4oc4ccccc45)nc(-n4c5ccccc5c5ccccc54)n3)cc2)cc1 ; c1ccc(-c2ccc(-c3nc(-c4c(-c5c(-c6ccccc6)ccc6c5oc5ccccc56)ccc5c4oc4ccccc45)nc(-n4c5ccccc5c5ccccc54)n3)cc2)cc1 ; c1ccc(-c2cccc(-c3nc(-c4cc(-c5c(-c6ccccc6)ccc6c5oc5ccccc56)cc5c4oc4ccccc45)nc(-n4c5ccccc5c5ccccc54)n3)c2)cc1] 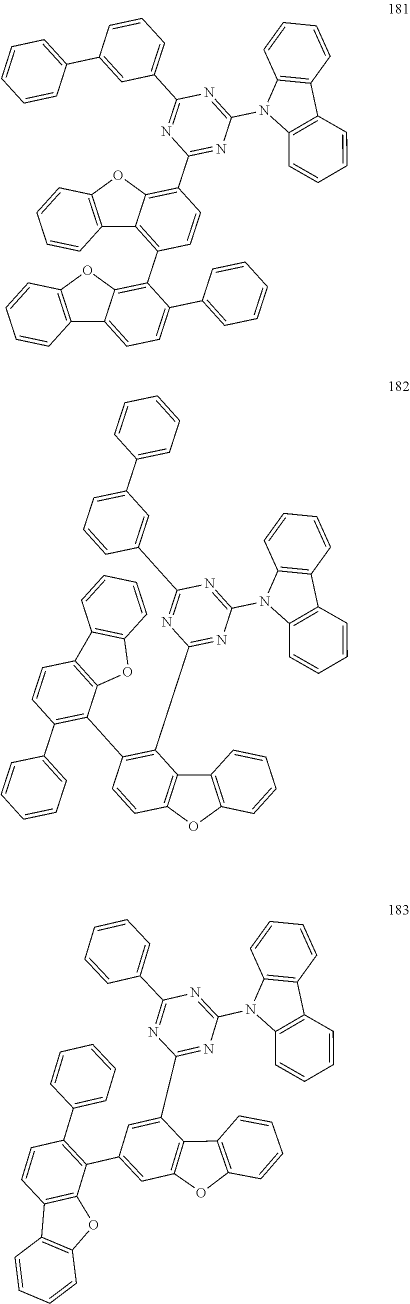 [SMILES: c1ccc(-c2cccc(-c3nc(-c4ccc(-c5c(-c6ccccc6)ccc6c5oc5ccccc56)c5c4oc4ccccc45)nc(-n4c5ccccc5c5ccccc54)n3)c2)cc1 ; c1ccc(-c2cccc(-c3nc(-c4c(-c5c(-c6ccccc6)ccc6c5oc5ccccc56)ccc5oc6ccccc6c45)nc(-n4c5ccccc5c5ccccc54)n3)c2)cc1 ; c1ccc(-c2nc(-c3cc(-c4c(-c5ccccc5)ccc5c4oc4ccccc45)cc4oc5ccccc5c34)nc(-n3c4ccccc4c4ccccc43)n2)cc1] 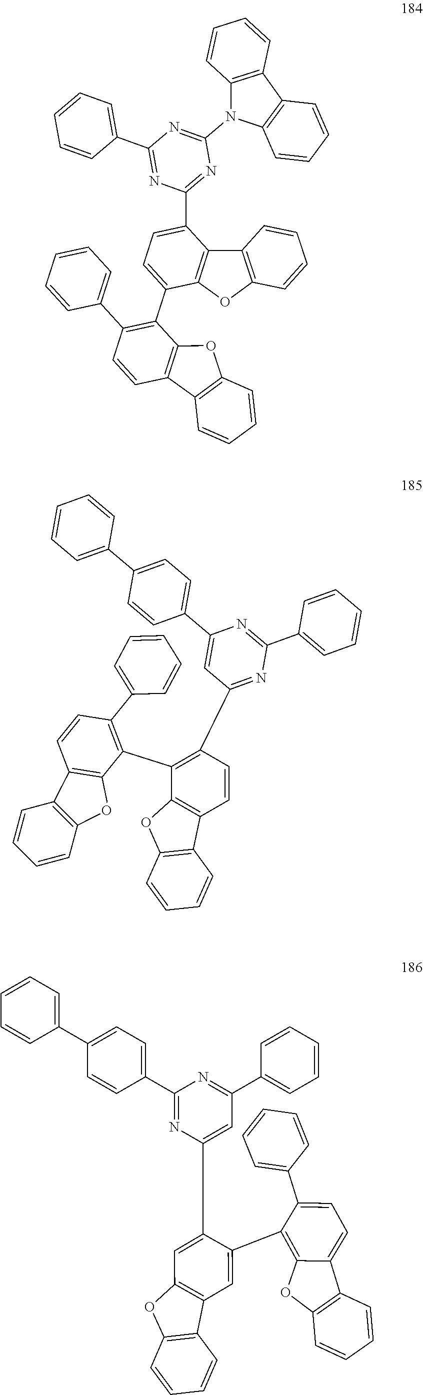 [SMILES: c1ccc(-c2nc(-c3ccc(-c4c(-c5ccccc5)ccc5c4oc4ccccc45)c4oc5ccccc5c34)nc(-n3c4ccccc4c4ccccc43)n2)cc1 ; c1ccc(-c2ccc(-c3cc(-c4ccc5c(oc6ccccc65)c4-c4c(-c5ccccc5)ccc5c4oc4ccccc45)nc(-c4ccccc4)n3)cc2)cc1 ; c1ccc(-c2ccc(-c3nc(-c4ccccc4)cc(-c4cc5oc6ccccc6c5cc4-c4c(-c5ccccc5)ccc5c4oc4ccccc45)n3)cc2)cc1] 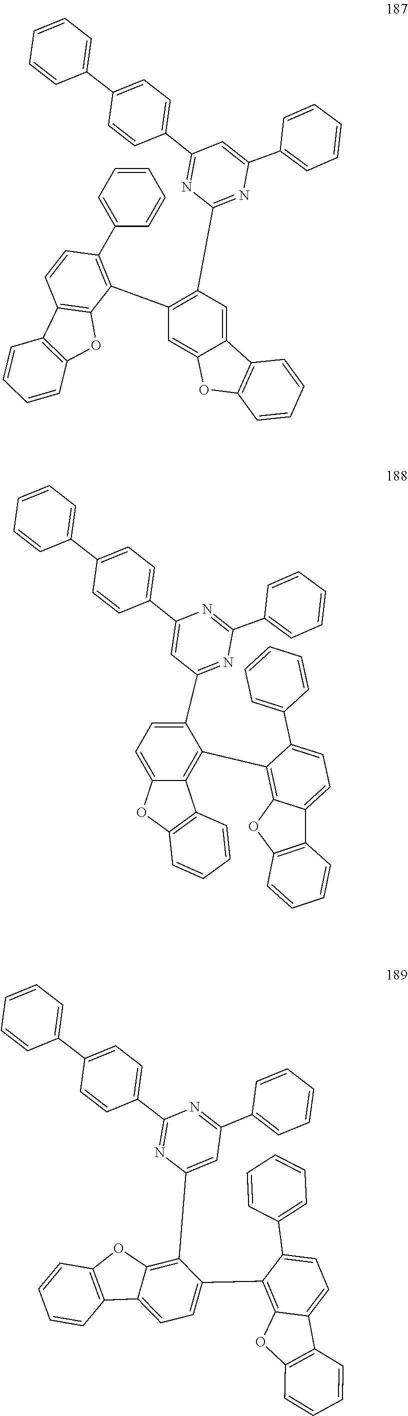 [SMILES: c1ccc(-c2ccc(-c3cc(-c4ccccc4)nc(-c4cc5c(cc4-c4c(-c6ccccc6)ccc6c4oc4ccccc46)oc4ccccc45)n3)cc2)cc1 ; c1ccc(-c2ccc(-c3cc(-c4ccc5oc6ccccc6c5c4-c4c(-c5ccccc5)ccc5c4oc4ccccc45)nc(-c4ccccc4)n3)cc2)cc1 ; c1ccc(-c2ccc(-c3nc(-c4ccccc4)cc(-c4c(-c5c(-c6ccccc6)ccc6c5oc5ccccc56)ccc5c4oc4ccccc45)n3)cc2)cc1] 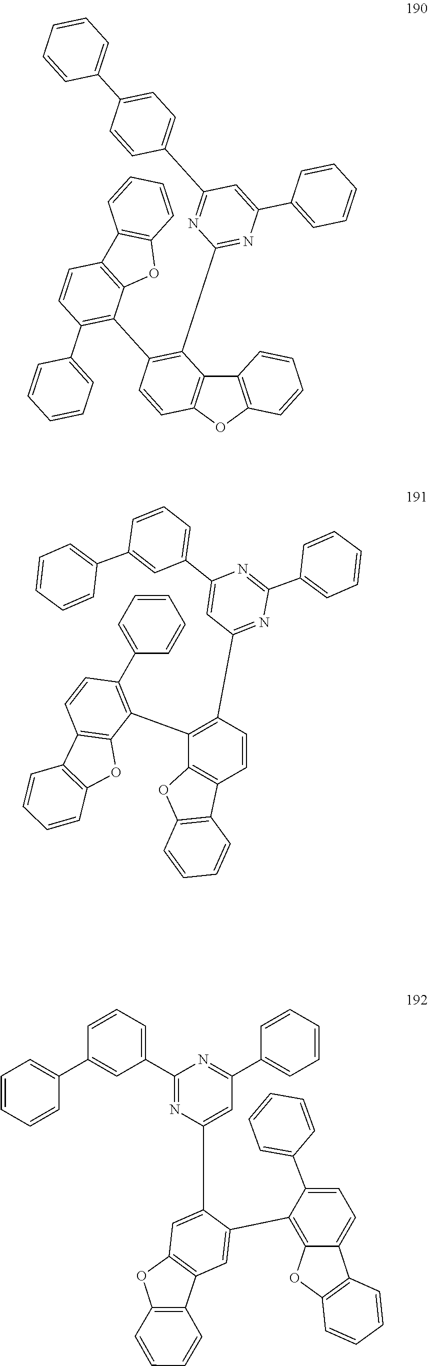 [SMILES: c1ccc(-c2ccc(-c3cc(-c4ccccc4)nc(-c4c(-c5c(-c6ccccc6)ccc6c5oc5ccccc56)ccc5oc6ccccc6c45)n3)cc2)cc1 ; c1ccc(-c2cccc(-c3cc(-c4ccc5c(oc6ccccc65)c4-c4c(-c5ccccc5)ccc5c4oc4ccccc45)nc(-c4ccccc4)n3)c2)cc1 ; c1ccc(-c2cccc(-c3nc(-c4ccccc4)cc(-c4cc5oc6ccccc6c5cc4-c4c(-c5ccccc5)ccc5c4oc4ccccc45)n3)c2)cc1] 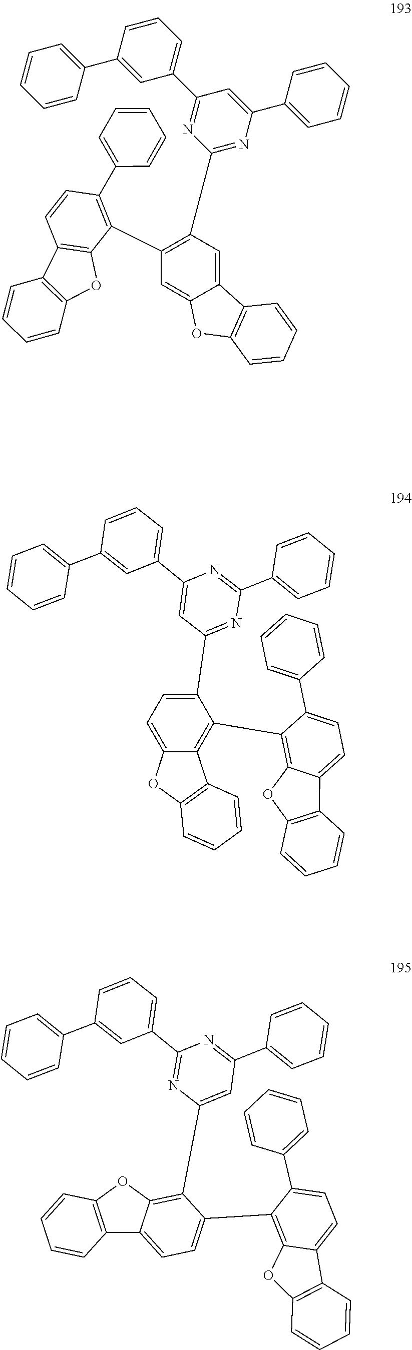 [SMILES: c1ccc(-c2cccc(-c3cc(-c4ccccc4)nc(-c4cc5c(cc4-c4c(-c6ccccc6)ccc6c4oc4ccccc46)oc4ccccc45)n3)c2)cc1 ; c1ccc(-c2cccc(-c3cc(-c4ccc5oc6ccccc6c5c4-c4c(-c5ccccc5)ccc5c4oc4ccccc45)nc(-c4ccccc4)n3)c2)cc1 ; c1ccc(-c2cccc(-c3nc(-c4ccccc4)cc(-c4c(-c5c(-c6ccccc6)ccc6c5oc5ccccc56)ccc5c4oc4ccccc45)n3)c2)cc1] 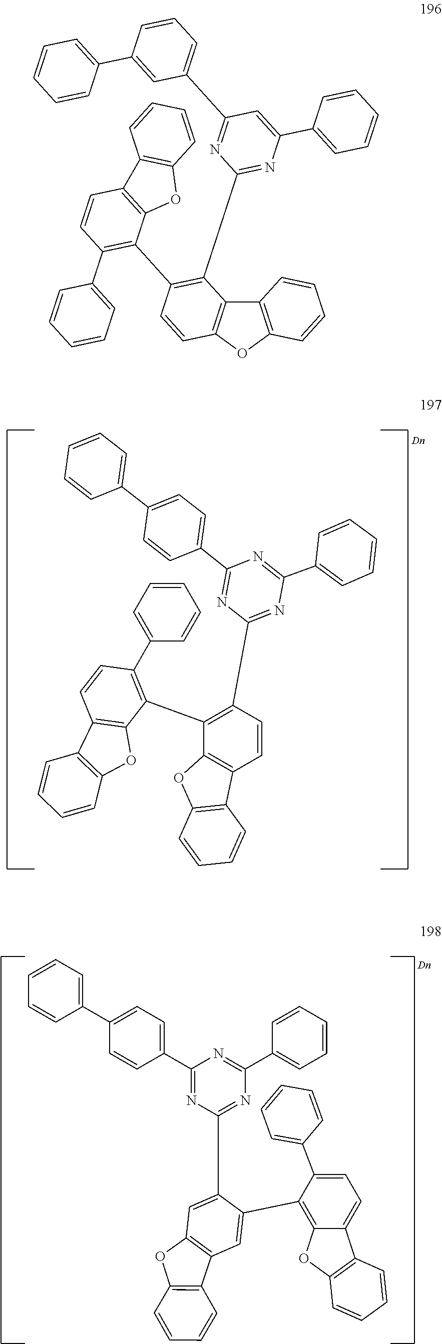 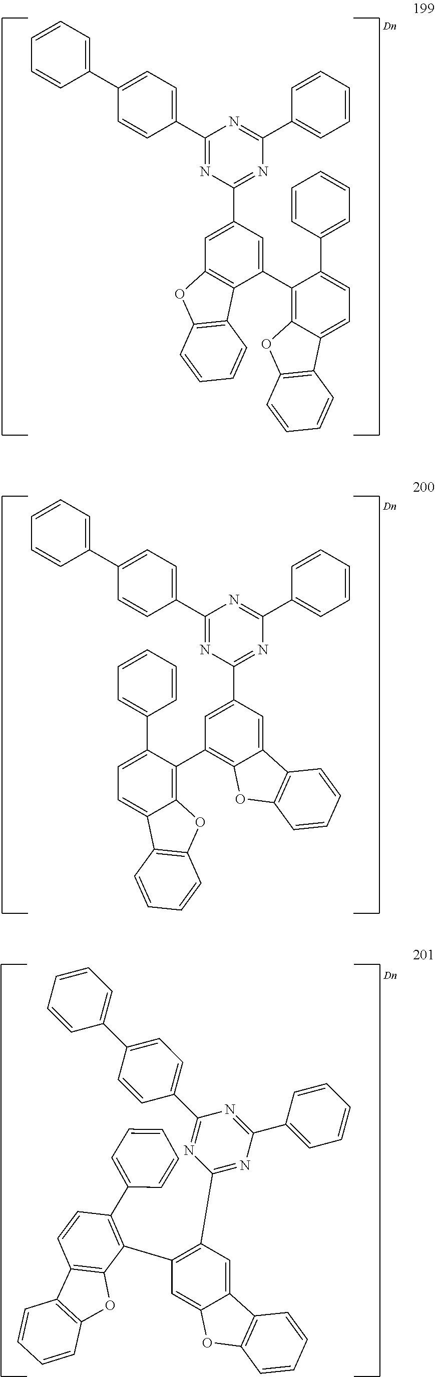 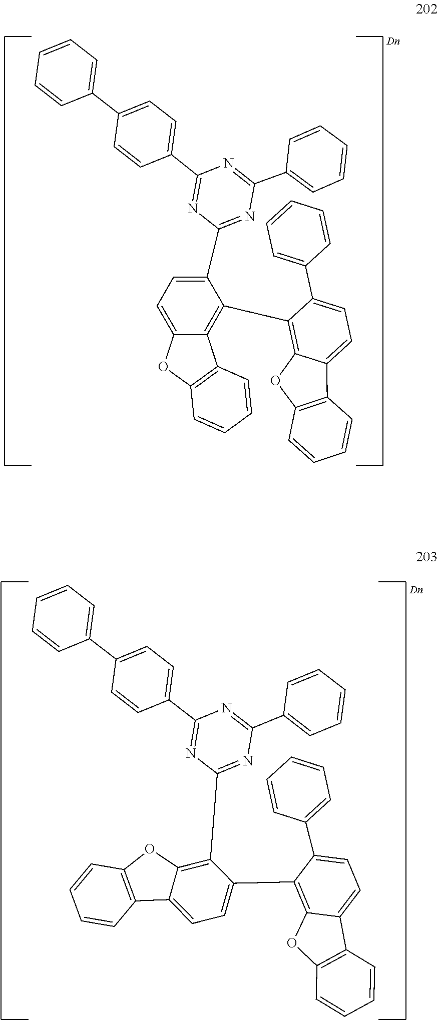 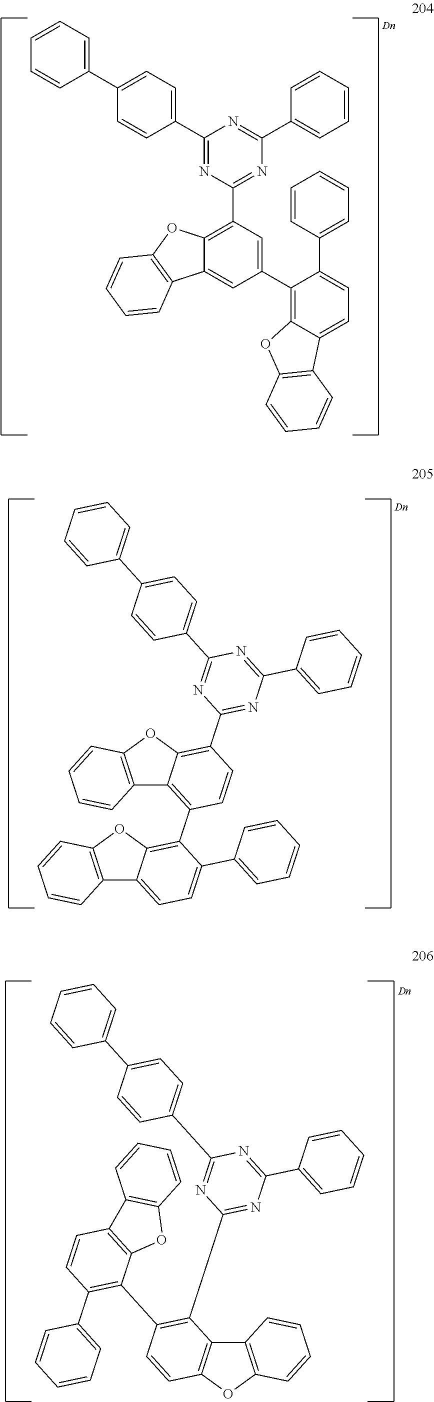 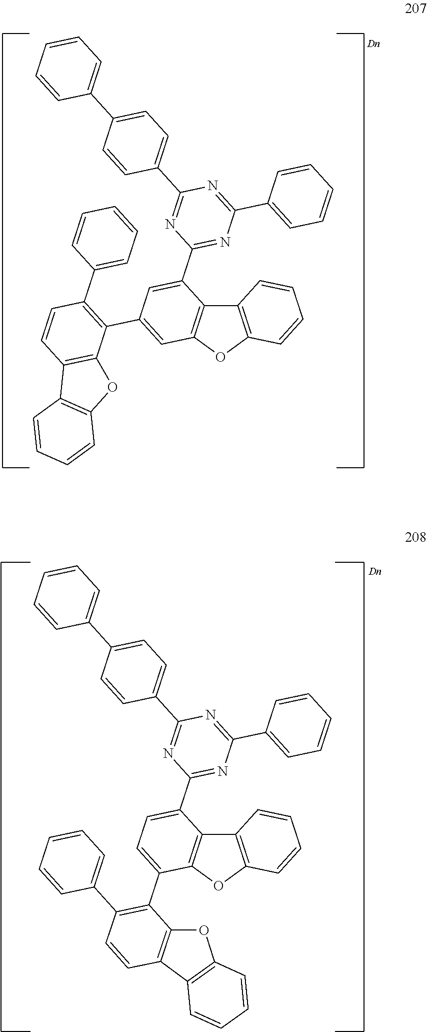
Dn refers to the number of deuterium atoms substituted, and n is an integer greater than or equal to 1.

9. A composition for an organic optoelectronic device, the composition comprising:
a first compound and a second compound,
wherein the first compound is the compound for an organic optoelectronic device as claimed in claim 1, and
the second compound is represented by Chemical Formula 2; a combination of Chemical Formula 3 and Chemical Formula 4; or Chemical Formula 5: 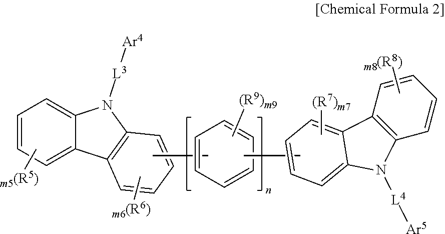
wherein, in Chemical Formula 2,
R⁵ to R⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁴ and Ar⁵ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
m5, m8, and m9 are each independently an integer of 1 to 4,
m6 and m7 are each independently an integer 1 to 3, and
n is an integer of 0 to 2; 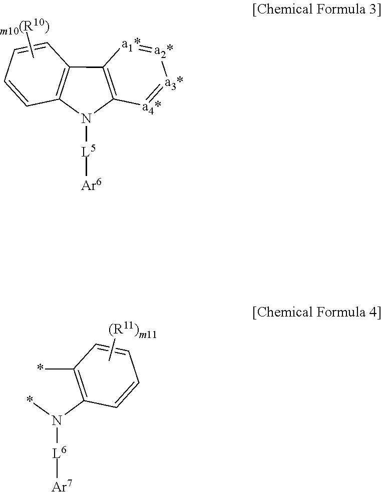
wherein, in Chemical Formula 3 and Chemical Formula 4,
two adjacent ones of a₁* to a₄* of Chemical Formula 3, are linking carbons linked at * of Chemical Formula 4,
the remaining two of a₁* to a₄* of Chemical Formula 3 that are not linked at * of Chemical Formula 4 are C-L^{a}-R^{d},
L^{a}, L⁵, and L⁶ are each independently a single bond or a substituted or unsubstituted C6 to C20 arylene group,
R^{d}, R¹⁰, and R¹¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁶ and Ar⁷ are each independently a substituted or unsubstituted C6 to C20 aryl group or a substituted or unsubstituted C2 to C30 heterocyclic group, and
m10 and m11 are each independently an integer 1 to 4; 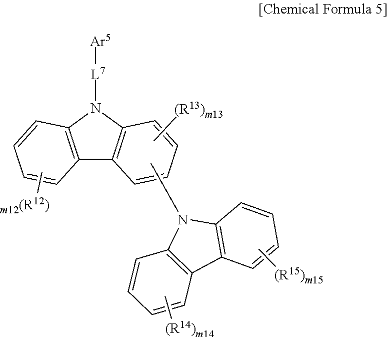
wherein, in Chemical Formula 5,
L⁷ is a single bond or a substituted or unsubstituted C6 to C20 arylene group,
R¹² to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁸ is a substituted or unsubstituted C6 to C20 aryl group or a substituted or unsubstituted C2 to C30 heterocyclic group,
m12, m14, and m15 are each independently an integer of 1 to 4, and
m13 is an integer of 1 to 3.

10. The composition for an organic optoelectronic device as claimed in claim 9, wherein:
the second compound is represented by a combination of Chemical Formula 3 and Chemical Formula 4, and
the combination of Chemical Formula 3 and Chemical Formula 4 is represented by Chemical Formula 3C: 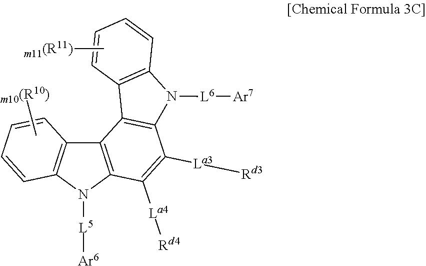
in Chemical Formula 3C,
L^{a3} and L^{a4} are a single bond,
R¹⁰, R¹¹, R^{d3}, and R^{d4} are each independently hydrogen, deuterium, or a substituted or unsubstituted C6 to C12 aryl group,
m10 and m11 are each independently an integer of 1 to 4, and
moieties *-L⁵-Ar⁶ and *-L⁶-Ar⁷ are each independently a moiety of Group I, 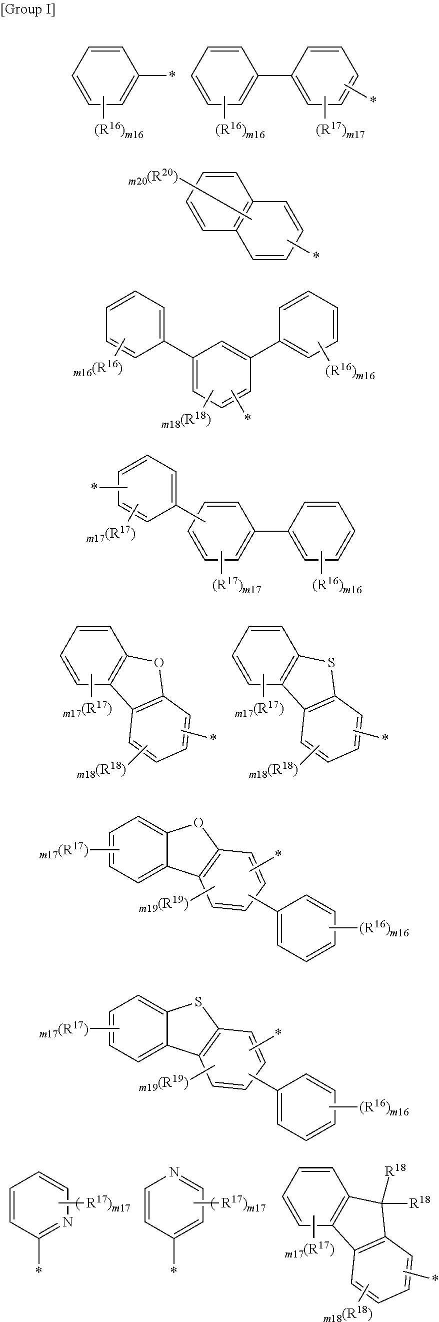 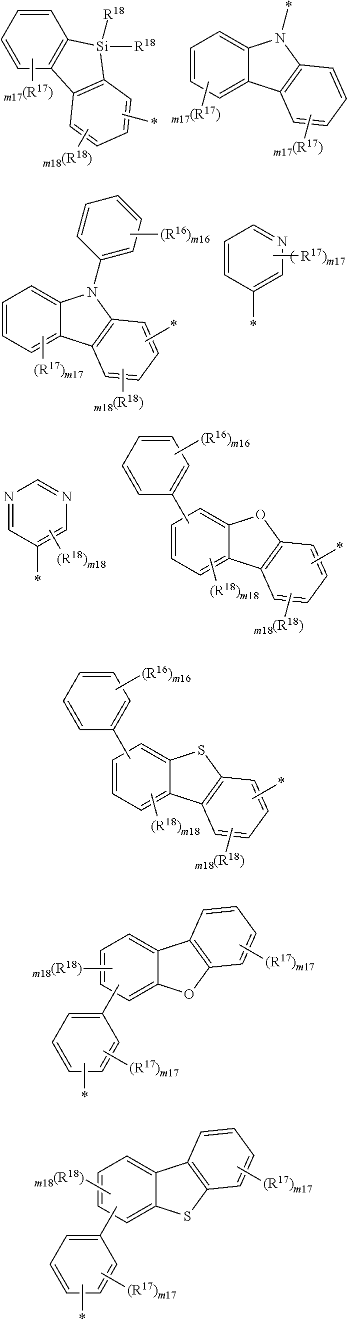 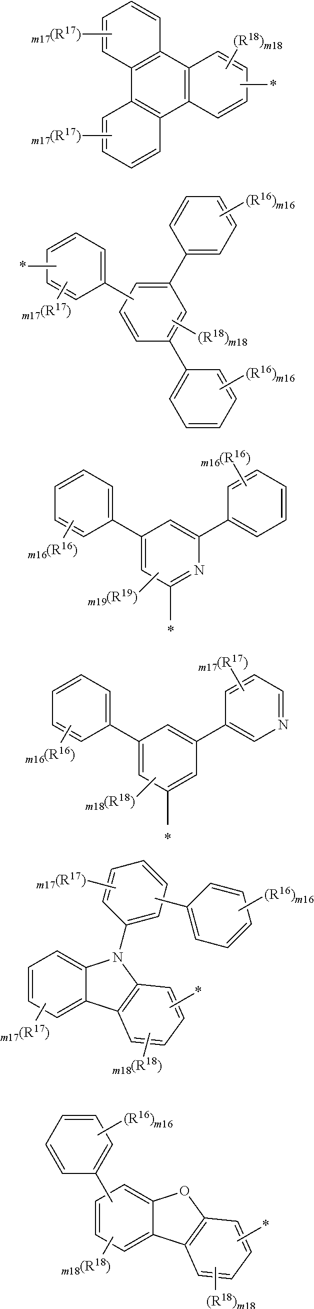 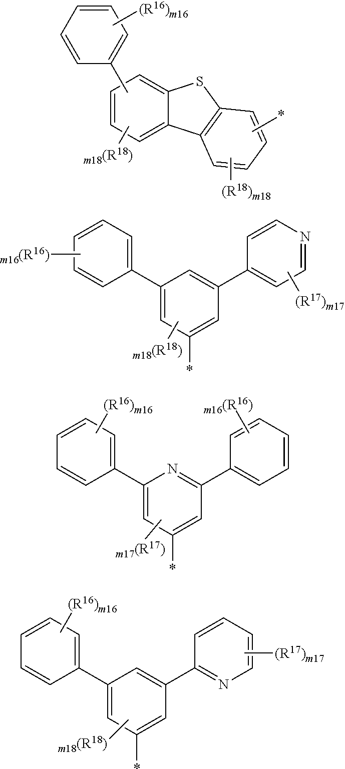
in Group I,
R¹⁶ to R²⁰ are each independently hydrogen, deuterium, a cyano group, a substituted or unsubstituted C1 to C10 alkyl group, or a substituted or unsubstituted C6 to C12 aryl group,
m16 is an integer of 1 to 5,
m17 is an integer of 1 to 4,
m18 is an integer of 1 to 3,
m19 is an integer of 1 or 2,
m20 is an integer of 1 to 7, and
* is a linking point.

11. An organic optoelectronic device, comprising:
an anode and a cathode facing each other, and
at least one organic layer between the anode and the cathode,
wherein the at least one organic layer includes the compound for the organic optoelectronic device as claimed in claim 1.

12. The organic optoelectronic device as claimed in claim 11, wherein:
the at least one organic layer includes a light emitting layer, and
the light emitting layer includes the compound for an organic optoelectronic device.

13. A display device comprising the organic optoelectronic device as claimed in claim 11.

14. An organic optoelectronic device, comprising:
an anode and a cathode facing each other, and
at least one organic layer between the anode and the cathode,
wherein the at least one organic layer includes the composition for the organic optoelectronic device as claimed in claim 9.

15. The organic optoelectronic device as claimed in claim 14, wherein:
the at least one organic layer includes a light emitting layer, and
the light emitting layer includes the composition for an organic optoelectronic device.

16. A display device comprising the organic optoelectronic device as claimed in claim 14.
